(19) United States
(12) Patent Application Publication
(10) Pub. No.: US 2024/0397814 A1
(43) Pub. Date: Nov. 28, 2024

(54) NOVEL COMPOUND AND ORGANIC LIGHT-EMITTING DEVICE COMPRISING THE SAME

(71) Applicant: LG CHEM, LTD., Seoul (KR)

(72) Inventors: Min Woo JUNG, Daejeon (KR); Dong Hoon LEE, Daejeon (KR); Miyeon HAN, Daejeon (KR); Seulchan PARK, Daejeon (KR); Hoon Jun KIM, Daejeon (KR); Hye Min CHO, Daejeon (KR); Hojung LEE, Daejeon (KR)

(21) Appl. No.: 18/833,787

(22) PCT Filed: Apr. 14, 2023
(86) PCT No.: PCT/KR2023/005050

§ 371 Date: Jul. 26, 2024

(30) Foreign Application Priority Data
Apr. 15, 2022 (KR) 10-2022-0046837
Apr. 13, 2023 (KR) 10-2023-0048984

(51) Int. Cl. H10K 85/60; C07D 405/14; C07D 409/14; C07D 491/048; C09K 11/06; H10K 50/11

(52) U.S. Cl. CPC H10K 85/654; C07D 405/14; C07D 409/14; C07D 491/048; C09K 11/06; H10K 85/615; H10K 85/657; H10K 85/6572; H10K 85/6574; H10K 85/6576; C09K 2211/1018; H10K 50/11

(57) ABSTRACT

A novel compound of Chemical Formula 1:

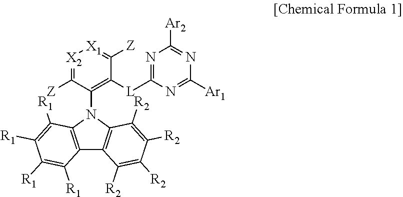

wherein: Ar₁ and Ar₂ are each independently a substituted or unsubstituted C₆₋₆₀ aryl or a substituted or unsubstituted C₂₋₆₀ heteroaryl containing one or more heteroatoms selected from the group consisting of N, O and S; each Z is independently hydrogen or deuterium; one of X₁ and X₂ is carbon (C) connected to a substituent of the following Chemical Formula 2, and the other is CH or CD:

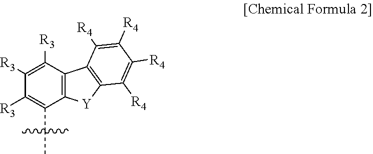

Y is O or S; and the other substituents are as defined in the specification; and an organic light emitting device including the same. When the compound of Chemical Formula 1 is used as a material in an organic material layer of an organic light-emitting device, the device exhibits excellent characteristics in terms of efficiency and lifetime.

### CROSS-REFERENCE TO RELATED APPLICATION(S)

This application is a National Stage Application of International Application No. PCT/KR2023/005050 filed on Apr. 14, 2023, which claims the benefit of Korean Patent Application No. 10-2022-0046837 filed on Apr. 15, 2022 and Korean Patent Application No. 10-2023-0048984 filed on Apr. 13, 2023 in the Korean Intellectual Property Office, the entire contents of which are incorporated herein by reference.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

Meanwhile, recently, in order to reduce process costs, an organic light emitting device using a solution process, particularly an inkjet process, has been developed instead of a conventional deposition process. In the initial stage of development, attempts have been made to develop organic light emitting devices by coating all organic light emitting device layers by a solution process, but current technology has limitations. Therefore, only HIL, HTL, and EML are processed in a layer device structure by a solution process, and a hybrid process utilizing traditional deposition processes is being studied as a subsequent process.

Accordingly, the present disclosure provides a novel material for organic light emitting devices capable of being deposited by a solution process while being used for an organic light emitting device.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of the following Chemical Formula 1:

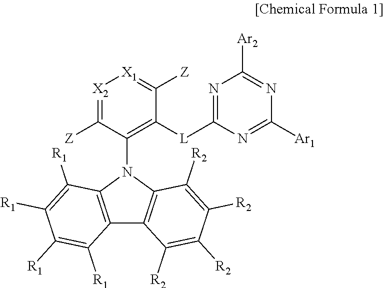

wherein, in Chemical Formula 1:
each R₁ is independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S, or two adjacent R₁s are connected with each other to form a substituted or unsubstituted C₆₋₆₀ aromatic ring, or a substituted or unsubstituted C₂₋₆₀ heteroaromatic ring containing one or more heteroatoms selected from the group consisting of N, O and S;
each R₂ is independently hydrogen or deuterium, provided that at least one of the R₂s is deuterium;
one of X₁ and X₂ is carbon (C) connected to a substituent represented by the following Chemical Formula 2, and the other is CH or CD;
Ar₁ is a substituted or unsubstituted C₆₋₆₀ aryl or a substituted or unsubstituted C₂₋₆₀ heteroaryl containing one or more heteroatoms selected from the group consisting of N, O and S;
Ar₂ is a substituted or unsubstituted C₆₋₆₀ aryl or a substituted or unsubstituted C₂₋₆₀ heteroaryl containing one or more heteroatoms selected from the group consisting of N, O and S;
each Z is independently hydrogen or deuterium;
L is a direct bond or a substituted or unsubstituted C₆₋₆₀ arylene;

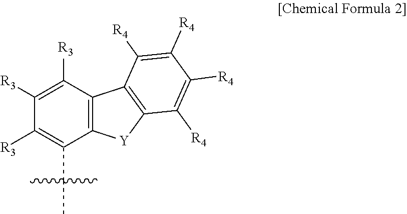

wherein, in Chemical Formula 2:
Y is O or S;

each R₃ is independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S; and
each R₄ is independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S.

According to another aspect of the present disclosure, provided is an organic light emitting device comprising: a first electrode; a second electrode that is provided opposite to the first electrode; and an organic material layer that is provided between the first electrode and the second electrode, wherein the organic material layer comprises the compound of Chemical Formula 1. Specifically, the organic material layer comprising the compound can be a light emitting layer.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device. In particular, the compound of Chemical Formula 1 can be used as hole injection, hole transport, hole injection and transport, electron blocking, light emission, electron transport, or electron injection material.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 7, an electron injection and transport layer 8, and a cathode 4.

### DETAILED DESCRIPTION

Hereinafter, embodiments of the present disclosure will be described in more detail to facilitate understanding of the invention.

### DEFINITION OF TERMS

As used herein, the notation

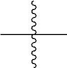

and [symbol] mean a bond connected to another substituent group, and “D” means deuterium.

As used herein, the term “substituted or unsubstituted” means being unsubstituted or substituted with one or more substituents selected from the group consisting of deuterium, a halogen group, a cyano group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, or a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenylyl group can be an aryl group, or it can also be interpreted as a substituent in which two phenyl groups are connected.

In the present disclosure, the carbon number of a carbonyl group is not particularly limited, but is preferably 1 to 40. Specifically, it can be a compound group having the following structure, but is not limited thereto:

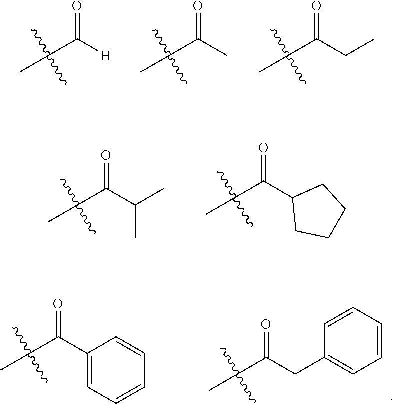

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, it can be a group having the following structure, but is not limited thereto:

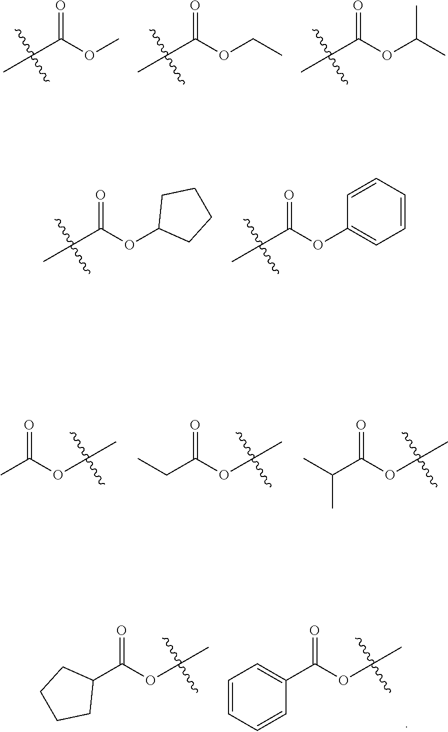

In the present disclosure, the carbon number of an imide group is not particularly limited, but is preferably 1 to 25. Specifically, it can be a group having the following structure, but is not limited thereto:

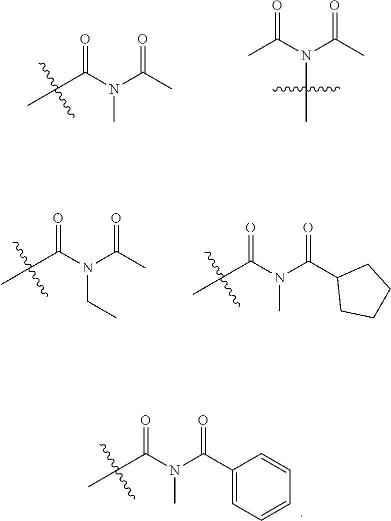

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to yet another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohectylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, a fluorenyl group, and the like, but is not limited thereto.

In the present disclosure, the fluorenyl group can be substituted, and two substituent groups can be linked with each other to form a spiro structure. In the case where the fluorenyl group is substituted,

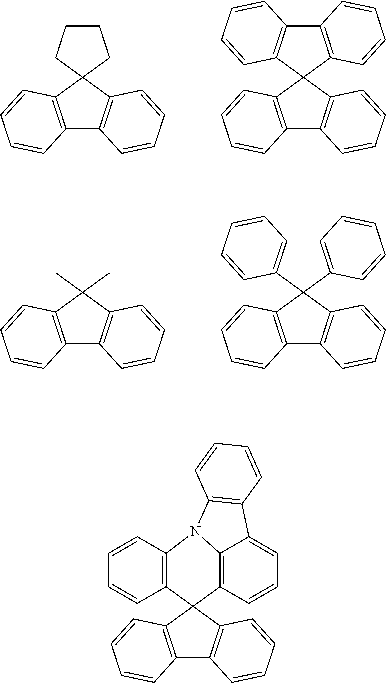

and the like can be formed. However, the structure is not limited thereto.

In the present disclosure, a heteroaryl is a heteroaryl containing one or more of O, N, Si and S as a heteroatom, and the carbon number thereof is not particularly limited, but is preferably 2 to 60. Examples of the heteroaryl group include xanthene, thioxanthene, a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsilyl group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heteroaryl. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl can be applied, except that the heterocycle is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means “unsubstituted or mono to the maximum number of substitutable hydrogens have been substituted with deuterium.” In one example, the term “phenanthryl unsubstituted or substituted with deuterium” can be understood as meaning “phenanthryl unsubstituted or substituted with 1 to 9 deuterium atoms”, considering that the maximum number of hydrogens that can be substituted with deuterium in the phenanthryl structure is 9.

Further, the term “deuterated structure” is meant to encompass a compound, a divalent linking group or a monovalent substituent of all structures in which at least one hydrogen is replaced with deuterium. In one example, a deuterated structure of phenyl can be understood to refer to monovalent substituents of all structures in which at least one substitutable hydrogen in a phenyl group is replaced with a deuterium as follows.

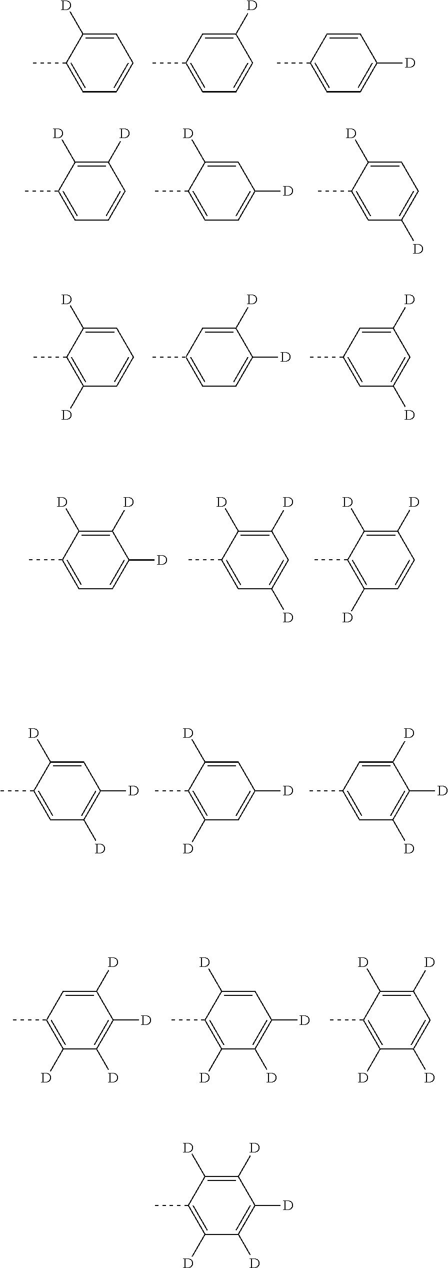

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can be present in the compound is calculated as a percentage. Therefore, “deuterium substitution rate” or “degree of deuteration” of a compound being “K %” means that K % of the hydrogen atoms substitutable with deuterium in the compound are substituted with deuterium.

At this time, the “deuterium substitution rate” or “degree of deuteration” can be measured according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GC/MS (Gas Chromatography/Mass Spectrometry), or the like. More specifically, when using MALDI-TOF MS, the “deuterium substitution rate” or “degree of deuteration” can be obtained by determining the number of substituted deuterium atoms in the compound through MALDI-TOF MS analysis, and then calculating the ratio of the number of substituted deuterium atoms to the total number of hydrogen atoms that can be present in the compound as a percentage.

### (Compound)

According to one embodiment of the present disclosure, provided is the compound of Chemical Formula 1.

In Chemical Formula 1, each R₁ is independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S, or two adjacent R₁s are connected with each other to form a substituted or unsubstituted C₆₋₆₀ aromatic ring, or a substituted or unsubstituted C₂₋₆₀ heteroaromatic ring containing one or more heteroatoms selected from the group consisting of N, O and S.

For example, each R₁ can be independently hydrogen, deuterium, or a C₆₋₁₂ aryl that is unsubstituted or substituted with at least one deuterium.

More specifically, for example, each R₁ can be independently hydrogen, deuterium, or phenyl that is unsubstituted or substituted with deuterium.

Specifically, the Chemical Formula 1 can be a compound of any one of the following Chemical Formulas 3a to 3o:

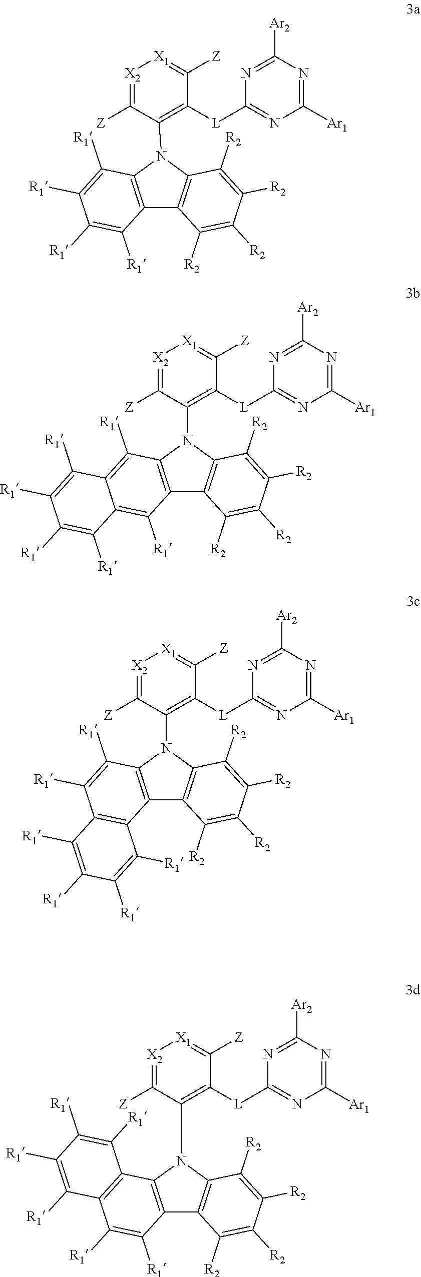

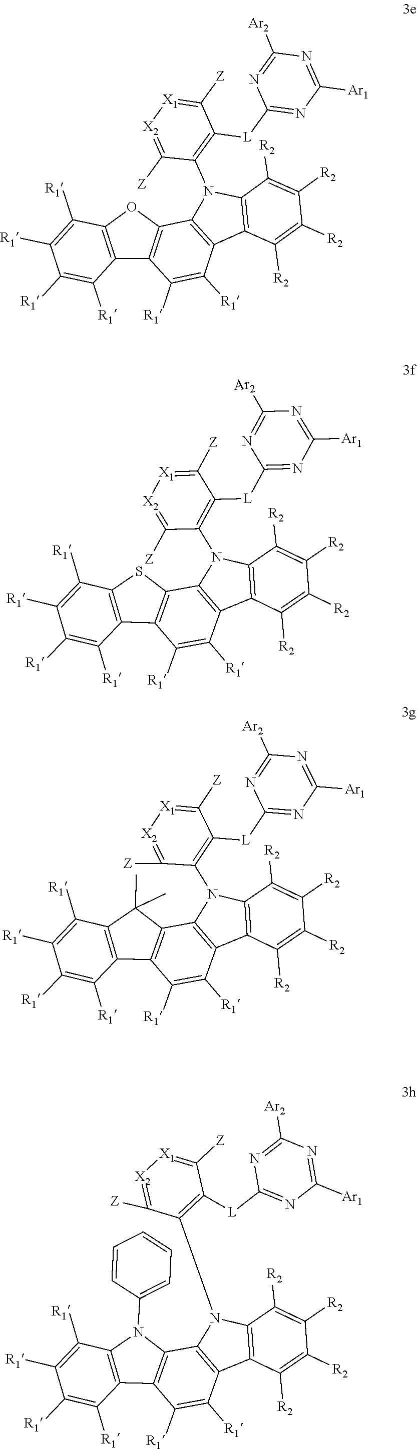

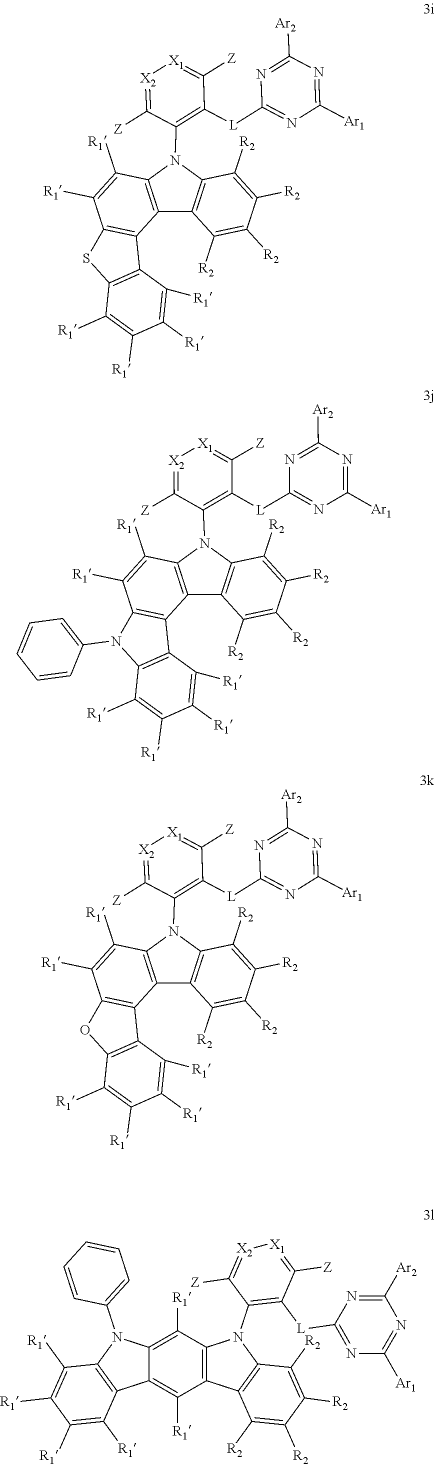

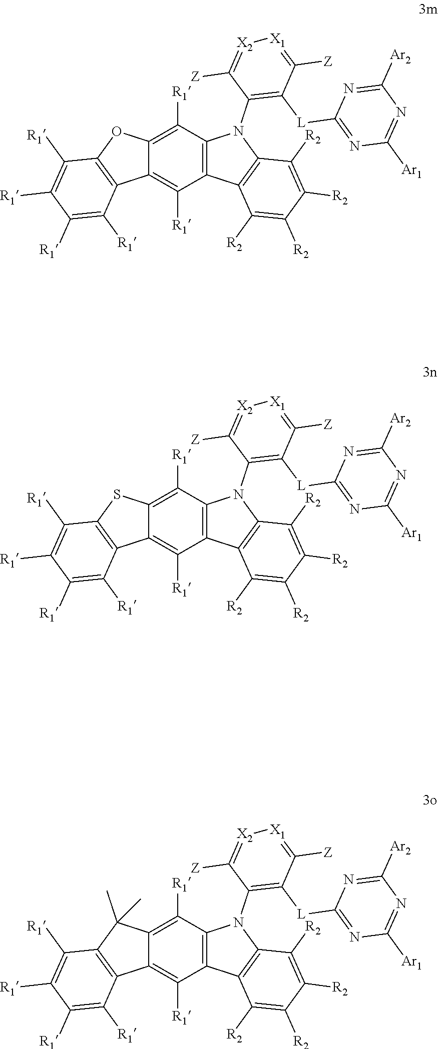

wherein,
Ar₁, Ar₂, Z, X₁, X₂, R₁′, R₂ and L are as defined in Chemical Formula 1.

In Chemical Formulas 3a to 3o, each R₁′ are independently hydrogen, deuterium, or phenyl, wherein the phenyl can be unsubstituted or substituted with at least one deuterium.

In Chemical Formula 1, each R₂ is independently hydrogen or deuterium, provided that at least one of R₂s is deuterium.

In Chemical Formula 1, Ar₁ can be a substituted or unsubstituted C₆₋₆₀ aryl or a substituted or unsubstituted C₂₋₆₀ heteroaryl containing one or more heteroatoms selected from the group consisting of N, O and S.

Specifically, Ar₁ is phenyl, biphenylyl, terphenylyl, naphthylphenyl, naphthyl, phenanthryl, dibenzofuranyl, dibenzothiophenyl, phenylcarbazolyl, or N-carbazolyl, wherein the Ar₁ can be unsubstituted or substituted with at least one deuterium.

In Chemical Formula 1, Ar₂ is a substituted or unsubstituted C₆₋₆₀ aryl or a substituted or unsubstituted C₂₋₆₀ heteroaryl containing one or more heteroatoms selected from the group consisting of N, O and S.

Specifically, Ar₂ is phenyl, biphenylyl, terphenylyl, naphthylphenyl, naphthyl, phenanthryl, dibenzofuranyl, dibenzothiophenyl, phenylcarbazolyl, or N-carbazolyl, wherein the Ar₂ can be unsubstituted or substituted with at least one deuterium.

In Chemical Formula 1, L can be a direct bond or a substituted or unsubstituted C₆₋₆₀ arylene. Specifically, L can be a direct bond.

In Chemical Formula 1, each Z is independently hydrogen or deuterium.

In Chemical Formula 1, one of X₁ and X₂ is carbon (C) connected to a substituent represented by the following Chemical Formula 2, and the other is CH or CD:

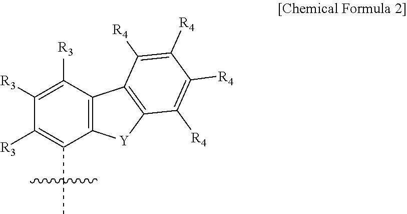

wherein, in Chemical Formula 2, Y can be O or S.

In Chemical Formula 2, each R₃ can be independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S.

Specifically, each R₃ can be independently hydrogen, deuterium, or phenyl that is unsubstituted or substituted with deuterium.

In Chemical Formula 2, each R₄ can be independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S. Specifically, each R₄ can be independently hydrogen, deuterium, or phenyl that is unsubstituted with deuterium.

Further, the substituent

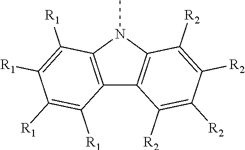

can be any one selected from the group consisting of the following:

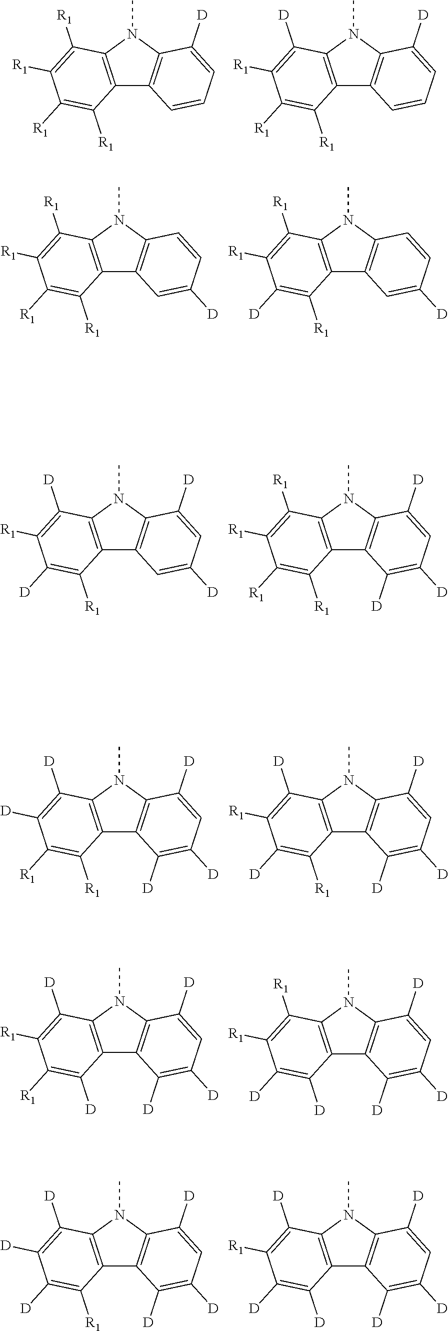

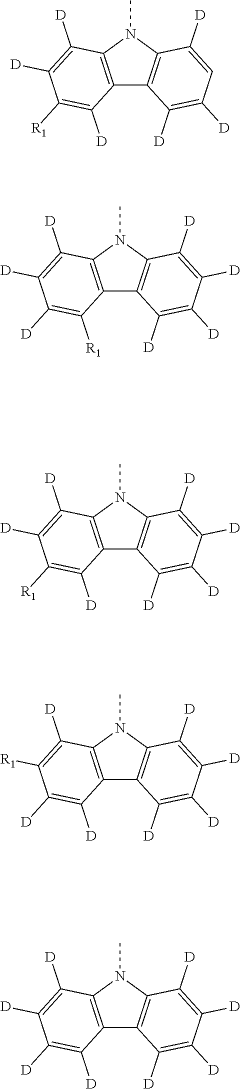

wherein,
R₁ is as defined in Chemical Formula 1.

For example, each R₁ can be independently hydrogen, deuterium, or phenyl that is unsubstituted or substituted with 1, 2, 3, 4, or 5 deuteriums.

Representative examples of the compound of Chemical Formula 1 are as follows:

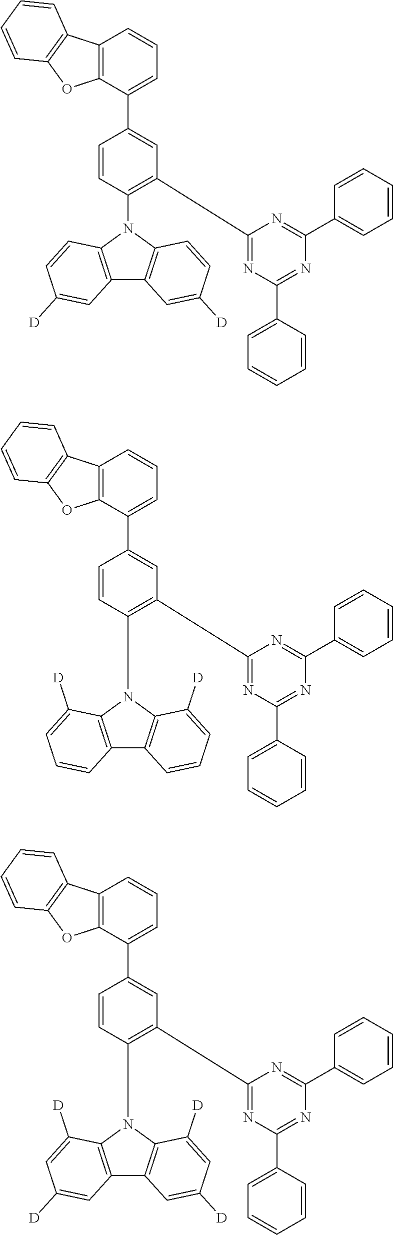 [SMILES: [2H]c1ccc2c(c1)c1cc([2H])ccc1n2-c1ccc(-c2cccc3c2oc2ccccc23)cc1-c1nc(-c2ccccc2)nc(-c2ccccc2)n1 ; [2H]c1cccc2c3cccc([2H])c3n(-c3ccc(-c4cccc5c4oc4ccccc45)cc3-c3nc(-c4ccccc4)nc(-c4ccccc4)n3)c12 ; [2H]c1cc([2H])c2c(c1)c1cc([2H])cc([2H])c1n2-c1ccc(-c2cccc3c2oc2ccccc23)cc1-c1nc(-c2ccccc2)nc(-c2ccccc2)n1]

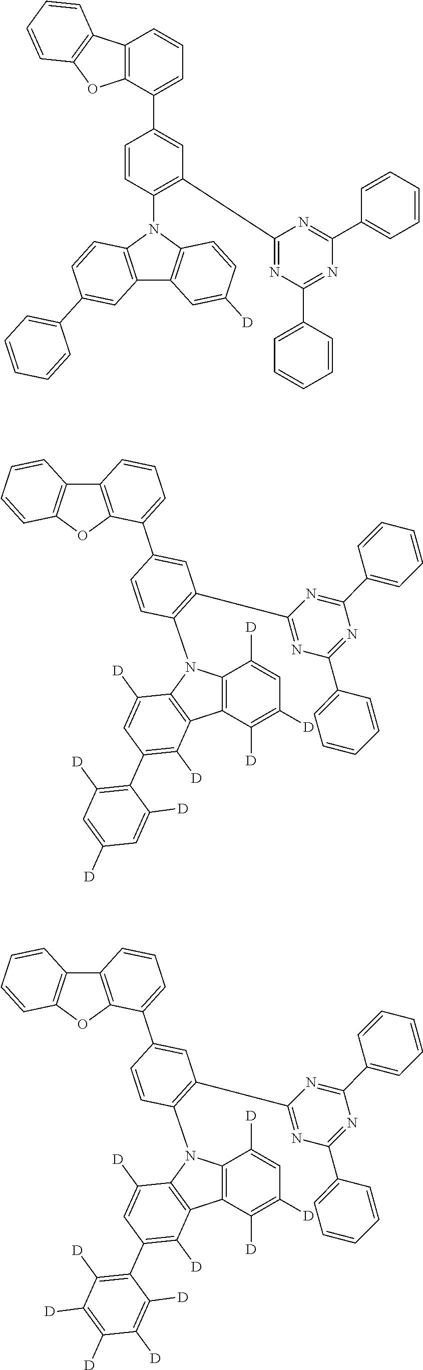 [SMILES: [2H]c1ccc2c(c1)c1cc(-c3ccccc3)ccc1n2-c1ccc(-c2cccc3c2oc2ccccc23)cc1-c1nc(-c2ccccc2)nc(-c2ccccc2)n1 ; [2H]c1cc([2H])c(-c2cc([2H])c3c(c2[2H])c2c([2H])c([2H])cc([2H])c2n3-c2ccc(-c3cccc4c3oc3ccccc34)cc2-c2nc(-c3ccccc3)nc(-c3ccccc3)n2)c([2H])c1 ; [2H]c1cc([2H])c2c(c1[2H])c1c([2H])c(-c3c([2H])c([2H])c([2H])c([2H])c3[2H])cc([2H])c1n2-c1ccc(-c2cccc3c2oc2ccccc23)cc1-c1nc(-c2ccccc2)nc(-c2ccccc2)n1]

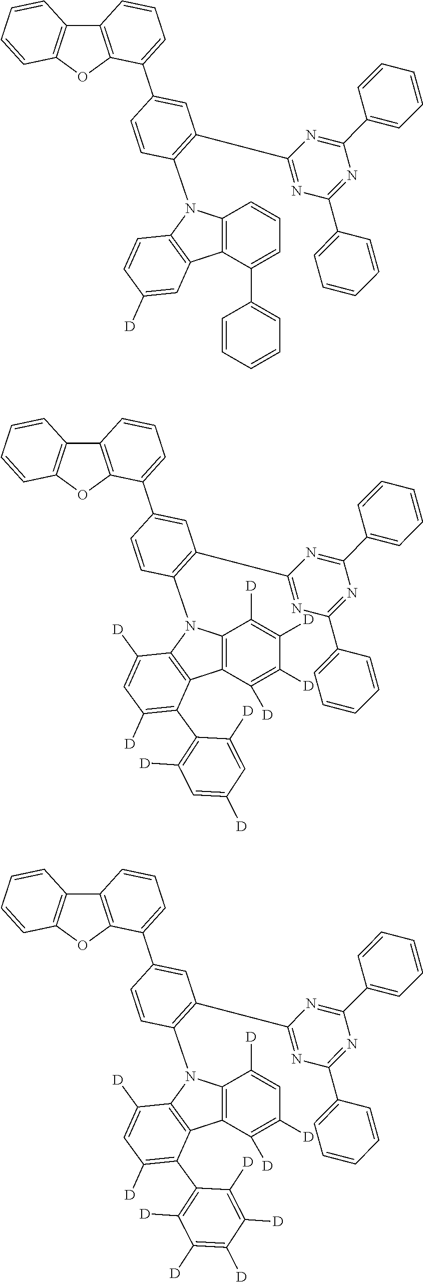 [SMILES: [2H]c1ccc2c(c1)c1c(-c3ccccc3)cccc1n2-c1ccc(-c2cccc3c2oc2ccccc23)cc1-c1nc(-c2ccccc2)nc(-c2ccccc2)n1 ; [2H]c1cc([2H])c(-c2c([2H])cc([2H])c3c2c2c([2H])c([2H])c([2H])c([2H])c2n3-c2ccc(-c3cccc4c3oc3ccccc34)cc2-c2nc(-c3ccccc3)nc(-c3ccccc3)n2)c([2H])c1 ; [2H]c1cc([2H])c2c(c1[2H])c1c(-c3c([2H])c([2H])c([2H])c([2H])c3[2H])c([2H])cc([2H])c1n2-c1ccc(-c2cccc3c2oc2ccccc23)cc1-c1nc(-c2ccccc2)nc(-c2ccccc2)n1]

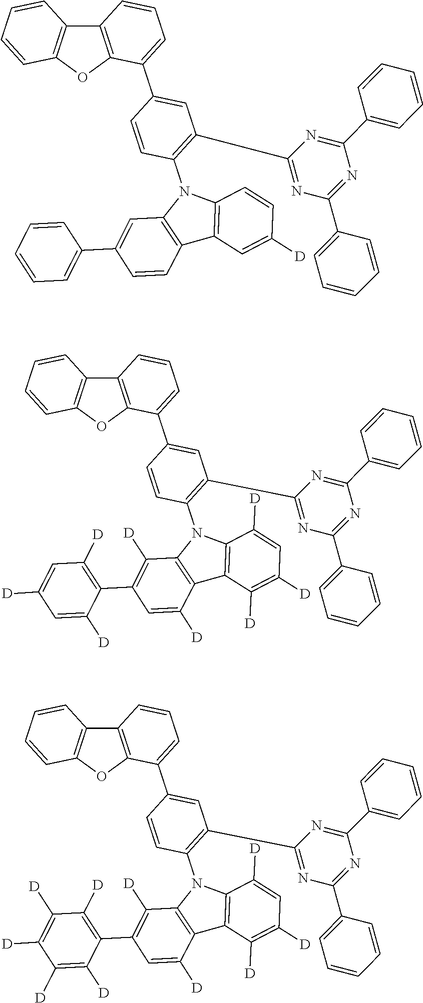 [SMILES: [2H]c1ccc2c(c1)c1ccc(-c3ccccc3)cc1n2-c1ccc(-c2cccc3c2oc2ccccc23)cc1-c1nc(-c2ccccc2)nc(-c2ccccc2)n1 ; [2H]c1cc([2H])c(-c2cc([2H])c3c4c([2H])c([2H])cc([2H])c4n(-c4ccc(-c5cccc6c5oc5ccccc56)cc4-c4nc(-c5ccccc5)nc(-c5ccccc5)n4)c3c2[2H])c([2H])c1 ; [2H]c1cc([2H])c2c(c1[2H])c1c([2H])cc(-c3c([2H])c([2H])c([2H])c([2H])c3[2H])c([2H])c1n2-c1ccc(-c2cccc3c2oc2ccccc23)cc1-c1nc(-c2ccccc2)nc(-c2ccccc2)n1]

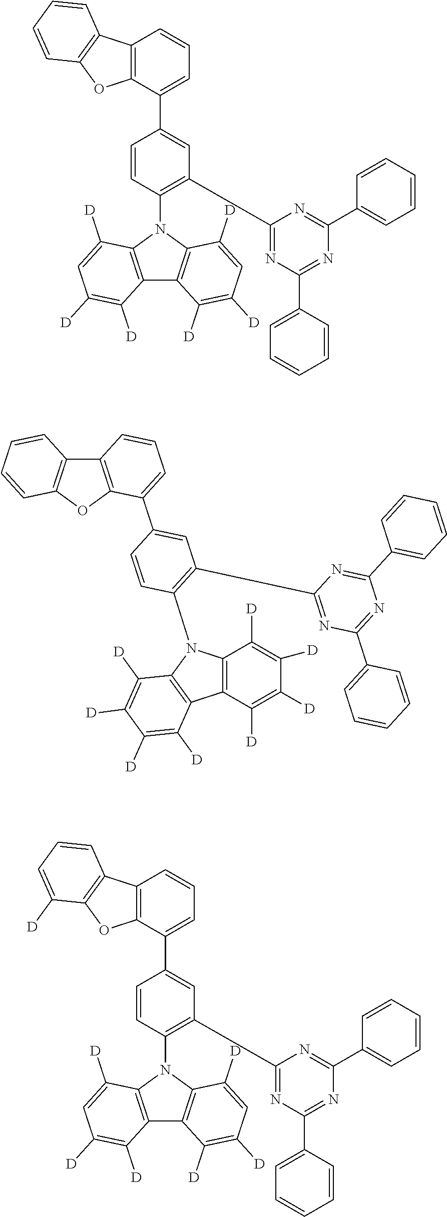 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2cccc3c2oc2ccccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2c([2H])cccc23)cc1-c1nc(-c2ccccc2)nc(-c2ccccc2)n1]

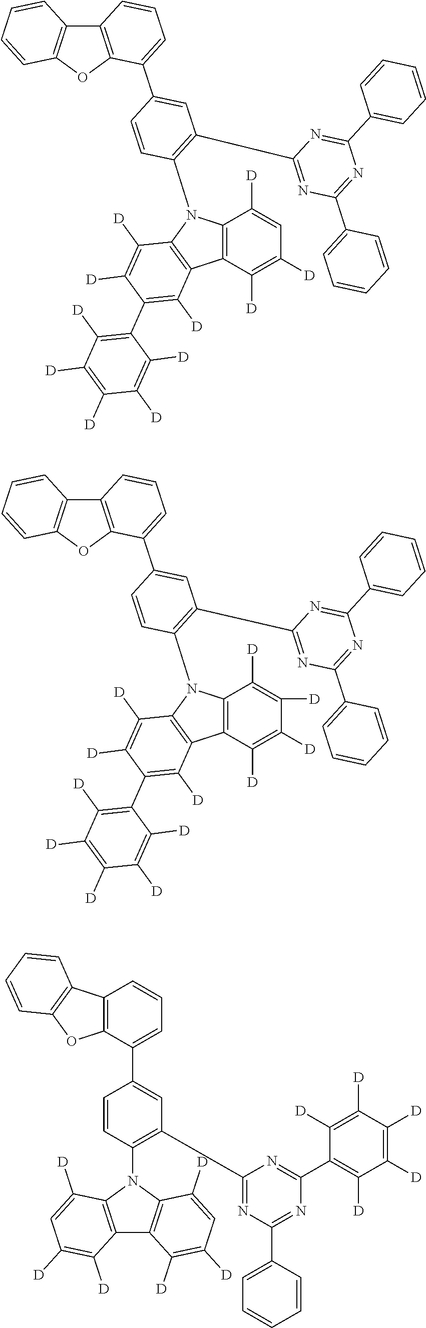 [SMILES: [2H]c1cc([2H])c2c(c1[2H])c1c([2H])c(-c3c([2H])c([2H])c([2H])c([2H])c3[2H])c([2H])c([2H])c1n2-c1ccc(-c2cccc3c2oc2ccccc23)cc1-c1nc(-c2ccccc2)nc(-c2ccccc2)n1 ; [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3ccccc3)n2)c([2H])c1[2H] ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2c([2H])c([2H])c([2H])c([2H])c2[2H])n1]

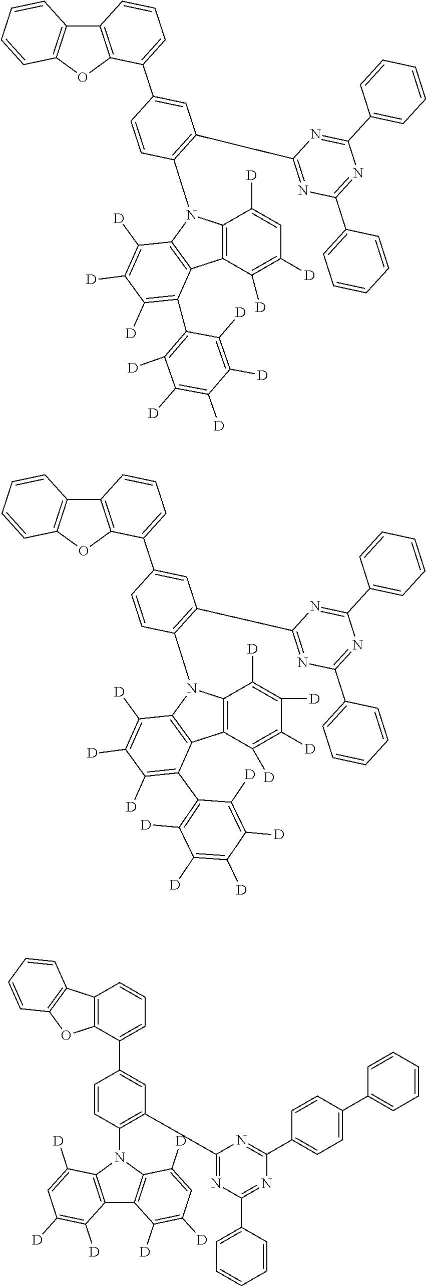 [SMILES: [2H]c1cc([2H])c2c(c1[2H])c1c(-c3c([2H])c([2H])c([2H])c([2H])c3[2H])c([2H])c([2H])c([2H])c1n2-c1ccc(-c2cccc3c2oc2ccccc23)cc1-c1nc(-c2ccccc2)nc(-c2ccccc2)n1 ; [2H]c1c([2H])c([2H])c(-c2c([2H])c([2H])c([2H])c3c2c2c([2H])c([2H])c([2H])c([2H])c2n3-c2ccc(-c3cccc4c3oc3ccccc34)cc2-c2nc(-c3ccccc3)nc(-c3ccccc3)n2)c([2H])c1[2H] ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3ccccc3)cc2)n1]

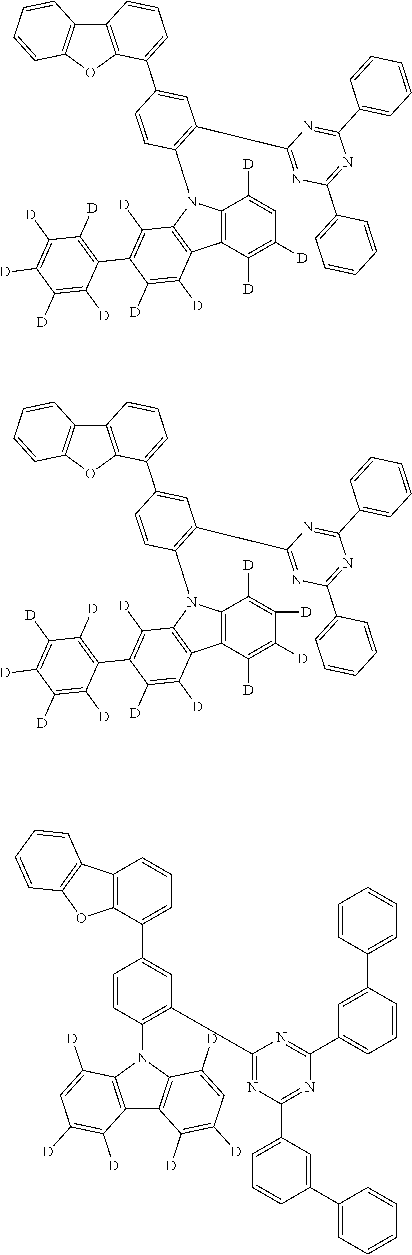 [SMILES: [2H]c1cc([2H])c2c(c1[2H])c1c([2H])c([2H])c(-c3c([2H])c([2H])c([2H])c([2H])c3[2H])c([2H])c1n2-c1ccc(-c2cccc3c2oc2ccccc23)cc1-c1nc(-c2ccccc2)nc(-c2ccccc2)n1 ; [2H]c1c([2H])c([2H])c(-c2c([2H])c([2H])c3c4c([2H])c([2H])c([2H])c([2H])c4n(-c4ccc(-c5cccc6c5oc5ccccc56)cc4-c4nc(-c5ccccc5)nc(-c5ccccc5)n4)c3c2[2H])c([2H])c1[2H] ; [2H]c1cc([2H])c2c(c1[2H])c1c([2H])c([2H])cc([2H])c1n2-c1ccc(-c2cccc3c2oc2ccccc23)cc1-c1nc(-c2cccc(-c3ccccc3)c2)nc(-c2cccc(-c3ccccc3)c2)n1]

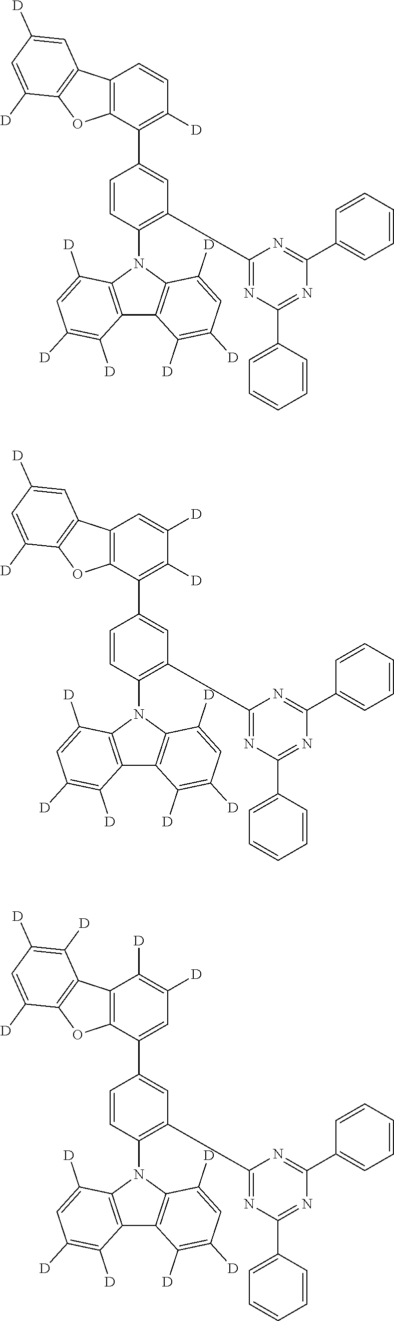 [SMILES: [2H]c1cc([2H])c2oc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)c([2H])ccc3c2c1 ; [2H]c1cc([2H])c2oc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)c([2H])c([2H])cc3c2c1 ; [2H]c1cc([2H])c2oc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)cc([2H])c([2H])c3c2c1[2H]]

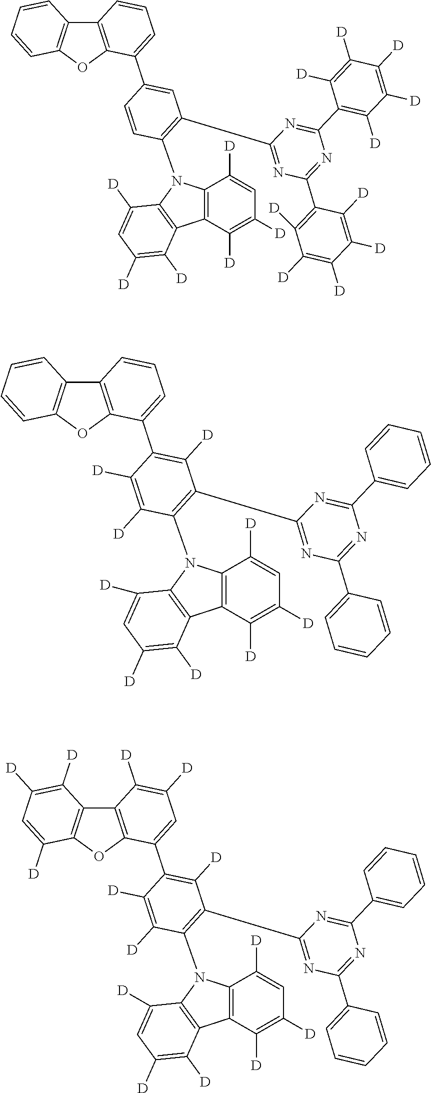 [SMILES: [2H]c1cc([2H])c2c(c1[2H])c1c([2H])c([2H])cc([2H])c1n2-c1ccc(-c2cccc3c2oc2ccccc23)cc1-c1nc(-c2c([2H])c([2H])c([2H])c([2H])c2[2H])nc(-c2c([2H])c([2H])c([2H])c([2H])c2[2H])n1 ; [2H]c1cc([2H])c2c(c1[2H])c1c([2H])c([2H])cc([2H])c1n2-c1c([2H])c([2H])c(-c2cccc3c2oc2ccccc23)c([2H])c1-c1nc(-c2ccccc2)nc(-c2ccccc2)n1 ; [2H]c1cc([2H])c2oc3c(-c4c([2H])c([2H])c(-n5c6c([2H])cc([2H])c([2H])c6c6c([2H])c([2H])cc([2H])c65)c(-c5nc(-c6ccccc6)nc(-c6ccccc6)n5)c4[2H])cc([2H])c([2H])c3c2c1[2H]]

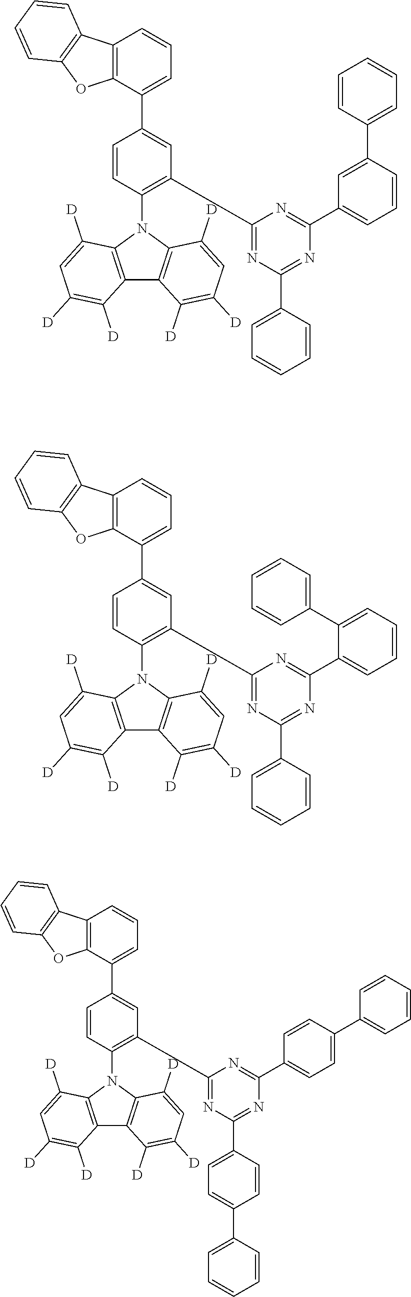 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc(-c3ccccc3)c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccccc2-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccc(-c3ccccc3)cc2)nc(-c2ccc(-c3ccccc3)cc2)n1]

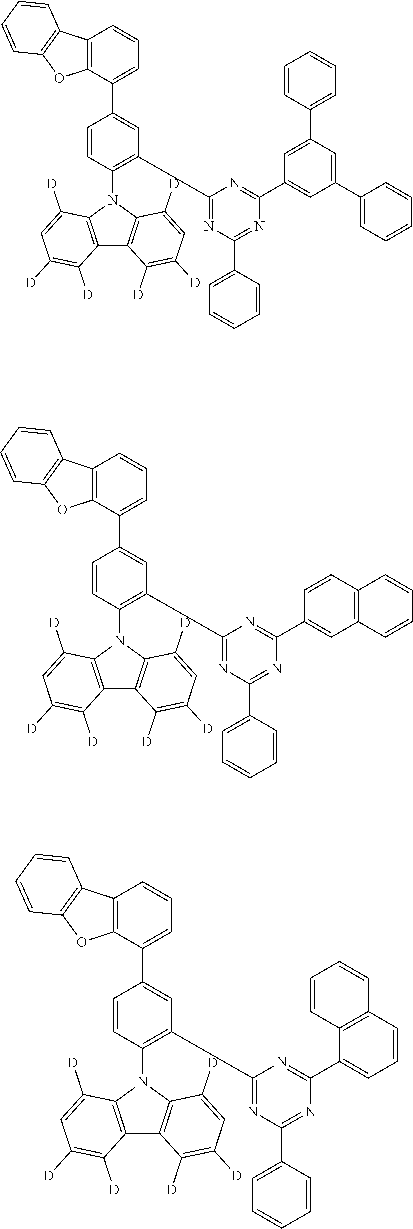 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cc(-c3ccccc3)cc(-c3ccccc3)c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3ccccc3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3ccccc23)n1]

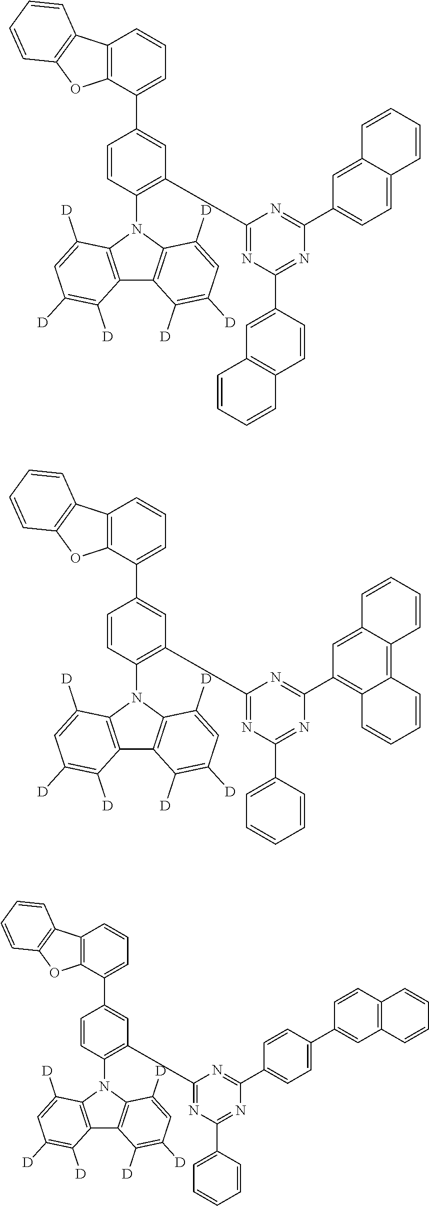 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccc3ccccc3c2)nc(-c2ccc3ccccc3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cc3ccccc3c3ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3ccc4ccccc4c3)cc2)n1]

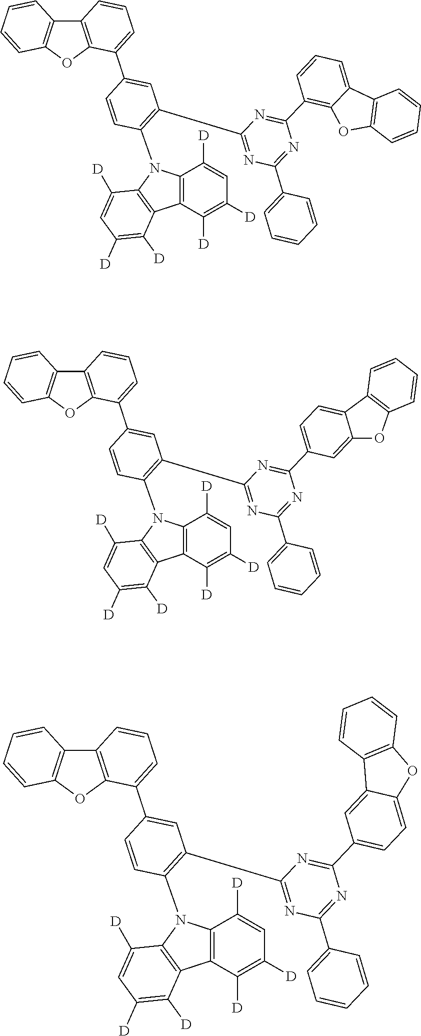 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c2oc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3oc4ccccc4c3c2)n1]

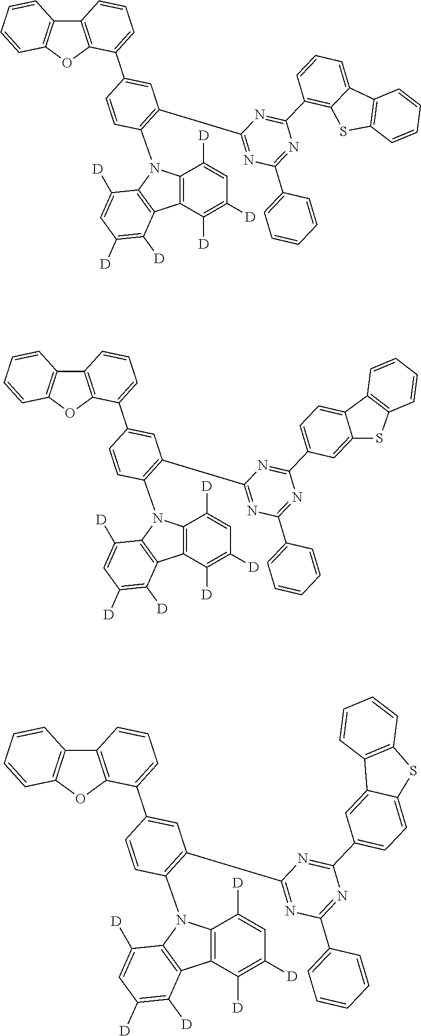 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c2sc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c(c2)sc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3sc4ccccc4c3c2)n1]

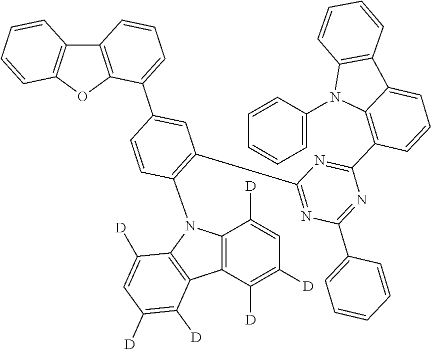 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c4ccccc4n(-c4ccccc4)c23)n1]

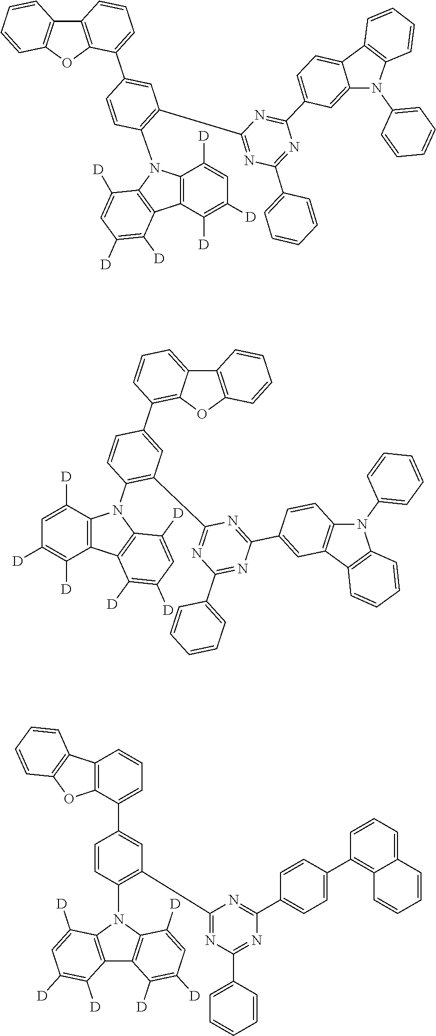 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c4ccccc4n(-c4ccccc4)c3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c(c2)c2ccccc2n3-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3cccc4ccccc34)cc2)n1]

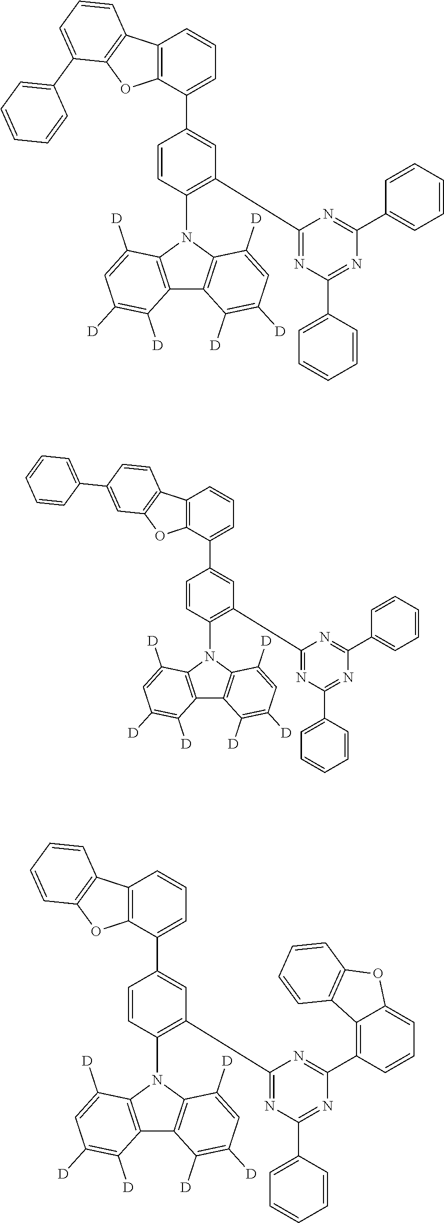 [SMILES: [2H]c1cc([2H])c2c(c1[2H])c1c([2H])c([2H])cc([2H])c1n2-c1ccc(-c2cccc3c2oc2c(-c4ccccc4)cccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2cc(-c4ccccc4)ccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3oc4ccccc4c23)n1]

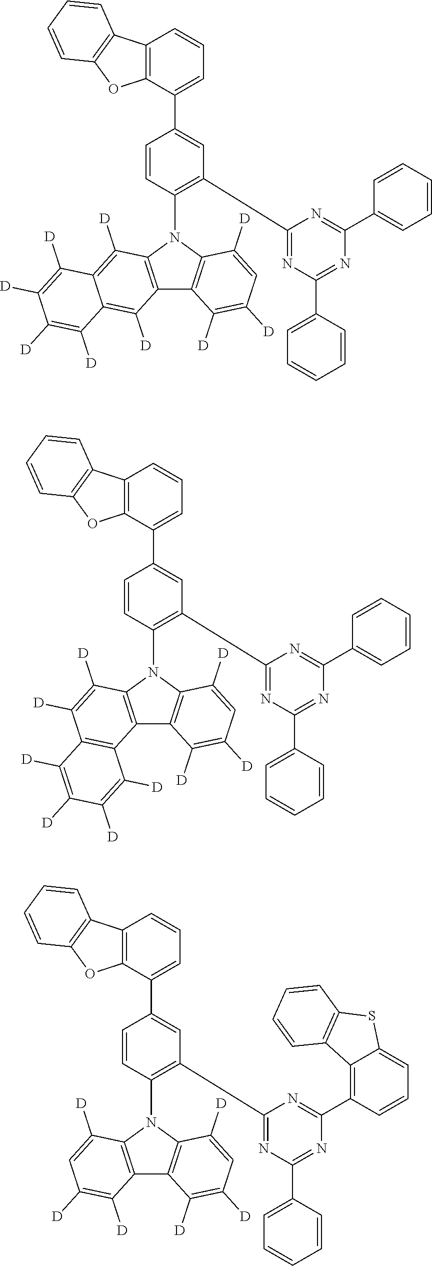 [SMILES: [2H]c1cc([2H])c2c(c1[2H])c1c([2H])c3c([2H])c([2H])c([2H])c([2H])c3c([2H])c1n2-c1ccc(-c2cccc3c2oc2ccccc23)cc1-c1nc(-c2ccccc2)nc(-c2ccccc2)n1 ; [2H]c1cc([2H])c2c(c1[2H])c1c3c([2H])c([2H])c([2H])c([2H])c3c([2H])c([2H])c1n2-c1ccc(-c2cccc3c2oc2ccccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3sc4ccccc4c23)n1]

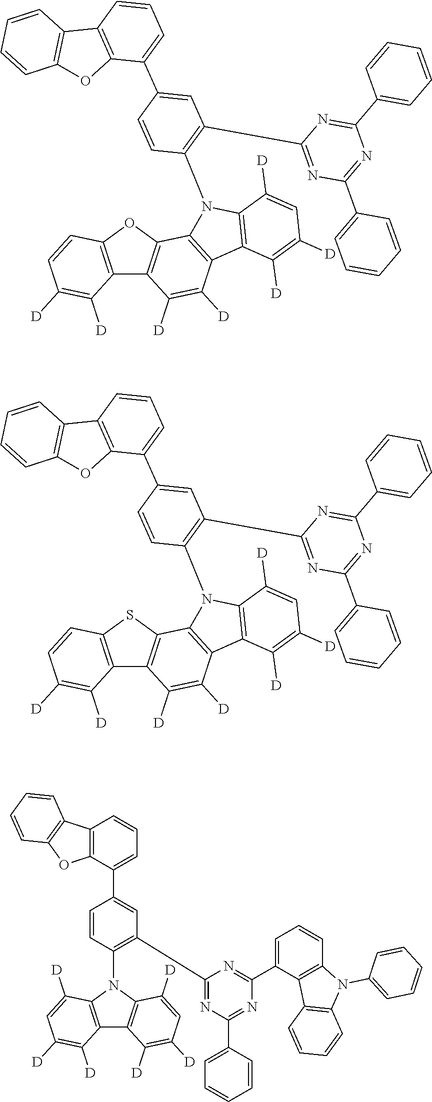 [SMILES: [2H]c1ccc2oc3c(c([2H])c([2H])c4c5c([2H])c([2H])cc([2H])c5n(-c5ccc(-c6cccc7c6oc6ccccc67)cc5-c5nc(-c6ccccc6)nc(-c6ccccc6)n5)c34)c2c1[2H] ; [2H]c1ccc2sc3c(c([2H])c([2H])c4c5c([2H])c([2H])cc([2H])c5n(-c5ccc(-c6cccc7c6oc6ccccc67)cc5-c5nc(-c6ccccc6)nc(-c6ccccc6)n5)c34)c2c1[2H] ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c2c2ccccc2n3-c2ccccc2)n1]

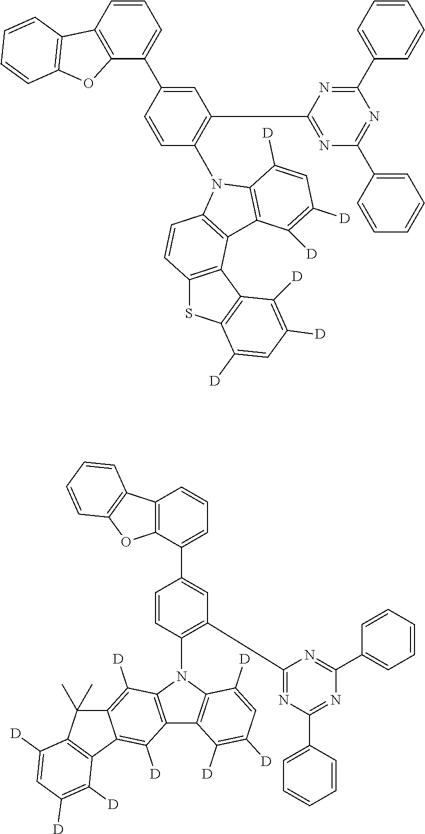 [SMILES: [2H]c1cc([2H])c2sc3ccc4c(c5c([2H])c([2H])cc([2H])c5n4-c4ccc(-c5cccc6c5oc5ccccc56)cc4-c4nc(-c5ccccc5)nc(-c5ccccc5)n4)c3c2c1[2H] ; [2H]c1cc([2H])c2c(c1[2H])-c1c(c([2H])c3c(c1[2H])c1c([2H])c([2H])cc([2H])c1n3-c1ccc(-c3cccc4c3oc3ccccc34)cc1-c1nc(-c3ccccc3)nc(-c3ccccc3)n1)C2(C)C]

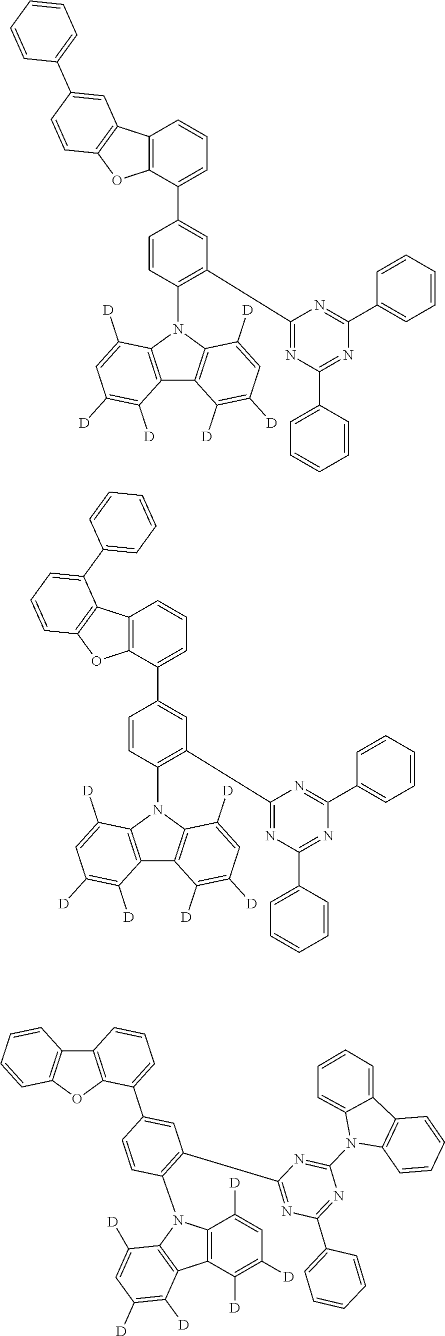 [SMILES: [2H]c1cc([2H])c2c(c1[2H])c1c([2H])c([2H])cc([2H])c1n2-c1ccc(-c2cccc3c2oc2ccc(-c4ccccc4)cc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2cccc(-c4ccccc4)c23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-n2c3ccccc3c3ccccc32)n1]

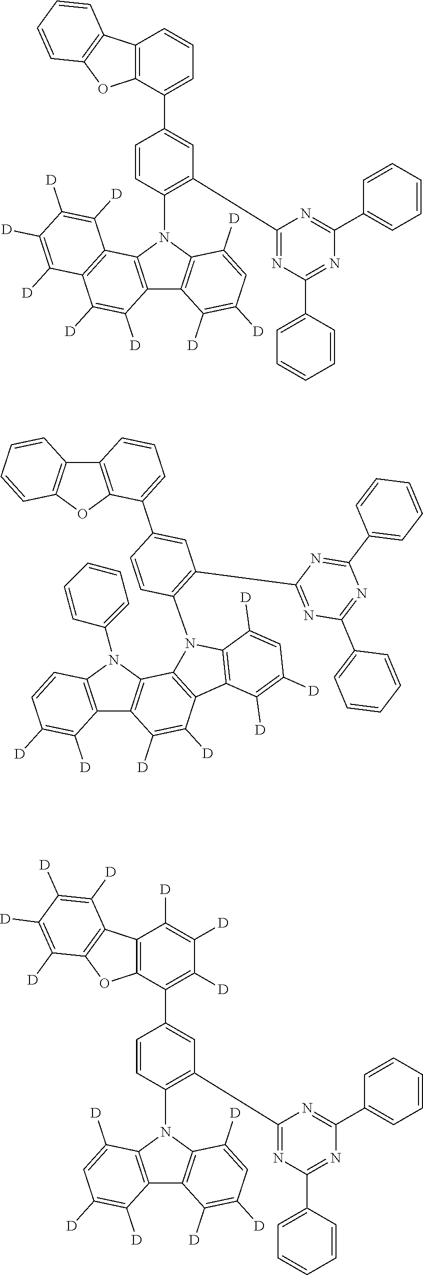 [SMILES: [2H]c1cc([2H])c2c(c1[2H])c1c([2H])c([2H])c3c([2H])c([2H])c([2H])c([2H])c3c1n2-c1ccc(-c2cccc3c2oc2ccccc23)cc1-c1nc(-c2ccccc2)nc(-c2ccccc2)n1 ; [2H]c1ccc2c(c1[2H])c1c([2H])c([2H])c3c4c([2H])c([2H])cc([2H])c4n(-c4ccc(-c5cccc6c5oc5ccccc56)cc4-c4nc(-c5ccccc5)nc(-c5ccccc5)n4)c3c1n2-c1ccccc1 ; [2H]c1cc([2H])c2c(c1[2H])c1c([2H])c([2H])cc([2H])c1n2-c1ccc(-c2c([2H])c([2H])c([2H])c3c2oc2c([2H])c([2H])c([2H])c([2H])c23)cc1-c1nc(-c2ccccc2)nc(-c2ccccc2)n1]

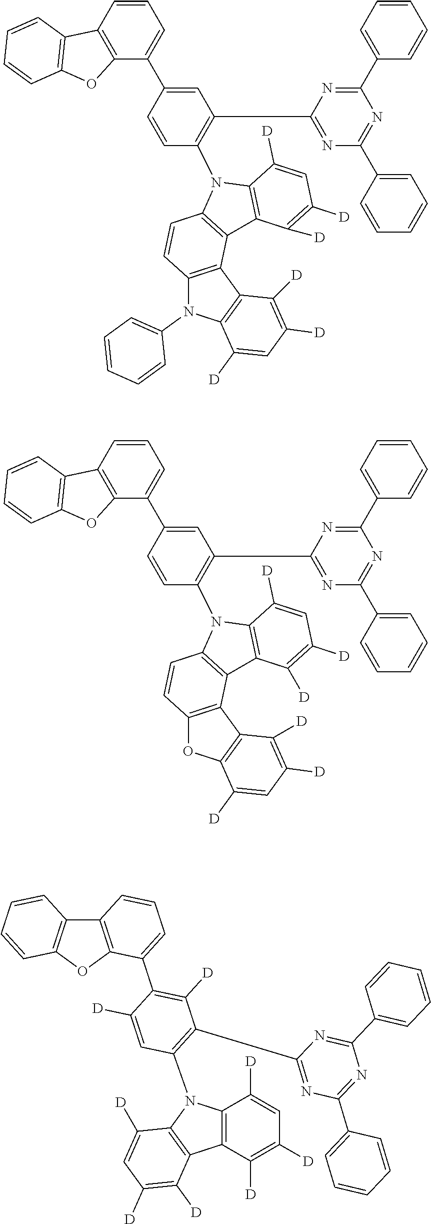 [SMILES: [2H]c1cc([2H])c2c(c1[2H])c1c3c4c([2H])c([2H])cc([2H])c4n(-c4ccc(-c5cccc6c5oc5ccccc56)cc4-c4nc(-c5ccccc5)nc(-c5ccccc5)n4)c3ccc1n2-c1ccccc1 ; [2H]c1cc([2H])c2oc3ccc4c(c5c([2H])c([2H])cc([2H])c5n4-c4ccc(-c5cccc6c5oc5ccccc56)cc4-c4nc(-c5ccccc5)nc(-c5ccccc5)n4)c3c2c1[2H] ; [2H]c1cc(-n2c3c([2H])cc([2H])c([2H])c3c3c([2H])c([2H])cc([2H])c32)c(-c2nc(-c3ccccc3)nc(-c3ccccc3)n2)c([2H])c1-c1cccc2c1oc1ccccc12]

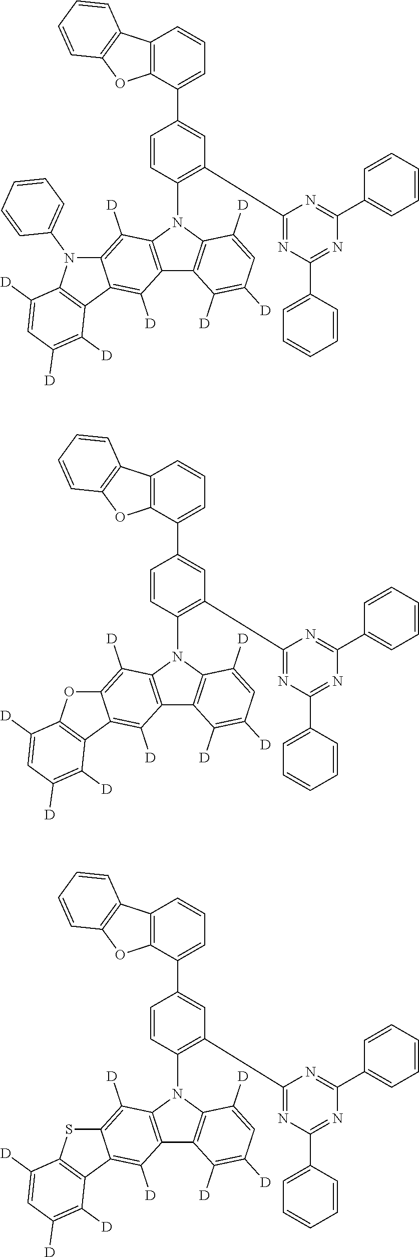 [SMILES: [2H]c1cc([2H])c2c(c1[2H])c1c([2H])c3c4c([2H])c([2H])cc([2H])c4n(-c4ccc(-c5cccc6c5oc5ccccc56)cc4-c4nc(-c5ccccc5)nc(-c5ccccc5)n4)c3c([2H])c1n2-c1ccccc1 ; [2H]c1cc([2H])c2oc3c([2H])c4c(c([2H])c3c2c1[2H])c1c([2H])c([2H])cc([2H])c1n4-c1ccc(-c2cccc3c2oc2ccccc23)cc1-c1nc(-c2ccccc2)nc(-c2ccccc2)n1 ; [2H]c1cc([2H])c2sc3c([2H])c4c(c([2H])c3c2c1[2H])c1c([2H])c([2H])cc([2H])c1n4-c1ccc(-c2cccc3c2oc2ccccc23)cc1-c1nc(-c2ccccc2)nc(-c2ccccc2)n1]

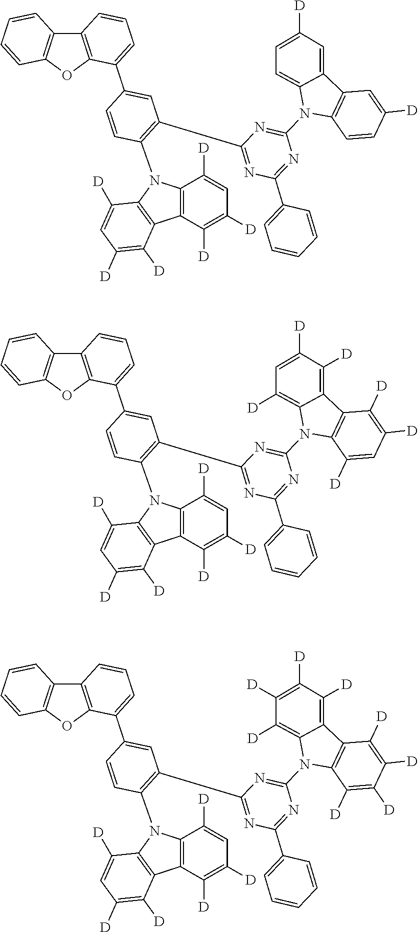 [SMILES: [2H]c1ccc2c(c1)c1cc([2H])ccc1n2-c1nc(-c2ccccc2)nc(-c2cc(-c3cccc4c3oc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1nc(-c2ccccc2)nc(-c2cc(-c3cccc4c3oc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-n2c3c([2H])c([2H])c([2H])c([2H])c3c3c([2H])c([2H])c([2H])c([2H])c32)n1]

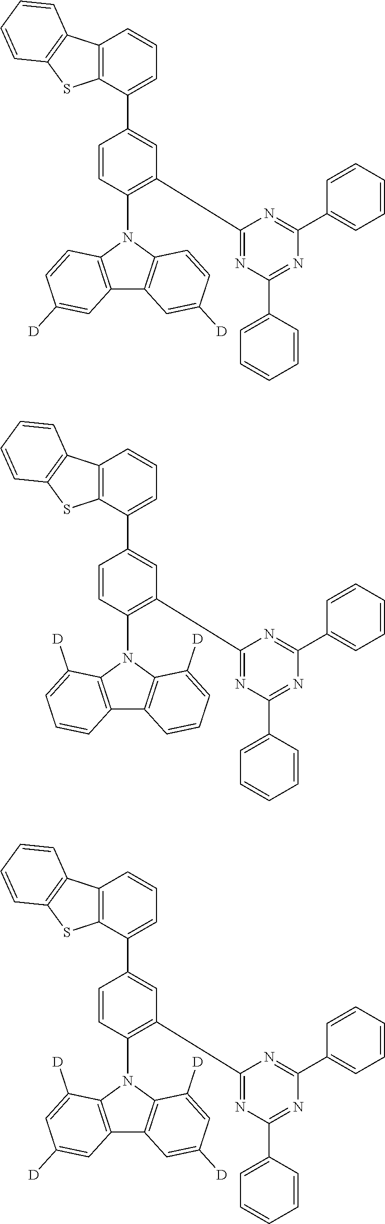 [SMILES: [2H]c1ccc2c(c1)c1cc([2H])ccc1n2-c1ccc(-c2cccc3c2sc2ccccc23)cc1-c1nc(-c2ccccc2)nc(-c2ccccc2)n1 ; [2H]c1cccc2c3cccc([2H])c3n(-c3ccc(-c4cccc5c4sc4ccccc45)cc3-c3nc(-c4ccccc4)nc(-c4ccccc4)n3)c12 ; [2H]c1cc([2H])c2c(c1)c1cc([2H])cc([2H])c1n2-c1ccc(-c2cccc3c2sc2ccccc23)cc1-c1nc(-c2ccccc2)nc(-c2ccccc2)n1]

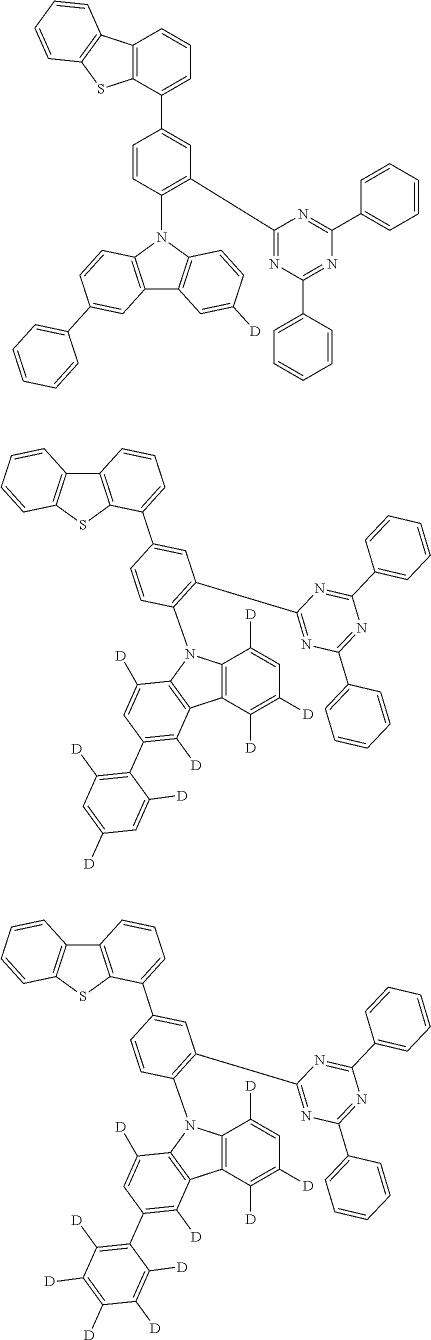 [SMILES: [2H]c1ccc2c(c1)c1cc(-c3ccccc3)ccc1n2-c1ccc(-c2cccc3c2sc2ccccc23)cc1-c1nc(-c2ccccc2)nc(-c2ccccc2)n1 ; [2H]c1cc([2H])c(-c2cc([2H])c3c(c2[2H])c2c([2H])c([2H])cc([2H])c2n3-c2ccc(-c3cccc4c3sc3ccccc34)cc2-c2nc(-c3ccccc3)nc(-c3ccccc3)n2)c([2H])c1 ; [2H]c1cc([2H])c2c(c1[2H])c1c([2H])c(-c3c([2H])c([2H])c([2H])c([2H])c3[2H])cc([2H])c1n2-c1ccc(-c2cccc3c2sc2ccccc23)cc1-c1nc(-c2ccccc2)nc(-c2ccccc2)n1]

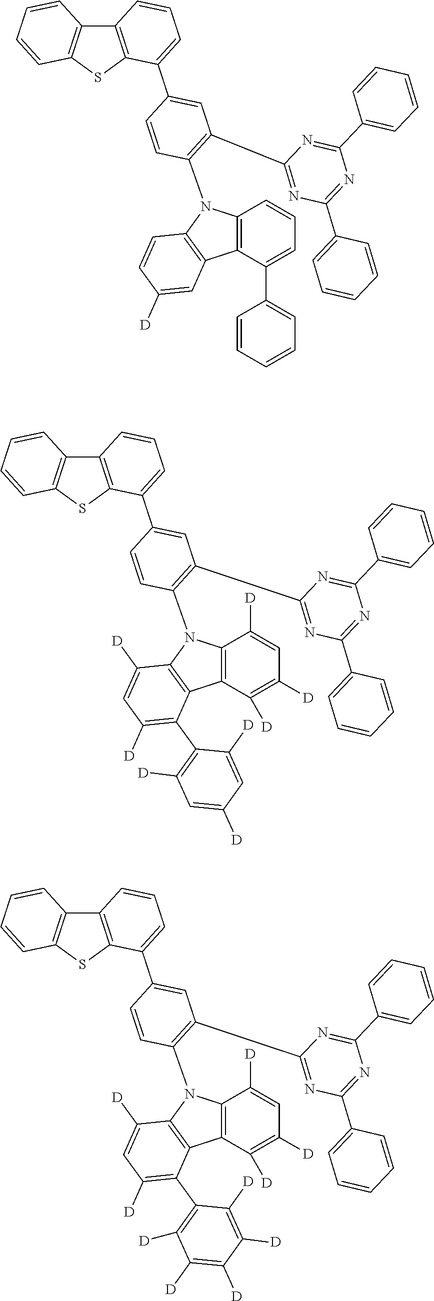 [SMILES: [2H]c1ccc2c(c1)c1c(-c3ccccc3)cccc1n2-c1ccc(-c2cccc3c2sc2ccccc23)cc1-c1nc(-c2ccccc2)nc(-c2ccccc2)n1 ; [2H]c1cc([2H])c(-c2c([2H])cc([2H])c3c2c2c([2H])c([2H])cc([2H])c2n3-c2ccc(-c3cccc4c3sc3ccccc34)cc2-c2nc(-c3ccccc3)nc(-c3ccccc3)n2)c([2H])c1 ; [2H]c1cc([2H])c2c(c1[2H])c1c(-c3c([2H])c([2H])c([2H])c([2H])c3[2H])c([2H])cc([2H])c1n2-c1ccc(-c2cccc3c2sc2ccccc23)cc1-c1nc(-c2ccccc2)nc(-c2ccccc2)n1]

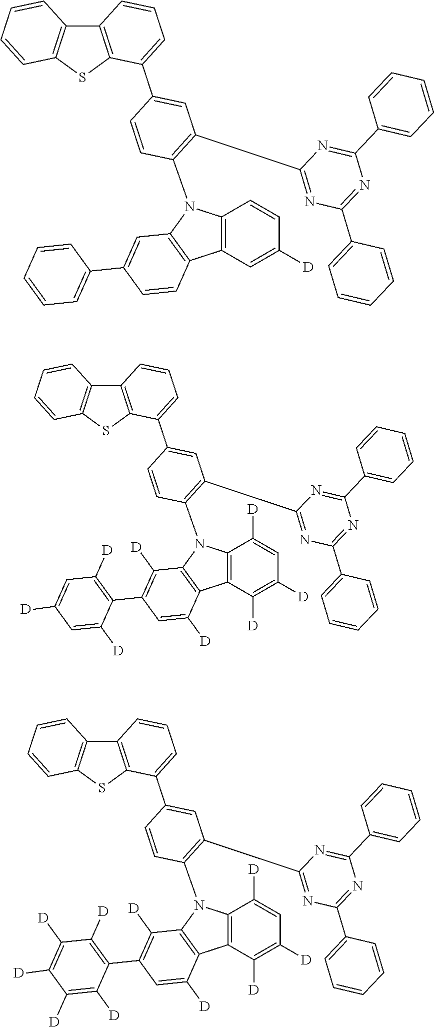 [SMILES: [2H]c1ccc2c(c1)c1ccc(-c3ccccc3)cc1n2-c1ccc(-c2cccc3c2sc2ccccc23)cc1-c1nc(-c2ccccc2)nc(-c2ccccc2)n1 ; [2H]c1cc([2H])c(-c2cc([2H])c3c4c([2H])c([2H])cc([2H])c4n(-c4ccc(-c5cccc6c5sc5ccccc56)cc4-c4nc(-c5ccccc5)nc(-c5ccccc5)n4)c3c2[2H])c([2H])c1 ; [2H]c1cc([2H])c2c(c1[2H])c1c([2H])cc(-c3c([2H])c([2H])c([2H])c([2H])c3[2H])c([2H])c1n2-c1ccc(-c2cccc3c2sc2ccccc23)cc1-c1nc(-c2ccccc2)nc(-c2ccccc2)n1]

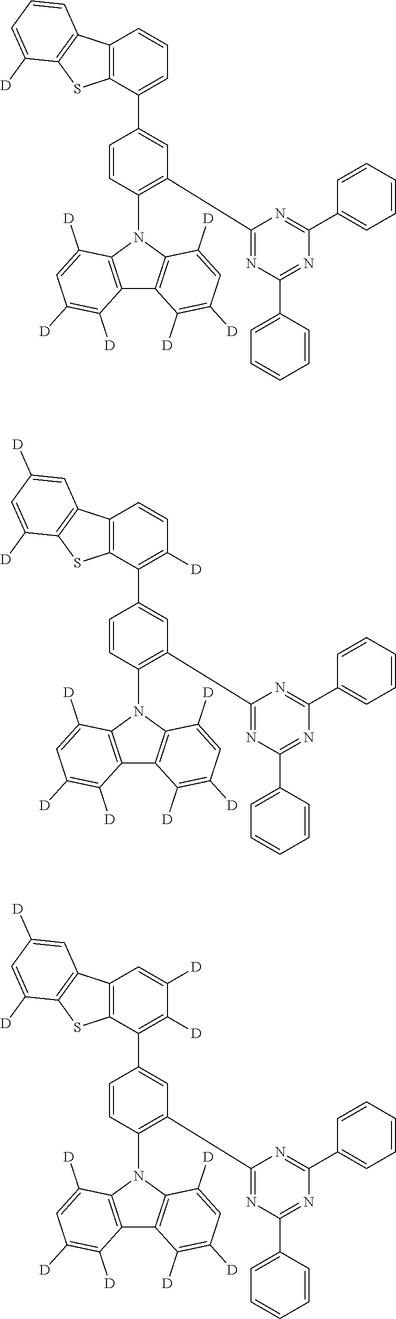 [SMILES: [2H]c1cc([2H])c2c(c1[2H])c1c([2H])c([2H])cc([2H])c1n2-c1ccc(-c2cccc3c2sc2c([2H])cccc23)cc1-c1nc(-c2ccccc2)nc(-c2ccccc2)n1 ; [2H]c1cc([2H])c2sc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)c([2H])ccc3c2c1 ; [2H]c1cc([2H])c2sc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)c([2H])c([2H])cc3c2c1]

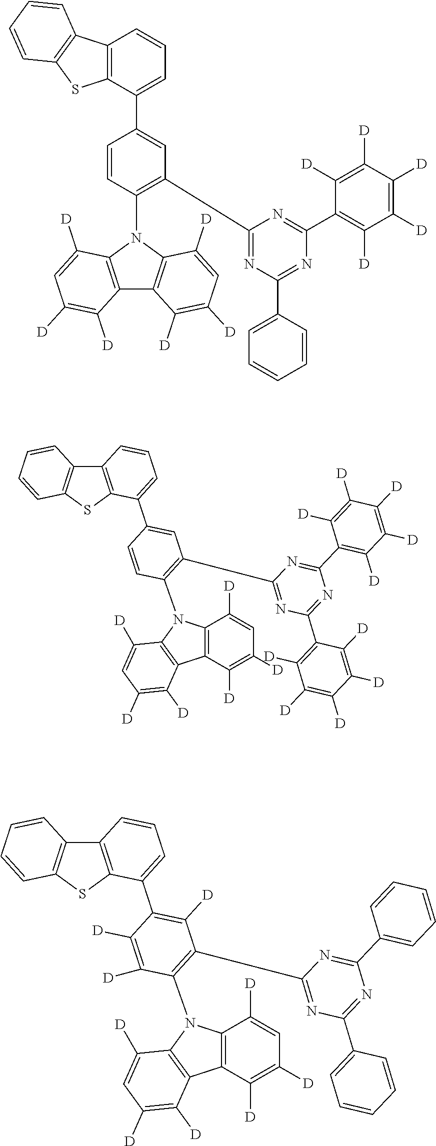 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2c([2H])c([2H])c([2H])c([2H])c2[2H])n1 ; [2H]c1cc([2H])c2c(c1[2H])c1c([2H])c([2H])cc([2H])c1n2-c1ccc(-c2cccc3c2sc2ccccc23)cc1-c1nc(-c2c([2H])c([2H])c([2H])c([2H])c2[2H])nc(-c2c([2H])c([2H])c([2H])c([2H])c2[2H])n1 ; [2H]c1cc([2H])c2c(c1[2H])c1c([2H])c([2H])cc([2H])c1n2-c1c([2H])c([2H])c(-c2cccc3c2sc2ccccc23)c([2H])c1-c1nc(-c2ccccc2)nc(-c2ccccc2)n1]

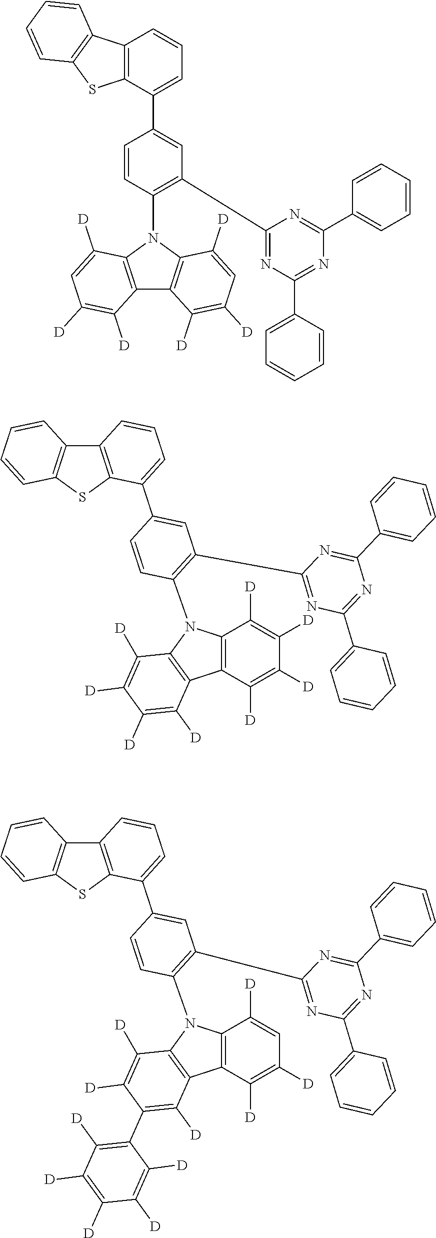 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2cccc3c2sc2ccccc23)cc1-c1nc(-c2ccccc2)nc(-c2ccccc2)n1 ; [2H]c1cc([2H])c2c(c1[2H])c1c([2H])c(-c3c([2H])c([2H])c([2H])c([2H])c3[2H])c([2H])c([2H])c1n2-c1ccc(-c2cccc3c2sc2ccccc23)cc1-c1nc(-c2ccccc2)nc(-c2ccccc2)n1]

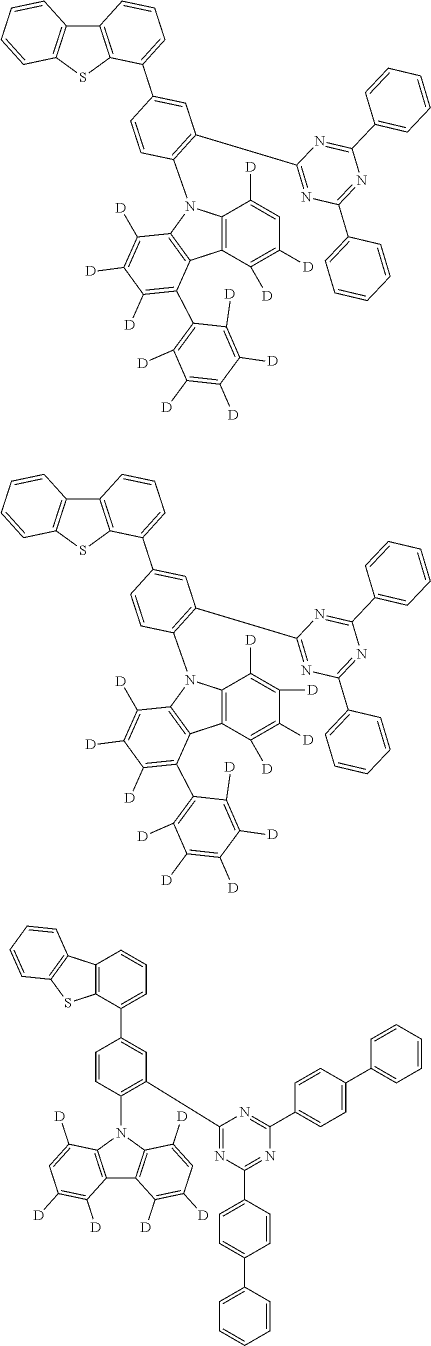 [SMILES: [2H]c1cc([2H])c2c(c1[2H])c1c(-c3c([2H])c([2H])c([2H])c([2H])c3[2H])c([2H])c([2H])c([2H])c1n2-c1ccc(-c2cccc3c2sc2ccccc23)cc1-c1nc(-c2ccccc2)nc(-c2ccccc2)n1 ; [2H]c1c([2H])c([2H])c(-c2c([2H])c([2H])c([2H])c3c2c2c([2H])c([2H])c([2H])c([2H])c2n3-c2ccc(-c3cccc4c3sc3ccccc34)cc2-c2nc(-c3ccccc3)nc(-c3ccccc3)n2)c([2H])c1[2H] ; [2H]c1cc([2H])c2c(c1[2H])c1c([2H])c([2H])cc([2H])c1n2-c1ccc(-c2cccc3c2sc2ccccc23)cc1-c1nc(-c2ccc(-c3ccccc3)cc2)nc(-c2ccc(-c3ccccc3)cc2)n1]

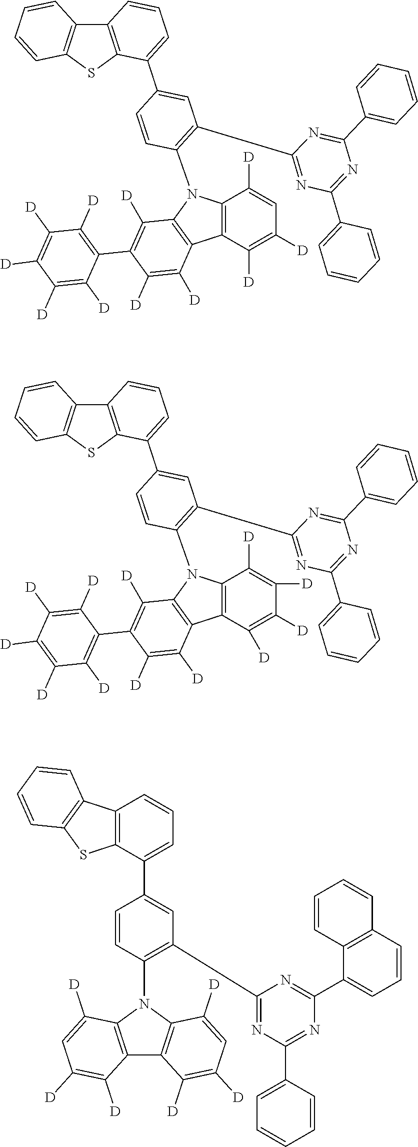 [SMILES: [2H]c1cc([2H])c2c(c1[2H])c1c([2H])c([2H])c(-c3c([2H])c([2H])c([2H])c([2H])c3[2H])c([2H])c1n2-c1ccc(-c2cccc3c2sc2ccccc23)cc1-c1nc(-c2ccccc2)nc(-c2ccccc2)n1 ; [2H]c1c([2H])c([2H])c(-c2c([2H])c([2H])c3c4c([2H])c([2H])c([2H])c([2H])c4n(-c4ccc(-c5cccc6c5sc5ccccc56)cc4-c4nc(-c5ccccc5)nc(-c5ccccc5)n4)c3c2[2H])c([2H])c1[2H] ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3ccccc23)n1]

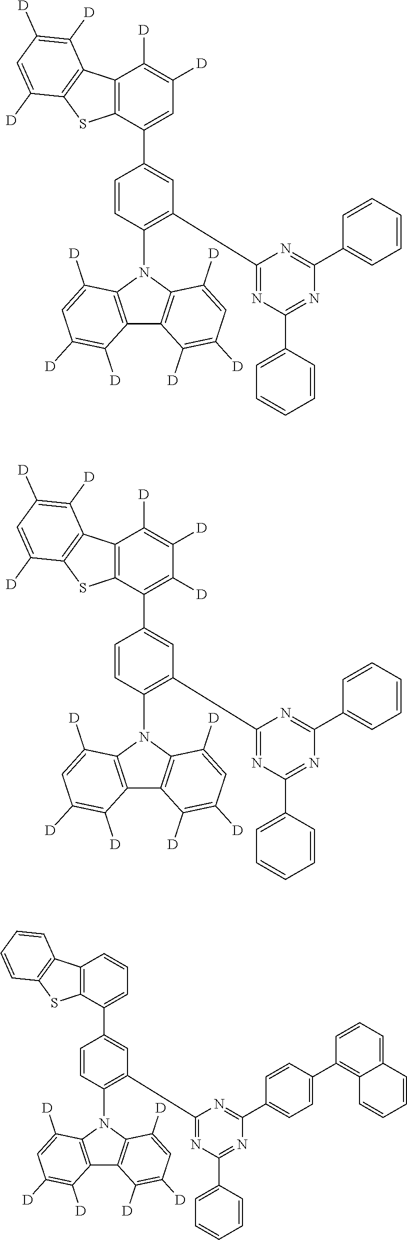 [SMILES: [2H]c1cc([2H])c2sc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)cc([2H])c([2H])c3c2c1[2H] ; [2H]c1cc([2H])c2sc3c(-c4ccc(-n5c6c([2H])cc([2H])c([2H])c6c6c([2H])c([2H])cc([2H])c65)c(-c5nc(-c6ccccc6)nc(-c6ccccc6)n5)c4)c([2H])c([2H])c([2H])c3c2c1[2H] ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc(-c3cccc4ccccc34)cc2)n1]

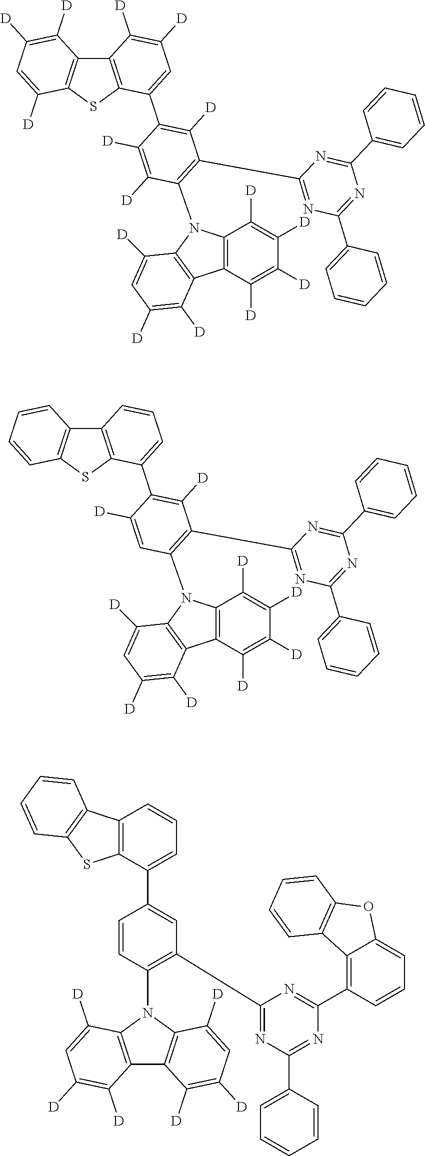 [SMILES: [2H]c1cc([2H])c2sc3c(-c4c([2H])c([2H])c(-n5c6c([2H])cc([2H])c([2H])c6c6c([2H])c([2H])c([2H])c([2H])c65)c(-c5nc(-c6ccccc6)nc(-c6ccccc6)n5)c4[2H])cc([2H])c([2H])c3c2c1[2H] ; [2H]c1cc(-n2c3c([2H])cc([2H])c([2H])c3c3c([2H])c([2H])c([2H])c([2H])c32)c(-c2nc(-c3ccccc3)nc(-c3ccccc3)n2)c([2H])c1-c1cccc2c1sc1ccccc12 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3oc4ccccc4c23)n1]

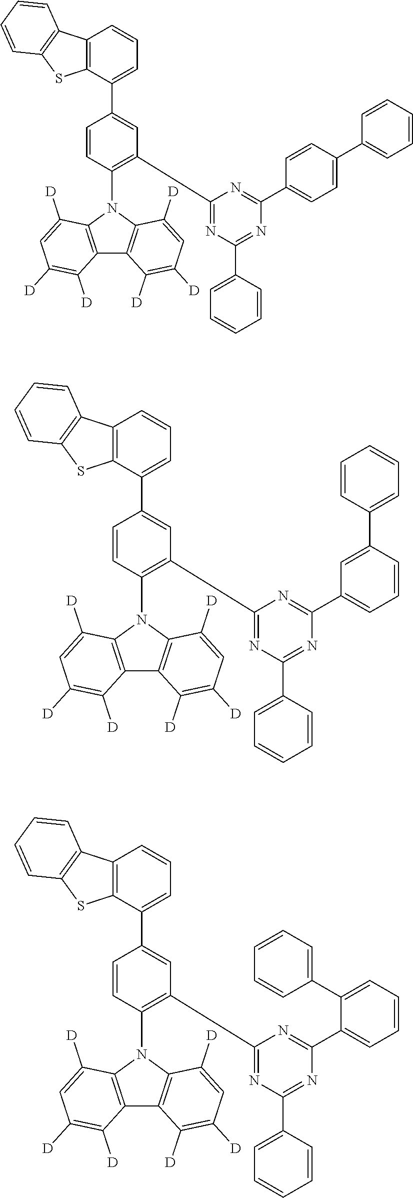 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc(-c3ccccc3)cc2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc(-c3ccccc3)c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccccc2-c2ccccc2)n1]

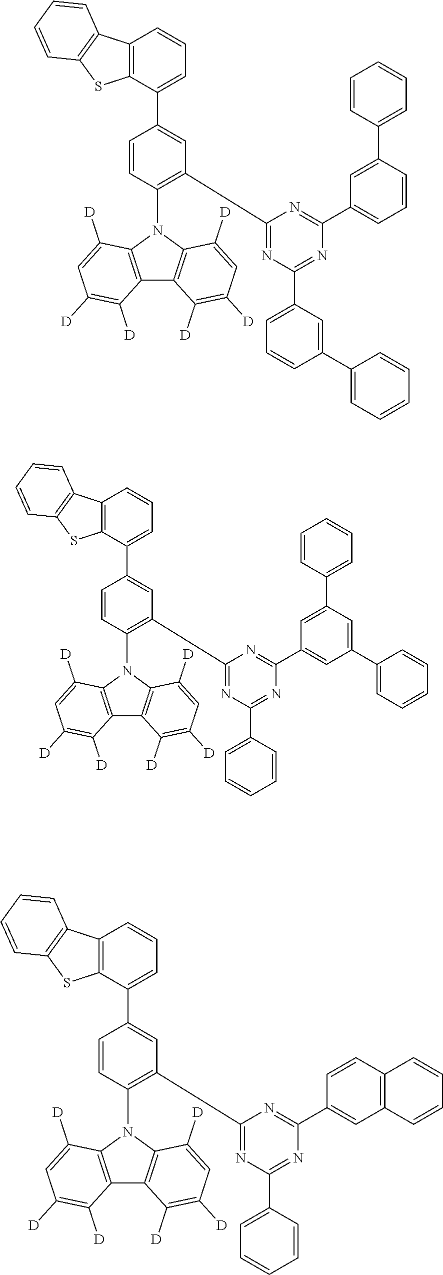 [SMILES: [2H]c1cc([2H])c2c(c1[2H])c1c([2H])c([2H])cc([2H])c1n2-c1ccc(-c2cccc3c2sc2ccccc23)cc1-c1nc(-c2cccc(-c3ccccc3)c2)nc(-c2cccc(-c3ccccc3)c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cc(-c3ccccc3)cc(-c3ccccc3)c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3ccccc3c2)n1]

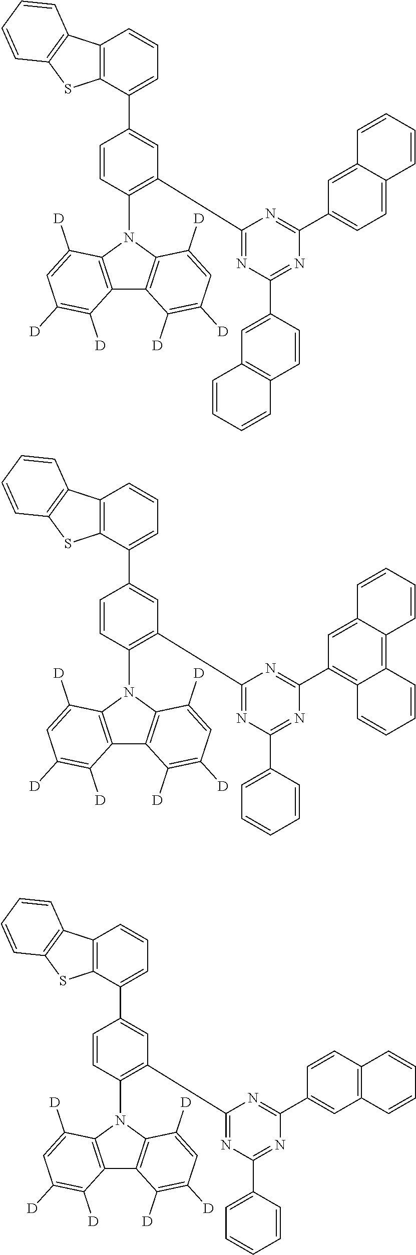 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccc3ccccc3c2)nc(-c2ccc3ccccc3c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cc3ccccc3c3ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3ccccc3c2)n1]

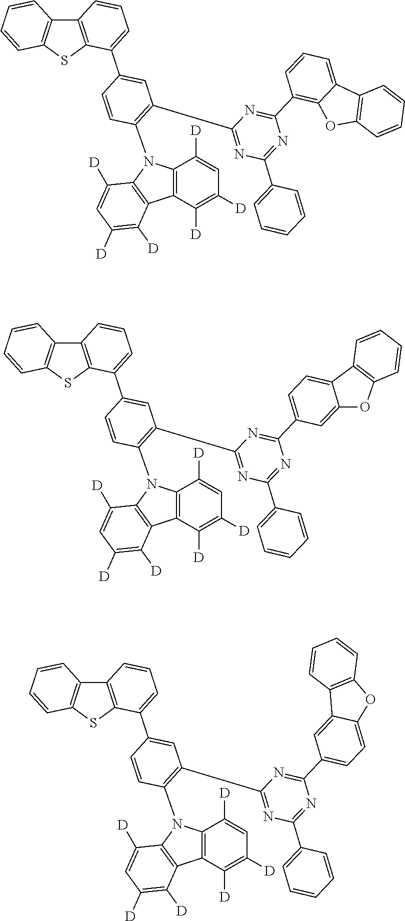 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2oc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3oc4ccccc4c3c2)n1]

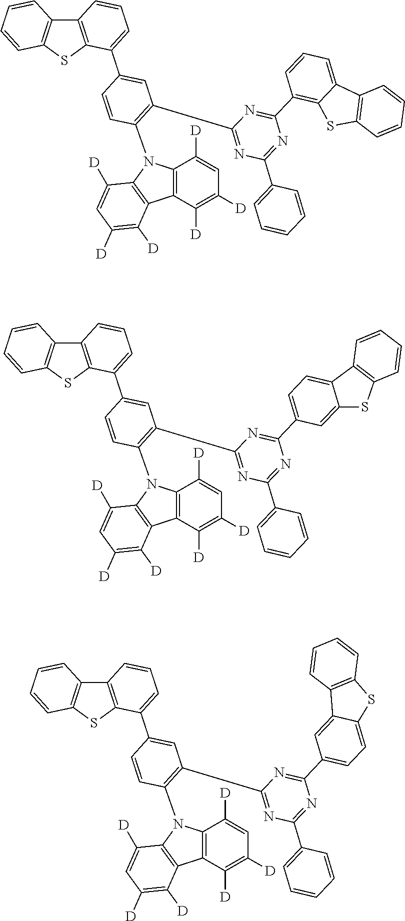 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2sc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)sc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3sc4ccccc4c3c2)n1]

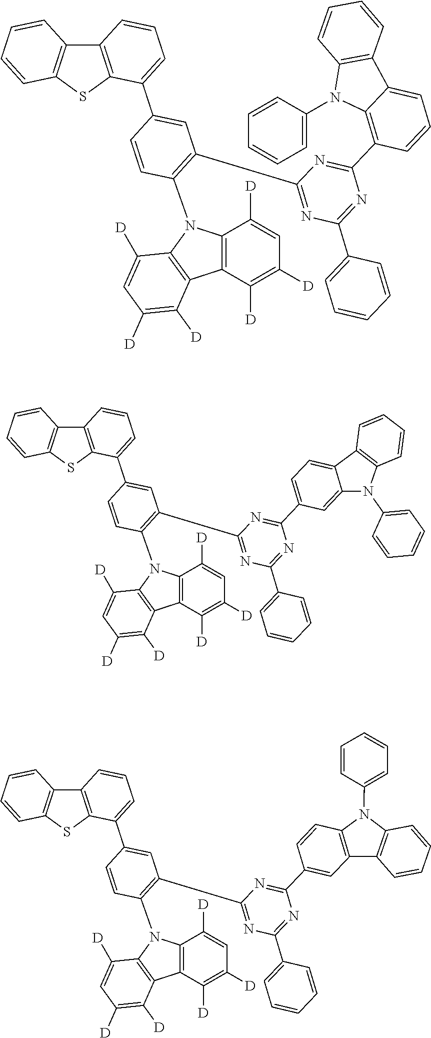 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c4ccccc4n(-c4ccccc4)c23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c4ccccc4n(-c4ccccc4)c3c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)c2ccccc2n3-c2ccccc2)n1]

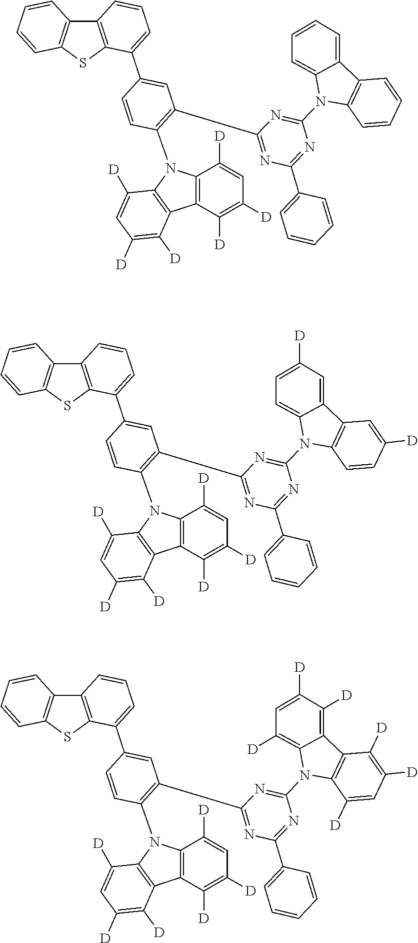 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-n2c3ccccc3c3ccccc32)n1 ; [2H]c1ccc2c(c1)c1cc([2H])ccc1n2-c1nc(-c2ccccc2)nc(-c2cc(-c3cccc4c3sc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1nc(-c2ccccc2)nc(-c2cc(-c3cccc4c3sc3ccccc34)ccc2-n2c3c([2H])cc([2H])c([2H])c3c3c([2H])c([2H])cc([2H])c32)n1]

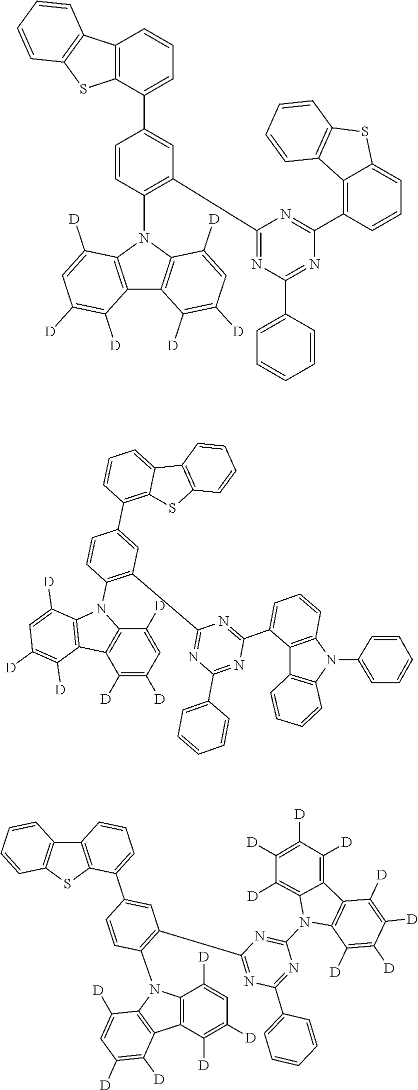 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3sc4ccccc4c23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2c2ccccc2n3-c2ccccc2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-n2c3c([2H])c([2H])c([2H])c([2H])c3c3c([2H])c([2H])c([2H])c([2H])c32)n1]

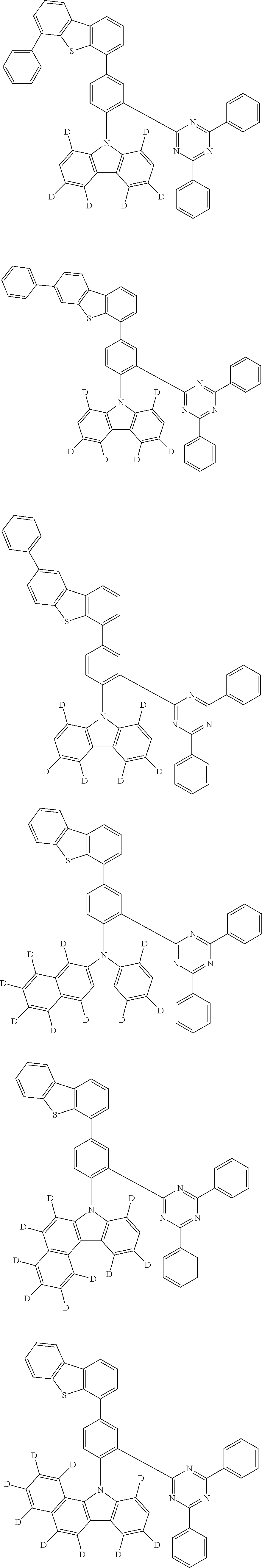

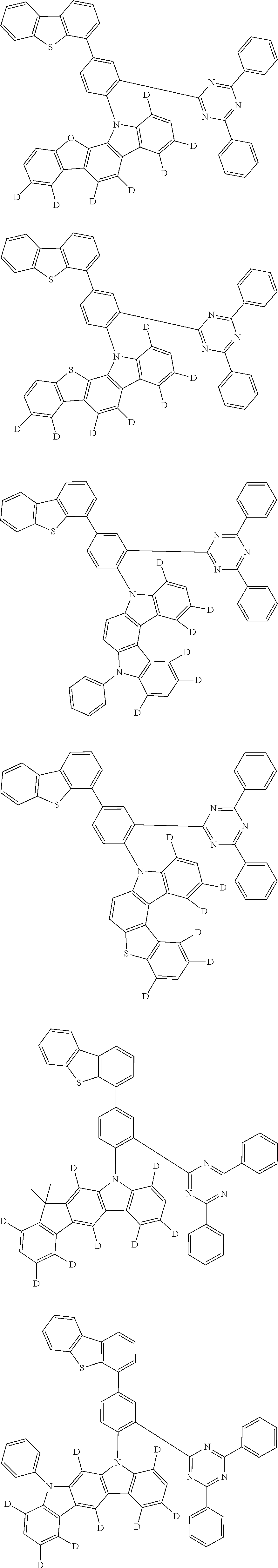

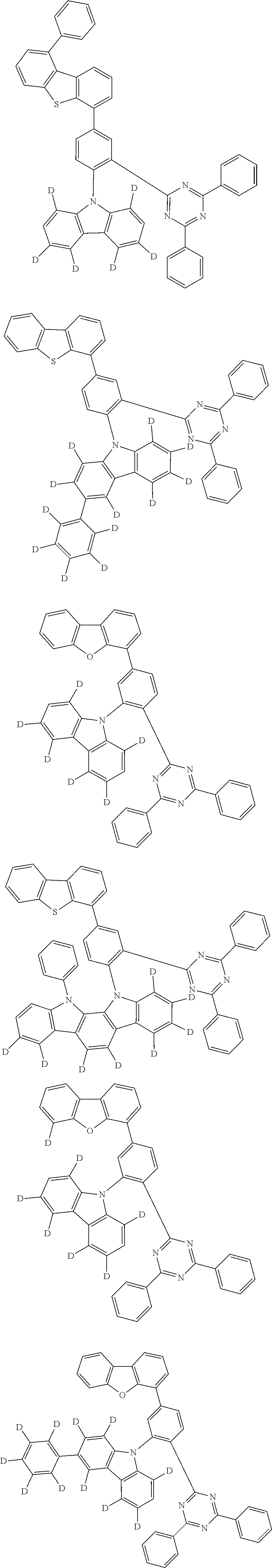

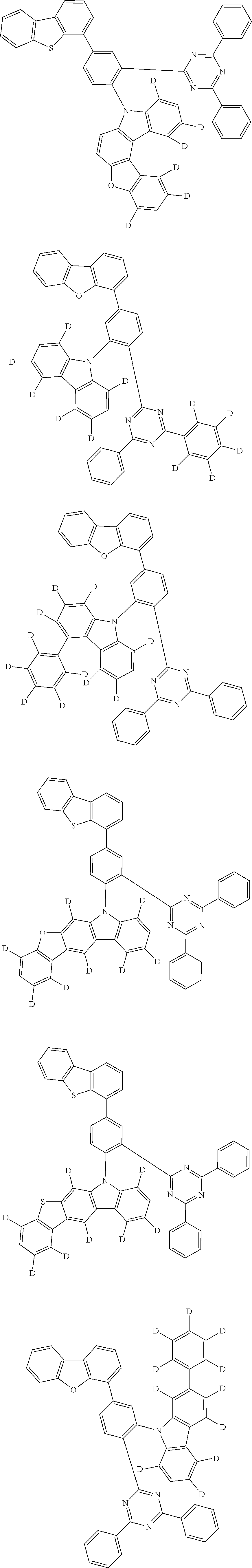

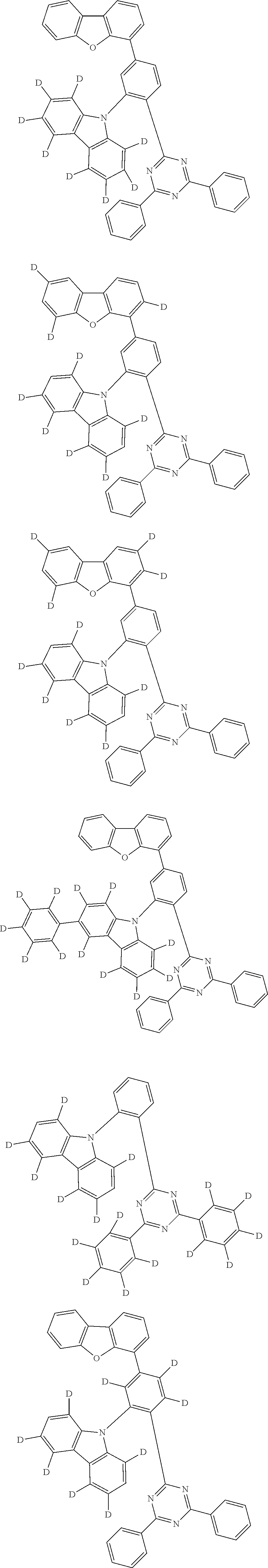

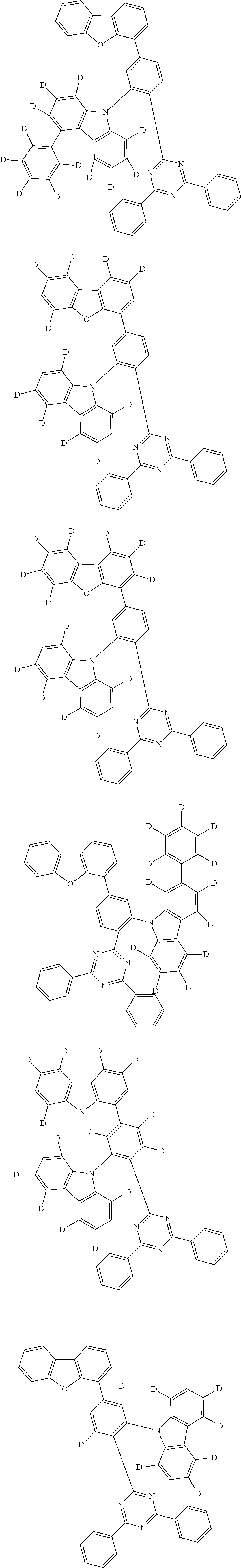

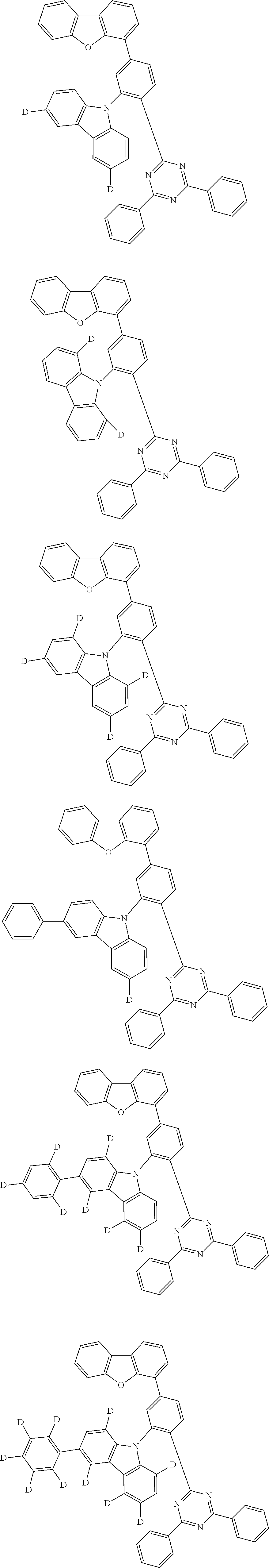

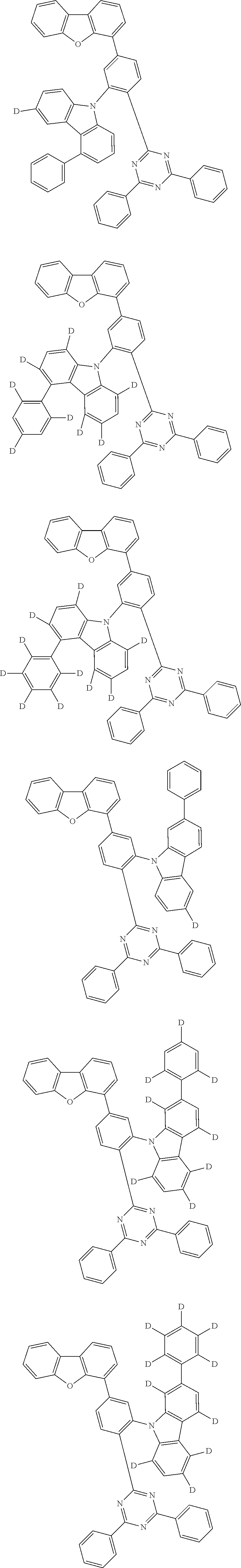

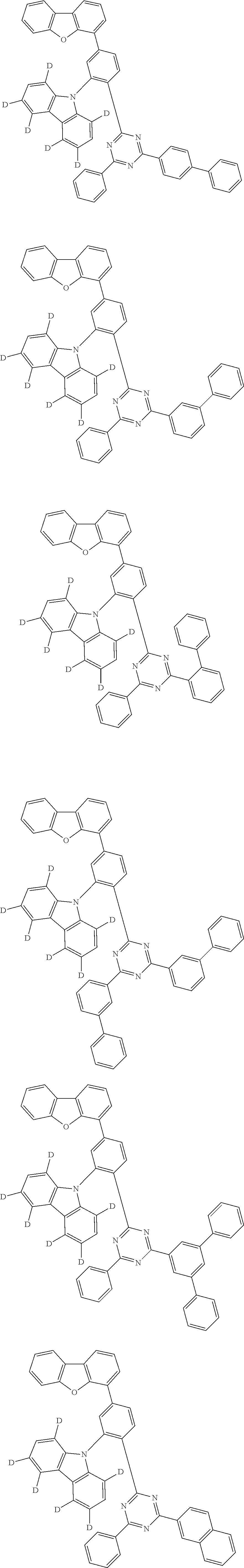

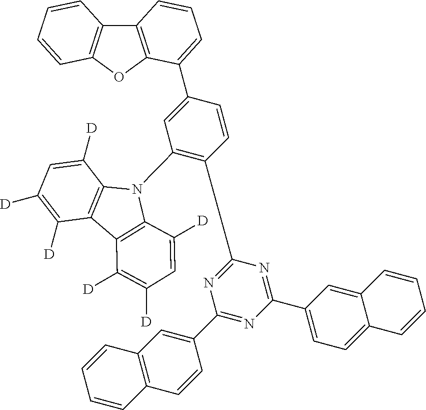 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccc3ccccc3c2)nc(-c2ccc3ccccc3c2)n1]

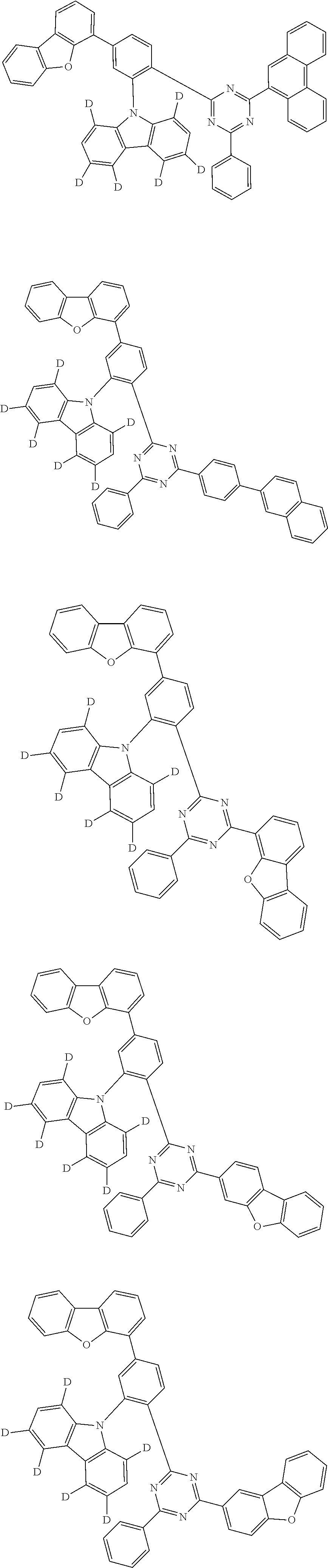

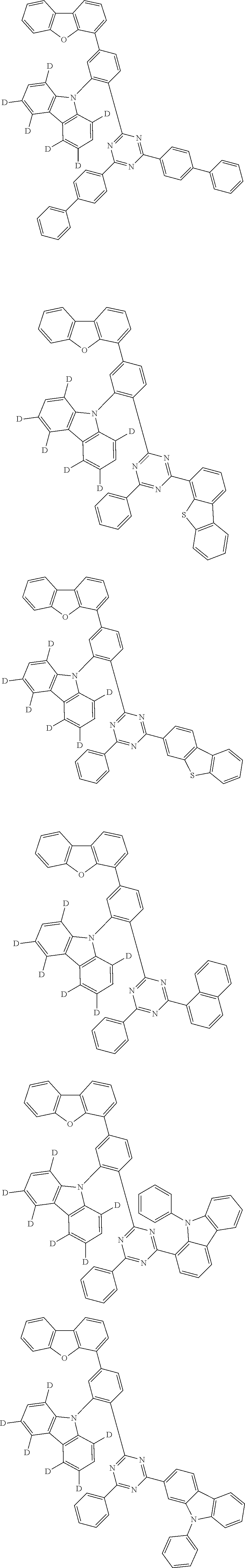

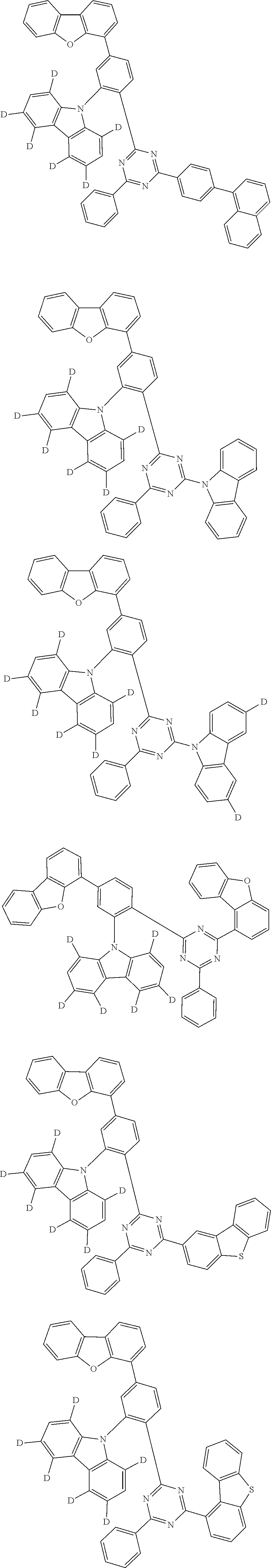

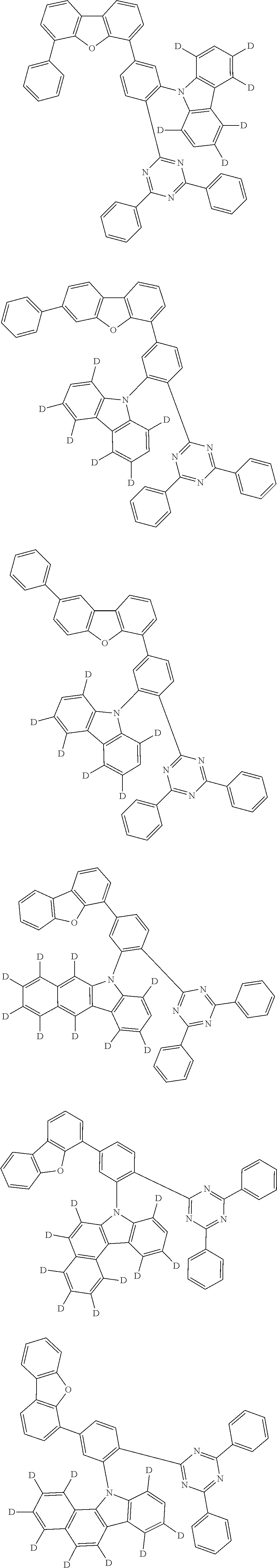

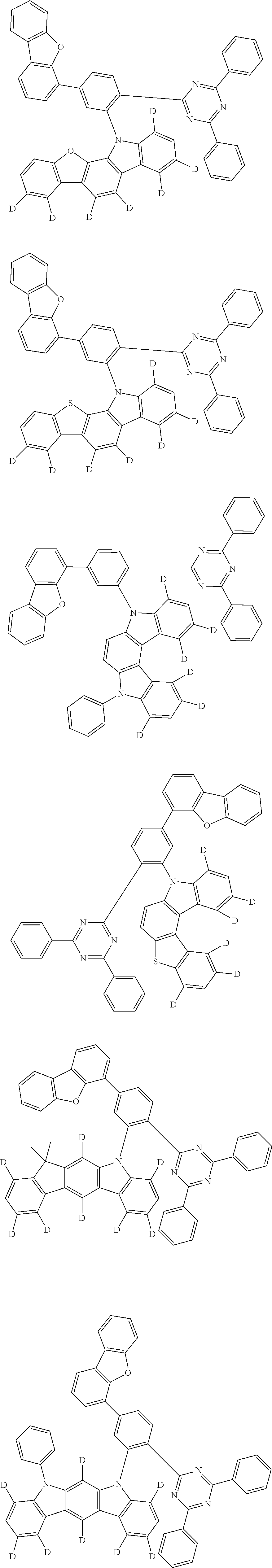

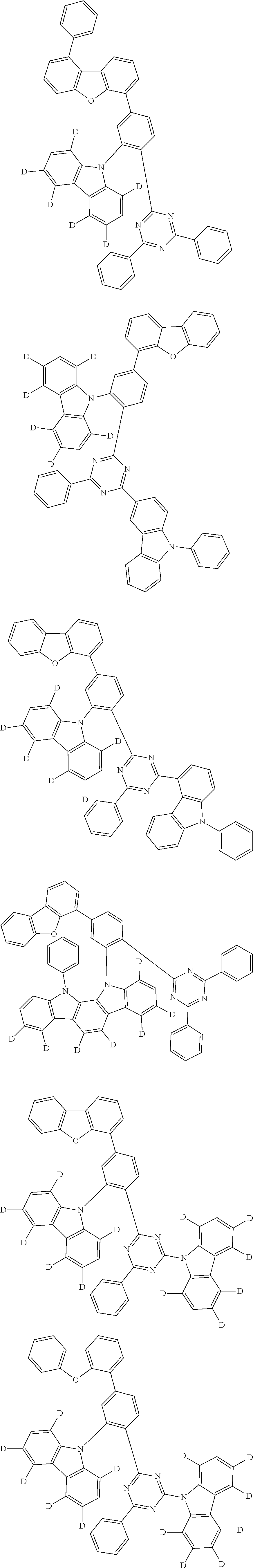

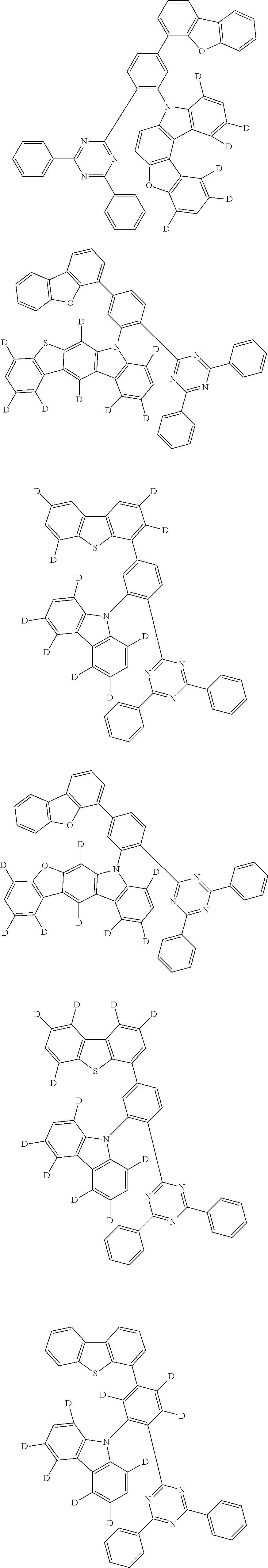

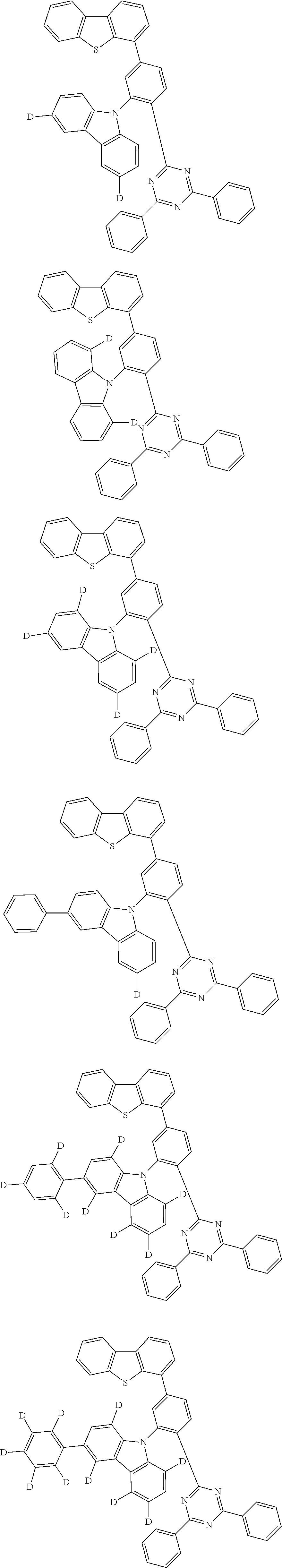

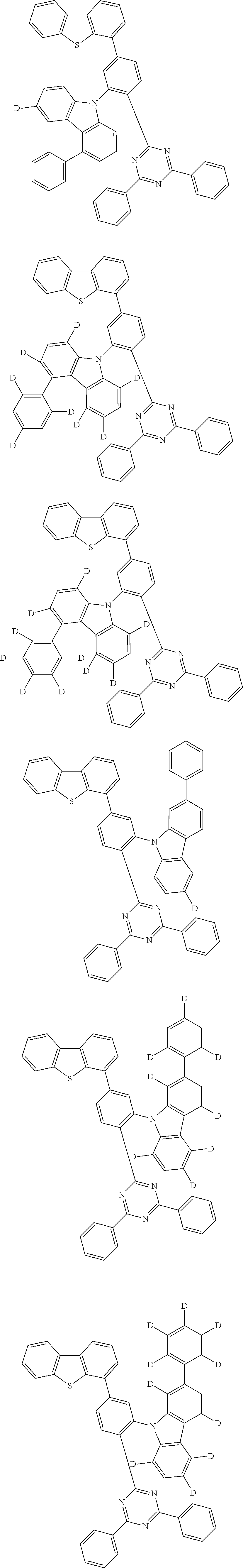

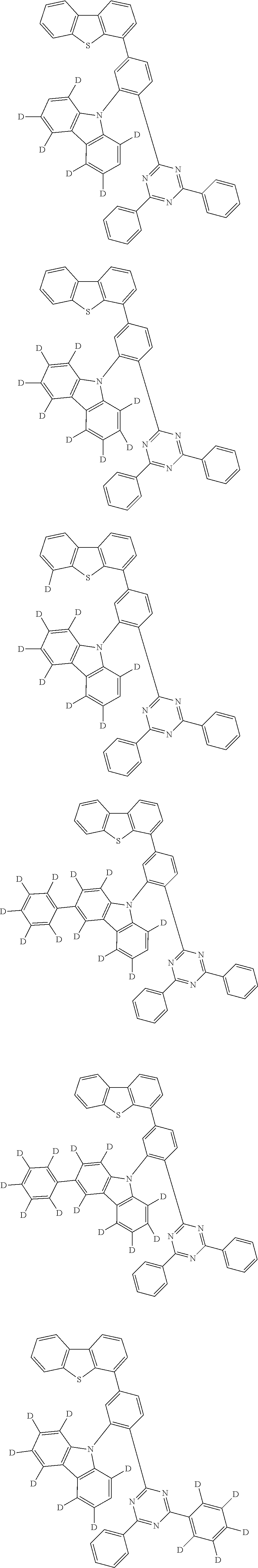

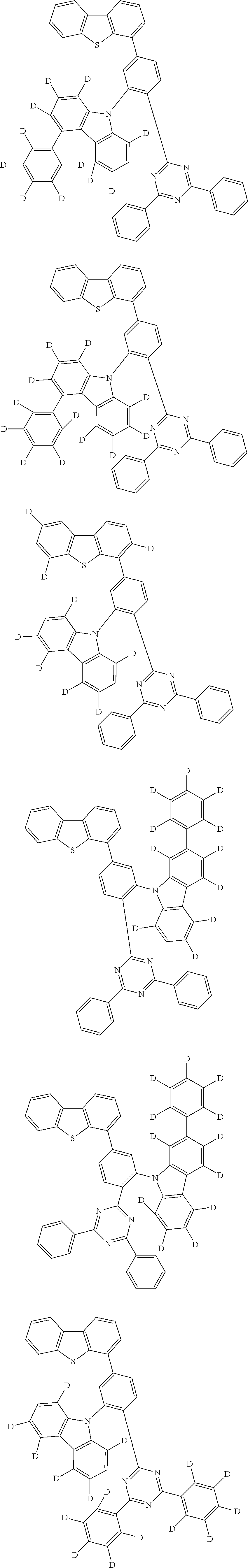

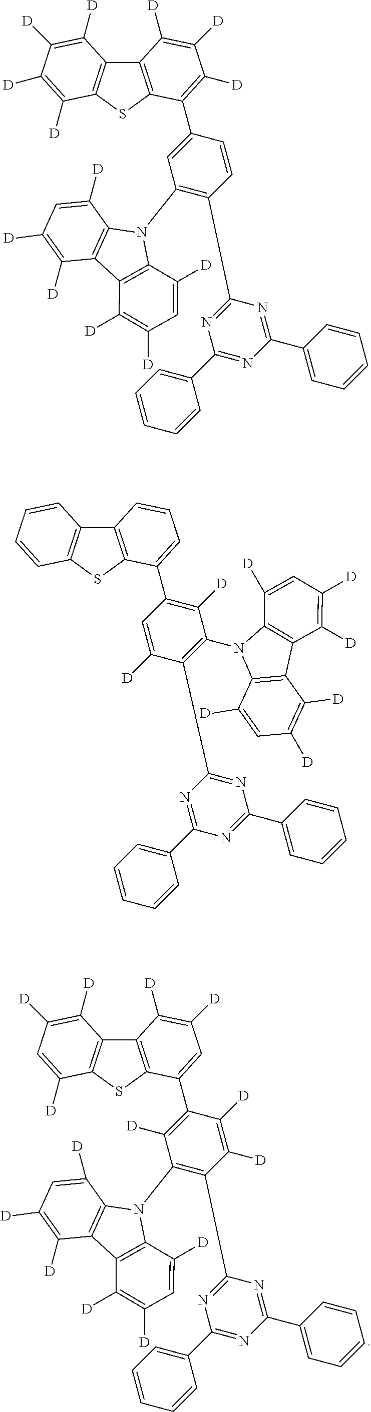 [SMILES: [2H]c1cc([2H])c2c(c1[2H])c1c([2H])c([2H])cc([2H])c1n2-c1cc(-c2c([2H])c([2H])c([2H])c3c2sc2c([2H])c([2H])c([2H])c([2H])c23)ccc1-c1nc(-c2ccccc2)nc(-c2ccccc2)n1 ; [2H]c1cc(-c2cccc3c2sc2ccccc23)c([2H])c(-n2c3c([2H])cc([2H])c([2H])c3c3c([2H])c([2H])cc([2H])c32)c1-c1nc(-c2ccccc2)nc(-c2ccccc2)n1 ; [2H]c1cc([2H])c2sc3c(-c4c([2H])c([2H])c(-c5nc(-c6ccccc6)nc(-c6ccccc6)n5)c(-n5c6c([2H])cc([2H])c([2H])c6c6c([2H])c([2H])cc([2H])c65)c4[2H])cc([2H])c([2H])c3c2c1[2H]]

Further, the above compound may not contain deuterium, or may contain at least one deuterium.

When the above compound contains deuterium, the deuterium substitution rate of the compound can be 1% to 100%. Specifically, the deuterium substitution rate of the compound can be 5% or more, 10% or more, 20% or more, 25% or more, 30% or more, 40% or more, or 50% or more, and 100% or less, 90% or less, 80% or less, or 70% or less.

In one example, the compound may not contain deuterium, or may contain 1 to 40 deuterium atoms. Specifically, when the compound contains deuterium, it can contain 1 or more, 3 or more, 5 or more, 7 or more, 10 or more, 12 or more, 15 or more, 18 or more, 20 or more, 22 or more, or 25 or more, and 40 or less, 38 or less, 36 or less, 34 or less, 32 or less, 30 or less, 28 or less, or 26 or less deuterium atoms.

Meanwhile, according to the present disclosure, provided is a method for preparing the compound of Chemical Formula 1 as shown in the following Reaction Scheme 1, 2, 3 or 4 as an example.

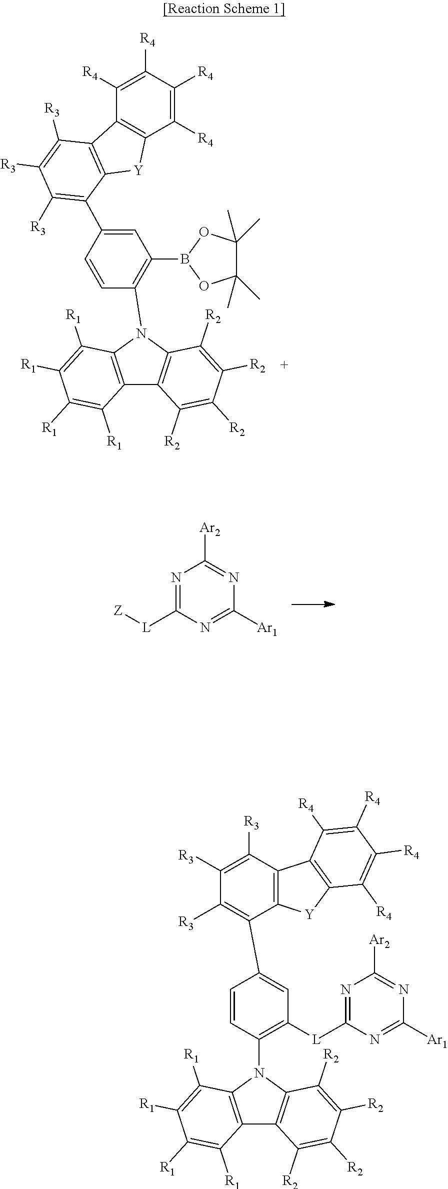

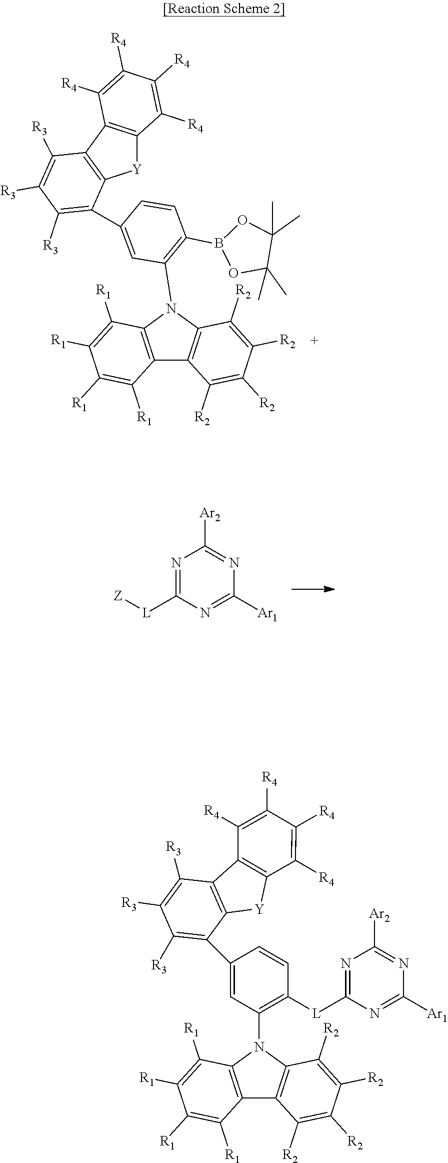

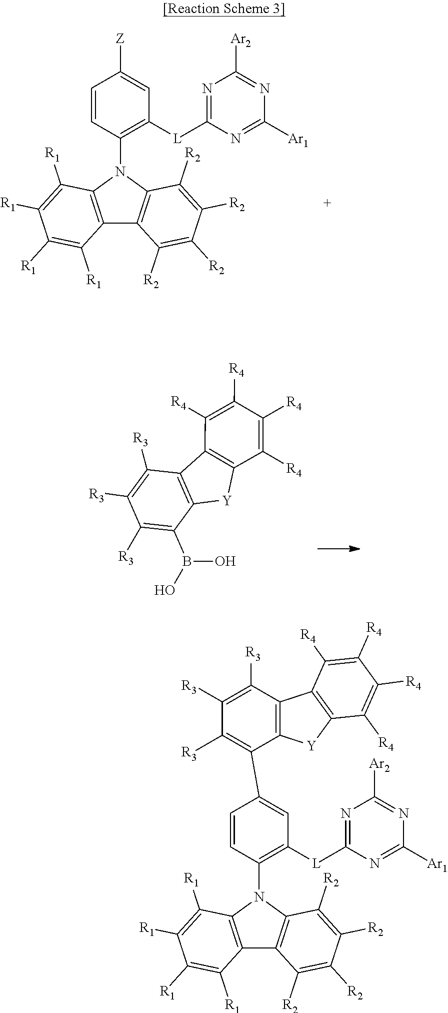

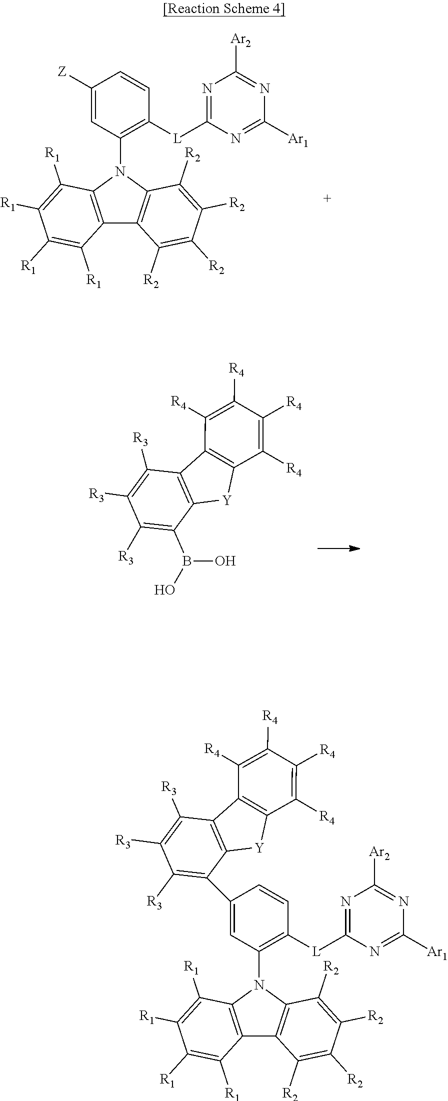

Reaction Scheme 1 or 2 is a reaction scheme in which Chemical Formula 2 is positioned at X₁ in Chemical Formula 1 and Chemical Formula 2 is positioned at X₂ in Chemical Formula 1, respectively. Reaction Scheme 3 or 4 is also a reaction scheme in which Chemical Formula 2 is positioned at X₁ in Chemical Formula 1 and Chemical Formula 2 is positioned at X₂ in Chemical Formula 1, respectively.

In Reaction Scheme 1, 2, 3 or 4, R₁, R₂, Ar₁, Ar₂, and L are as defined in Chemical Formula 1, and Y, R₃, and R₄ are as defined in Chemical Formula 2. Further, in Reaction Scheme 1, 2, 3 or 4, Z is a halogen, preferably chloro or bromo.

Reaction Scheme 1, 2, 3 or 4 can be performed by stirring and refluxing the reactants in tetrahydrofuran under a nitrogen atmosphere, then adding potassium carbonate and tetrakis(triphenylphosphine)palladium thereto, filtering the resulting solid and dissolving it in chloroform, then separating the organic layer, adding anhydrous magnesium sulfate thereto, stirring and filtering the mixture, and recrystallizing the result from ethyl acetate to obtain a solid compound.

### (Organic Light Emitting Device)

Further, according to the present disclosure, provided is an organic light emitting device comprising a compound of Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, a hole blocking layer, an electron transport layer, an electron injection layer and the like as the organic material layer. However, the structure of the organic light emitting device is not limited thereto, and it can include a smaller number of organic layers.

Further, the organic material layer can include a hole injection layer, a hole transport layer, or a layer that simultaneously performs hole injection and hole transport, wherein the hole injection layer, the hole transport layer, or the layer that simultaneously performs hole injection and hole transport can include the compound of Chemical Formula 1.

Further, the organic material layer can include a light emitting layer, wherein the light emitting layer can include the compound of Chemical Formula 1.

Further, the organic material layer can include a hole blocking layer, an electron transport layer, an electron injection layer, or a layer that simultaneously performs electron transport and electron injection, wherein the hole blocking layer, the electron transport layer, the electron injection layer, or the layer that simultaneously performs electron transport and electron injection can include the compound of Chemical Formula 1.

Further, the organic material layer can include a light emitting layer, and an electron injection and transport layer, wherein the electron injection and transport layer can include the compound of Chemical Formula 1.

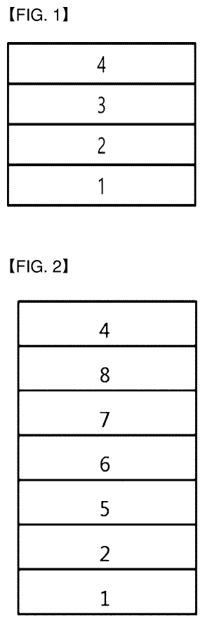

Further, the organic light emitting device according to the present disclosure can be a normal type organic light emitting device in which an anode, one or more organic material layers, and a cathode are sequentially stacked on a substrate. Further, the organic light emitting device according to the present disclosure can be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 7, an electron injection and transport layer 8, and a cathode 4. In such a structure, the compound of Chemical Formula 1 can be included in the light emitting layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking an anode, an organic material layer and a cathode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrile-hexaazatriphenylene-based organic material, a quinacridone-based organic material, a perylene-based organic material, anthraquinone, polyaniline and polythiophene-based conductive compound, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive compound, a block copolymer in which a conjugate portion and a non-conjugate portion are present together, and the like, but are not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

The electron blocking layer means a layer provided between the hole transport layer and the light emitting layer in order to prevent the electrons injected in the cathode from being transferred to the hole transport layer without being recombined in the light emitting layer, which can also be referred to as an electron inhibition layer. The electron blocking layer is preferably a material having the smaller electron affinity than the electron transport layer. Preferably, the compound of Chemical Formula 1 can be included as a material of the electron blocking layer.

The light emitting layer can include a host material and a dopant material. The compound of Chemical Formula 1 can be used as the host material. Further, the host material that can be additionally used includes a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material is suitably a material which can receive electrons well from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxy-quinolinato lithium, bis(8-hydroxyquinolinato)zinc, bis(8-hydroxyquinolinato)-copper, bis(8-hydroxyquinolinato)manganese, tris(8-hydroxyquinolinato)-aluminum, tris(2-methyl-8-hydroxyquinolinato)aluminum, tris(8-hydroxy-quinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

On the other hand, in the present disclosure, the “electron injection and transport layer” is a layer that performs both the roles of the electron injection layer and the electron transport layer, and the materials that perform the roles of each layer can be used alone or in combination, but is not limited thereto. Preferably, the compound of Chemical Formula 1 can be included as a material for the electron injection and transport layer.

The organic light emitting device according to the present disclosure can be a bottom emission device, a top emission device, or a double-sided light emitting device, and particularly, can be a bottom emission device that requires relatively high luminous efficiency.

In addition, the compound according to the present disclosure can be included in an organic solar cell or an organic transistor in addition to an organic light emitting device.

The preparation of the compound of Chemical Formula 1 and the organic light emitting device including the same will be described in detail in the following examples. However, these examples are presented for illustrative purposes only, and are not intended to limit the scope of the present disclosure.

### [Synthesis Example 1] Synthesis of Compound A-3

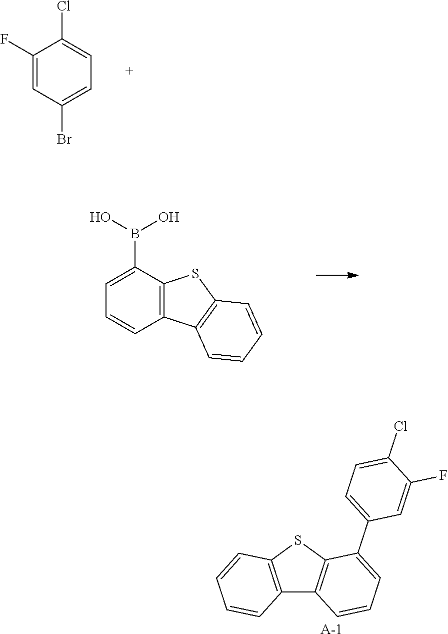

4-Bromo-1-chloro-2-fluorobenzene (100 g, 481 mmol) and dibenzo[b,d]thiophen-4-ylboronic acid (109.7 g, 481 mmol) were added to 2000 ml of tetrahydrofuran under a nitrogen atmosphere, and the mixture was stirred and refluxed. Then, potassium carbonate (199.4 g, 1442.9 mmol) was dissolved in 199 ml of water and added thereto, and the mixture was sufficiently stirred, and then tetrakis(triphenylphosphine)palladium (16.7 g, 14.4 mmol) was added. After reacting for 3 hours, the reaction mixture was cooled to room temperature, and then the organic layer and the aqueous layer were separated, and the organic layer was distilled. This was again dissolved in 2848 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 95.4 g of Compound A-1 as a grey solid (Yield: 67%, MS: [M+H]⁺=297).

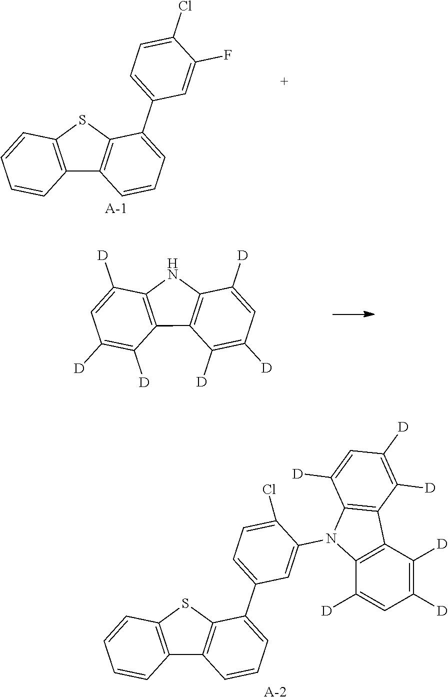

A-1 (100 g, 320.5 mmol) and 9H-carbazole-1,3,4,5,6,8-d6 (55.5 g, 320.5 mmol) were added to 2000 ml of xylene under a nitrogen atmosphere, and the mixture was stirred and refluxed. Then, sodium tert-butoxide (92.4 g, 961.5 mmol) was added thereto, sufficiently stirred, and then bis(tri-tert-butylphosphine)palladium(0) (4.9 g, 9.6 mmol) was added. After reacting for 4 hours, the reaction mixture was cooled to room temperature, and then the resulting solid was filtered. The solid was added to and dissolved in 4472 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 96.9 g of Compound A-2 as a white solid (Yield: 65%, MS: [M+H]⁺=466.1).

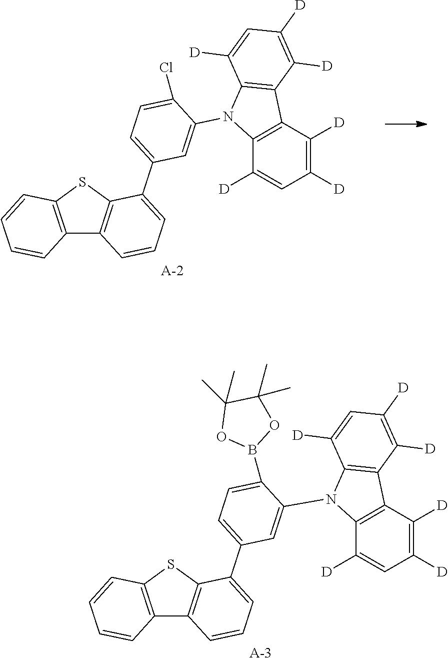

A-2 (50 g, 107.5 mmol) and bis(pinacolato)diboron (30.1 g, 118.2 mmol) were added to 1000 ml of Diox under a nitrogen atmosphere, and the mixture was stirred and refluxed. Then, potassium acetate (31 g, 322.5 mmol) was added thereto, sufficiently stirred, and then palladiumdibenzylideneacetone-palladium (1.9 g, 3.2 mmol) and tricyclohexylphosphine (1.8 g, 6.4 mmol) were added. After reacting for 4 hours, the reaction mixture was cooled to room temperature, and then the organic layer was subjected to filtration treatment to remove a salt, and then the filtered organic layer was distilled. This was added again to and dissolved in 599 ml of chloroform, and washed twice with water. The organic layer was then separated, to which anhydrous magnesium sulfate was added, stirred, then filtered, and the filtrate was distilled under reduced pressure. The concentrated compound was recrystallized from chloroform and ethanol to give 52.7 g of Compound A-3 as a grey solid (Yield: 88%, MS: [M+H]⁺=558.3).

### [Synthesis Example 2] Synthesis of Compound B-3

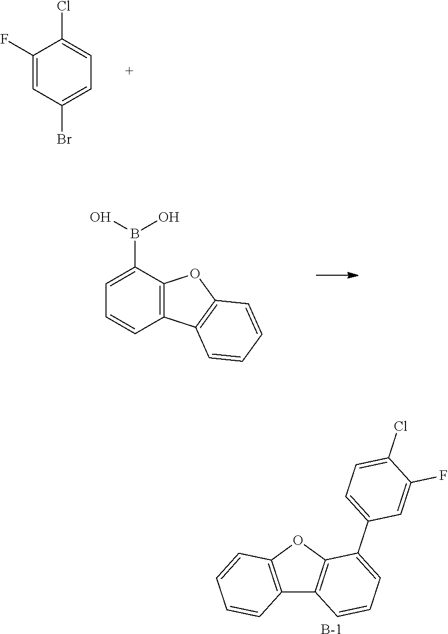

4-Bromo-1-chloro-2-fluorobenzene (100 g, 481 mmol) and dibenzo[b,d]furan-4-ylboronic acid (102 g, 481 mmol) were added to 2000 ml of tetrahydrofuran under a nitrogen atmosphere, and the mixture was stirred and refluxed. Then, potassium carbonate (199.4 g, 1442.9 mmol) was dissolved in 199 ml of water and added thereto, and the mixture was sufficiently stirred, and then tetrakis(triphenylphosphine)palladium (16.7 g, 14.4 mmol) was added. After reacting for 1 hour, the reaction mixture was cooled to room temperature, and then the organic layer and the aqueous layer were separated, and the organic layer was distilled. This was again dissolved in 2030 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 55.8 g of Compound B-1 as a grey solid (Yield: 55%, MS: [M+H]⁺=212.1).

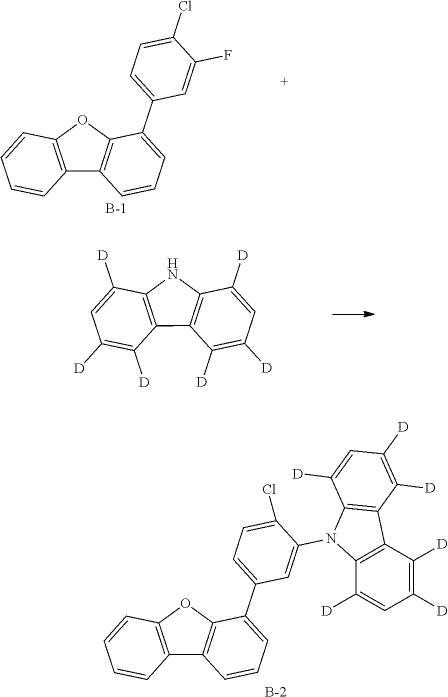

B-1 (100 g, 473.8 mmol) and 9H-carbazole-1,3,4,5,6,8-d6 (82 g, 473.8 mmol) were added to 2000 ml of xylene under a nitrogen atmosphere, and the mixture was stirred and refluxed. Then, sodium tert-butoxide (136.6 g, 1421.4 mmol) was added thereto, sufficiently stirred, and then bis(tri-tert-butylphosphine)palladium(0) (7.3 g, 14.2 mmol) was added. After reacting for 3 hours, the reaction mixture was cooled to room temperature, and then the resulting solid was filtered. The solid was added to and dissolved in 6384 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 108.5 g of Compound B-2 as a white solid (Yield: 51%, MS: [M+H]⁺=450.2).

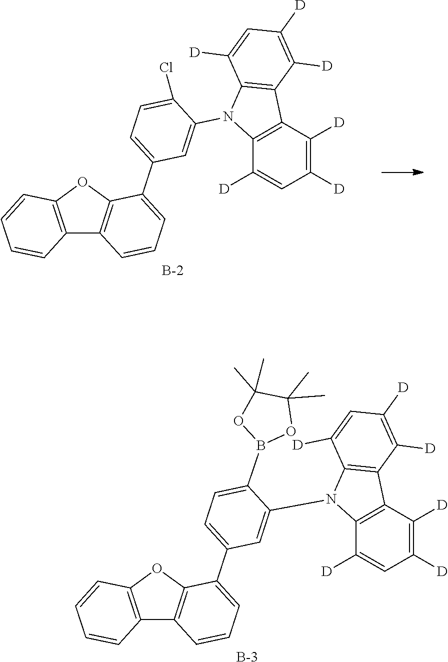

B-2 (50 g, 107.5 mmol) and bis(pinacolato)diboron (30.1 g, 118.2 mmol) were added to 1000 ml of Diox under a nitrogen atmosphere, and the mixture was stirred and refluxed. Then, potassium acetate (31 g, 322.5 mmol) was added thereto, sufficiently stirred, and then palladium-dibenzylideneacetonepalladium (1.9 g, 3.2 mmol) and tricyclohexylphosphine (1.8 g, 6.4 mmol) were added. After reacting for 5 hours, the reaction mixture was cooled to room temperature, and then the organic layer was subjected to filtration treatment to remove a salt, and then the filtered organic layer was distilled. This was added again to and dissolved in 599 ml of chloroform, and washed twice with water. The organic layer was then separated, to which anhydrous magnesium sulfate was added, stirred, then filtered, and the filtrate was distilled under reduced pressure. The concentrated compound was recrystallized from chloroform and ethanol to give 53.9 g of Compound B-3 as a grey solid (Yield: 90%, MS: [M+H]⁺=558.3),

### [Synthesis Example 3] Synthesis of Compound C-2

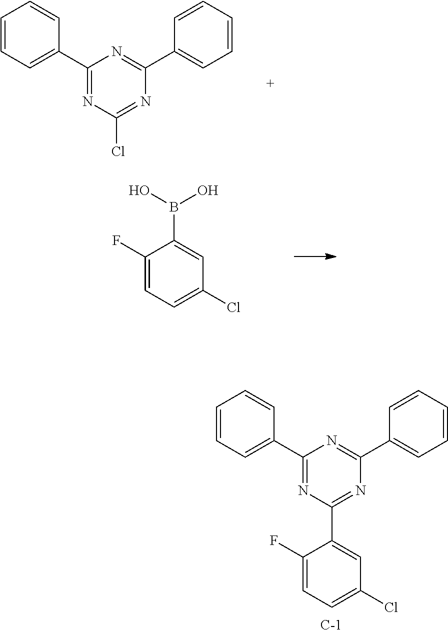

2-Chloro-4,6-diphenyl-1,3,5-triazine (100 g, 374.4 mmol) and (5-chloro-2-fluorophenyl)boronic acid (65.2 g, 374.4 mmol) were added to 2000 ml of tetrahydrofuran under a nitrogen atmosphere, and the mixture was stirred and refluxed. Then, potassium carbonate (155.3 g, 1123.3 mmol) was dissolved in 155 ml of water and added thereto, and the mixture was sufficiently stirred, and then tetrakis(triphenylphosphine)palladium (13 g, 11.2 mmol) was added. After reacting for 1 hour, the reaction mixture was cooled to room temperature, and then the organic layer and the aqueous layer were separated, and the organic layer was distilled. This was again dissolved in 2704 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 90.6 g of Compound C-1 as a grey solid (Yield: 67%, MS: [M+H]⁺=362.1).

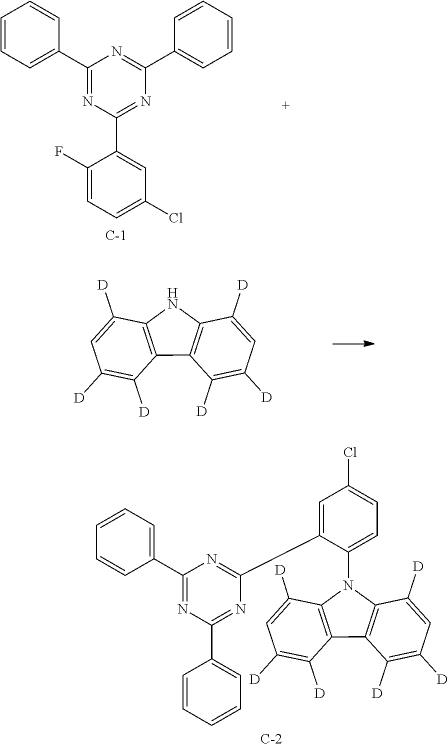

C-1 (100 g, 276.9 mmol) and 9H-carbazole-1,3,4,5,6,8-d6 (47.9 g, 276.9 mmol) were added to 2000 ml of xylene under a nitrogen atmosphere, and the mixture was stirred and refluxed. Then, sodium tert-butoxide (79.9 g, 830.8 mmol) was added thereto, sufficiently stirred, and then bis(tri-tert-butylphosphine)palladium(0) (4.2 g, 8.3 mmol) was added. After reacting for 3 hours, the reaction mixture was cooled to room temperature, and then the resulting solid was filtered. The solid was added to and dissolved in 4272 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 71.2 g of Compound C-2 as a white solid (Yield: 50%, MS: [M+H]⁺=515.2).

### [Synthesis Example 4] Synthesis of Compound D-1

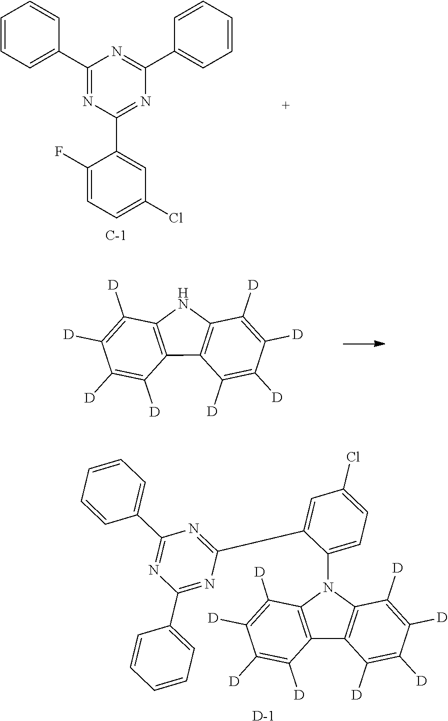

C-1 (100 g, 276.9 mmol) and 9H-carbazole-1,2,3,4,5,6,7,8-d8 (48.5 g, 276.9 mmol) were added to 2000 ml of xylene under a nitrogen atmosphere, and the mixture was stirred and refluxed. Then, sodium tert-butoxide (79.9 g, 830.8 mmol) was added thereto, sufficiently stirred, and then bis(tri-tert-butylphosphine)palladium(0) (4.2 g, 8.3 mmol) was added. After reacting for 2 hours, the reaction mixture was cooled to room temperature, and then the resulting solid was filtered. The solid was added to and dissolved in 4289 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 94.4 g of Compound D-1 as a white solid (Yield: 66%, MS: [M+H]⁺=517.2).

### [Synthesis Example 5] Synthesis of Compound E-1

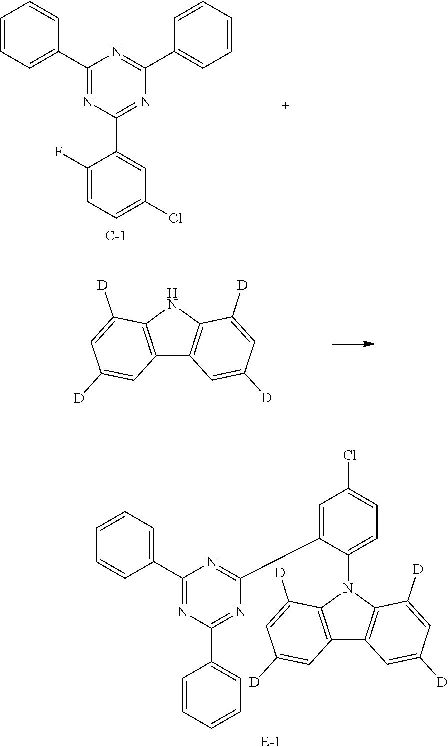

C-1 (100 g, 276.9 mmol) and 9H-carbazole-1,3,6,8-d4 (47.4 g, 276.9 mmol) were added to 2000 ml of xylene under a nitrogen atmosphere, and the mixture was stirred and refluxed. Then, sodium tert-butoxide (79.9 g, 830.8 mmol) was added thereto, sufficiently stirred, and then bis(tri-tert-butylphosphine)palladium(0) (4.2 g, 8.3 mmol) was added. After reacting for 4 hours, the reaction mixture was cooled to room temperature, and then the resulting solid was filtered. The solid was added to and dissolved in 4255 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 103.5 g of Compound E-1 as a white solid (Yield: 73%, MS: [M+H]⁺=513.2).

### [Synthesis Example 6] Synthesis of Compound F-1

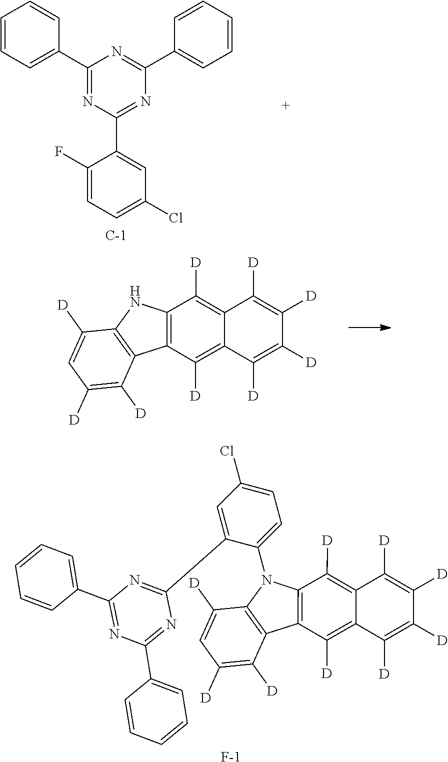

C-1 (100 g, 276.9 mmol) and 5H-benzo[b]carbazole-1,2,4,6,7,8,9,10,11-d9 (62.6 g, 276.9 mmol) were added to 2000 ml of xylene under a nitrogen atmosphere, and the mixture was stirred and refluxed. Then, sodium tert-butoxide (79.9 g, 830.8 mmol) was added thereto, sufficiently stirred, and then bis(tri-tert-butylphosphine)palladium(0) (4.2 g, 8.3 mmol) wase added. After reacting for 2 hours, the reaction mixture was cooled to room temperature, and then the resulting solid was filtered. The solid was added to and dissolved in 4713 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 121 g of Compound F-1 as a white solid (Yield: 77%, MS: [M+H]⁺=568.2).

### [Synthesis Example 7] Synthesis of Compound G-1

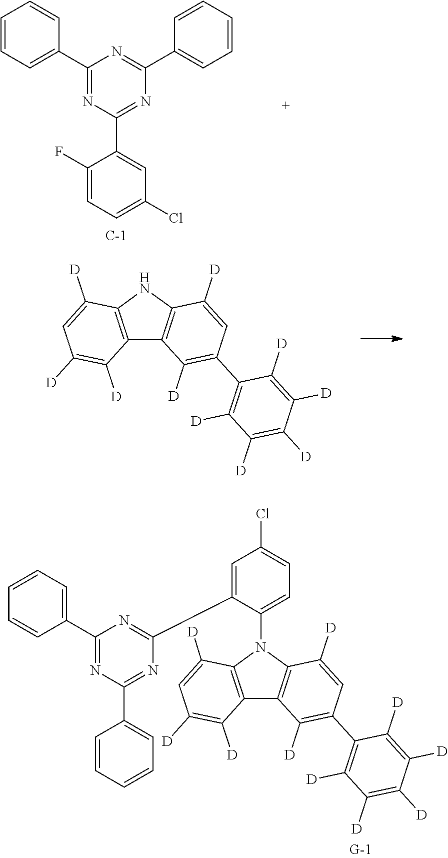

C-1 (100 g, 473.8 mmol) and 3-(phenyl-d5)-9H-carbazole-1,4,5,6,8-d5 (120 g, 473.8 mmol) were added to 2000 ml of xylene under a nitrogen atmosphere, and the mixture was stirred and refluxed. Then, sodium tert-butoxide (136.6 g, 1421.4 mmol) was added thereto, sufficiently stirred, and then bis(tri-tert-butylphosphine)palladium(0) (7.3 g, 14.2 mmol) was added. After reacting for 4 hours, the reaction mixture was cooled to room temperature, and then the resulting solid was filtered. The solid was added to and dissolved in 8447 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 180.2 g of Compound G-1 as a white solid (Yield: 64%, MS: [M+H]⁺=595.2).

### [Synthesis Example 8] Synthesis of Compound H-1

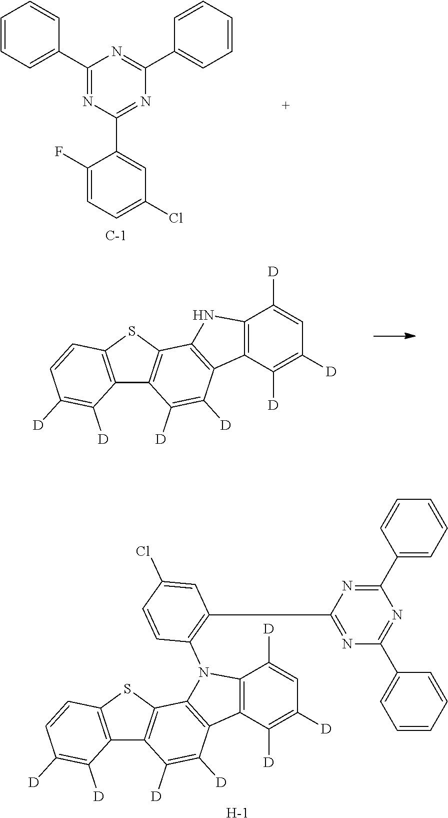

C-1 (100 g, 276.9 mmol) and 12H-benzo[4,5]thieno[2,3-a]carbazole-1,3,4,5,6,7,8-d7 (77.6 g, 276.9 mmol) were added to 2000 ml of xylene under a nitrogen atmosphere, and the mixture was stirred and refluxed. Then, sodium tert-butoxide (79.9 g, 830.8 mmol) was added thereto, sufficiently stirred, and then bis(tri-tert-butylphosphine)palladium(0) (4.2 g, 8.3 mmol) was added. After reacting for 1 hour, the reaction mixture was cooled to room temperature, and then the resulting solid was filtered. The solid was added to and dissolved in 5161 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 129 g of Compound H-1 as a white solid (Yield: 75%, MS: [M+H]⁺=622.2).

### [Synthesis Example 9] Synthesis of Compound I-3

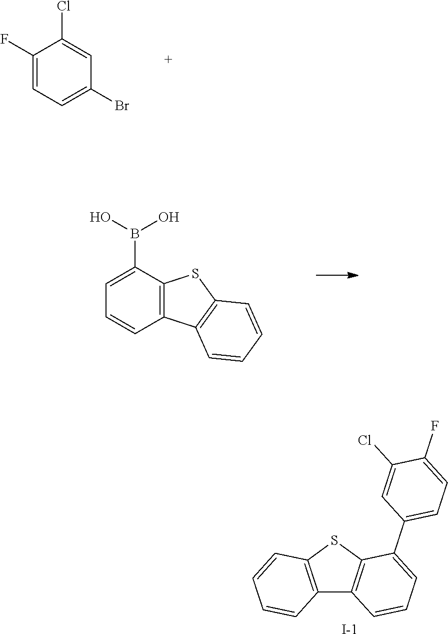 [SMILES: Fc1ccc(Br)cc1Cl ; OB(O)c1cccc2c1sc1ccccc12 ; Fc1ccc(-c2cccc3c2sc2ccccc23)cc1Cl ; [I-]]

5-Bromo-1-chloro-2-fluorobenzene (100 g, 481 mmol) and dibenzo[b,d]thiophen-4-ylboronic acid (109.7 g, 481 mmol) were added to 2000 ml of tetrahydrofuran under a nitrogen atmosphere, and the mixture was stirred and refluxed. Then, potassium carbonate (199.4 g, 1442.9 mmol) was dissolved in 199 ml of water and added thereto, and the mixture was sufficiently stirred, and then tetrakis(triphenylphosphine)palladium (16.7 g, 14.4 mmol) was added. After reacting for 3 hours, the reaction mixture was cooled to room temperature, and then the organic layer and the aqueous layer were separated, and the organic layer was distilled. This was again dissolved in 2848 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 101.1 g of Compound I-1 (Yield: 71%, MS: [M+H]⁺=297).

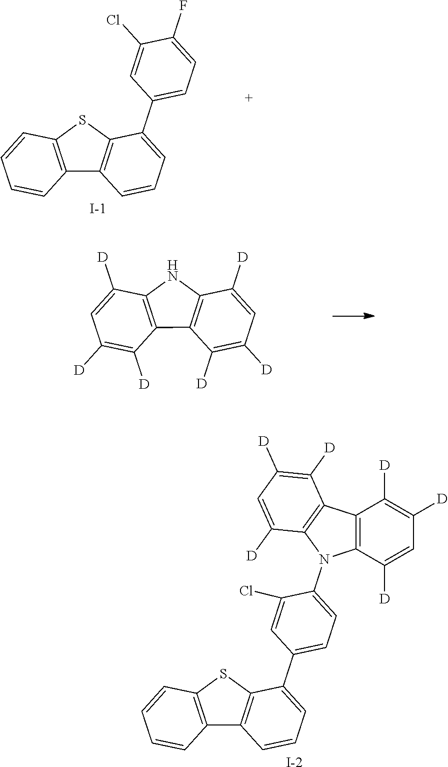

I-1 (100 g, 337.8 mmol) and 9H-carbazole-1,3,4,5,6,8-d6 (77 g, 337.8 mmol) were added to 2000 ml of xylene under a nitrogen atmosphere, and the mixture was stirred and refluxed. Then, sodium tert-butoxide (97.4 g, 1013.4 mmol) was added thereto, sufficiently stirred, and then bis(tri-tert-butylphosphine)palladium(0) (5.2 g, 10.1 mmol) was added. After reacting for 4 hours, the reaction mixture was cooled to room temperature, and then the resulting solid was filtered. The solid was added to and dissolved in 4713 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 97.4 g of Compound I-2 as a white solid (Yield: 62%, MS: [M+H]⁺=466.1).

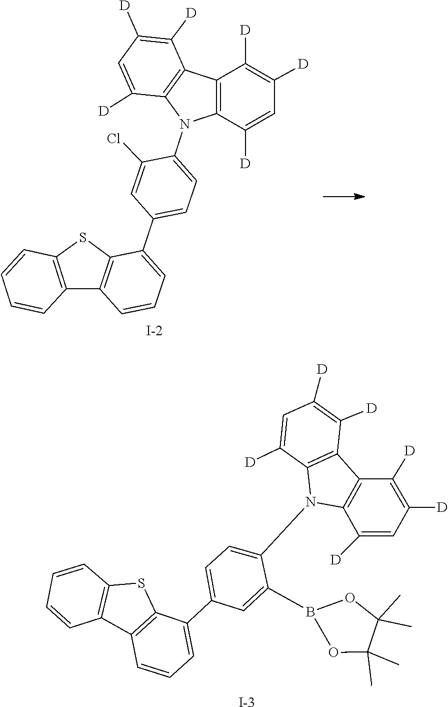

I-2 (50 g, 107.5 mmol) and bis(pinacolato)diboron (30.1 g, 118.2 mmol) were added to 1000 ml of Diox under a nitrogen atmosphere, and the mixture was stirred and refluxed. Then, potassium acetate (31 g, 322.5 mmol) was added thereto, sufficiently stirred, and then palladiumdibenzylideneacetone-palladium (1.9 g, 3.2 mmol) and tricyclohexylphosphine (1.8 g, 6.4 mmol) were added. After reacting for 3 hours, the reaction mixture was cooled to room temperature, and then the organic layer was subjected to filtration treatment to remove a salt, and then the filtered organic layer was distilled. This was added again to and dissolved in 599 ml of chloroform, and washed twice with water. The organic layer was then separated, to which anhydrous magnesium sulfate was added, stirred, then filtered, and the filtrate was distilled under reduced pressure. The concentrated compound was recrystallized from chloroform and ethanol to give 52.1 g of Compound I-3 as a grey solid (Yield: 87%, MS: [M+H]⁺=558.3).

### [Synthesis Example 10] Synthesis of Compound J-3

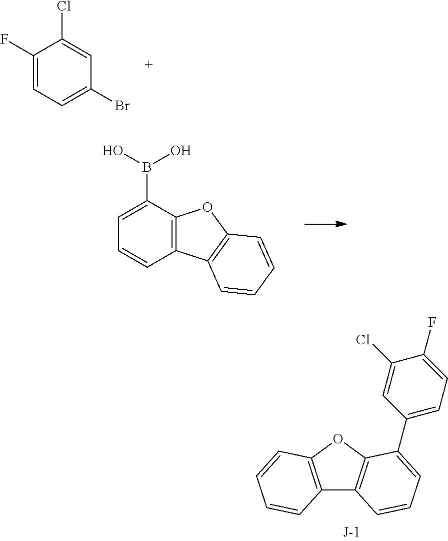

5-Bromo-1-chloro-2-fluorobenzene (100 g, 481 mmol) and dibenzo[b,d]furan-4-ylboronic acid (102 g, 481 mmol) were added to 2000 ml of tetrahydrofuran under a nitrogen atmosphere, and the mixture was stirred and refluxed. Then, potassium carbonate (199.4 g, 1442.9 mmol) was dissolved in 199 ml of water and added thereto, and the mixture was sufficiently stirred, and then tetrakis(triphenylphosphine)-palladium (16.7 g, 14.4 mmol) was added. After reacting for 2 hours, the reaction mixture was cooled to room temperature, and then the organic layer and the aqueous layer were separated, and the organic layer was distilled. This was again dissolved in 2030 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 55.8 g of Compound J-1 as a grey solid (Yield: 55%, MS: [M+H]⁺=212.1).

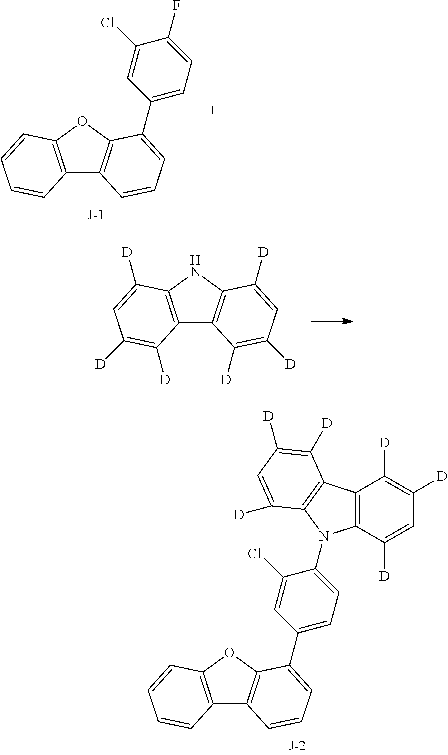 [SMILES: Fc1ccc(-c2cccc3c2oc2ccccc23)cc1Cl ; [2H]c1cc([2H])c2[nH]c3c([2H])cc([2H])c([2H])c3c2c1[2H] ; [2H]c1cc([2H])c2c(c1[2H])c1c([2H])c([2H])cc([2H])c1n2-c1ccc(-c2cccc3c2oc2ccccc23)cc1Cl]

J-1 (100 g, 473.8 mmol) and 9H-carbazole-1,3,4,5,6,8-d6 (82 g, 473.8 mmol) were added to 2000 ml of xylene under a nitrogen atmosphere, and the mixture was stirred and refluxed. Then, sodium tert-butoxide (136.6 g, 1421.4 mmol) was added thereto, sufficiently stirred, and then bis(tri-tert-butylphosphine)palladium(0) (7.3 g, 14.2 mmol) was added. After reacting for 3 hours, the reaction mixture was cooled to room temperature, and then the resulting solid was filtered. The solid was added to and dissolved in 6384 mL of chloroform, washed twice with water, and the organic layer was separated, to which anhydrous magnesium sulfate was added, stirred, then filtered, and the filtrate was distilled under reduced pressure. The concentrated compound was purified by a silica gel column chromatography using chloroform and ethyl acetate to give 117 g of Compound J-2 as a white solid (Yield: 55%, MS: [M+H]⁺=450.2).

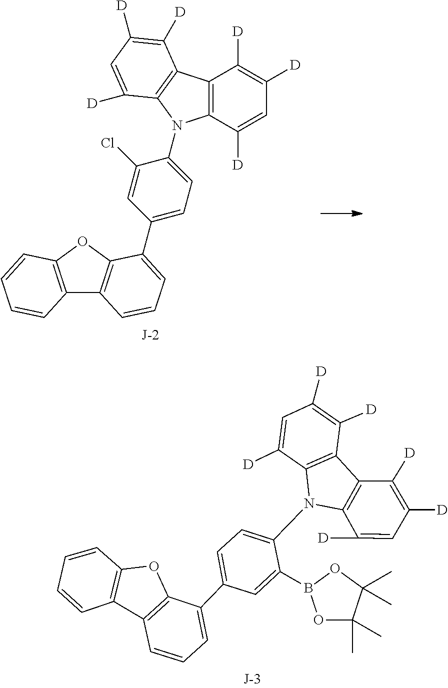 [SMILES: [2H]c1cc([2H])c2c(c1[2H])c1c([2H])c([2H])cc([2H])c1n2-c1ccc(-c2cccc3c2oc2ccccc23)cc1Cl ; [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1]

J-2 (50 g, 111.3 mmol) and bis(pinacolato)diboron (31.1 g, 122.5 mmol) were added to 1000 ml of Diox under a nitrogen atmosphere, and the mixture was stirred and refluxed. Then, potassium acetate (32.1 g, 334 mmol) was added thereto, sufficiently stirred, and then palladiumdibenzylideneacetone-palladium (1.9 g, 3.3 mmol) and tricyclohexylphosphine (1.9 g, 6.7 mmol) were added. After reacting for 3 hours, the reaction mixture was cooled to room temperature, and then the organic layer was subjected to filtration treatment to remove a salt, and then the filtered organic layer was distilled. This was added again to and dissolved in 603 ml of chloroform, and washed twice with water. The organic layer was then separated, to which anhydrous magnesium sulfate was added, stirred, then filtered, and the filtrate was distilled under reduced pressure. The concentrated compound was recrystallized from chloroform and ethanol to give 39.2 g of Compound J-3 as a grey solid (Yield: 65%, MS: [M+H]⁺=542.3).

### [Preparation Example 1] Synthesis of Compound 1

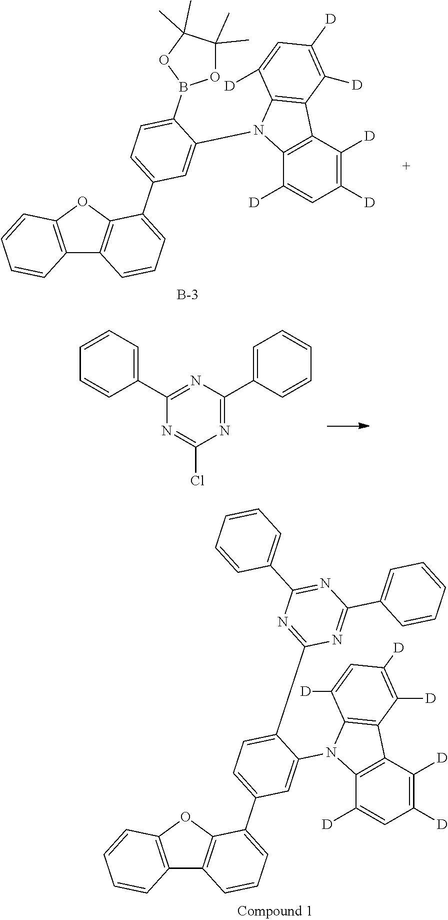

B-3 (20 g, 37 mmol) and 2-chloro-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1194 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 13.1 g of Compound 1 as a white solid (Yield: 55%, MS: [M+H]⁺=647.3).

### [Preparation Example 2] Synthesis of Compound 2

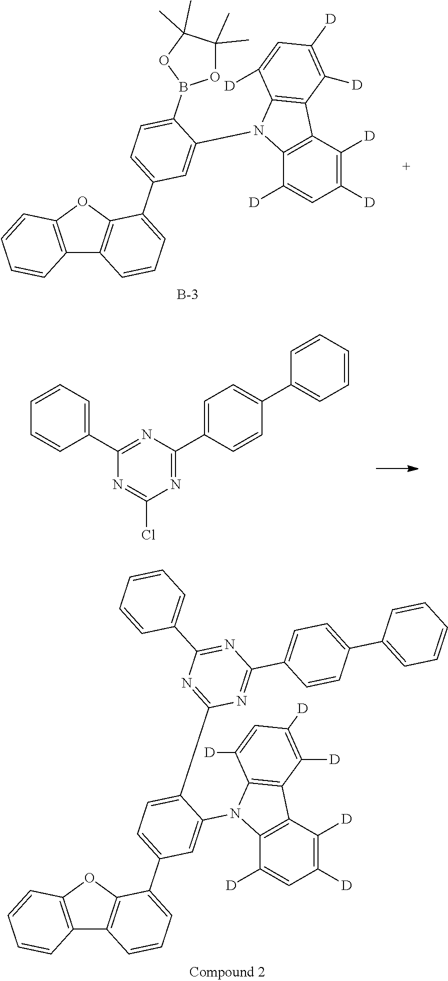

B-3 (20 g, 37 mmol) and 2-([1,1′-biphenyl]-4-yl)-4-chloro-6-phenyl-1,3,5-triazine (12.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 2 hours, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15.2 g of Compound 2 as a white solid (Yield: 57%, MS: [M+H]⁺=723.3).

### [Preparation Example 3] Synthesis of Compound 3

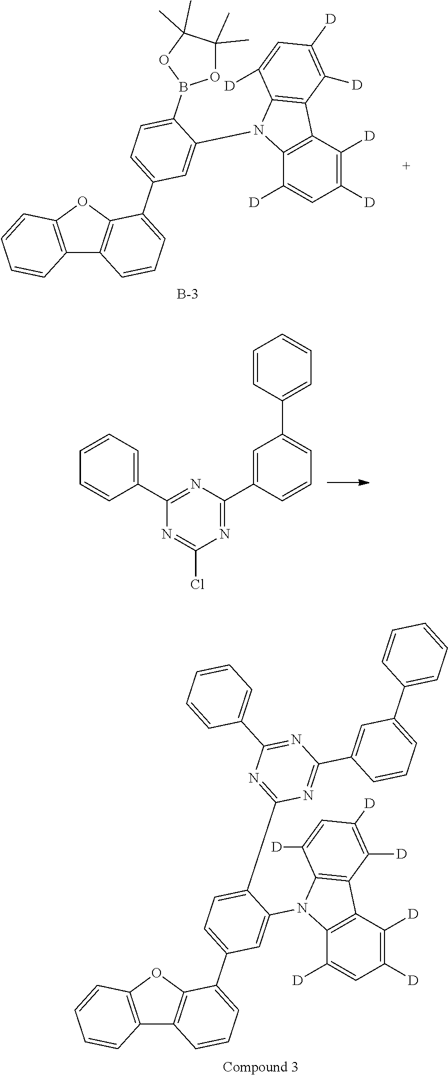

B-3 (20 g, 37 mmol) and 2-([1,1′-biphenyl]-3-yl)-4-chloro-6-phenyl-1,3,5-triazine (12.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 2 hours, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.7 g of Compound 3 as a white solid (Yield: 55%, MS: [M+H]⁺=723.3).

### [Preparation Example 4] Synthesis of Compound 4

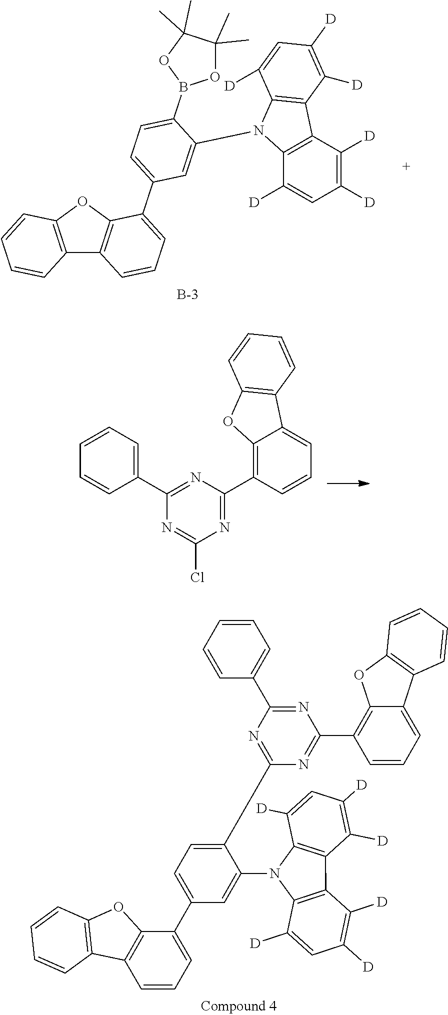

B-3 (20 g, 37 mmol) and 2-chloro-4-(dibenzo[b,d]furan-4-yl)-6-phenyl-1,3,5-triazine (13.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 2 hours, the reaction mixture was cooled to room temperature, and then the resulting solid was filtered. The solid was added to and dissolved in 1360 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18 g of Compound 4 as a white solid (Yield: 66%, MS: [M+H]⁺=737.3).

### [Preparation Example 5] Synthesis of Compound 5

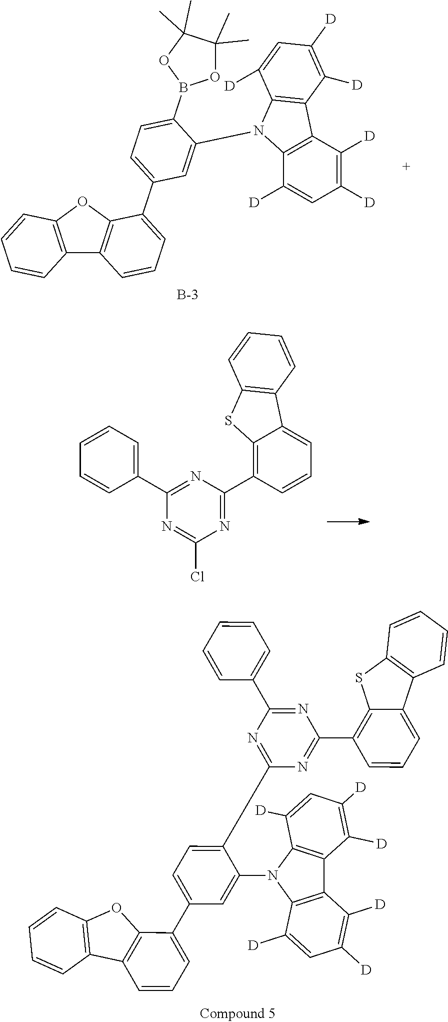

B-3 (20 g, 37 mmol) and 2-chloro-4-(dibenzo[b,d]thiophen-4-yl)-6-phenyl-1,3,5-triazine (13.8 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1366 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.9 g of Compound 5 as a white solid (Yield: 73%, MS: [M+H]⁺=740.2).

### [Preparation Example 6] Synthesis of Compound 6

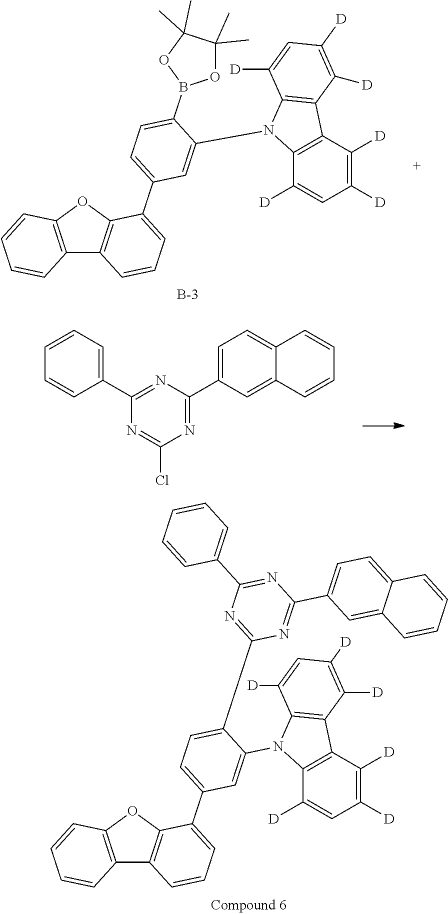

B-3 (20 g, 37 mmol) and 2-chloro-4-(naphthalen-2-yl)-6-phenyl-1,3,5-triazine (11.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1286 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 12.9 g of Compound 6 as a white solid (Yield: 50%, MS: [M+H]⁺=697.3).

### [Preparation Example 7] Synthesis of Compound 7

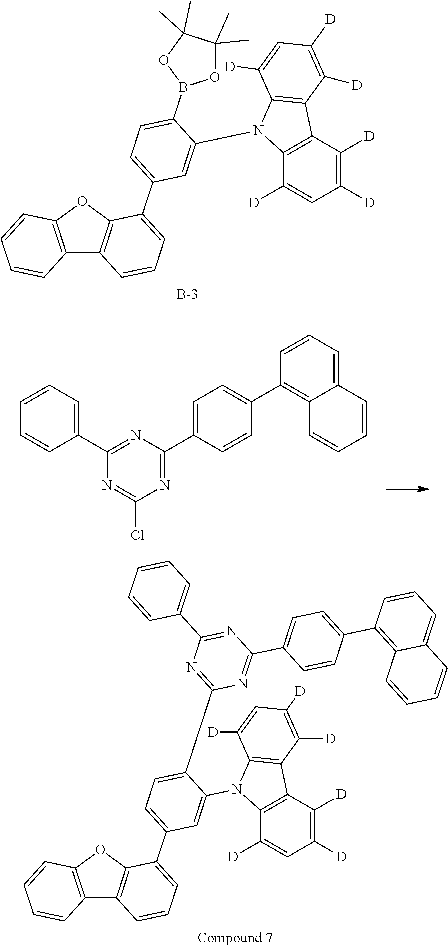

B-3 (20 g, 37 mmol) and 2-chloro-4-(4-(naphthalen-1-yl)phenyl)-6-phenyl-1,3,5-triazine (14.5 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1427 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.6 g of Compound 7 as a white solid (Yield: 51%, MS: [M+H]⁺=773.3).

### [Preparation Example 8] Synthesis of Compound 8

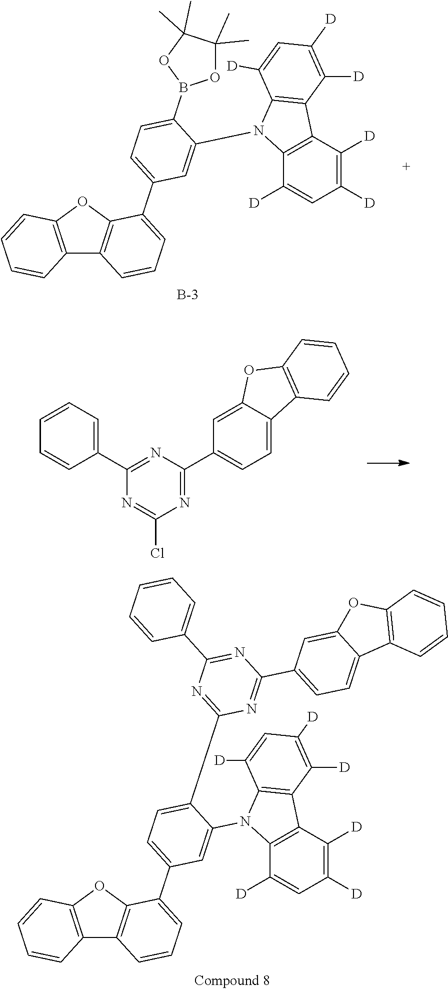

B-3 (20 g, 37 mmol) and 2-chloro-4-(dibenzo[b,d]furan-3-yl)-6-phenyl-1,3,5-triazine (13.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1360 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 16.6 g of Compound 8 as a white solid (Yield: 61%, MS: [M+H]⁺=737.3).

### [Preparation Example 9] Synthesis of Compound 9

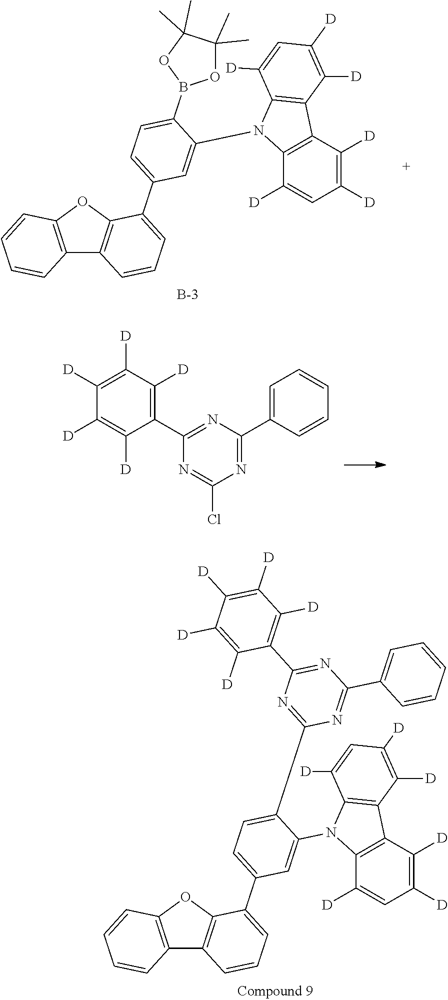

B-3 (20 g, 37 mmol) and 2-chloro-4-phenyl-6-(phenyl-d5)-1,3,5-triazine (14.3 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1203 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 13 g of Compound 9 as a white solid (Yield: 54%, MS: [M+H]⁺=652.3).

### [Preparation Example 10] Synthesis of Compound 10

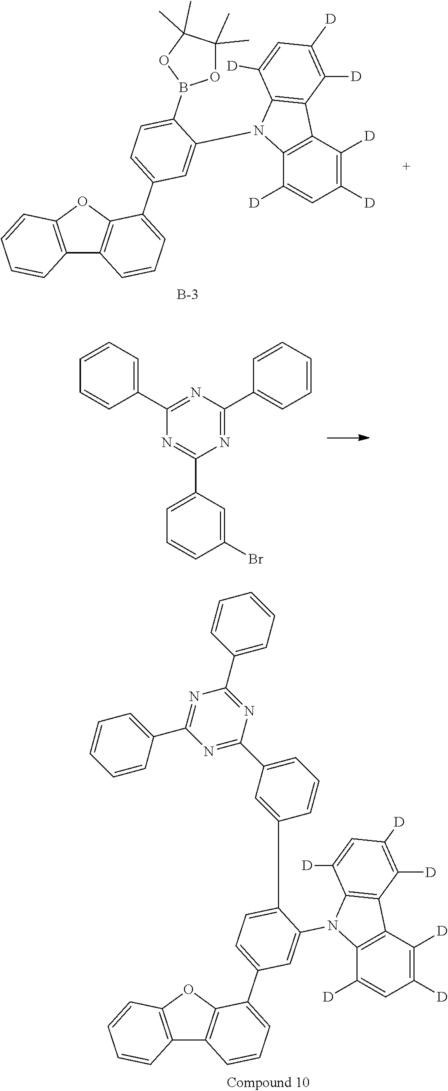

B-3 (20 g, 37 mmol) and 2-(3-bromophenyl)-4,6-diphenyl-1,3,5-triazine (10.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18.7 g of Compound 10 as a white solid (Yield: 70%, MS: [M+H]⁺=723.3).

### [Preparation Example 11] Synthesis of Compound 11

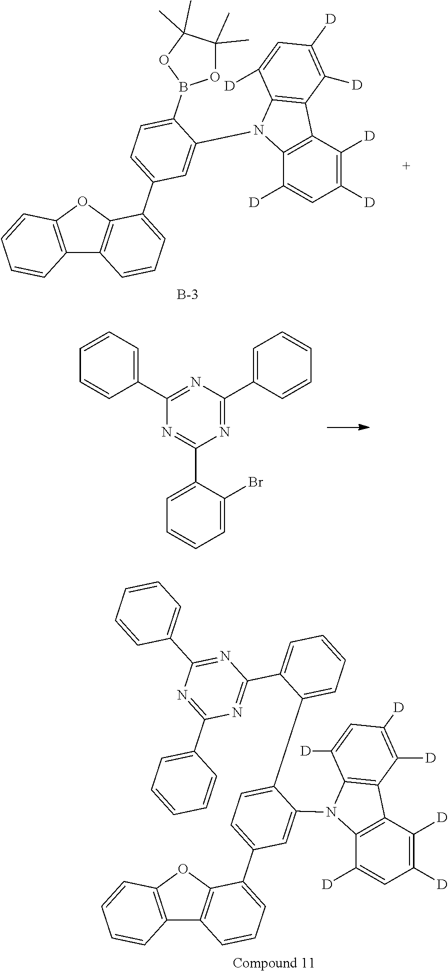

B-3 (20 g, 37 mmol) and 2-(2-bromophenyl)-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 16.5 g of Compound 11 as a white solid (Yield: 62%, MS: [M+H]⁺=723.3).

### [Preparation Example 12] Synthesis of Compound 12

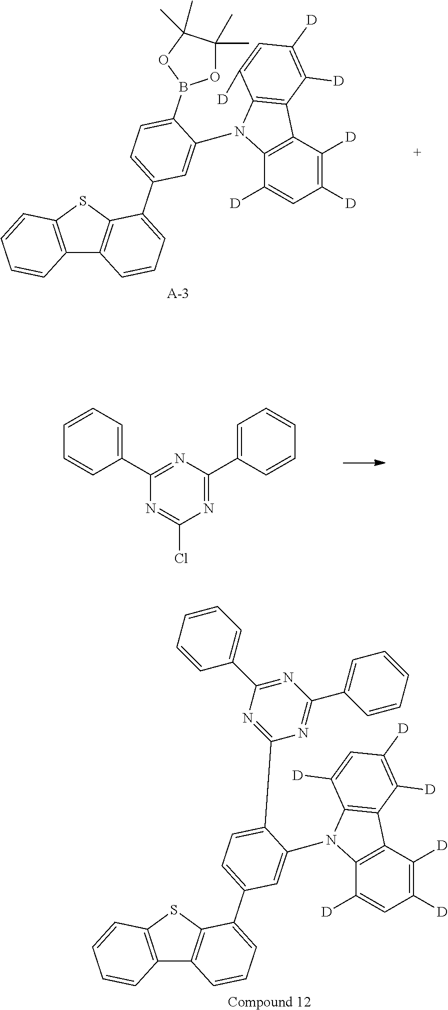

A-3 (20 g, 35.9 mmol) and 2-chloro-4,6-diphenyl-1,3,5-triazine (9.6 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 2 hours, the reaction mixture was cooled to room temperature, and then the resulting solid was filtered. The solid was added to and dissolved in 1160 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 13.2 g of Compound 12 as a white solid (Yield: 57%, MS: [M+H]⁺=647.3).

### [Preparation Example 13] Synthesis of Compound 13

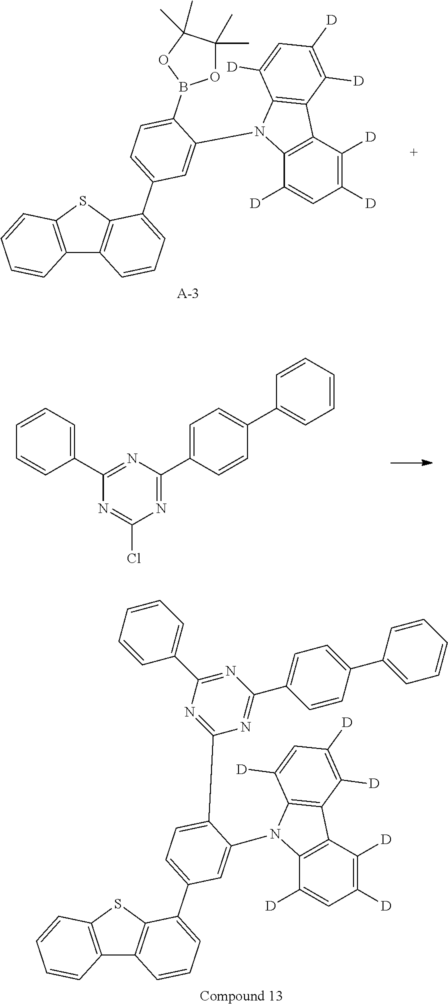

A-3 (20 g, 35.9 mmol) and 2-([1,1′-biphenyl]-4-yl)-4-chloro-6-phenyl-1,3,5-triazine (12.3 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 1 hour, the reaction mixture was cooled to room temperature, and then the resulting solid was filtered. The solid was added to and dissolved in 1296 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 20 g of Compound 13 as a white solid (Yield: 77%, MS: [M+H]⁺=723.3).

### [Preparation Example 14] Synthesis of Compound 14

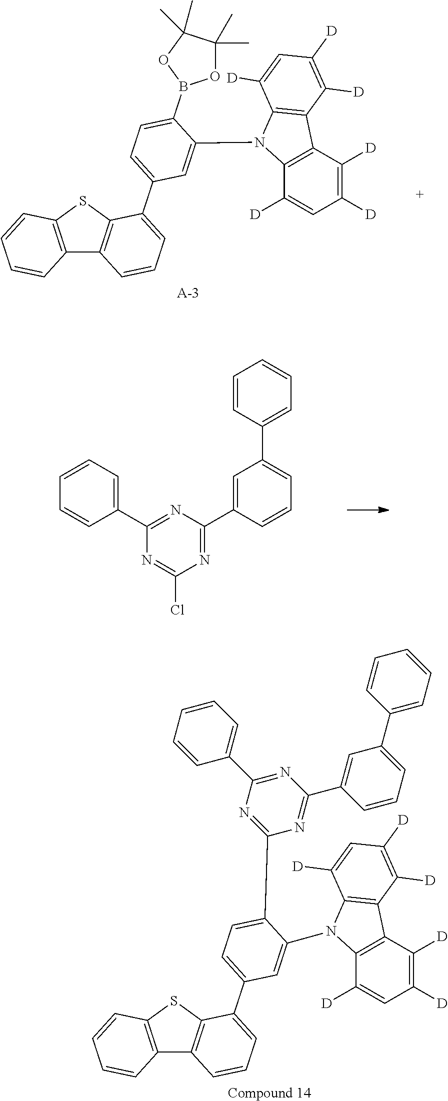

A-3 (20 g, 35.9 mmol) and 2-([1,1′-biphenyl]-4-yl)-3-chloro-6-phenyl-1,3,5-triazine (12.3 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 2 hours, the reaction mixture was cooled to room temperature, and then the resulting solid was filtered. The solid was added to and dissolved in 1296 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 20.7 g of Compound 14 as a white solid (Yield: 80%, MS: [M+H]⁺=723.3).

### [Preparation Example 15] Synthesis of Compound 15

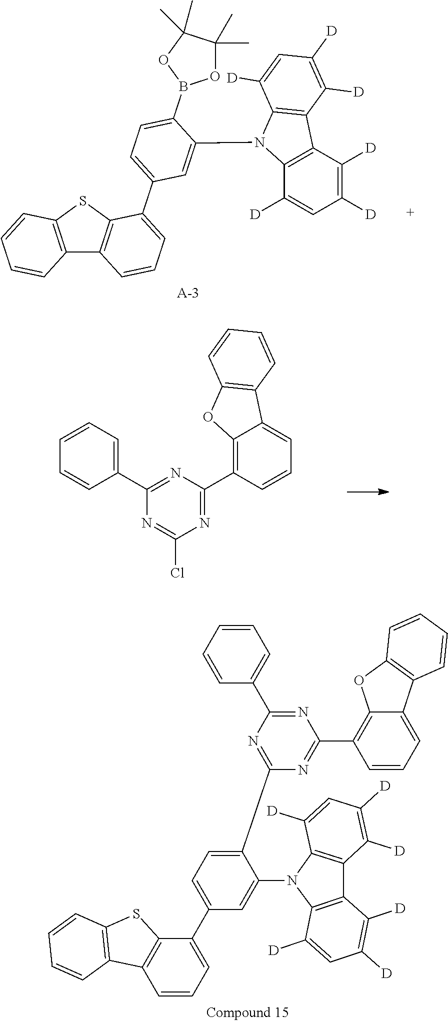

A-3 (20 g, 35.9 mmol) and 2-chloro-4-(dibenzo[b,d]furan-4-yl)-6-phenyl-1,3,5-triazine (12.8 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 3 hours, the reaction mixture was cooled to room temperature, and then the resulting solid was filtered. The solid was added to and dissolved in 1321 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 20.1 g of Compound 15 as a white solid (Yield: 76%, MS: [M+H]⁺=737.3).

### [Preparation Example 16] Synthesis of Compound 16

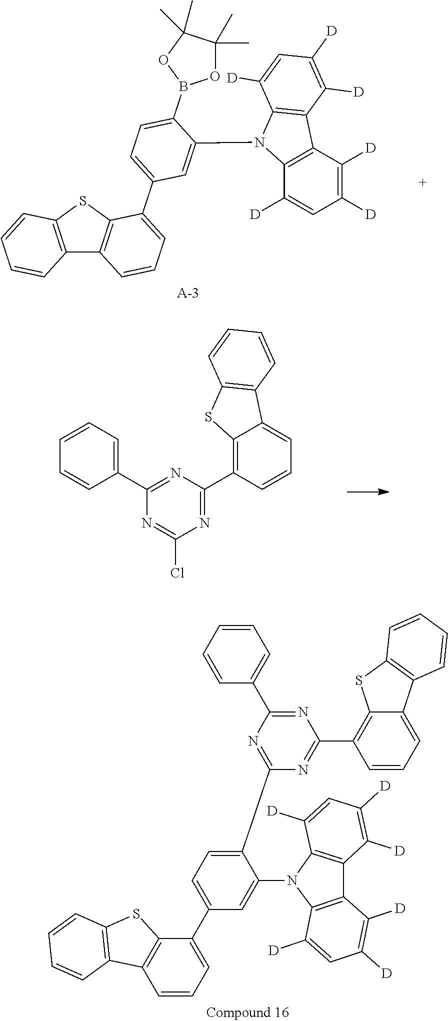

A-3 (20 g, 35.9 mmol) and 2-chloro-4-(dibenzo[b,d]thiophen-4-yl)-6-phenyl-1,3,5-triazine (13.4 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 2 hours, the reaction mixture was cooled to room temperature, and then the resulting solid was filtered. The solid was added to and dissolved in 1327 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18.6 g of Compound 16 as a white solid (Yield: 70%, MS: [M+H]⁺=740.2).

### [Preparation Example 17] Synthesis of Compound 17

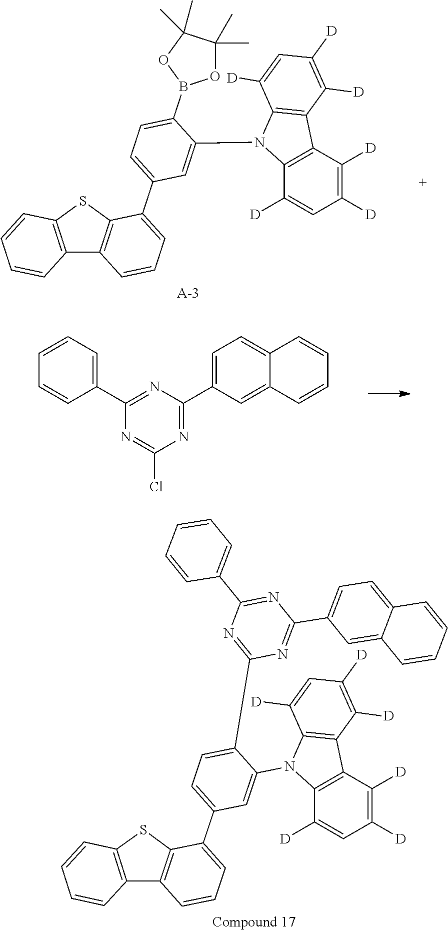

A-3 (20 g, 35.9 mmol) and 2-chloro-4-(naphthalen-2-yl)-6-phenyl-1,3,5-triazine (11.4 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 3 hours, the reaction mixture was cooled to room temperature, and then the resulting solid was filtered. The solid was added to and dissolved in 1278 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 13 g of Compound 17 as a white solid (Yield: 51%, MS: [M+H]⁺=713.3).

### [Preparation Example 18] Synthesis of Compound 18

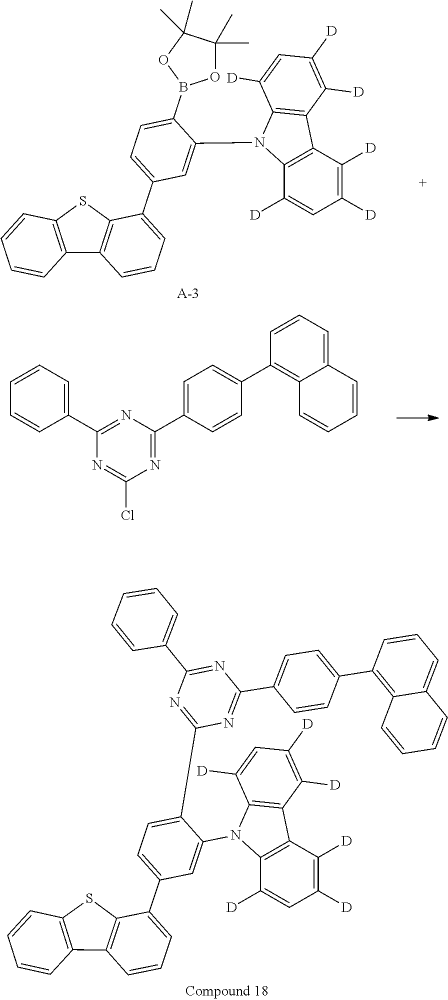

A-3 (20 g, 35.9 mmol) and 2-chloro-4-(4-(naphthalen-1-yl)phenyl)-6-phenyl-1,3,5-triazine (14.1 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 1 hour, the reaction mixture was cooled to room temperature, and then the resulting solid was filtered. The solid was added to and dissolved in 1415 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.8 g of Compound 18 as a white solid (Yield: 70%, MS: [M+H]⁺=789.3).

### [Preparation Example 19] Synthesis of Compound 19

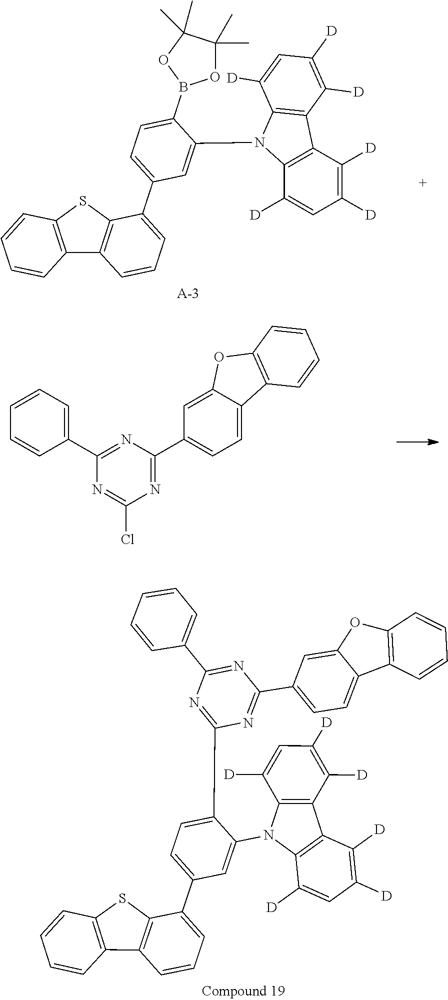

A-3 (20 g, 35.9 mmol) and 2-chloro-4-(dibenzo[b,d] furan-3-yl)-6-phenyl-1,3,5-triazine (12.8 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 1 hour, the reaction mixture was cooled to room temperature, and then the resulting solid was filtered. The solid was added to and dissolved in 1350 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17 g of Compound 19 as a white solid (Yield: 63%, MS: [M+H]⁺=753.3).

### [Preparation Example 20] Synthesis of Compound 20

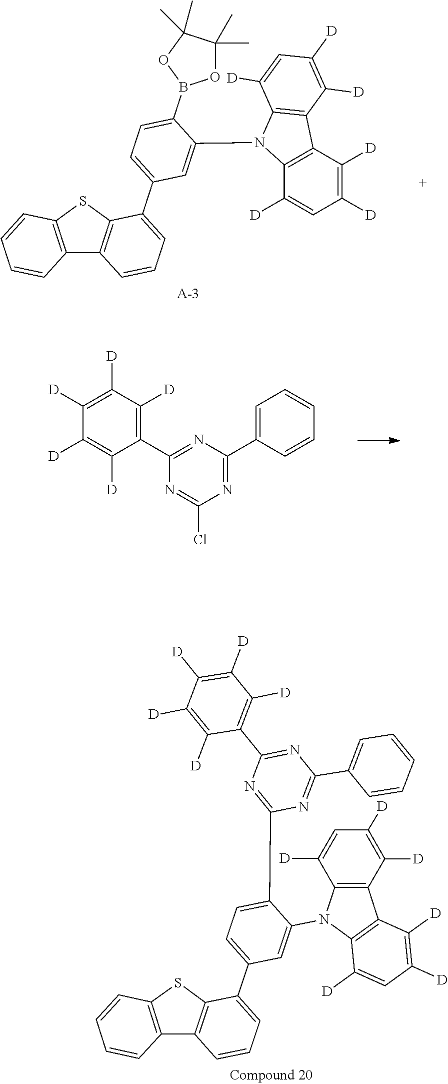

A-3 (20 g, 37 mmol) and 2-chloro-4-phenyl-6-(phenyl-d5)-1,3,5-triazine (14.3 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1233 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 16 g of Compound 20 as a white solid (Yield: 65%, MS: [M+H]⁺=668.3).

### [Preparation Example 21] Synthesis of Compound 21

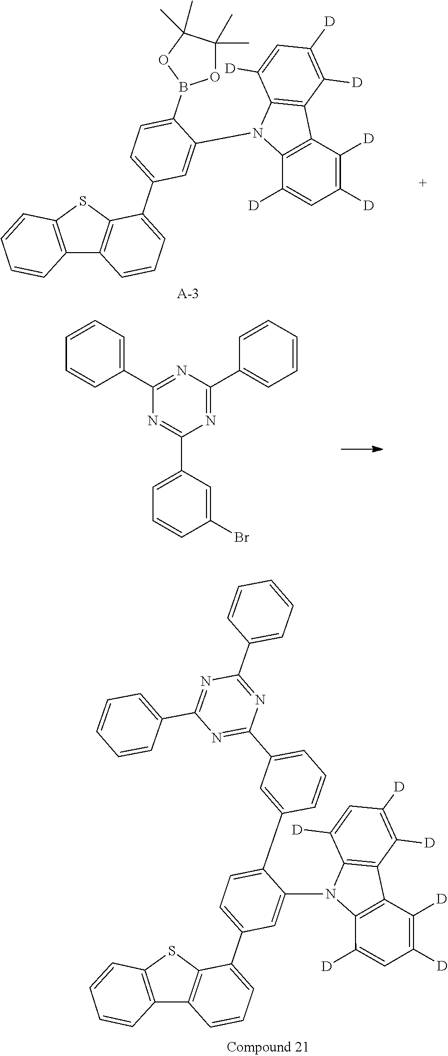

A-3 (20 g, 37 mmol) and 2-(3-bromophenyl)-4,6-diphenyl-1,3,5-triazine (10.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 2 hours, the reaction mixture was cooled to room temperature, and then the resulting solid was filtered. The solid was added to and dissolved in 1364 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17.5 g of Compound 21 as a white solid (Yield: 64%, MS: [M+H]⁺=739.3).

### [Preparation Example 22] Synthesis of Compound 22

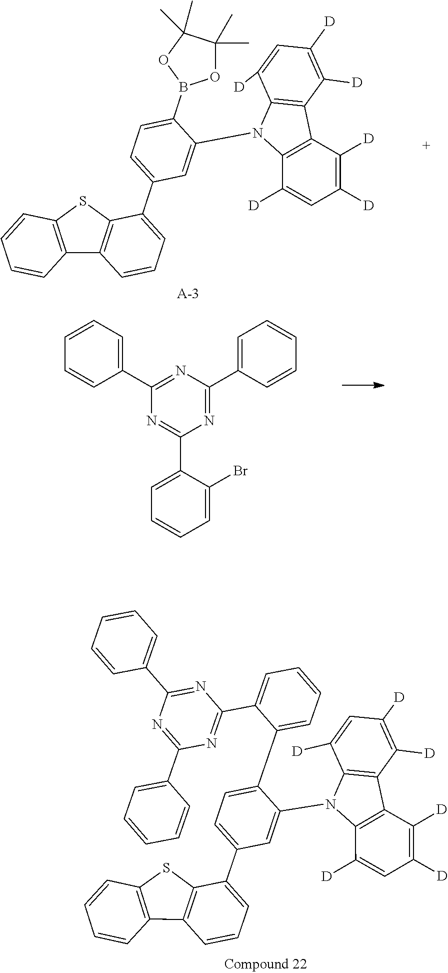

A-3 (20 g, 37 mmol) and 2-(2-bromophenyl)-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 3 hours, the reaction mixture was cooled to room temperature, and then the resulting solid was filtered. The solid was added to and dissolved in 1364 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 21.6 g of Compound 22 as a white solid (Yield: 79%, MS: [M+H]⁺=739.3).

### [Preparation Example 23] Synthesis of Compound 23

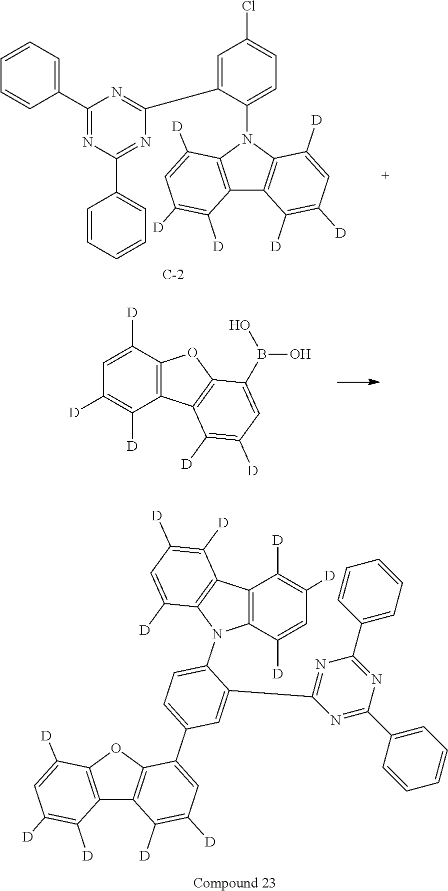

C-2 (20 g, 38.9 mmol) and (dibenzo[b,d]furan-4-yl-1,2,6,8,9-d5)boronic acid (8.4 g, 38.9 mmol) were added to 400 ml of tetrahydrofuran under a nitrogen atmosphere, and the mixture was stirred and refluxed. Then, potassium carbonate (16.1 g, 116.7 mmol) was dissolved in 16 ml of water and added thereto, sufficiently stirred, and then tetrakis(triphenylphosphine)-palladium (1.3 g, 1.2 mmol) was added. After reacting for 2 hours, the reaction mixture was cooled to room temperature, and then the resulting solid was filtered. The solid was added to and dissolved in 1230 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.2 g of Compound 23 as a white solid (Yield: 78%, MS: [M+H]⁺=633.3).

### [Preparation Example 24] Synthesis of Compound 24

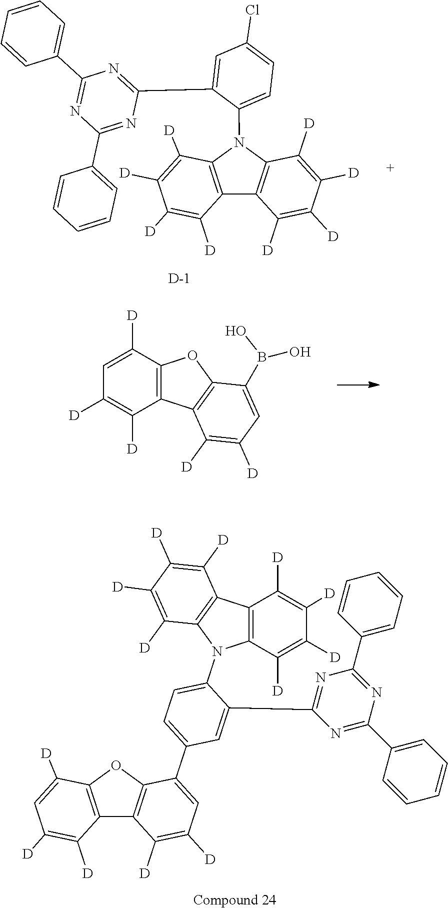

D-1 (20 g, 38.7 mmol) and (dibenzo[b,d]furan-4-yl-1,2,6,8,9-d5)boronic acid (8.4 g, 38.7 mmol) were added to 400 ml of tetrahydrofuran under a nitrogen atmosphere, and the mixture was stirred and refluxed. Then, potassium carbonate (16 g, 116 mmol) was dissolved in 16 ml of water and added thereto, sufficiently stirred, and then tetrakis(triphenylphosphine)-palladium (1.3 g, 1.2 mmol) was added. After reacting for 3 hours, the reaction mixture was cooled to room temperature, and then the resulting solid was filtered. The solid was added to and dissolved in 1255 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15.6 g of Compound 24 as a white solid (Yield: 62%, MS: [M+H]⁺=650.3).

### [Preparation Example 25] Synthesis of Compound 25

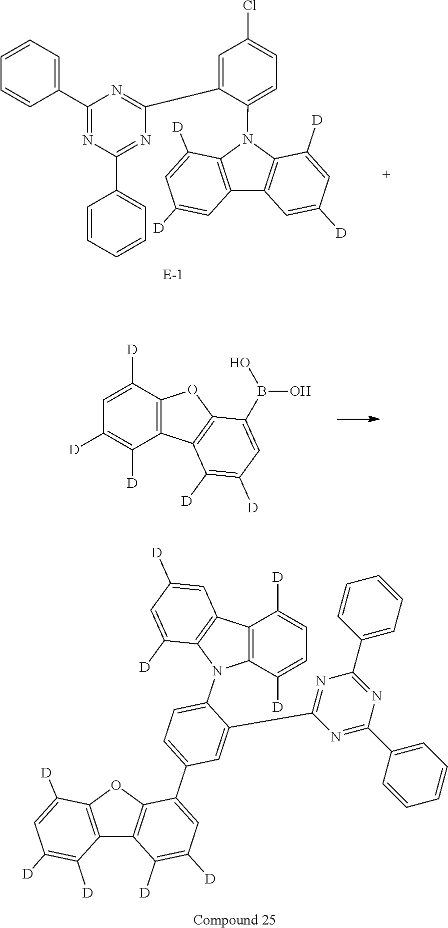

E-1 (20 g, 39 mmol) and (dibenzo[b,d]furan-4-yl-1,2,6,8,9-d5)boronic acid (8.5 g, 39 mmol) were added to 400 ml of tetrahydrofuran under a nitrogen atmosphere, and the mixture was stirred and refluxed. Then, potassium carbonate (16.2 g, 117.1 mmol) was dissolved in 16 ml of water and added thereto, sufficiently stirred, and then tetrakis(triphenylphosphine)palladium (1.4 g, 1.2 mmol) was added. After reacting for 3 hours, the reaction mixture was cooled to room temperature, and then the resulting solid was filtered. The solid was added to and dissolved in 1281 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15.6 g of Compound 25 as a white solid (Yield: 61%, MS: [M+H]⁺=657.3).

### [Preparation Example 26] Synthesis of Compound 26

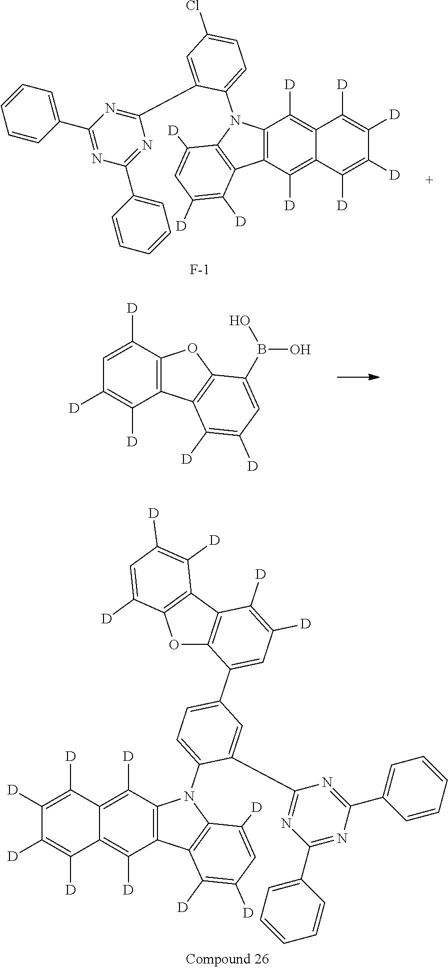

F-1 (20 g, 35.3 mmol) and (dibenzo[b,d]furan-4-yl-1,2,6,8,9-d5)boronic acid (7.7 g, 35.3 mmol) were added to 400 ml of tetrahydrofuran under a nitrogen atmosphere, and the mixture was stirred and refluxed. Then, potassium carbonate (14.6 g, 105.8 mmol) was dissolved in 15 ml of water and added thereto, sufficiently stirred, and then tetrakis(triphenylphosphine)palladium (1.2 g, 1.1 mmol) was added. After reacting for 3 hours, the reaction mixture was cooled to room temperature, and then the resulting solid was filtered. The solid was added to and dissolved in 1242 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 16.4 g of Compound 26 as a white solid (Yield: 66%, MS: [M+H]⁺=705.3).

### [Preparation Example 27] Synthesis of Compound 27

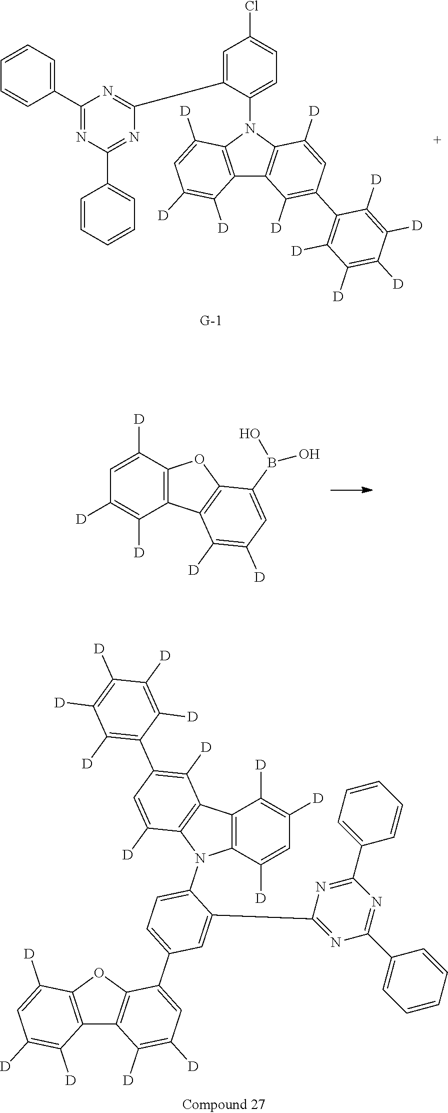 [SMILES: [2H]c1cc([2H])c2c(c1[2H])c1c([2H])c(-c3c([2H])c([2H])c([2H])c([2H])c3[2H])cc([2H])c1n2-c1ccc(Cl)cc1CCc1nc(-c2ccccc2)nc(-c2ccccc2)n1 ; [2H]c1cc([2H])c2oc3c(B(O)O)cc([2H])c([2H])c3c2c1[2H] ; [2H]c1cc([2H])c2oc3c(-c4ccc(-n5c6c([2H])cc([2H])c([2H])c6c6c([2H])c(-c7c([2H])c([2H])c([2H])c([2H])c7[2H])cc([2H])c65)c(-c5nc(-c6ccccc6)nc(-c6ccccc6)n5)c4)cc([2H])c([2H])c3c2c1[2H]]

G-1 (20 g, 33.7 mmol) and (dibenzo[b,d]furan-4-yl-1,2,6,8,9-d5)boronic acid (7.3 g, 33.7 mmol) were added to 400 ml of tetrahydrofuran under a nitrogen atmosphere, and the mixture was stirred and refluxed. Then, potassium carbonate (14 g, 101 mmol) was dissolved in 14 ml of water and added thereto, sufficiently stirred, and then tetrakis(triphenylphosphine)-palladium (1.2 g, 1 mmol) was added. After reacting for 2 hours, the reaction mixture was cooled to room temperature, and then the resulting solid was filtered. The solid was added to and dissolved in 1231 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 16.2 g of Compound 27 as a white solid (Yield: 66%, MS: [M+H]⁺=732.4).

### [Preparation Example 28] Synthesis of Compound 28

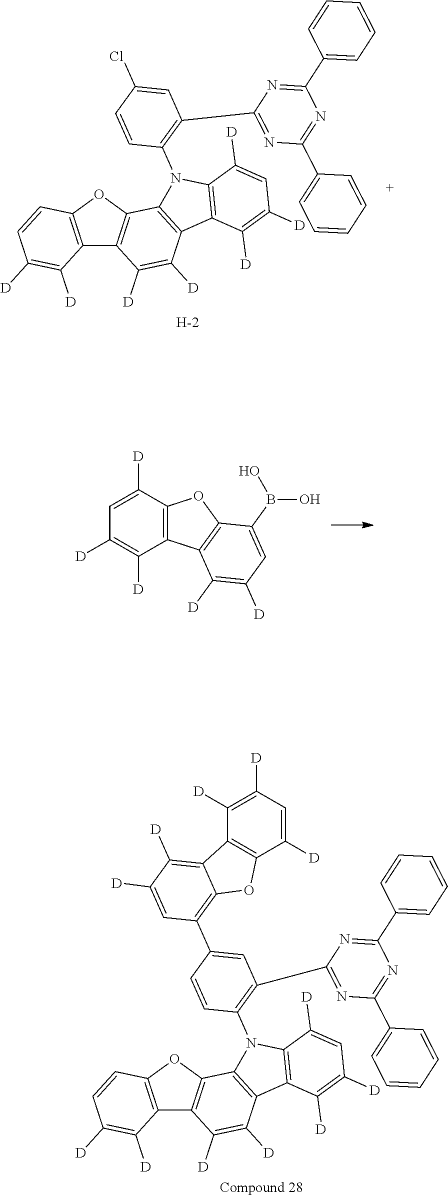

H-2 (20 g, 33 mmol) and (dibenzo[b,d]furan-4-yl-1,2,6,8,9-d5)boronic acid (7.2 g, 33 mmol) were added to 400 ml of tetrahydrofuran under a nitrogen atmosphere, and the mixture was stirred and refluxed. Then, potassium carbonate (13.7 g, 99.1 mmol) was dissolved in 14 ml of water and added thereto, sufficiently stirred, and then tetrakis(triphenylphosphine)palladium (1.1 g, 1 mmol) was added. After reacting for 1 hour, the reaction mixture was cooled to room temperature, and then the resulting solid was filtered. The solid was added to and dissolved in 1227 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18.9 g Compound 28 as a white solid (Yield: 77%, MS: [M+H]⁺=743.3).

### [Preparation Example 29] Synthesis of Compound 29

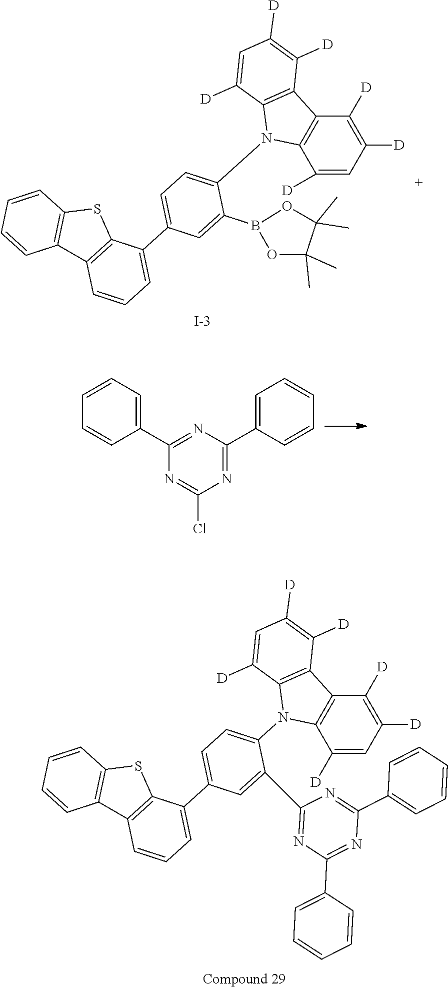

I-3 (20 g, 35.9 mmol) and 2-chloro-4,6-diphenyl-1,3,5-triazine (9.6 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 2 hours, the reaction mixture was cooled to room temperature, and then the resulting solid was filtered. The solid was added to and dissolved in 1160 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.1 g of Compound 29 as a white solid (Yield: 61%, MS: [M+H]⁺=647.3).

### [Preparation Example 30] Synthesis of Compound 30

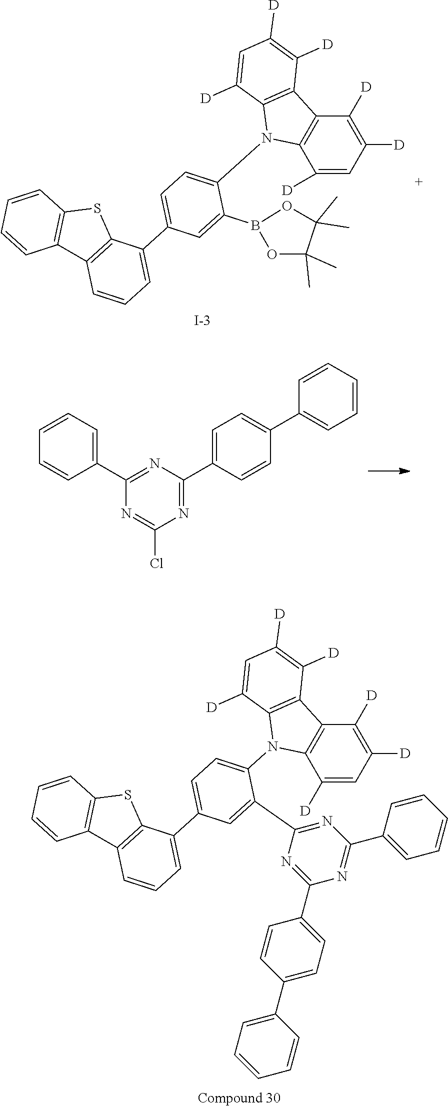

I-3 (20 g, 35.9 mmol) and 2-([1,1′-biphenyl]-4-yl)-4-chloro-6-phenyl-1,3,5-triazine (12.3 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 3 hours, the reaction mixture was cooled to room temperature, and then the resulting solid was filtered. The solid was added to and dissolved in 1296 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.2 g of Compound 30 as a white solid (Yield: 74%, MS: [M+H]⁺=723.3).

### [Preparation Example 31] Synthesis of Compound 31

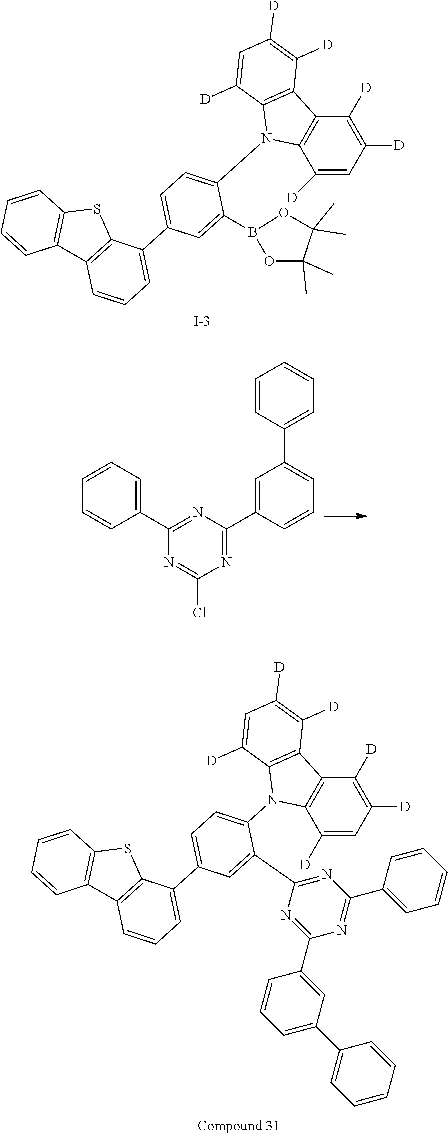

I-3 (20 g, 35.9 mmol) and 2-([1,1′-biphenyl]-3-yl)-4-chloro-6-phenyl-1,3,5-triazine (12.3 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 2 hours, the reaction mixture was cooled to room temperature, and then the resulting solid was filtered. The solid was added to and dissolved in 1296 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 20.7 g of Compound 31 as a white solid (Yield: 80%, MS: [M+H]⁺=723.3).

### [Preparation Example 32] Synthesis of Compound 32

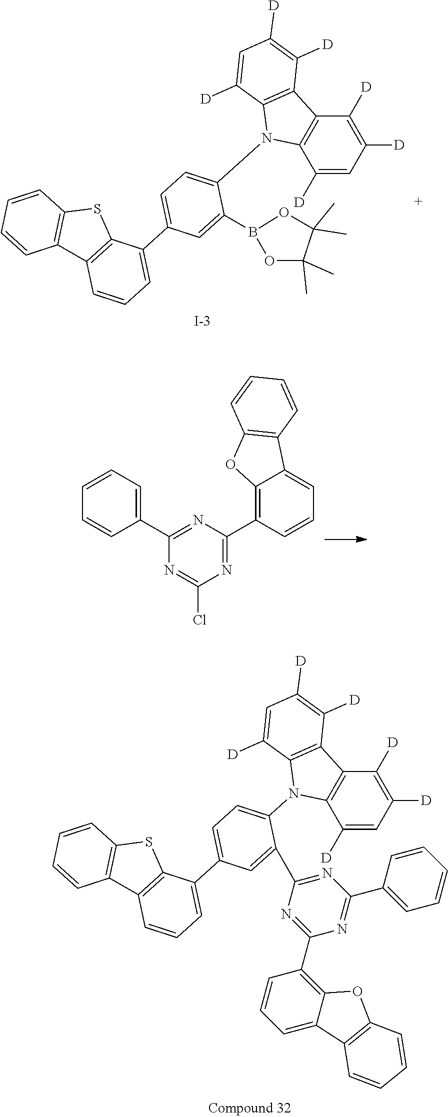

I-3 (20 g, 35.9 mmol) and 2-chloro-4-(dibenzo[b,d]furan-4-yl)-6-phenyl-1,3,5-triazine (12.8 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium(1.2 g, 1.1 mmol) was added. After reacting for 2 hours, the reaction mixture was cooled to room temperature, and then the resulting solid was filtered. The solid was added to and dissolved in 1321 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17.2 g of Compound 32 as a white solid (Yield: 65%, MS: [M+H]⁺=737.3).

### [Preparation Example 33] Synthesis of Compound 33

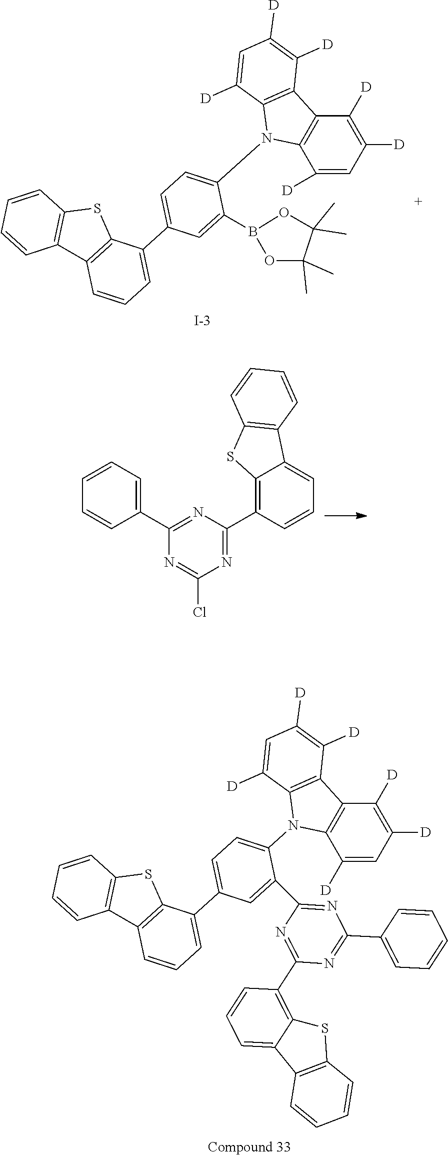

I-3 (20 g, 35.9 mmol) and 2-chloro-4-(dibenzo[b,d]thiophen-4-yl)-6-phenyl-1,3,5-triazine (13.4 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 3 hours, the reaction mixture was cooled to room temperature, and then the resulting solid was filtered. The solid was added to and dissolved in 1327 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 20.2 g of Compound 33 as a white solid (Yield: 76%, MS: [M+H]⁺=740.2).

### [Preparation Example 34] Synthesis of Compound 34

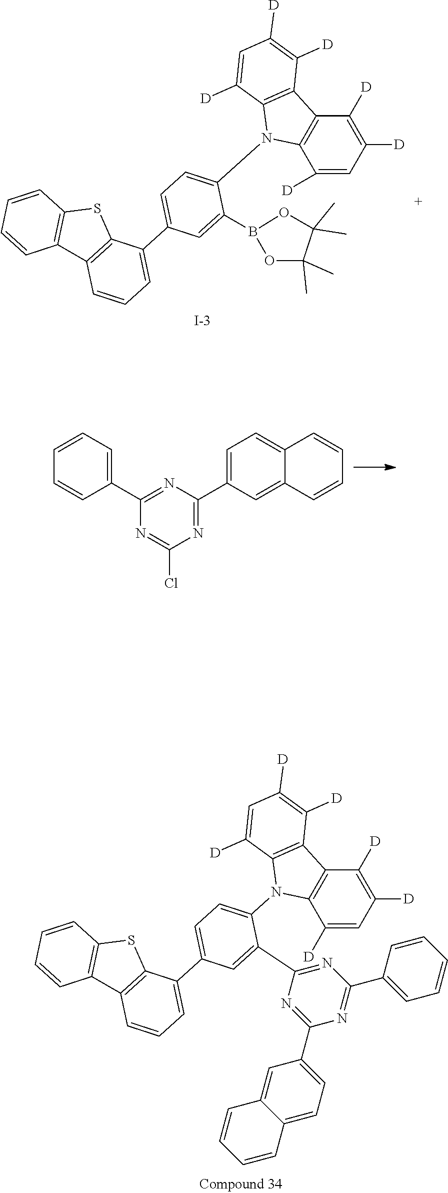

I-3 (20 g, 35.9 mmol) and 2-chloro-4-(naphthalen-2-yl)-6-phenyl-1,3,5-triazine (11.4 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)-palladium (1.2 g, 1.1 mmol) was added. After reacting for 2 hours, the reaction mixture was cooled to room temperature, and then the resulting solid was filtered. The solid was added to and dissolved in 1278 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.1 g of Compound 34 as a white solid (Yield: 55%, MS: [M+H]⁺=713.3).

### [Preparation Example 35] Synthesis of Compound 35

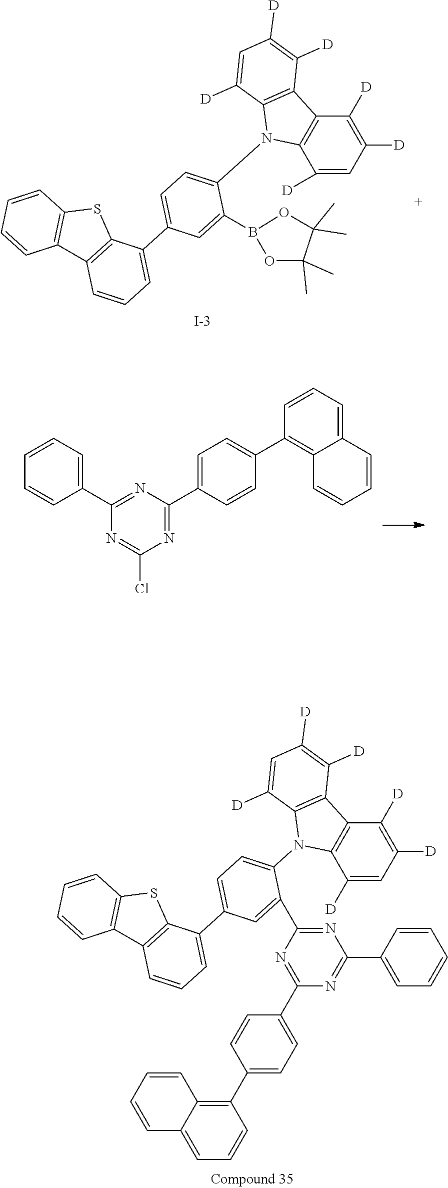

I-3 (20 g, 35.9 mmol) and 2-chloro-4-(4-(naphthalen-1-yl)phenyl)-6-phenyl-1,3,5-triazine (14.1 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 1 hour, the reaction mixture was cooled to room temperature, and then the resulting solid was filtered. The solid was added to and dissolved in 1415 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15.8 g of Compound 35 as a white solid (Yield: 56%, MS: [M+H]⁺=789.3).

### [Preparation Example 36] Synthesis of Compound 36

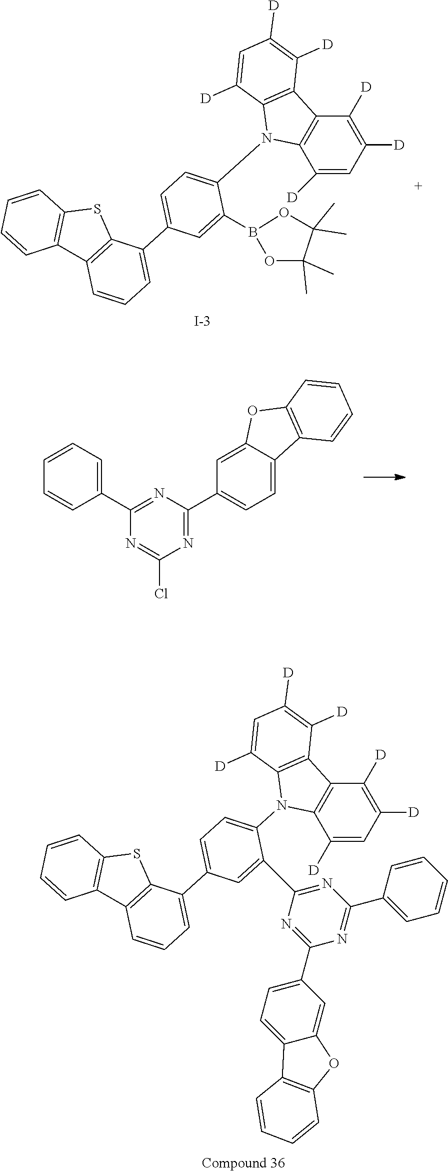

I-3 (20 g, 35.9 mmol) and 2-chloro-4-(dibenzo[b,d]furan-3-yl)-6-phenyl-1,3,5-triazine (12.8 g, 35.9 mmol) were added to 400 ml of tetrahydrofuran under a nitrogen atmosphere, and the mixture was stirred and refluxed. Then, potassium carbonate (14.9 g, 107.7 mmol) was dissolved in 15 ml of water and added thereto, sufficiently stirred, and then tetrakis(triphenylphosphine)palladium (1.2 g, 1.1 mmol) was added. After reacting for 3 hours, the reaction mixture was cooled to room temperature, and then the resulting solid was filtered. The solid was added to and dissolved in 1350 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.4 g of Compound 36 as a white solid (Yield: 72%, MS: [M+H]⁺=753.3).

### [Preparation Example 37] Synthesis of Compound 37

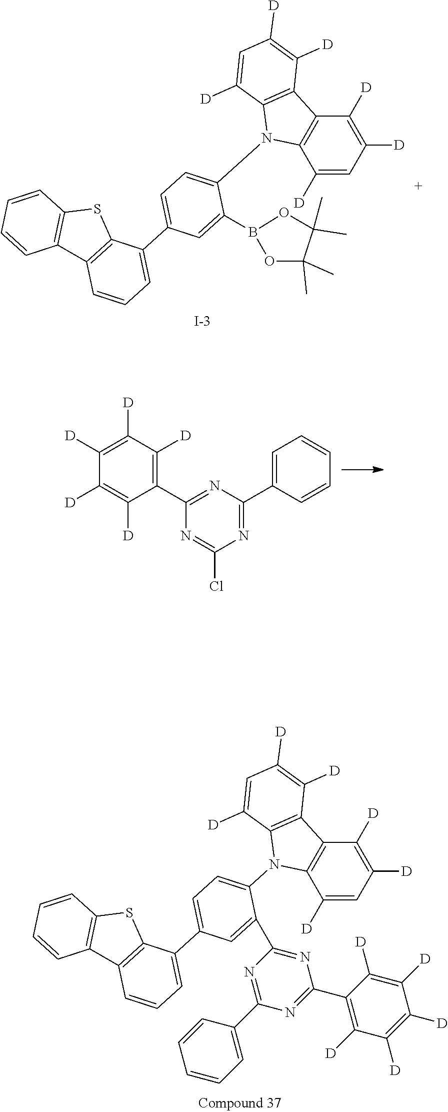

I-3 (20 g, 37 mmol) and 2-([1,1′-biphenyl]-3-yl)-4-chloro-6-phenyl-1,3,5-triazine (14.3 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1233 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15.5 g of Compound 37 as a white solid (Yield: 63%, MS: [M+H]⁺=668.3).

### [Preparation Example 38] Synthesis of Compound 38

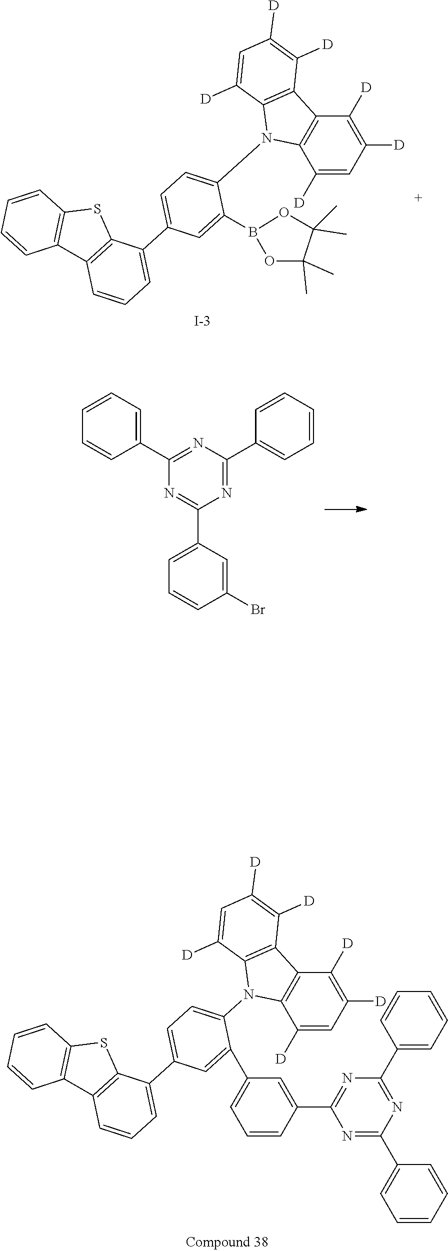

I-3 (20 g, 37 mmol) and 2-(3-bromophenyl)-4,6-diphenyl-1,3,5-triazine (10.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1364 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17.5 g of Compound 38 as a white solid (Yield: 64%, MS: [M+H]⁺=739.3).

### [Preparation Example 39] Synthesis of Compound 39

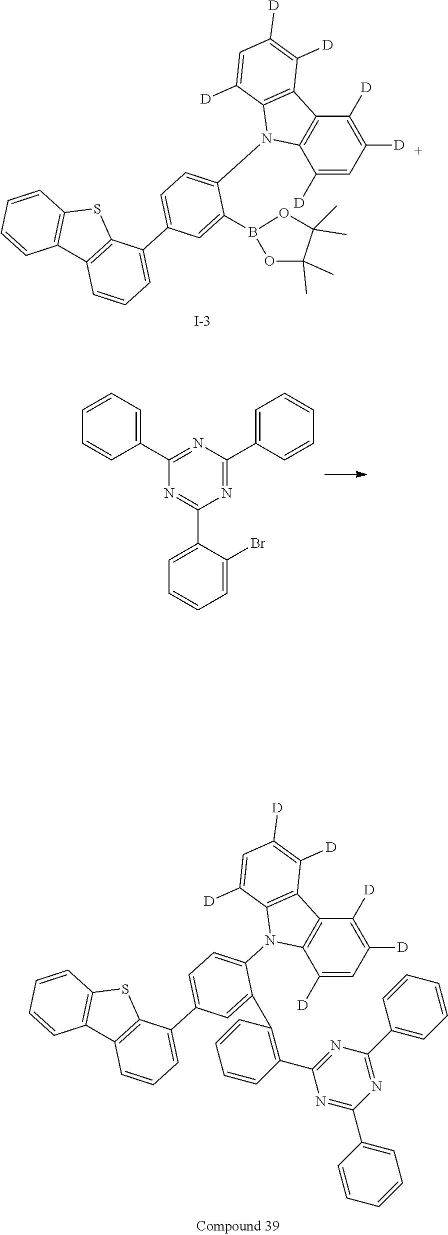

I-3 (20 g, 37 mmol) and 2-(2-bromophenyl)-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1364 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18 g of Compound 39 as a white solid (Yield: 66%, MS: [M+H]⁺=739.3).

### [Preparation Example 40] Synthesis of Compound 40

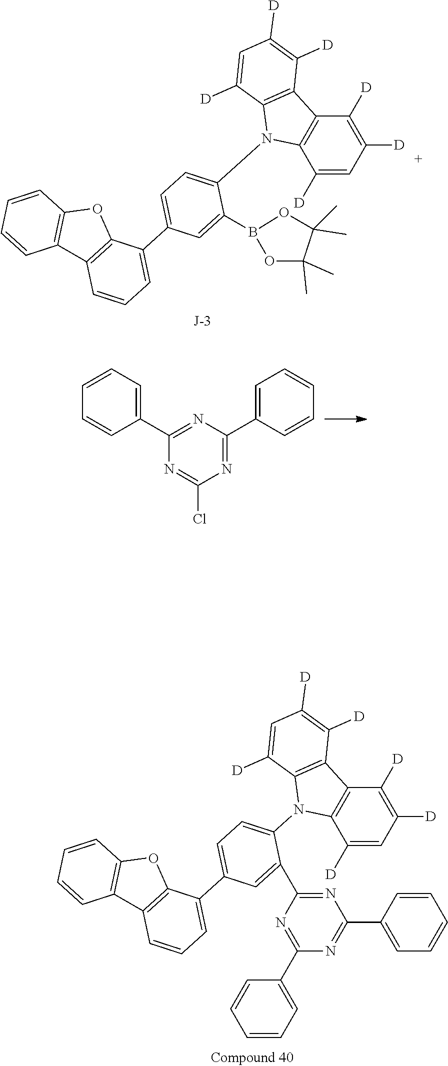 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; "J-3" ; Clc1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccccc2)n1]

J-3 (20 g, 37 mmol) and 2-chloro-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1194 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18.1 g of Compound 40 as a white solid (Yield: 76%, MS: [M+H]⁺=647.3).

### [Preparation Example 41] Synthesis of Compound 41

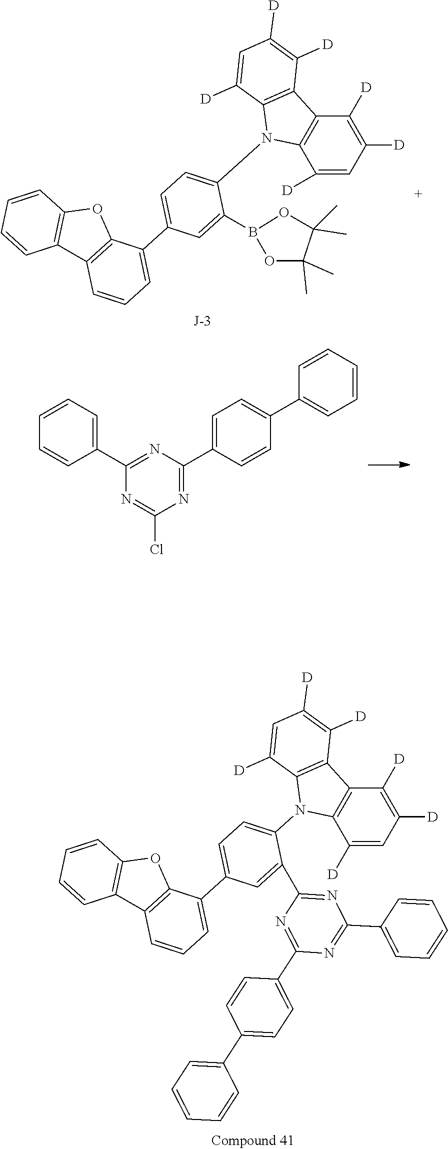 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; "J-3" ; Clc1nc(-c2ccccc2)nc(-c2ccc(-c3ccccc3)cc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3ccccc3)cc2)n1]

J-3 (20 g, 37 mmol) and 2-([1,1′-biphenyl]-4-yl)-4-chloro-6-phenyl-1,3,5-triazine (12.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 1 hour, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17.9 g of Compound 41 as a white solid (Yield: 67%, MS: [M+H]⁺=723.3).

### [Preparation Example 42] Synthesis of Compound 42

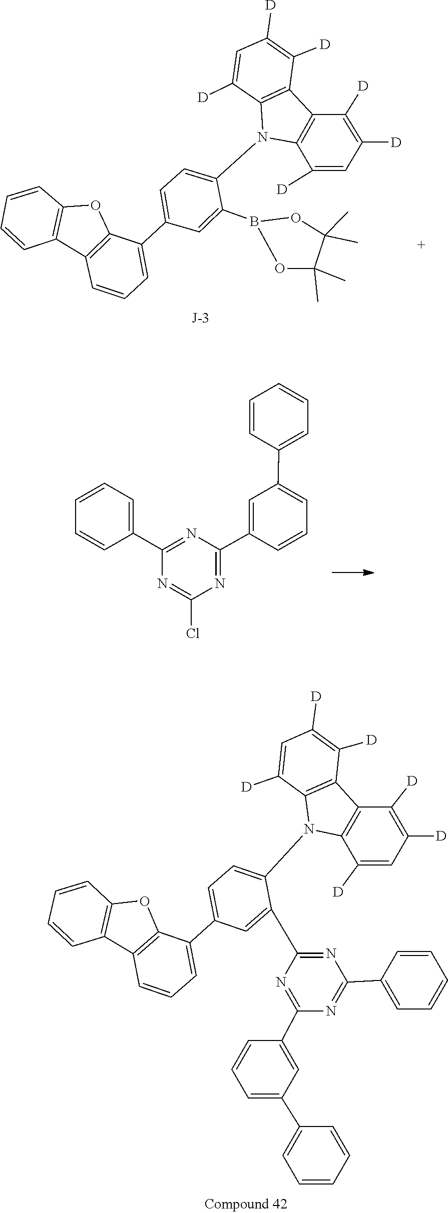 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2cccc(-c3ccccc3)c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc(-c3ccccc3)c2)n1]

J-3 (20 g, 37 mmol) and 2-([1,1′-biphenyl]-3-yl)-4-chloro-6-phenyl-1,3,5-triazine (12.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 1 hour, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.4 g of Compound 42 as a white solid (Yield: 54%, MS: [M+H]⁺=723.3).

### [Preparation Example 43] Synthesis of Compound 43

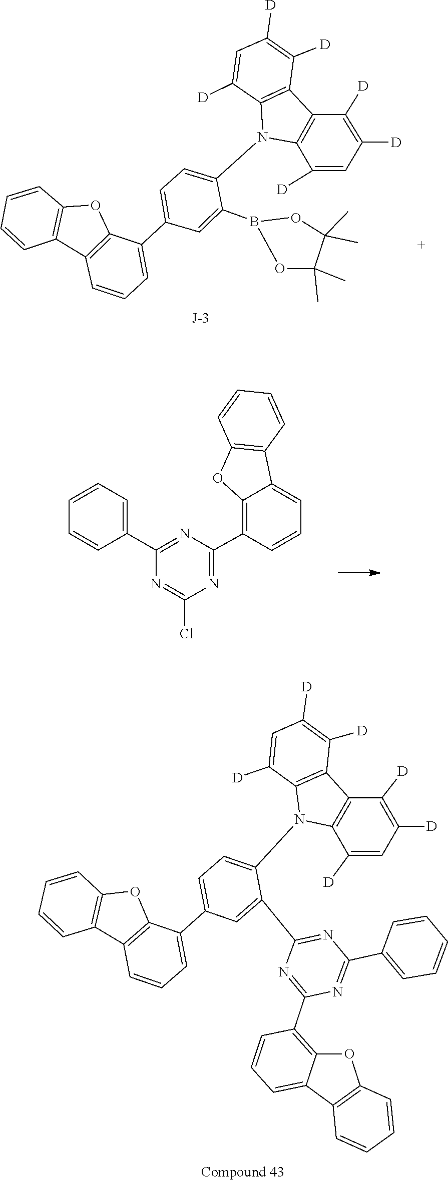 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2cccc3c2oc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c2oc2ccccc23)n1]

J-3 (20 g, 37 mmol) and 2-chloro-4-(dibenzo[b,d]furan-4-yl)-6-phenyl-1,3,5-triazine (13.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 1 hour, the reaction mixture was cooled to room temperature, and then the resulting solid was filtered. The solid was added to and dissolved in 1360 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 15 g of Compound 43 as a white solid (Yield: 55%, MS: [M+H]⁺=737.3).

### [Preparation Example 44] Synthesis of Compound 44

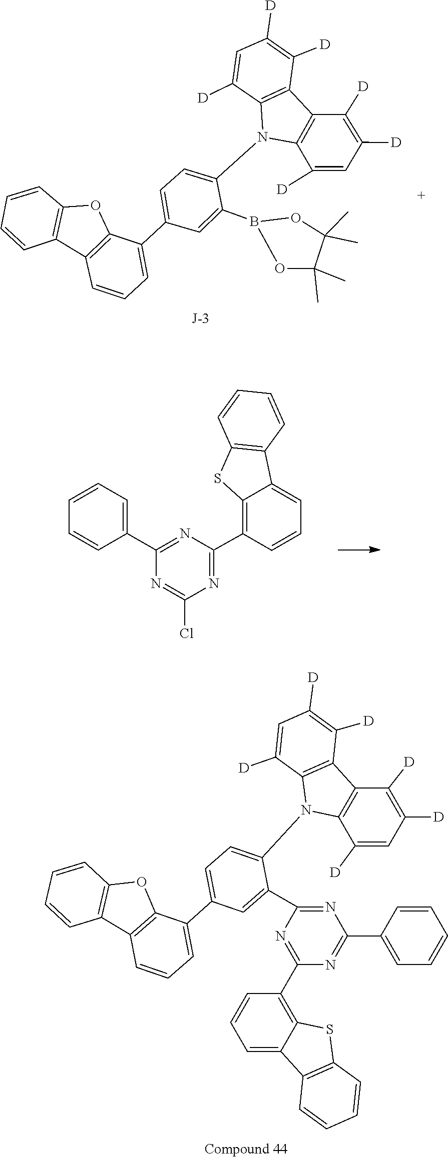 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2cccc3c2sc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c2sc2ccccc23)n1]

J-3 (20 g, 37 mmol) and 2-chloro-4-(dibenzo[b,d]thiophen-4-yl)-6-phenyl-1,3,5-triazine (13.8 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1366 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 17.2 g of Compound 44 as a white solid (Yield: 63%, MS: [M+H]⁺=740.2).

### [Preparation Example 45] Synthesis of Compound 45

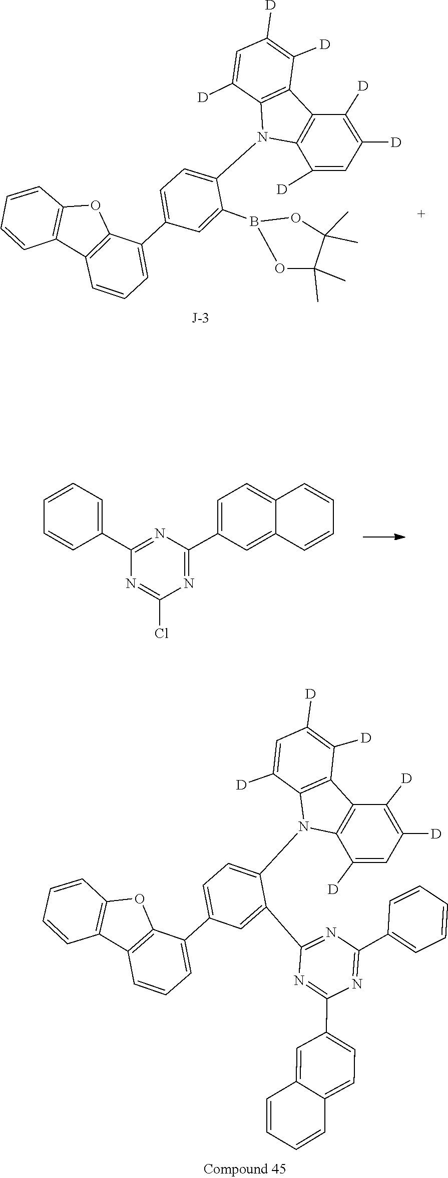 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2ccc3ccccc3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3ccccc3c2)n1]

J-3 (20 g, 37 mmol) and 2-chloro-4-(naphthalen-2-yl)-6-phenyl-1,3,5-triazine (11.7 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)-palladium (1.3 g, 1.1 mmol) was added. After reacting for 1 hour, the reaction mixture was cooled to room temperature, and then the resulting solid was filtered. The solid was added to and dissolved in 1286 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 13.1 g of Compound 45 as a white solid (Yield: 51%, MS: [M+H]⁺=697.3).

### [Preparation Example 46] Synthesis of Compound 46

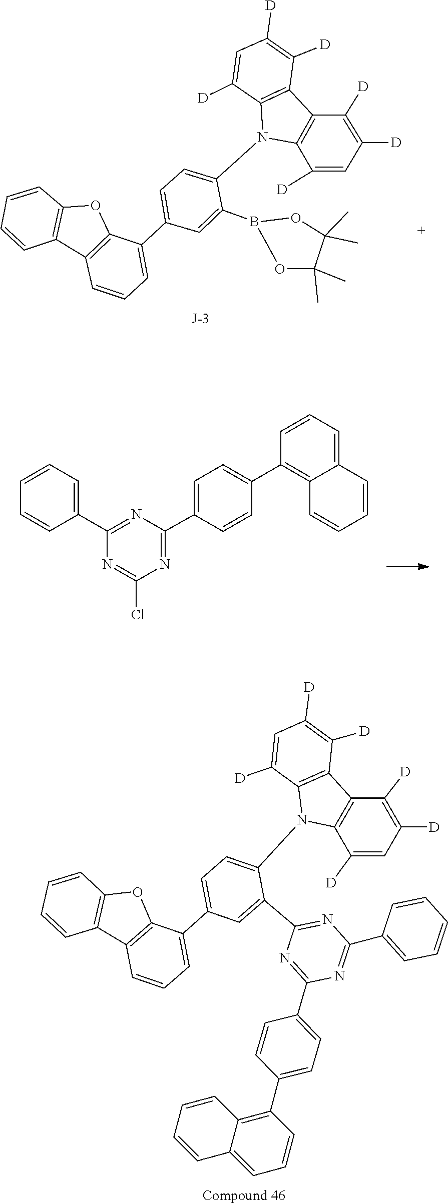 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2ccc(-c3cccc4ccccc34)cc2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3cccc4ccccc34)cc2)n1]

J-3 (20 g, 37 mmol) and 2-chloro-4-(4-(naphthalen-1-yl)phenyl)-6-phenyl-1,3,5-triazine (14.5 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1427 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 14.6 g of Compound 46 as a white solid (Yield: 51%, MS: [M+H]⁺=773.3).

### [Preparation Example 47] Synthesis of Compound 47

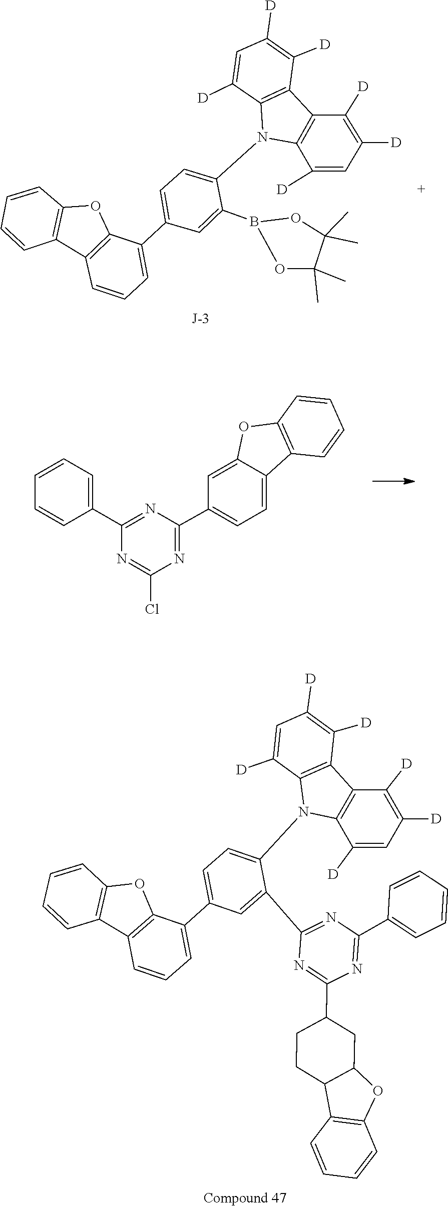 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Clc1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(C2CCC3c4ccccc4OC3C2)n1]

J-3 (20 g, 37 mmol) and 2-chloro-4-(dibenzo[b,d]furan-3-yl)-6-phenyl-1,3,5-triazine (13.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 1 hour, the reaction mixture was cooled to room temperature, and then the resulting solid was filtered. The solid was added to and dissolved in 1360 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.9 g of Compound 47 as a white solid (Yield: 73%, MS: [M+H]⁺=737.3).

### [Preparation Example 48] Synthesis of Compound 48

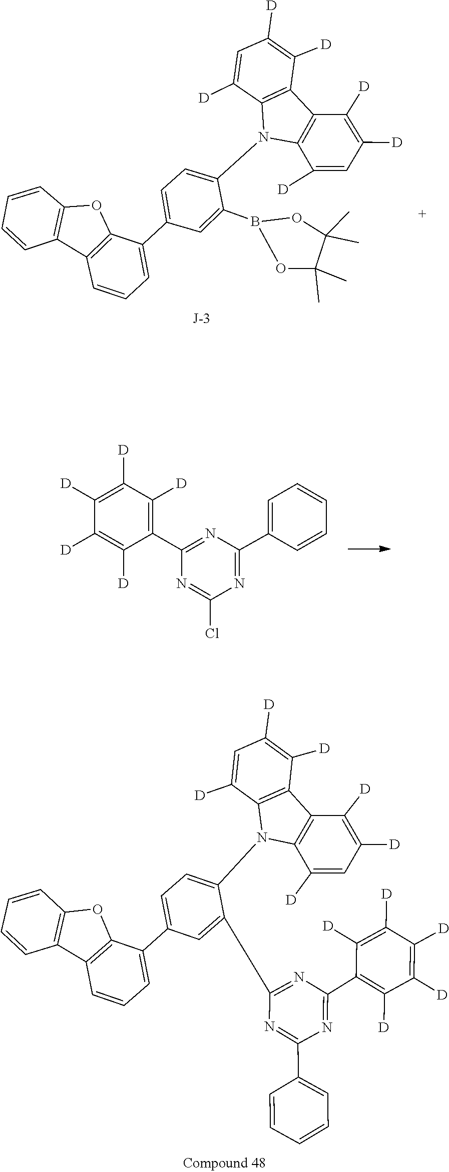 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; [2H]c1c([2H])c([2H])c(-c2nc(Cl)nc(-c3ccccc3)n2)c([2H])c1[2H] ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2c([2H])c([2H])c([2H])c([2H])c2[2H])n1]

J-3 (20 g, 37 mmol) and 2-chloro-4-phenyl-6-(phenyl-d5)-1,3,5-triazine (14.3 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1203 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 18.8 g of Compound 48 as a white solid (Yield: 78%, MS: [M+H]⁺=652.3).

### [Preparation Example 49] Synthesis of Compound 49

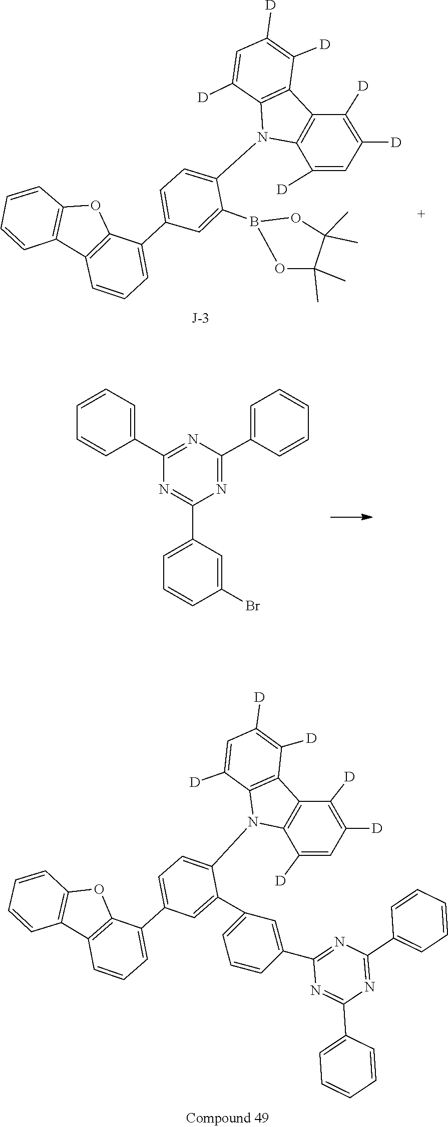 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Brc1cccc(-c2nc(-c3ccccc3)nc(-c3ccccc3)n2)c1 ; [2H]c1cc([2H])c2c(c1[2H])c1c([2H])c([2H])cc([2H])c1n2-c1ccc(-c2cccc3c2oc2ccccc23)cc1-c1cccc(-c2nc(-c3ccccc3)nc(-c3ccccc3)n2)c1]

J-3 (20 g, 37 mmol) and 2-(3-bromophenyl)-4,6-diphenyl-1,3,5-triazine (10.2 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 2 hours, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.5 g of Compound 49 as a white solid (Yield: 73%, MS: [M+H]⁺=723.3).

### [Preparation Example 50] Synthesis of Compound 50

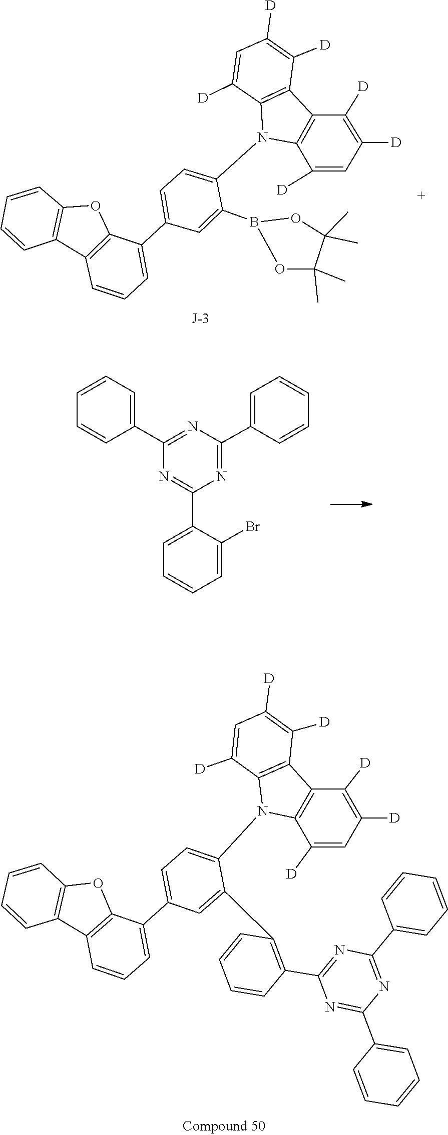 [SMILES: [2H]c1cc([2H])c2c(c1[2H])c1c([2H])c([2H])cc([2H])c1n2-c1ccc(-c2cccc3c2oc2ccccc23)cc1B1OC(C)(C)C(C)(C)O1 ; Brc1ccccc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2oc2ccccc23)cc1-c1ccccc1-c1nc(-c2ccccc2)nc(-c2ccccc2)n1]

J-3 (20 g, 37 mmol) and 2-(2-bromophenyl)-4,6-diphenyl-1,3,5-triazine (9.9 g, 37 mmol) were added to 400 ml of tetrahydrofuran under a nitrogen atmosphere, and the mixture was stirred and refluxed. Then, potassium carbonate (15.3 g, 110.9 mmol) was dissolved in 15 ml of water and added thereto, sufficiently stirred, and then tetrakis(triphenylphosphine)palladium (1.3 g, 1.1 mmol) was added. After reacting for 3 hours, the reaction mixture was cooled to room temperature, and then the resulting solid was filtered. The solid was added to and dissolved in 1334 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to give 19.5 g of Compound 50 as a white solid (Yield: 73%, MS: [M+H]⁺=723.3).

### [Preparation Example 51] Synthesis of Compound 51

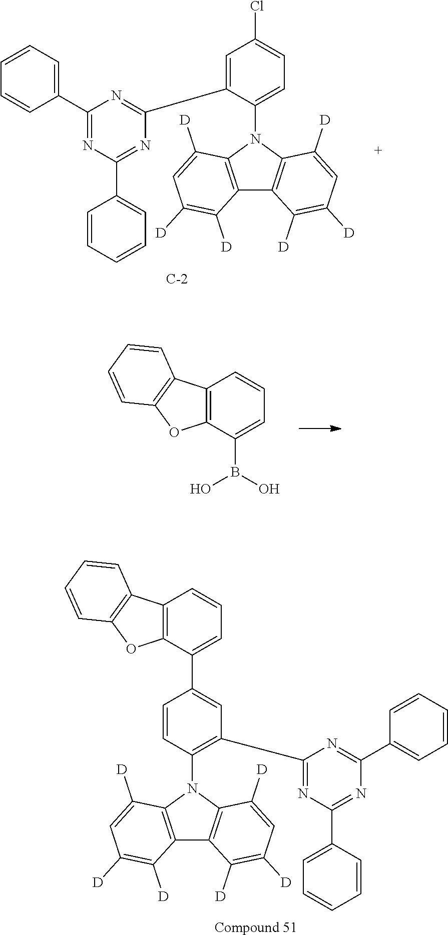

C-2 (20 g, 38.9 mmol) and dibenzo[b,d]furan-4-ylboronic acid (8.2 g, 38.9 mmol) were added to 600 ml of tetrahydrofuran under a nitrogen atmosphere, and the mixture was stirred and refluxed. Then, potassium carbonate (16.1 g, 116.7 mmol) was dissolved in 16 ml of water and added thereto, and the mixture was sufficiently stirred, and then tetrakis(triphenylphosphine)palladium (1.3 g, 1.2 mmol) was added. After reacting for 1 hour, the reaction mixture was cooled to room temperature, and then the organic layer and the aqueous layer were separated, and the organic layer was distilled. This was again dissolved in 1257 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 17.3 g of Compound 51 as a white solid (Yield: 69%, MS:[M+H]⁺=647.3).

### [Preparation Example 52] Synthesis of Compound 52

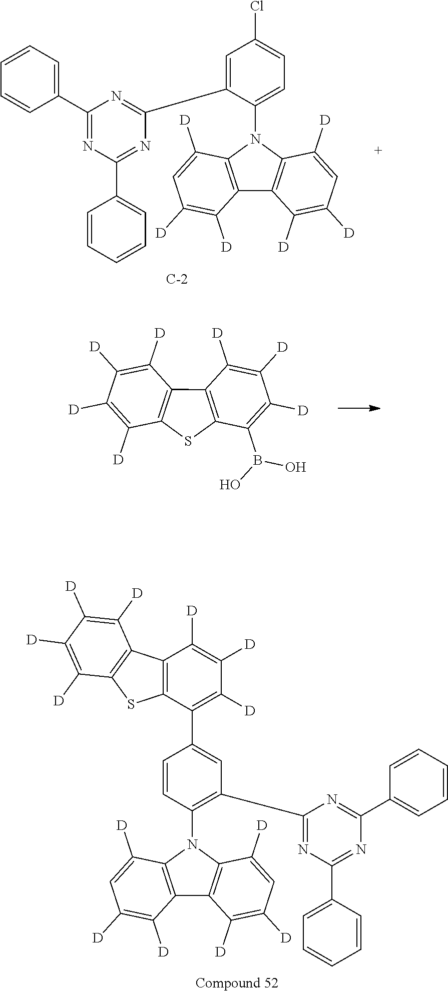

C-2 (20 g, 38.9 mmol) and (dibenzo[b,d]thiophen-4-yl-1,2,3,6,7,8,9-d7)boronic acid (9.1 g, 38.9 mmol) were added to 600 ml of tetrahydrofuran under a nitrogen atmosphere, and the mixture was stirred and refluxed. Then, potassium carbonate (16.1 g, 116.7 mmol) was dissolved in 16 ml of water and added thereto, and the mixture was sufficiently stirred, and then tetrakis(triphenylphosphine)palladium (1.3 g, 1.2 mmol) was added. After reacting for 3 hours, the reaction mixture was cooled to room temperature, and then the organic layer and the aqueous layer were separated, and the organic layer was distilled. This was again dissolved in 1302 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 15.1 g of Compound 52 as a while solid (Yield: 58%, MS:[M+H]⁺=670.3).

### [Preparation Example 53] Synthesis of Compound 53

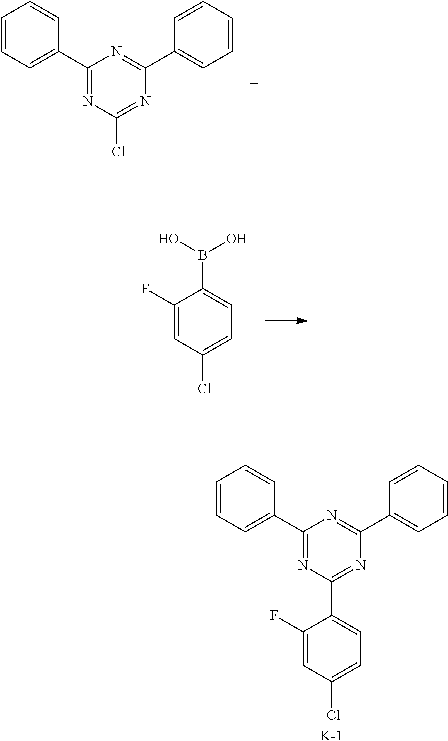

2-Chloro-4,6-diphenyl-1,3,5-triazine (100 g, 374.4 mmol) and (4-chloro-2-fluorophenyl)boronic acid (65.2 g, 374.4 mmol) were added to 2000 ml of tetrahydrofuran under a nitrogen atmosphere, and the mixture was stirred and refluxed. Then, potassium carbonate (155.3 g, 1123.3 mmol) was dissolved in 155 ml of water and added thereto, and the mixture was sufficiently stirred, and then tetrakis(triphenylphosphine)palladium (13 g, 11.2 mmol) was added. After reacting for 3 hours, the reaction mixture was cooled to room temperature, and then the organic layer and the aqueous layer were separated, and the organic layer was distilled. This was again dissolved in 6760 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 108.2 g of Compound K-1 as a white solid (Yield: 80%, MS:[M+H]⁺=362.1).

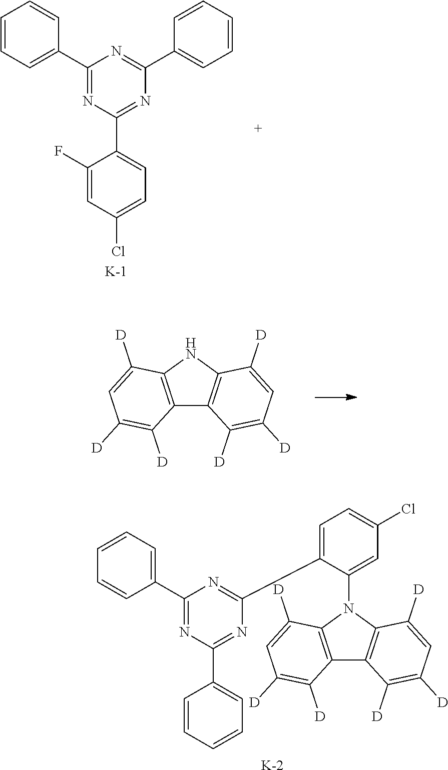

K-1 (50 g, 138.5 mmol) and 9H-carbazole-1,3,4,5,6,8-d6 (24 g, 138.5 mmol) were added to 1000 ml of dimethylformamide under a nitrogen atmosphere, and the mixture was stirred and refluxed. Then, potassium triphosphate (88.2 g, 415.4 mmol) was added thereto and sufficiently stirred. After reacting for 7 hours, the reaction mixture was cooled to room temperature and then the organic layer was subjected to filtration treatment to remove a salt, and then the filtered organic layer was distilled. This was added again to 712 ml of chloroform, dissolved and washed twice with water. The organic layer was then separated, to which anhydrous magnesium sulfate was added, stirred, then filtered, and the filtrate was distilled under reduced pressure. The concentrated compound was purified through a silica column using chloroform and ethyl acetate to give 38.4 g of Compound K-2 as a yellow solid (Yield: 54%, MS: [M+H]⁺=515.2).

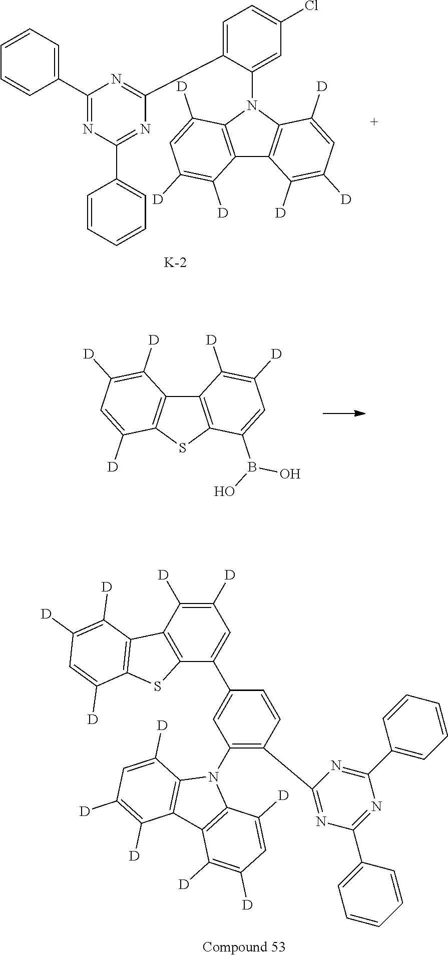

K-2 (20 g, 38.9 mmol) and (dibenzo[b,d]thiophen-4-yl-1,2,6,8,9-d5)boronic acid (9.1 g, 38.9 mmol) were added to 600 ml of tetrahydrofuran under a nitrogen atmosphere, and the mixture was stirred and refluxed. Then, potassium carbonate (16.1 g, 116.7 mmol) was dissolved in 16 ml of water and added thereto, and the mixture was sufficiently stirred, and then tetrakis(triphenylphosphine)palladium (1.3 g, 1.2 mmol) was added. After reacting for 2 hours, the reaction mixture was cooled to room temperature, and then the organic layer and the aqueous layer were separated, and the organic layer was distilled. This was again dissolved in 1298 mL of chloroform, washed twice with water, and then the organic layer was separated, to which anhydrous magnesium sulfate was added, stirred, then filtered, and the filtrate was distilled under reduced pressure. The concentrated compound was recrystallized from chloroform and ethanol to give 17.4 g of Compound 53 as a white solid (Yield: 67%, MS:[M+H]⁺=668.3).

### EXPERIMENTAL EXAMPLES

### Experimental Example 1

A glass substrate on which a thin film of ITO (indium tin oxide) was coated in a thickness of 1300 Å was put into distilled water containing a detergent dissolved therein and ultrasonically washed. In this case, the detergent used was a product commercially available from Fisher Co. and the distilled water was one which had been twice filtered by using a filter commercially available from Millipore Co. The ITO was cleaned for 30 minutes, and then ultrasonic cleaning was then repeated twice for 10 minutes by using distilled water. After the cleaning with distilled water was completed, the substrate was ultrasonically washed with the solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was thermally vacuum-deposited to a thickness of 50 Å to form a hole injection layer. The following compound HT-1 was thermally vacuum-deposited on the hole injection layer to a thickness of 250 Å to form a hole transport layer, and the following compound HT-2 was vacuum-deposited on the HT-1 deposited layer to a thickness of 50 Å to form an electron blocking layer. Then, Compound 1 prepared in the previous Preparation Example 1 as a light emitting layer, the following Compound YGH-1, and the following phosphorescence dopant YGD-1 were vacuum deposited at a weight ratio of 44:44:12 on the HT-2 deposited layer to form a light emitting layer with a thickness of 400 Å. The following Compound ET-1 was vacuum deposited on the light emitting layer to a thickness of 250 Å to form an electron transport layer, and the following Compound ET-2 and Li were vacuum deposited at a weight ratio of 98:2 on the electron transport layer to form an electron injection layer with a film thickness of 100 Å. Aluminum was deposited on the electron injection layer to a thickness of 1,000 Å to form a cathode.

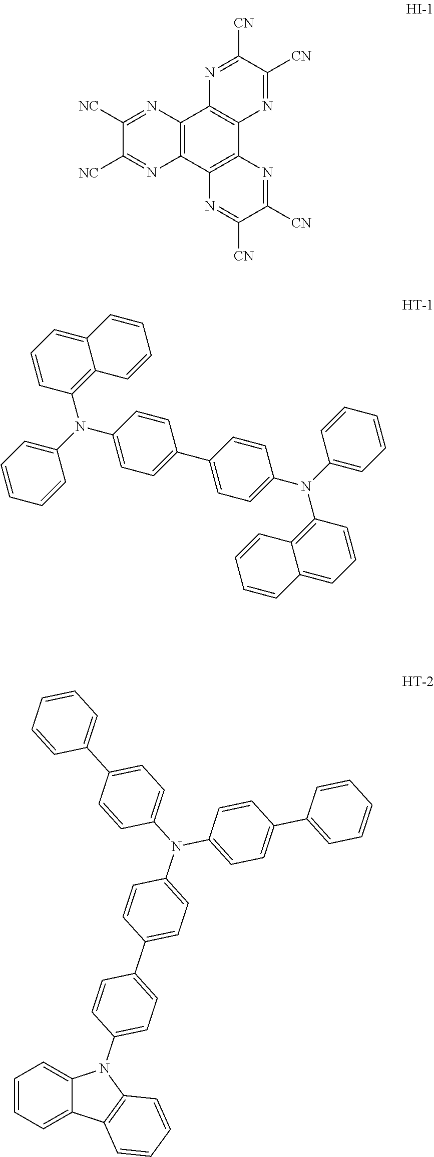 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccc(-n5c6ccccc6c6ccccc65)cc4)cc3)cc2)cc1]

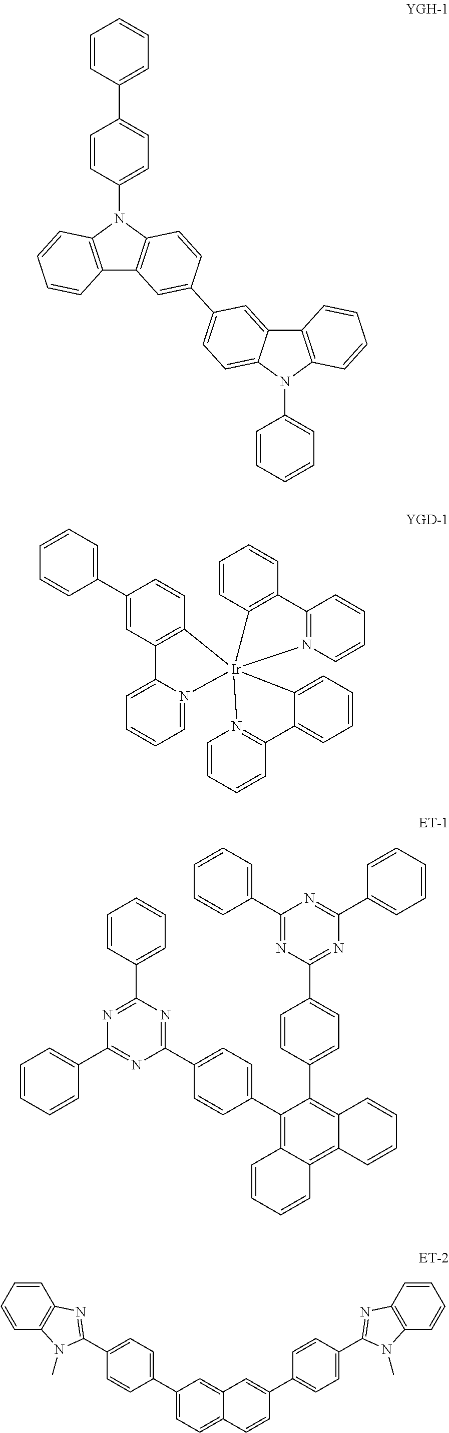

In the above-mentioned processes, the deposition rate of the organic material was maintained at 0.4˜0.7 Å/sec, the deposition rate of aluminum was maintained at 2 Å/sec, and the degree of vacuum during deposition was maintained at 1×10⁻⁷˜5×10⁻⁸ torr.

### Experimental Examples 2 to 53

The organic light emitting devices were manufactured in the same manner as in Experimental Example 1, except that in Experimental Example 1, Compounds shown in Tables 1 and 2 below were used instead of the Compound 1 of Preparation Example 1.

### Comparative Experimental Example 1

The organic light emitting device was manufactured in the same manner as in Experimental Example 1, except that in Experimental Example 1, Compounds shown in Table 1 below were used instead of the Compound 1 of Preparation Example 1. The Compound CE1 of Table 3 below are as follows.

### Comparative Experimental Examples 2 to 5

The organic light emitting devices were manufactured in the same manner as in Experimental Example 1, except that in Experimental Example 1, Compounds shown in Table 3 below were used instead of the Compound 1 of Preparation Example 1.

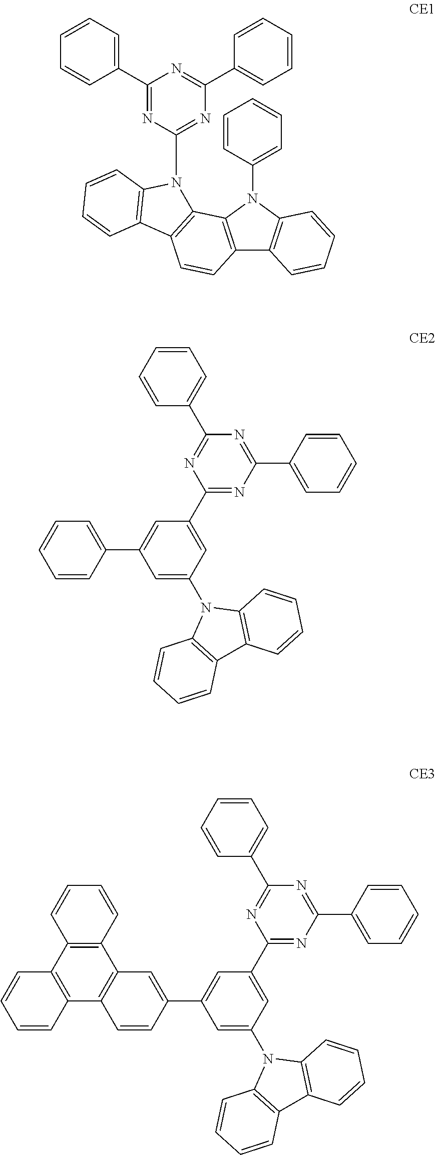 [SMILES: c1ccc(-c2nc(-c3ccccc3)nc(-n3c4ccccc4c4ccc5c6ccccc6n(-c6ccccc6)c5c43)n2)cc1 ; c1ccc(-c2cc(-c3nc(-c4ccccc4)nc(-c4ccccc4)n3)cc(-n3c4ccccc4c4ccccc43)c2)cc1 ; c1ccc(-c2nc(-c3ccccc3)nc(-c3cc(-c4ccc5c6ccccc6c6ccccc6c5c4)cc(-n4c5ccccc5c5ccccc54)c3)n2)cc1]

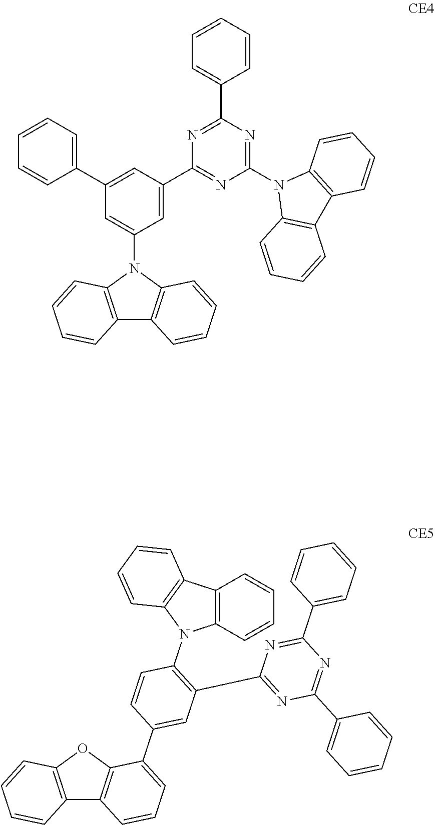 [SMILES: c1ccc(-c2cc(-c3nc(-c4ccccc4)nc(-n4c5ccccc5c5ccccc54)n3)cc(-n3c4ccccc4c4ccccc43)c2)cc1 ; c1ccc(-c2nc(-c3ccccc3)nc(-c3cc(-c4cccc5c4oc4ccccc45)ccc3-n3c4ccccc4c4ccccc43)n2)cc1]

The voltage and efficiency of the organic light emitting devices manufactured in Experimental Examples and Comparative Experimental Examples were measured at a current density of 10 mA/cm2, and the lifetime was measured at current density of 50 mA/cm2. The results are shown in Tables 1 to 3 below. At this time, T95 means the time required for the luminance to be reduced to 95% of the initial luminance.

TABLE 1

| | | | Efficiency | Color | Lifetime (h) |
|---|---|---|---|---|---|
| | | Voltage(V) | (Cd/A) | coordinate | (LT₉₅ at |
| | Compound | (@10 mA/cm²) | (@10 mA/cm²) | (x, y) | 50 mA/cm²) |
| Experimental | Compound 1 | 4.2 | 81 | 0.45, 0.53 | 250 |
| Example 1 | | | | | |
| Experimental | Compound 2 | 4.3 | 82 | 0.44, 0.54 | 260 |
| Example 2 | | | | | |
| Experimental | Compound 3 | 4.4 | 80 | 0.45, 0.54 | 280 |
| Example 3 | | | | | |
| Experimental | Compound 4 | 4.3 | 77 | 0.44, 0.53 | 260 |
| Example 4 | | | | | |
| Experimental | Compound 5 | 4.4 | 78 | 0.45, 0.53 | 290 |
| Example 5 | | | | | |
| Experimental | Compound 6 | 4.3 | 77 | 0.44, 0.54 | 220 |
| Example 6 | | | | | |
| Experimental | Compound 7 | 4.2 | 75 | 0.45, 0.53 | 210 |
| Example 7 | | | | | |
| Experimental | Compound 8 | 4.3 | 77 | 0.44, 0.54 | 280 |
| Example 8 | | | | | |
| Experimental | Compound 9 | 4.2 | 81 | 0.45, 0.54 | 270 |
| Example 9 | | | | | |
| Experimental | Compound 10 | 4.2 | 84 | 0.44, 0.53 | 220 |
| Example 10 | | | | | |
| Experimental | Compound 11 | 4.1 | 83 | 0.45, 0.54 | 210 |
| Example 11 | | | | | |
| Experimental | Compound 12 | 4.2 | 79 | 0.45, 0.53 | 280 |
| Example 12 | | | | | |
| Experimental | Compound 13 | 4.4 | 80 | 0.44, 0.54 | 295 |
| Example 13 | | | | | |
| Experimental | Compound 14 | 4.6 | 80 | 0.45, 0.54 | 315 |
| Example 14 | | | | | |
| Experimental | Compound 15 | 4.3 | 77 | 0.44, 0.53 | 290 |
| Example 15 | | | | | |
| Experimental | Compound 16 | 4.4 | 76 | 0.45, 0.53 | 325 |
| Example 16 | | | | | |
| Experimental | Compound 17 | 4.4 | 78 | 0.44, 0.54 | 250 |
| Example 17 | | | | | |
| Experimental | Compound 18 | 4.3 | 75 | 0.45, 0.53 | 230 |
| Example 18 | | | | | |
| Experimental | Compound 19 | 4.4 | 77 | 0.44, 0.54 | 315 |
| Example 19 | | | | | |
| Experimental | Compound 20 | 4.2 | 79 | 0.45, 0.54 | 300 |
| Example 20 | | | | | |
| Experimental | Compound 21 | 4.4 | 84 | 0.44, 0.53 | 250 |
| Example 21 | | | | | |
| Experimental | Compound 22 | 4.4 | 80 | 0.45, 0.54 | 240 |
| Example 22 | | | | | |
| Experimental | Compound 23 | 4.2 | 81 | 0.45, 0.53 | 270 |
| Example 23 | | | | | |
| Experimental | Compound 24 | 4.2 | 81 | 0.44, 0.54 | 280 |
| Example 24 | | | | | |
| Experimental | Compound 25 | 4.2 | 81 | 0.45, 0.54 | 260 |
| Example 25 | | | | | |

TABLE 2

| | | | Efficiency | Color | Lifetime (h) |
|---|---|---|---|---|---|
| | | Voltage (V) | (Cd/A) | coordinate | (LT₉₅ at |
| | Compound | (@10 mA/cm²) | (@10 mA/cm²) | (x, y) | 50 mA/cm²) |
| Experimental | Compound | 4.5 | 75 | 0.44, 0.53 | 280 |
| Example 26 | 26 | | | | |
| Experimental | Compound | 4.3 | 80 | 0.45, 0.53 | 250 |
| Example 27 | 27 | | | | |
| Experimental | Compound | 4.2 | 81 | 0.44, 0.54 | 280 |
| Example 28 | 28 | | | | |
| Experimental | Compound | 4.3 | 83 | 0.45, 0.53 | 265 |
| Example 29 | 29 | | | | |
| Experimental | Compound | 4.4 | 84 | 0.44, 0.54 | 280 |
| Example 30 | 30 | | | | |
| Experimental | Compound | 4.6 | 84 | 0.45, 0.54 | 300 |
| Example 31 | 31 | | | | |
| Experimental | Compound | 4.5 | 81 | 0.44, 0.53 | 275 |
| Example 32 | 32 | | | | |
| Experimental | Compound | 4.4 | 80 | 0.45, 0.53 | 310 |
| Example 33 | 33 | | | | |
| Experimental | Compound | 4.6 | 82 | 0.44, 0.54 | 235 |
| Example 34 | 34 | | | | |
| Experimental | Compound | 4.4 | 79 | 0.45, 0.53 | 240 |
| Example 35 | 35 | | | | |
| Experimental | Compound | 4.6 | 81 | 0.44, 0.54 | 310 |
| Example 36 | 36 | | | | |
| Experimental | Compound | 4.4 | 82 | 0.45, 0.54 | 280 |
| Example 37 | 37 | | | | |
| Experimental | Compound | 4.5 | 88 | 0.44, 0.53 | 240 |
| Example 38 | 38 | | | | |
| Experimental | Compound | 4.4 | 83 | 0.45, 0.54 | 210 |
| Example 39 | 39 | | | | |
| Experimental | Compound | 4.1 | 85 | 0.45, 0.53 | 215 |
| Example 40 | 40 | | | | |
| Experimental | Compound | 4.2 | 86 | 0.44, 0.54 | 220 |
| Example 41 | 41 | | | | |
| Experimental | Compound | 4.3 | 84 | 0.45, 0.54 | 240 |
| Example 42 | 42 | | | | |
| Experimental | Compound | 4.3 | 80 | 0.44, 0.53 | 220 |
| Example 43 | 43 | | | | |
| Experimental | Compound | 4.2 | 82 | 0.45, 0.53 | 250 |
| Example 44 | 44 | | | | |
| Experimental | Compound | 4.3 | 80 | 0.44, 0.54 | 190 |
| Example 45 | 45 | | | | |
| Experimental | Compound | 4.2 | 79 | 0.45, 0.53 | 180 |
| Example 46 | 46 | | | | |
| Experimental | Compound | 4.2 | 81 | 0.44, 0.54 | 240 |
| Example 47 | 47 | | | | |
| Experimental | Compound | 4.2 | 84 | 0.45, 0.54 | 230 |
| Example 48 | 48 | | | | |
| Experimental | Compound | 4.2 | 84 | 0.44, 0.53 | 190 |
| Example 49 | 49 | | | | |
| Experimental | Compound | 4.3 | 81 | 0.45, 0.54 | 200 |
| Example 50 | 50 | | | | |
| Experimental | Compound | 4.1 | 85 | 0.45, 0.53 | 240 |
| Example 51 | 51 | | | | |
| Experimental | Compound | 4.3 | 84 | 0.45, 0.53 | 260 |
| Example 52 | 52 | | | | |
| Experimental | Compound | 4.4 | 82 | 0.45, 0.54 | 315 |
| Example 53 | 53 | | | | |

TABLE 3

| | | | Efficiency | Color | Lifetime (h) |
|---|---|---|---|---|---|
| | | Voltage (V) | (Cd/A) | coordinate | (LT₉₅ at |
| | Compound | (@10 mA/cm²) | (@10 mA/cm²) | (x, y) | 50 mA/cm²) |
| Comparative | CE 1 | 4.2 | 74 | 0.45, 0.53 | 80 |
| Experimental | | | | | |
| Example 1 | | | | | |
| Comparative | CE 2 | 4.2 | 81 | 0.45, 0.53 | 130 |
| Experimental | | | | | |
| Example 2 | | | | | |
| Comparative | CE 3 | 4.5 | 74 | 0.44, 0.54 | 70 |
| Experimental | | | | | |
| Example 3 | | | | | |
| Comparative | CE 4 | 4.4 | 75 | 0.45, 0.54 | 90 |
| Experimental | | | | | |
| Example 4 | | | | | |
| Comparative | CE 5 | 4.2 | 81 | 0.45, 0.53 | 150 |
| Experimental | | | | | |
| Example 5 | | | | | |

As shown in Tables 1 to 3, it can be confirmed that when the compound of the present disclosure is used as a material for the light emitting layer, it exhibits excellent characteristics in terms of efficiency and lifetime compared to Comparative Experimental Examples. This appears that the electronic stability is increased while triazine and carbazole, dibenzofuran and dibenzothiophene are distributed in the molecule. In particular, the carbazole group has an excellent characteristic of increasing the lifetime when additional deuterium is substituted. This also appear to have increased electronic stability.

### DESCRIPTION OF SYMBOLS

| | |
|---|---|
| 1: substrate | 2: anode |
| 3: light emitting layer | 4: cathode |
| 5: hole injection layer | 6: hole transport layer |
| 7: light emitting layer | 8: electron injection and transport layer |

1. A compound of Chemical Formula 1: 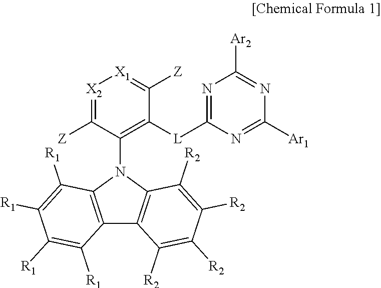
wherein, in Chemical Formula 1:
each R₁ is independently hydrogen deuterium, a substituted or unsubstituted C₆₋₆₀ aryl, or a substituted or unsubstituted C₂₋₆₀ heteroaryl containing one or more heteroatoms selected from the group consisting of N, O and S, or two adjacent R₁s are connected with each other to form a substituted or unsubstituted C₆₋₆₀ aromatic ring, or a substituted or unsubstituted C₂₋₆₀ heteroaromatic ring containing one or more heteroatoms selected from the group consisting of N, O and S;
each R₂ is independently hydrogen or deuterium, provided that at least one of R₂s is deuterium;
one of X₁ and X₂ is carbon (C) connected to a substituent represented by the following Chemical Formula 2, and the other is CH or CD;
Ar₁ is a substituted or unsubstituted C₆₋₆₀ aryl or a substituted or unsubstituted C₂₋₆₀ heteroaryl containing one or more heteroatoms selected from the group consisting of N, O and S;
Ar₂ is a substituted or unsubstituted C₆₋₆₀ aryl or a substituted or unsubstituted C₂₋₆₀ heteroaryl containing one or more heteroatoms selected from the group consisting of N, O and S;
each Z is independently hydrogen or deuterium;
L is a direct bond or a substituted or unsubstituted C₆₋₆₀ aryl; 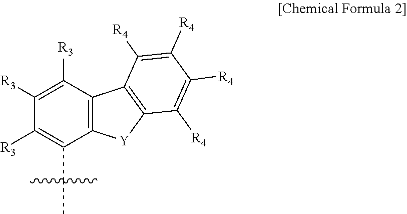
wherein, in Chemical Formula 2:
Y is O or S;
each R₃ is independently hydrogen, deuterium, a substituted or unsubstituted C₆₋₆₀ aryl, or a substituted or unsubstituted C₂₋₆₀ heteroaryl containing one or more heteroatoms selected from the group consisting of N, O and S; and
each R₄ is independently hydrogen deuterium, a substituted or unsubstituted C₆₋₆₀ aryl or a substituted or unsubstituted C₂₋₆₀ heteroaryl containing one or more heteroatoms selected from the group consisting of N, O and S.

2. The compound according to claim 1, wherein:
the compound of Chemical Formula 1 is a compound of any one of the following Chemical Formulas 3a to 3o: 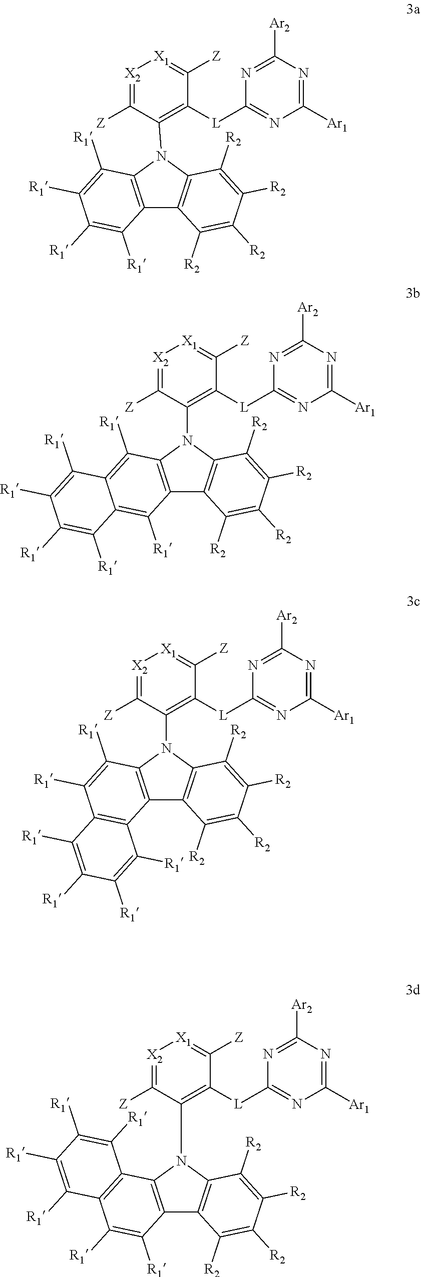 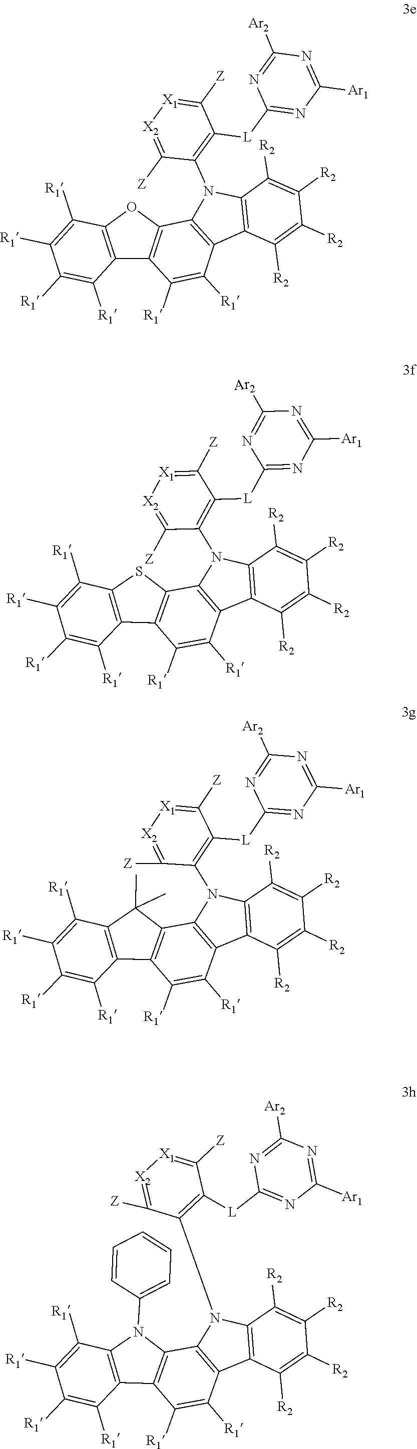 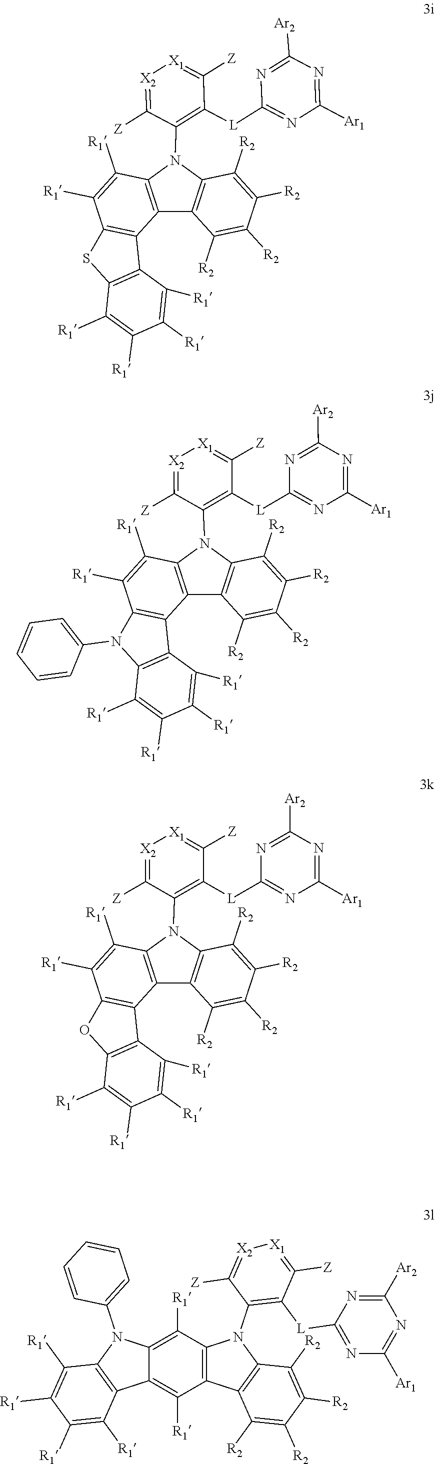 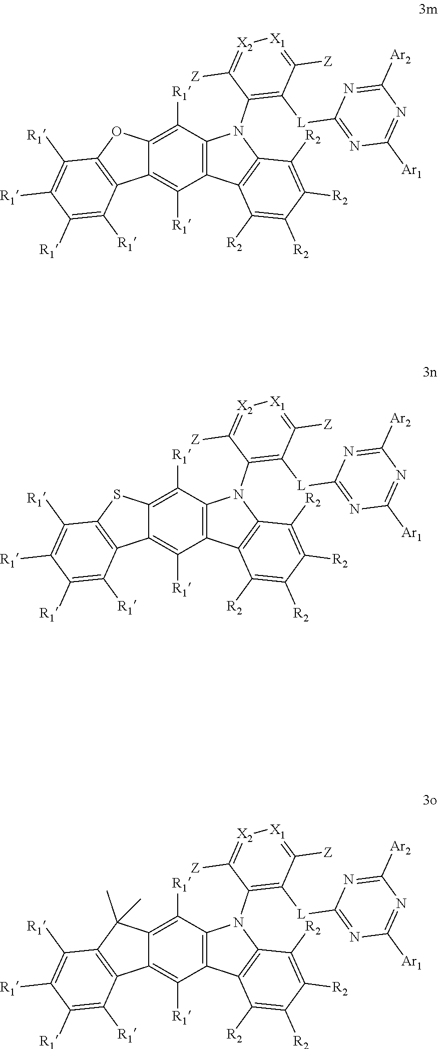
wherein:
Ar₁, Ar₂, Z, X₁, X₂, R₁′, R₂ and L are as defined in claim 1.

3. The compound according to claim 2, wherein:
in Chemical Formulas 3a to 3o,
each R₁′ is independently hydrogen, deuterium, or phenyl,
wherein the phenyl is unsubstituted or substituted with at least one deuterium.

4. The compound according to claim 1, wherein:
Ar₁ is phenyl, biphenylyl, terphenylyl, naphthylphenyl, naphthyl, phenanthryl, dibenzofuranyl, dibenzothiophenyl, phenylcarbazolyl, or N-carbazolyl,
wherein the Ar₁ is unsubstituted or substituted with at least one deuterium.

5. The compound according to claim 1, wherein:
Ar₂ is phenyl, biphenylyl, terphenylyl, naphthylphenyl, naphthyl, phenanthryl, dibenzofuranyl, dibenzothiophenyl, phenylcarbazolyl, or N-carbazolyl,
wherein the Ar₂ is unsubstituted or substituted with at least one deuterium.

6. The compound according to claim 1, wherein:
L is a direct bond.

7. The compound according to claim 1, wherein:
each R₃ is independently hydrogen, deuterium, or phenyl that is unsubstituted or substituted with deuterium.

8. The compound according to claim 1, wherein:
each R₄ is independently hydrogen, deuterium, or phenyl that is unsubstituted or substituted with deuterium.

9. The compound according to claim 1, wherein:
the substituent 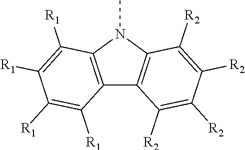
is any one substituent selected from among the following: 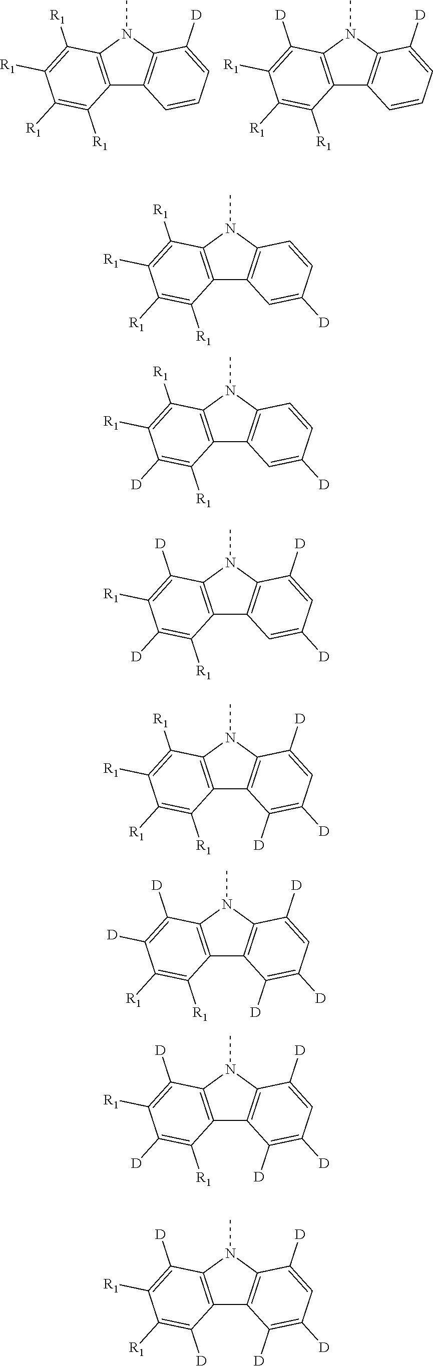 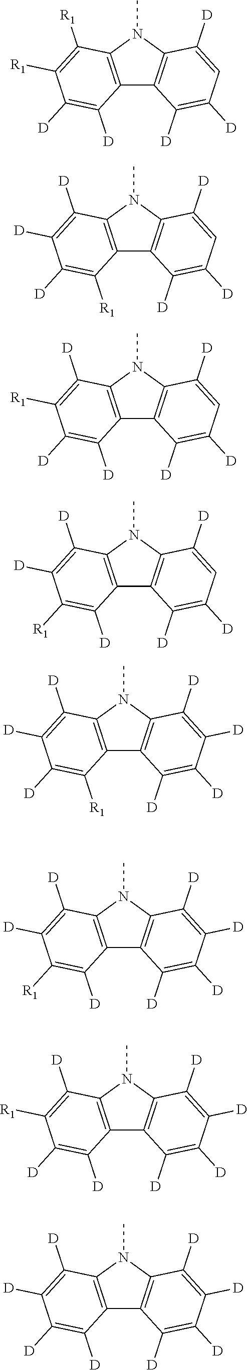
wherein R₁ is as defined in claim 1.

10. The compound according to claim 1, wherein:
the compound of Chemical Formula 1 is any one compound selected from among the following:

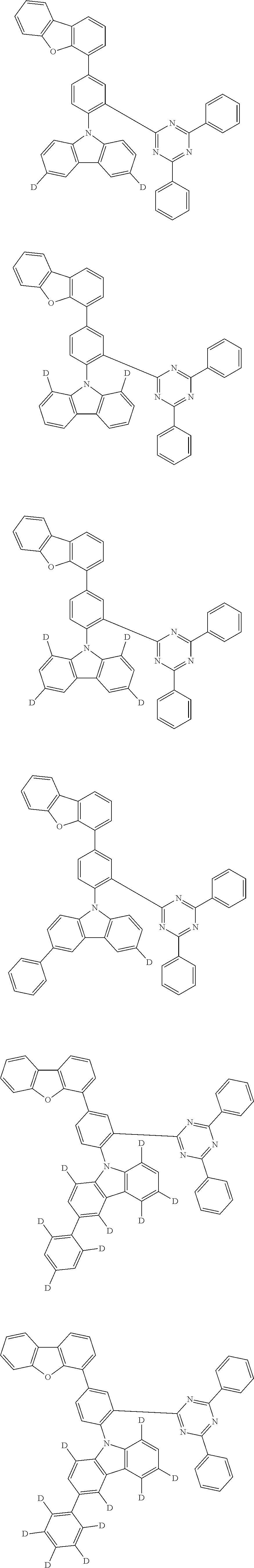

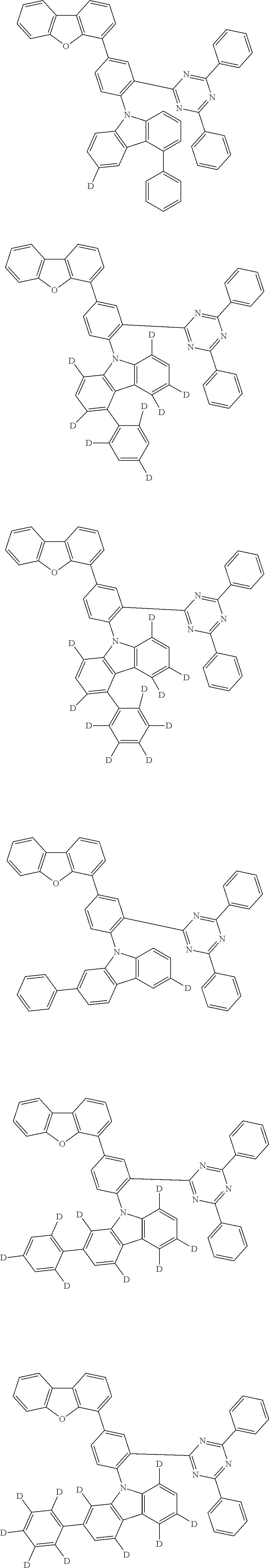

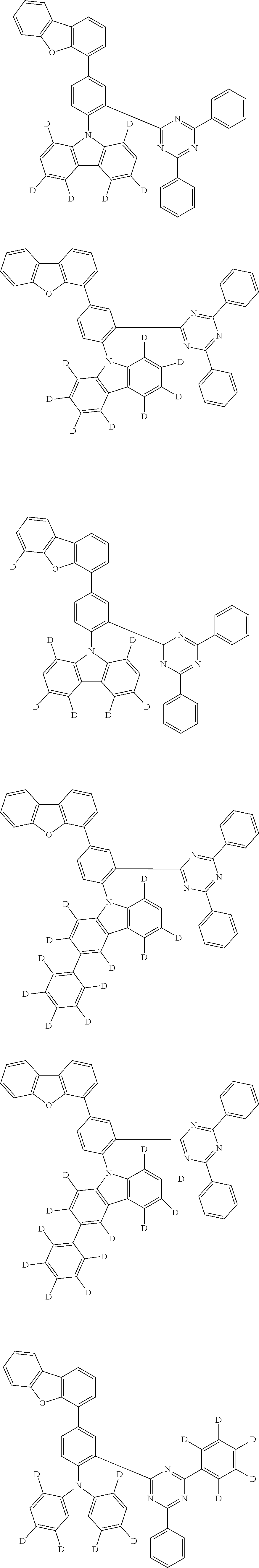

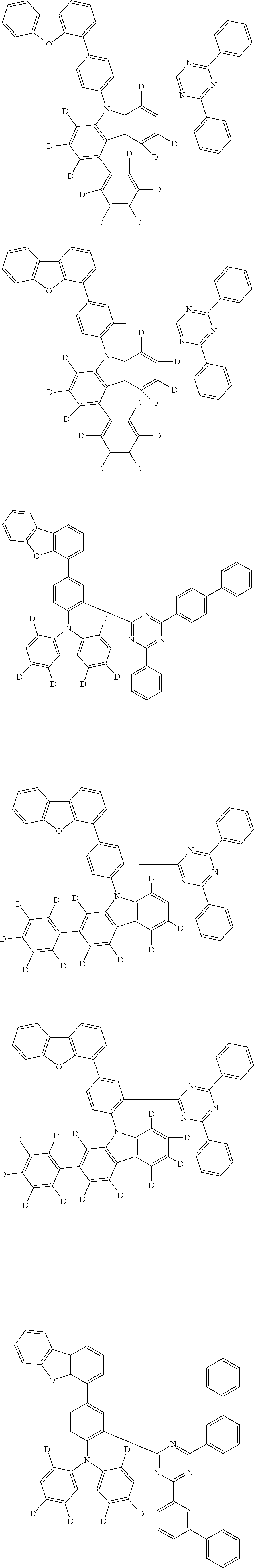

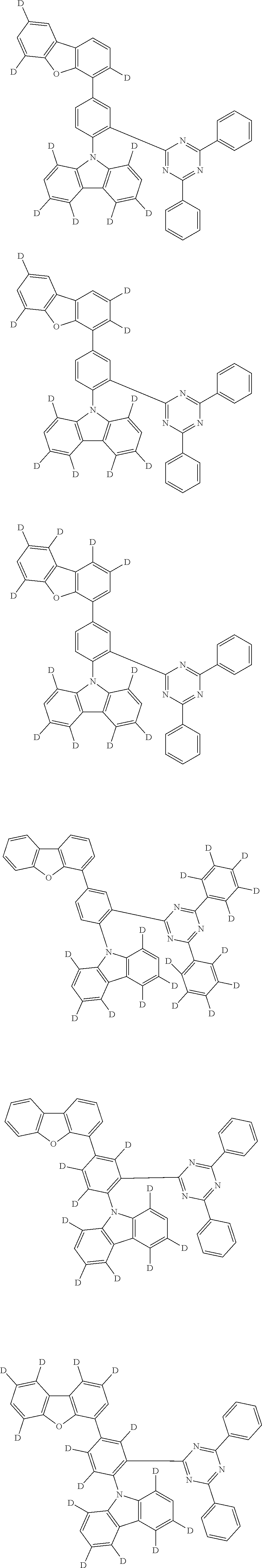

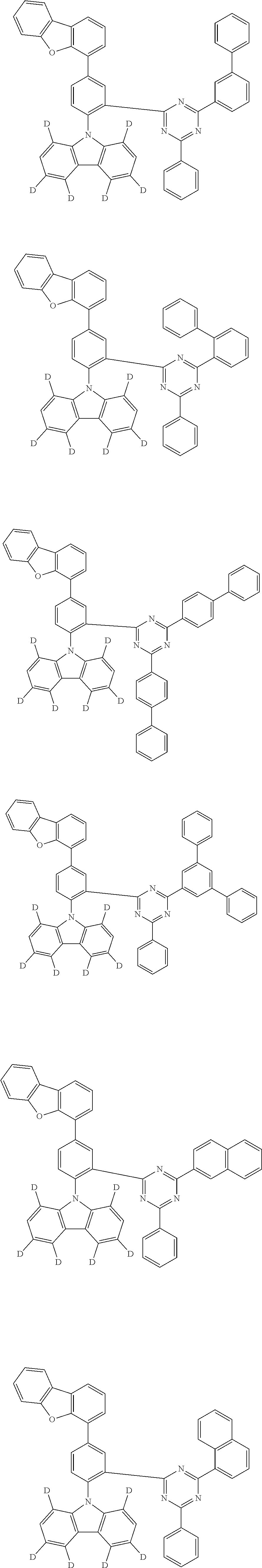

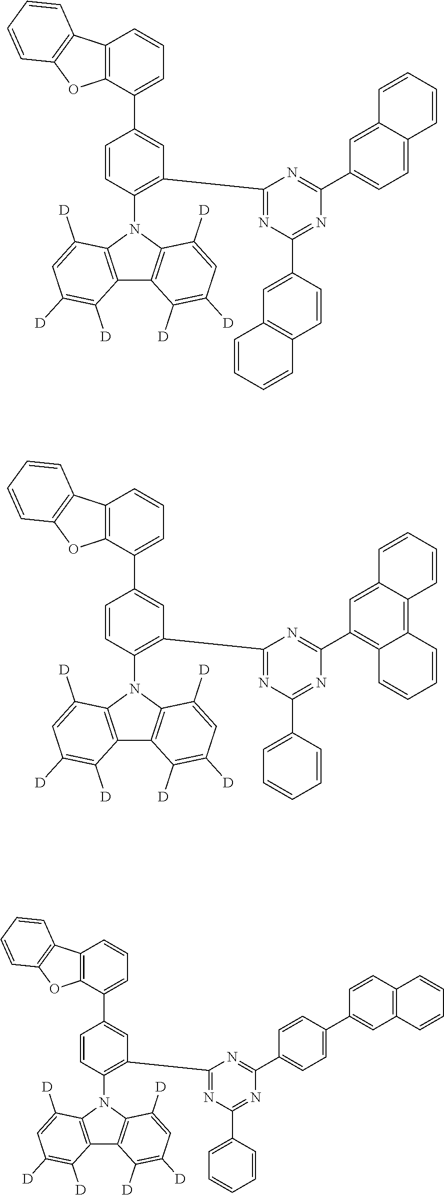 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccc3ccccc3c2)nc(-c2ccc3ccccc3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cc3ccccc3c3ccccc23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc(-c3ccc4ccccc4c3)cc2)n1]

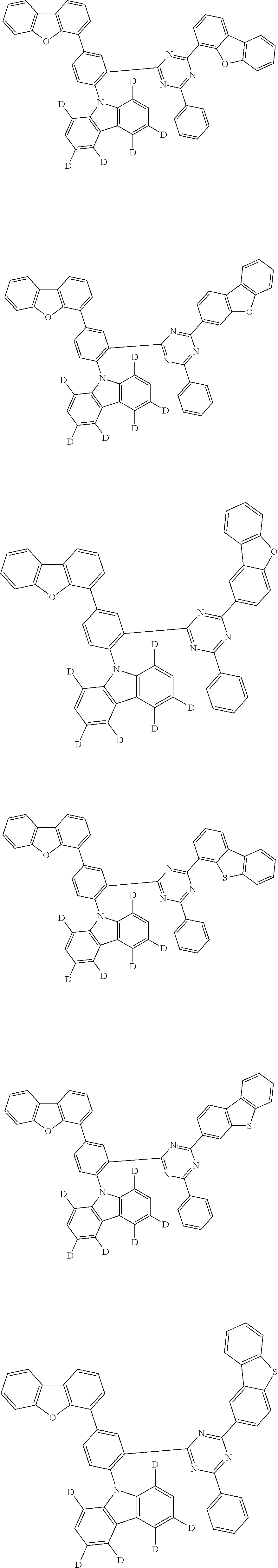

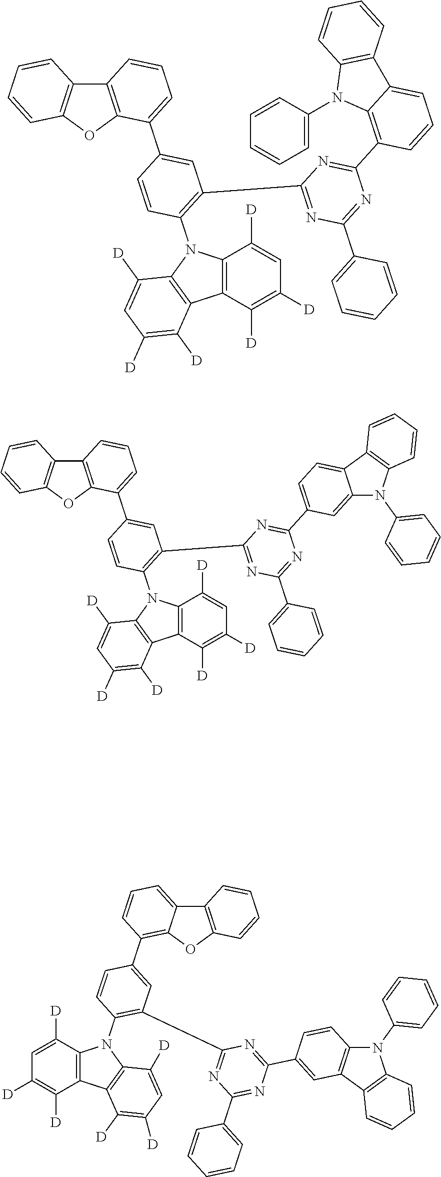 [SMILES: [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2cccc3c4ccccc4n(-c4ccccc4)c23)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c4ccccc4n(-c4ccccc4)c3c2)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-c2ccc3c(c2)c2ccccc2n3-c2ccccc2)n1]

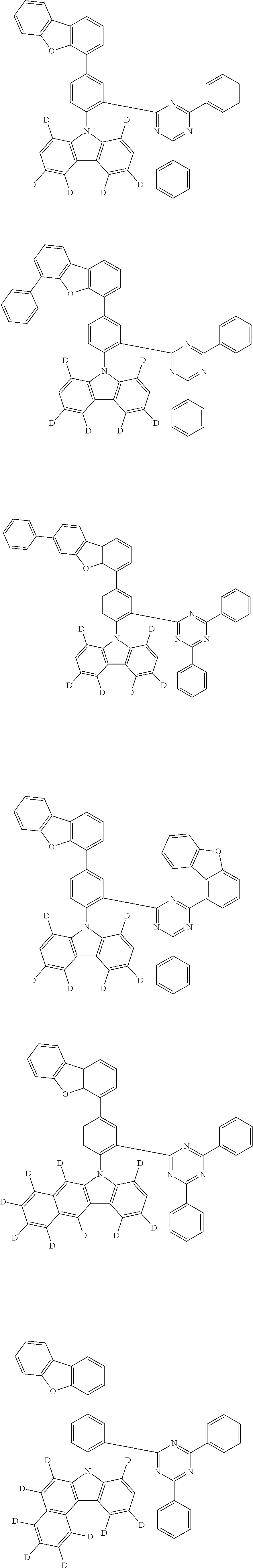

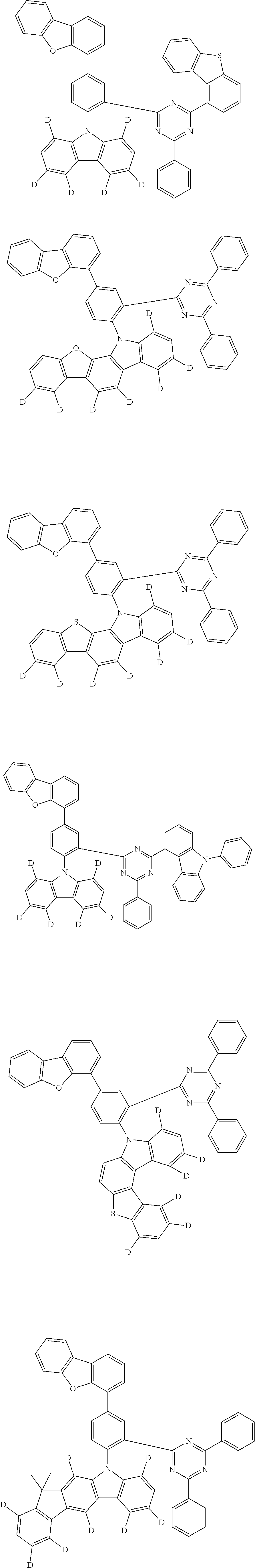

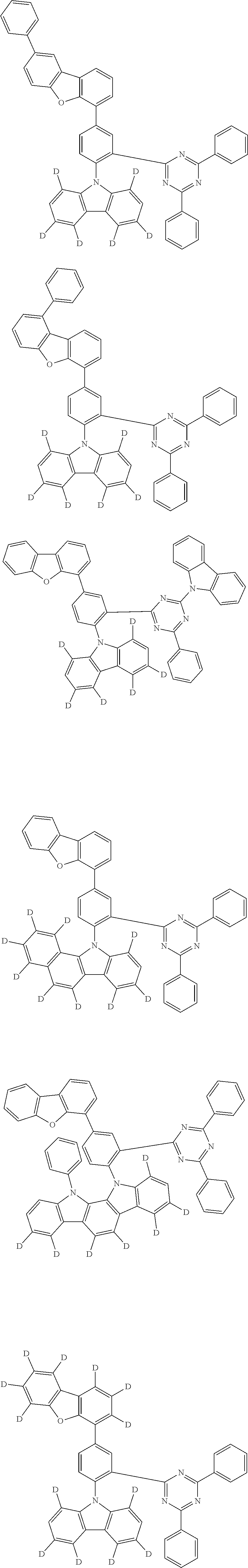

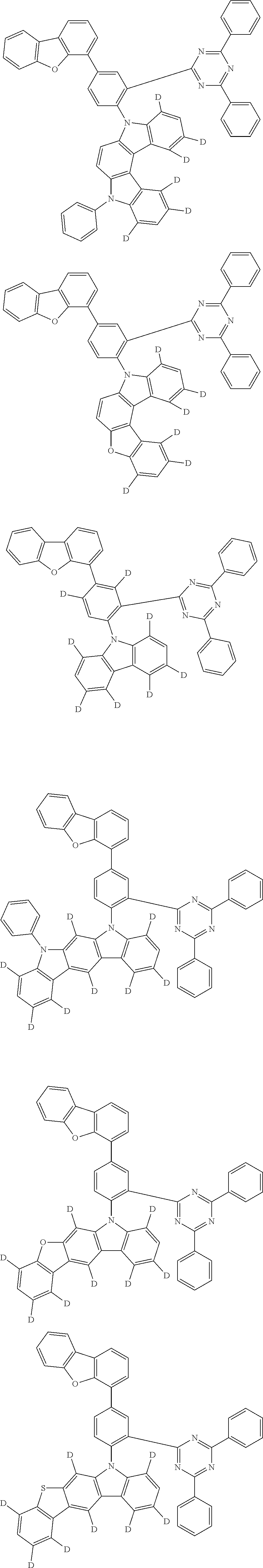

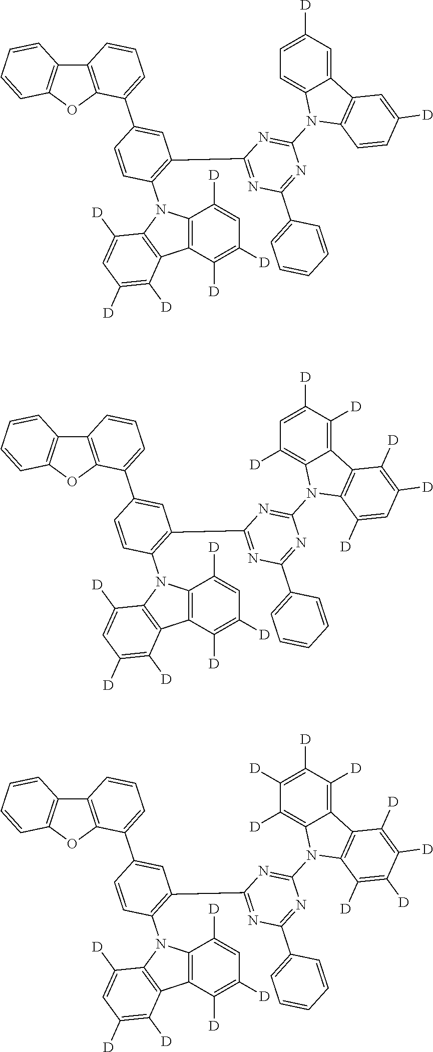 [SMILES: [2H]c1ccc2c(c1)c1cc([2H])ccc1n2-c1nc(-c2ccccc2)nc(-c2cc(-c3cccc4c3oc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1nc(-c2ccccc2)nc(-c2cc(-c3cccc4c3oc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1ccc(-c2cccc3c2oc2ccccc23)cc1-c1nc(-c2ccccc2)nc(-n2c3c([2H])c([2H])c([2H])c([2H])c3c3c([2H])c([2H])c([2H])c([2H])c32)n1]

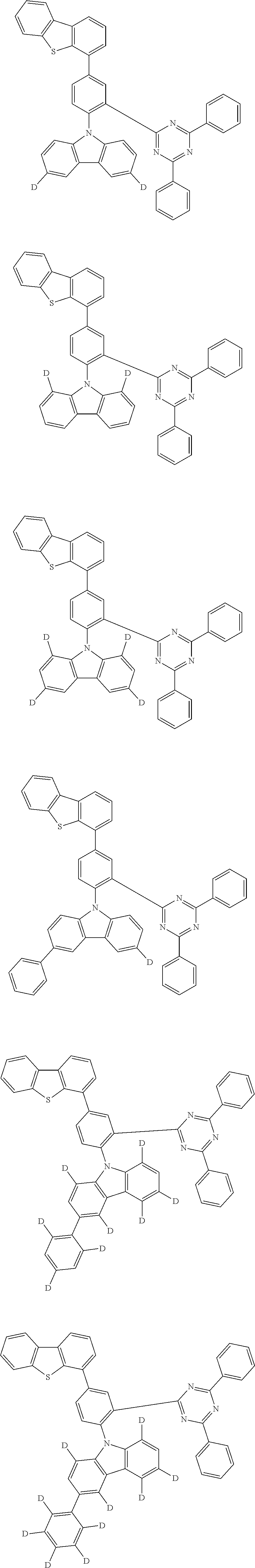

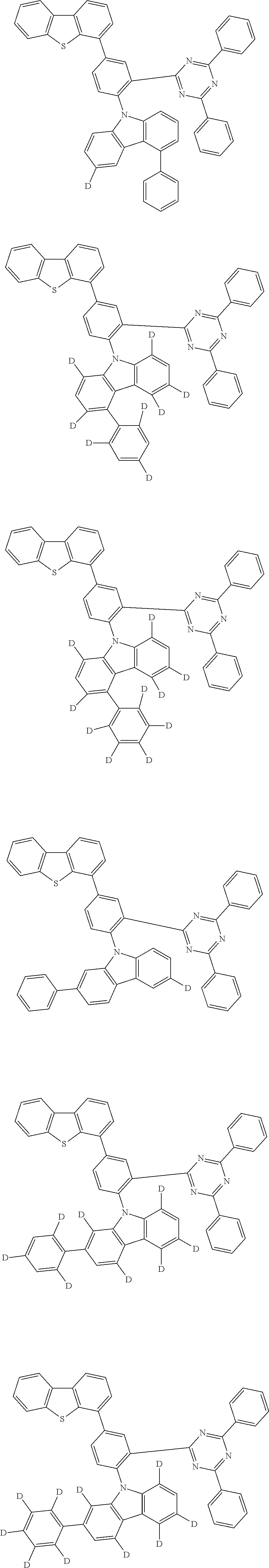

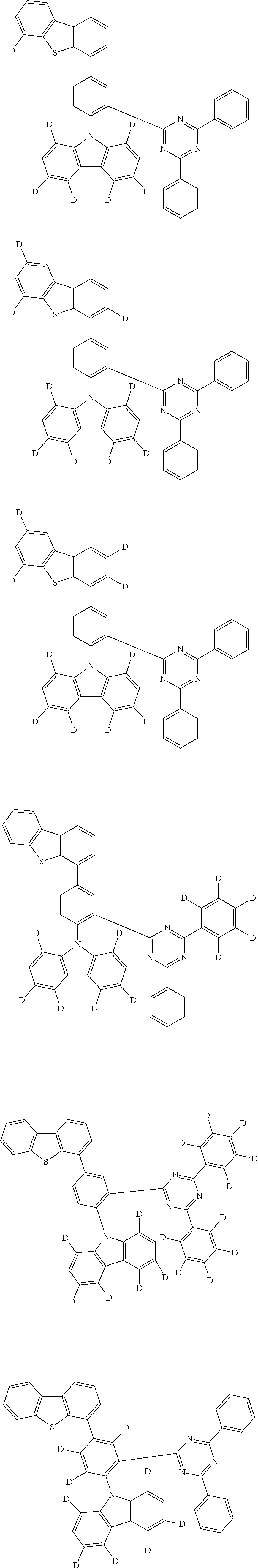

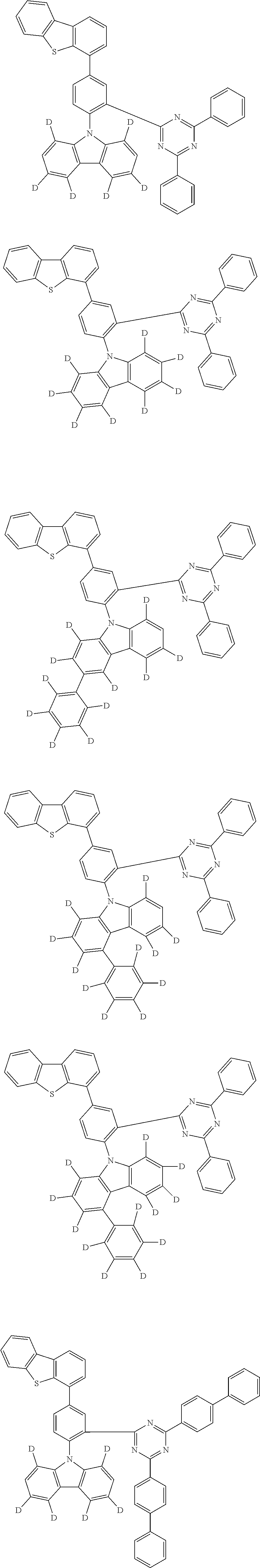

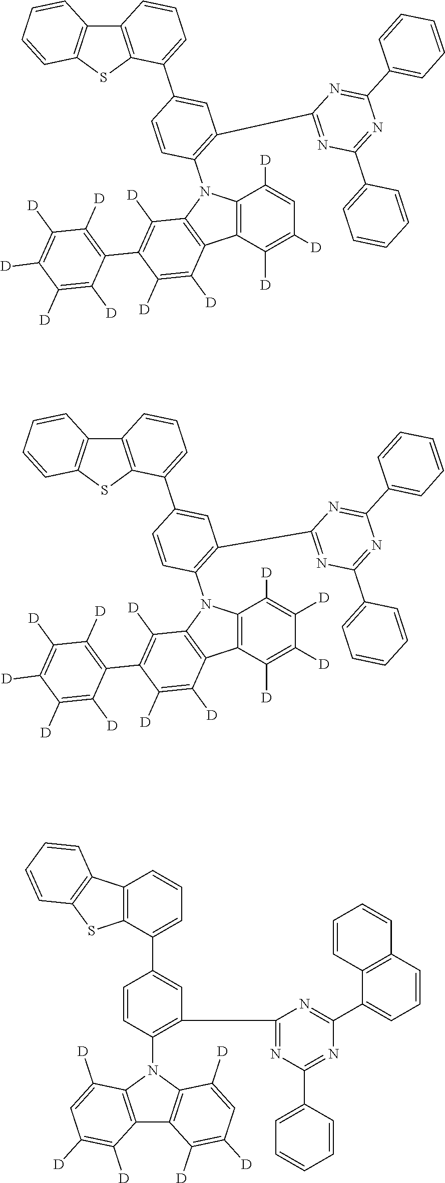 [SMILES: [2H]c1cc([2H])c2c(c1[2H])c1c([2H])c([2H])c(-c3c([2H])c([2H])c([2H])c([2H])c3[2H])c([2H])c1n2-c1ccc(-c2cccc3c2sc2ccccc23)cc1-c1nc(-c2ccccc2)nc(-c2ccccc2)n1 ; [2H]c1c([2H])c([2H])c(-c2c([2H])c([2H])c3c4c([2H])c([2H])c([2H])c([2H])c4n(-c4ccc(-c5cccc6c5sc5ccccc56)cc4-c4nc(-c5ccccc5)nc(-c5ccccc5)n4)c3c2[2H])c([2H])c1[2H] ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3ccccc23)n1]

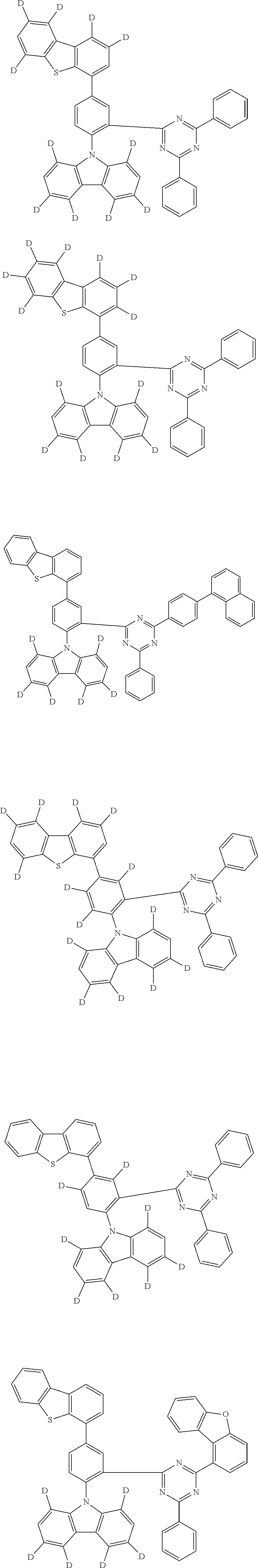

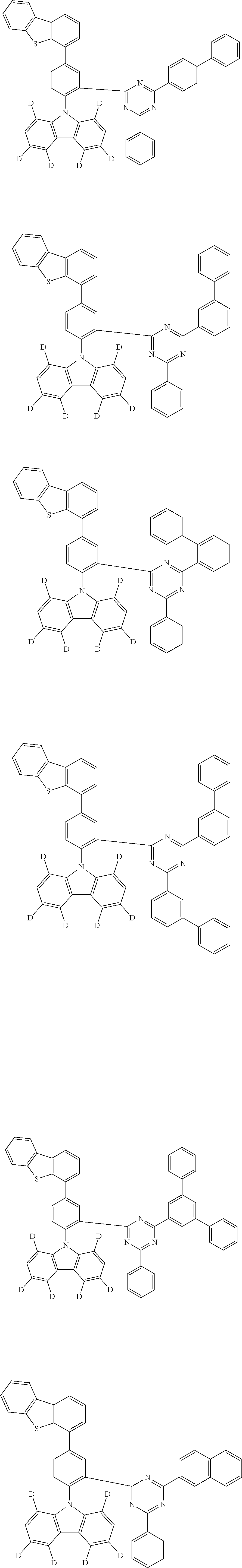

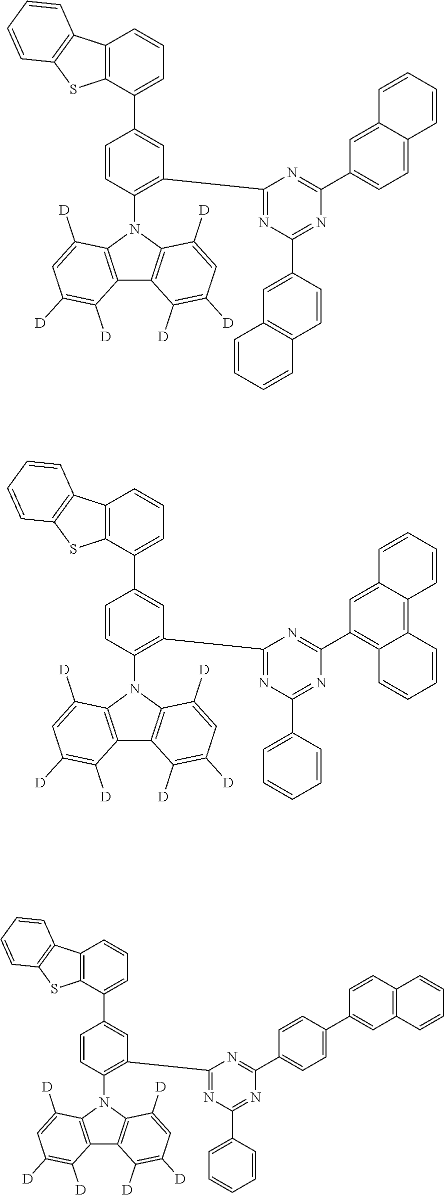 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccc3ccccc3c2)nc(-c2ccc3ccccc3c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cc3ccccc3c3ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc(-c3ccc4ccccc4c3)cc2)n1]

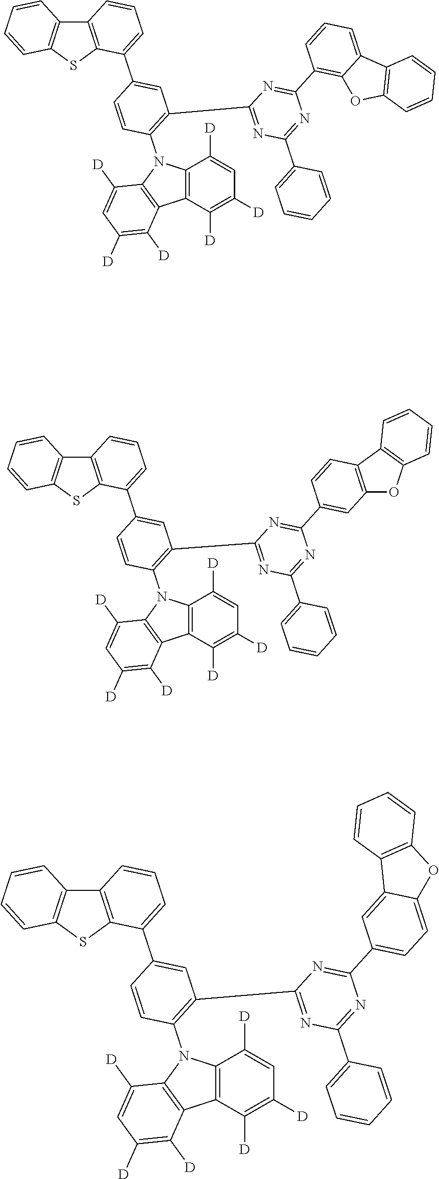 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2oc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3oc4ccccc4c3c2)n1]

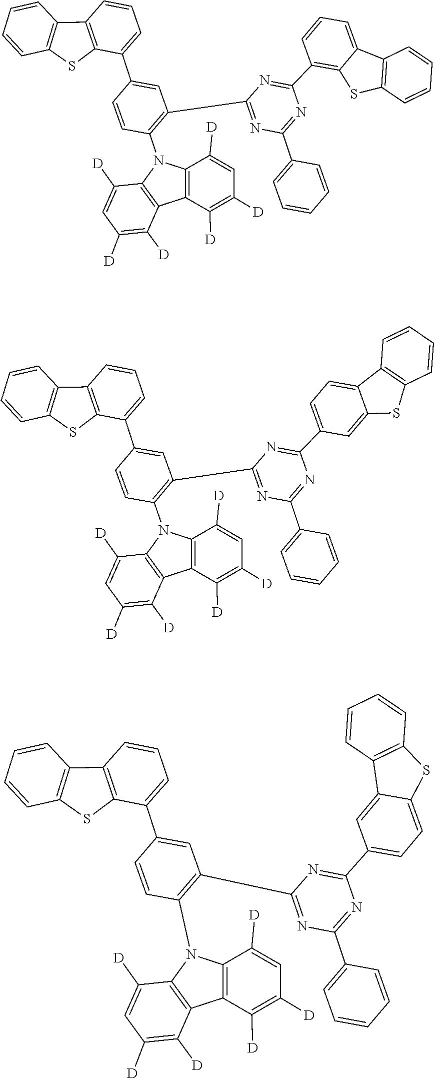 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2sc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)sc2ccccc23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3sc4ccccc4c3c2)n1]

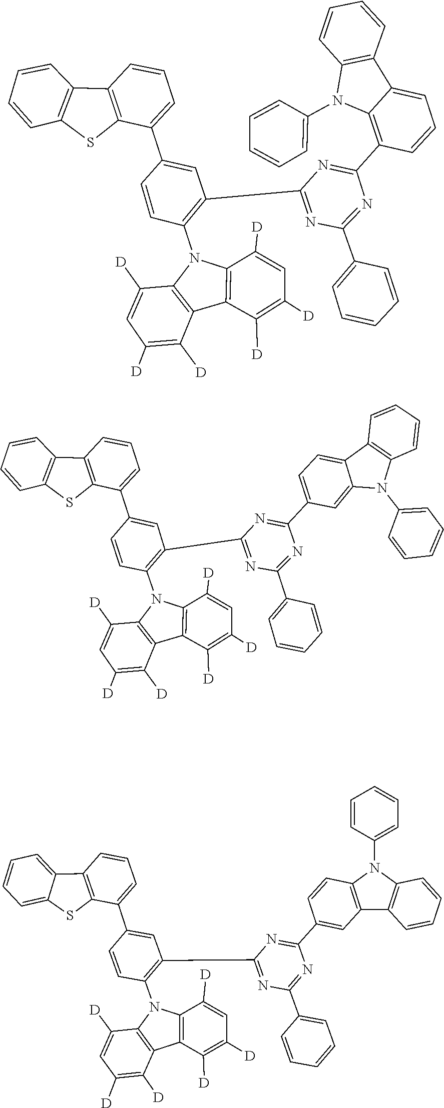 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c4ccccc4n(-c4ccccc4)c23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c4ccccc4n(-c4ccccc4)c3c2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccc3c(c2)c2ccccc2n3-c2ccccc2)n1]

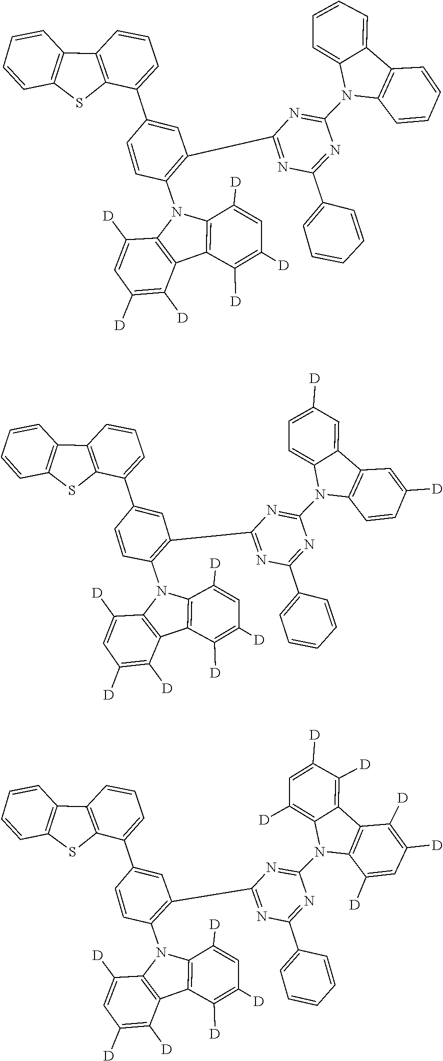 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-n2c3ccccc3c3ccccc32)n1 ; [2H]c1ccc2c(c1)c1cc([2H])ccc1n2-c1nc(-c2ccccc2)nc(-c2cc(-c3cccc4c3sc3ccccc34)ccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1nc(-c2ccccc2)nc(-c2cc(-c3cccc4c3sc3ccccc34)ccc2-n2c3c([2H])cc([2H])c([2H])c3c3c([2H])c([2H])cc([2H])c32)n1]

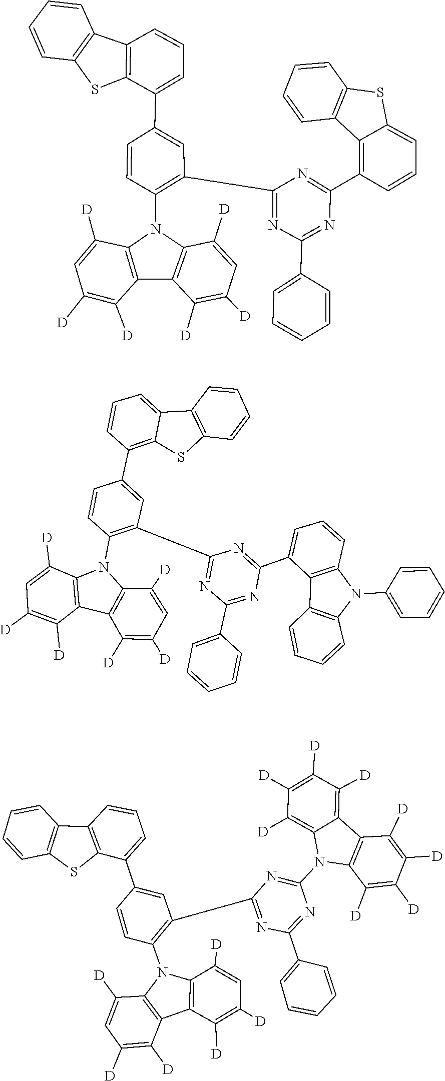 [SMILES: [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3sc4ccccc4c23)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2cccc3c2c2ccccc2n3-c2ccccc2)n1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-n2c3c([2H])c([2H])c([2H])c([2H])c3c3c([2H])c([2H])c([2H])c([2H])c32)n1]

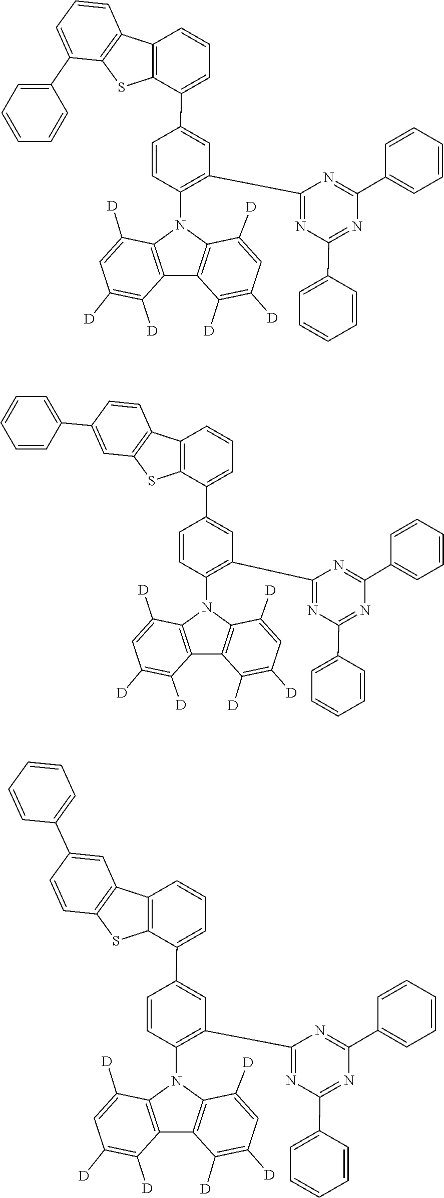 [SMILES: [2H]c1cc([2H])c2c(c1[2H])c1c([2H])c([2H])cc([2H])c1n2-c1ccc(-c2cccc3c2sc2c(-c4ccccc4)cccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2sc2cc(-c4ccccc4)ccc23)cc1-c1nc(-c2ccccc2)nc(-c2ccccc2)n1 ; [2H]c1cc([2H])c2c(c1[2H])c1c([2H])c([2H])cc([2H])c1n2-c1ccc(-c2cccc3c2sc2ccc(-c4ccccc4)cc23)cc1-c1nc(-c2ccccc2)nc(-c2ccccc2)n1]

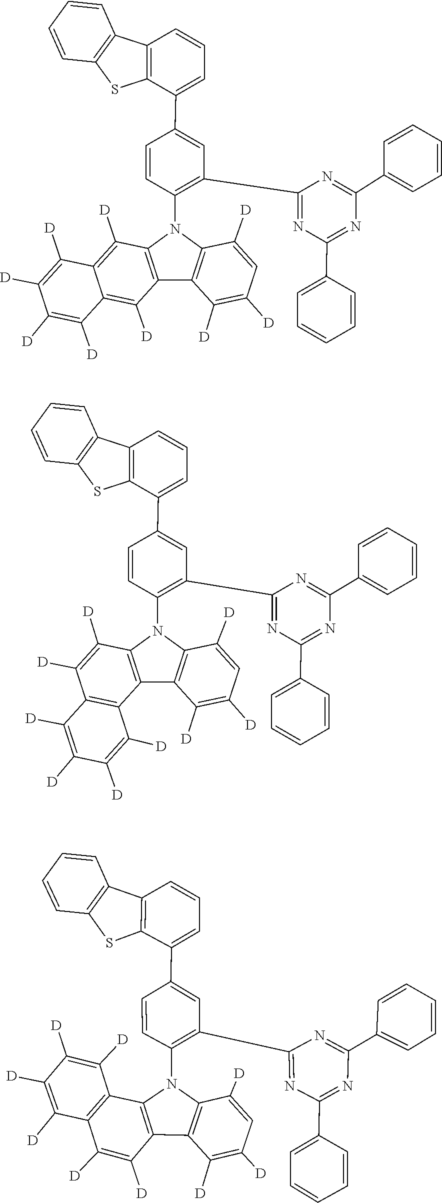 [SMILES: [2H]c1cc([2H])c2c(c1[2H])c1c([2H])c3c([2H])c([2H])c([2H])c([2H])c3c([2H])c1n2-c1ccc(-c2cccc3c2sc2ccccc23)cc1-c1nc(-c2ccccc2)nc(-c2ccccc2)n1 ; [2H]c1cc([2H])c2c(c1[2H])c1c3c([2H])c([2H])c([2H])c([2H])c3c([2H])c([2H])c1n2-c1ccc(-c2cccc3c2sc2ccccc23)cc1-c1nc(-c2ccccc2)nc(-c2ccccc2)n1 ; [2H]c1cc([2H])c2c(c1[2H])c1c([2H])c([2H])c3c([2H])c([2H])c([2H])c([2H])c3c1n2-c1ccc(-c2cccc3c2sc2ccccc23)cc1-c1nc(-c2ccccc2)nc(-c2ccccc2)n1]

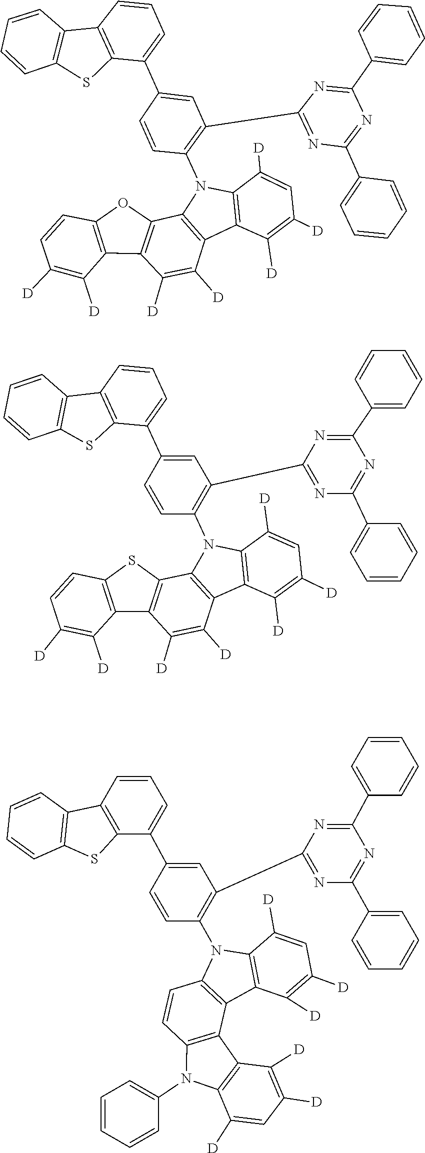 [SMILES: [2H]c1ccc2oc3c(c([2H])c([2H])c4c5c([2H])c([2H])cc([2H])c5n(-c5ccc(-c6cccc7c6sc6ccccc67)cc5-c5nc(-c6ccccc6)nc(-c6ccccc6)n5)c34)c2c1[2H] ; [2H]c1ccc2sc3c(c([2H])c([2H])c4c5c([2H])c([2H])cc([2H])c5n(-c5ccc(-c6cccc7c6sc6ccccc67)cc5-c5nc(-c6ccccc6)nc(-c6ccccc6)n5)c34)c2c1[2H] ; [2H]c1cc([2H])c2c(c1[2H])c1c3c4c([2H])c([2H])cc([2H])c4n(-c4ccc(-c5cccc6c5sc5ccccc56)cc4-c4nc(-c5ccccc5)nc(-c5ccccc5)n4)c3ccc1n2-c1ccccc1]

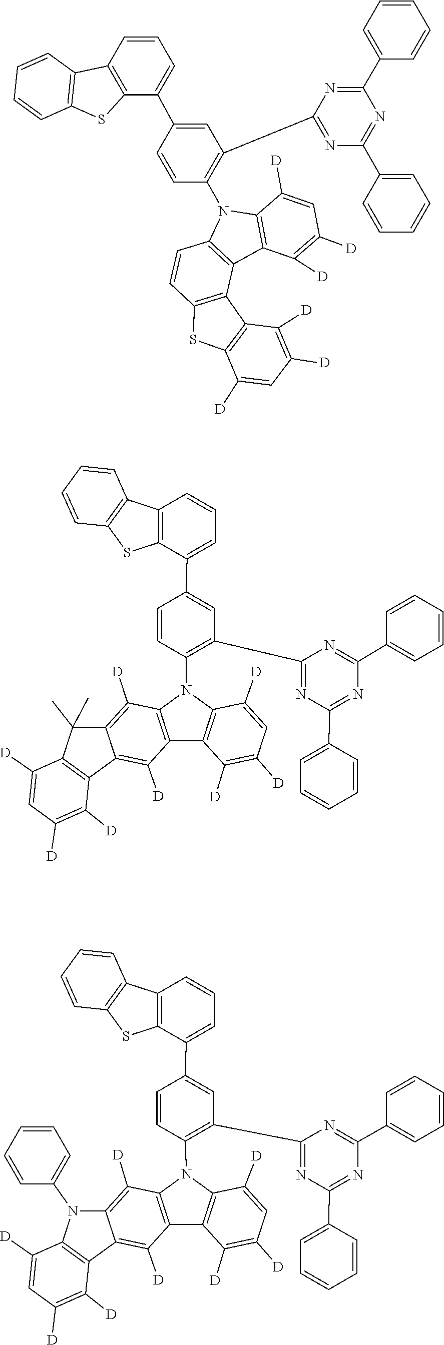 [SMILES: [2H]c1cc([2H])c2sc3ccc4c(c5c([2H])c([2H])cc([2H])c5n4-c4ccc(-c5cccc6c5sc5ccccc56)cc4-c4nc(-c5ccccc5)nc(-c5ccccc5)n4)c3c2c1[2H] ; [2H]c1cc([2H])c2c(c1[2H])-c1c(c([2H])c3c(c1[2H])c1c([2H])c([2H])cc([2H])c1n3-c1ccc(-c3cccc4c3sc3ccccc34)cc1-c1nc(-c3ccccc3)nc(-c3ccccc3)n1)C2(C)C ; [2H]c1cc([2H])c2c(c1[2H])c1c([2H])c3c4c([2H])c([2H])cc([2H])c4n(-c4ccc(-c5cccc6c5sc5ccccc56)cc4-c4nc(-c5ccccc5)nc(-c5ccccc5)n4)c3c([2H])c1n2-c1ccccc1]

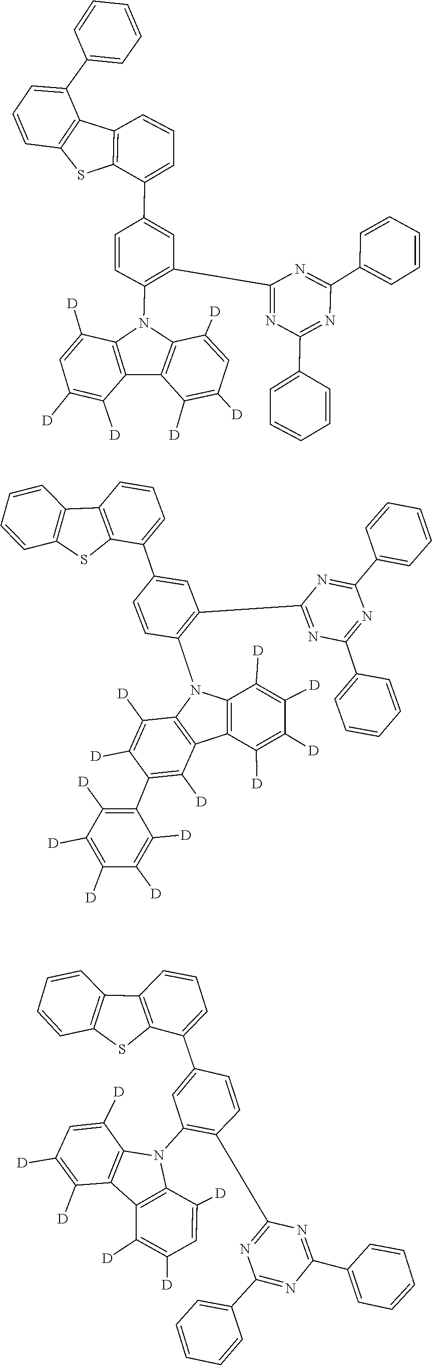

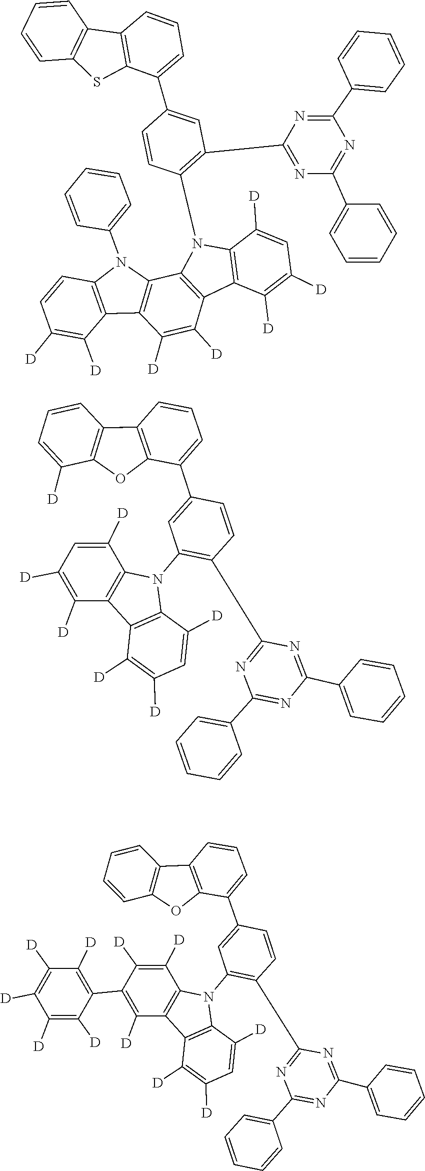

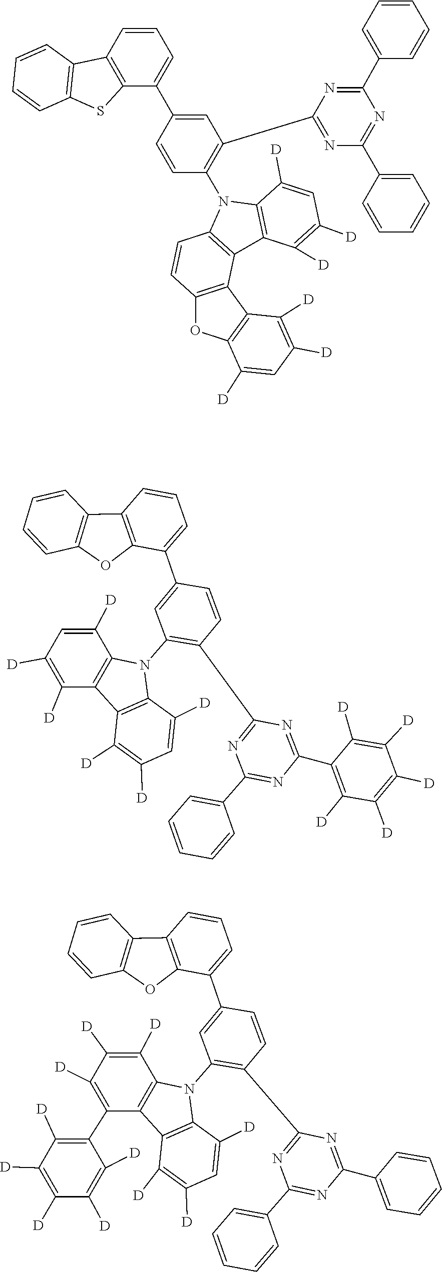 [SMILES: [2H]c1cc([2H])c2oc3ccc4c(c5c([2H])c([2H])cc([2H])c5n4-c4ccc(-c5cccc6c5sc5ccccc56)cc4-c4nc(-c5ccccc5)nc(-c5ccccc5)n4)c3c2c1[2H] ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2c([2H])c([2H])c([2H])c([2H])c2[2H])n1 ; [2H]c1cc([2H])c2c(c1[2H])c1c(-c3c([2H])c([2H])c([2H])c([2H])c3[2H])c([2H])c([2H])c([2H])c1n2-c1cc(-c2cccc3c2oc2ccccc23)ccc1-c1nc(-c2ccccc2)nc(-c2ccccc2)n1]

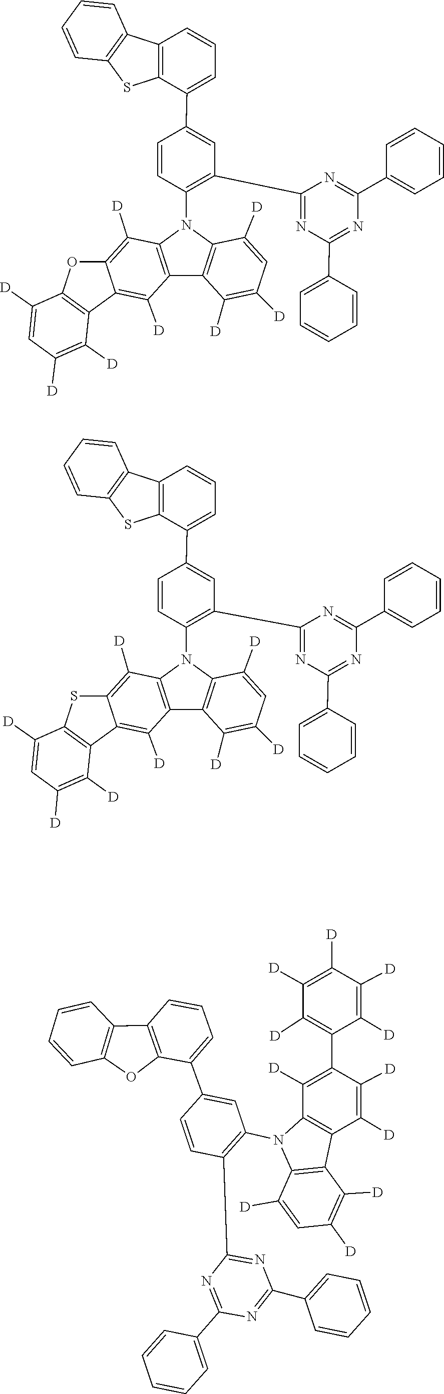 [SMILES: [2H]c1cc([2H])c2oc3c([2H])c4c(c([2H])c3c2c1[2H])c1c([2H])c([2H])cc([2H])c1n4-c1ccc(-c2cccc3c2sc2ccccc23)cc1-c1nc(-c2ccccc2)nc(-c2ccccc2)n1 ; [2H]c1cc([2H])c2sc3c([2H])c4c(c([2H])c3c2c1[2H])c1c([2H])c([2H])cc([2H])c1n4-c1ccc(-c2cccc3c2sc2ccccc23)cc1-c1nc(-c2ccccc2)nc(-c2ccccc2)n1 ; [2H]c1cc([2H])c2c(c1[2H])c1c([2H])c([2H])c(-c3c([2H])c([2H])c([2H])c([2H])c3[2H])c([2H])c1n2-c1cc(-c2cccc3c2oc2ccccc23)ccc1-c1nc(-c2ccccc2)nc(-c2ccccc2)n1]

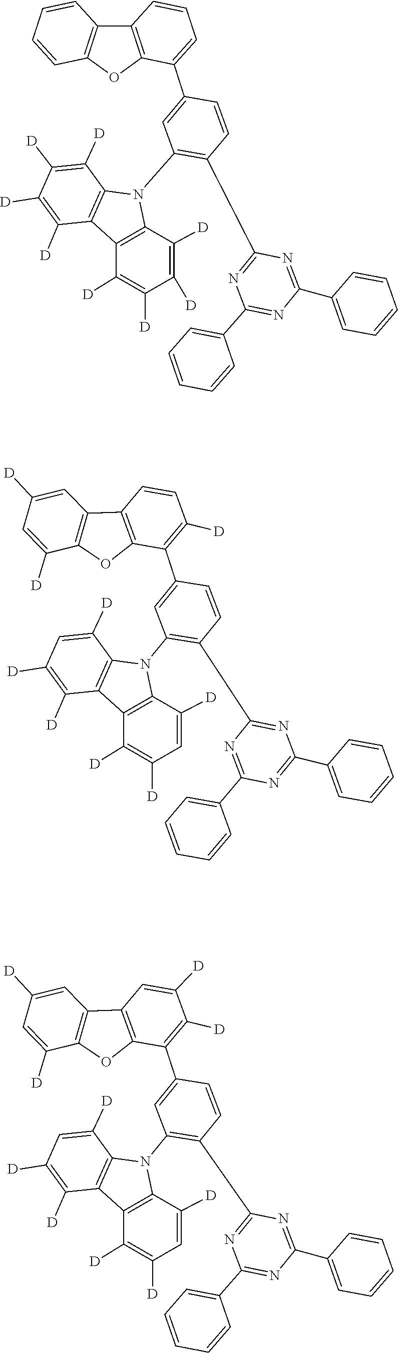 [SMILES: [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2ccccc2)n1 ; [2H]c1cc([2H])c2oc3c(-c4ccc(-c5nc(-c6ccccc6)nc(-c6ccccc6)n5)c(-n5c6c([2H])cc([2H])c([2H])c6c6c([2H])c([2H])cc([2H])c65)c4)c([2H])ccc3c2c1 ; [2H]c1cc([2H])c2oc3c(-c4ccc(-c5nc(-c6ccccc6)nc(-c6ccccc6)n5)c(-n5c6c([2H])cc([2H])c([2H])c6c6c([2H])c([2H])cc([2H])c65)c4)c([2H])c([2H])cc3c2c1]

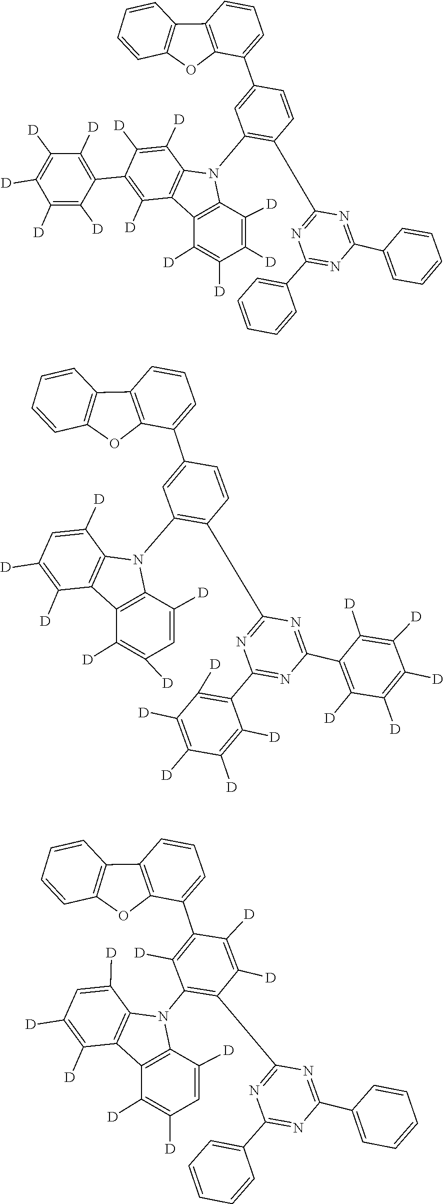 [SMILES: [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3ccccc3)n2)c([2H])c1[2H] ; [2H]c1cc([2H])c2c(c1[2H])c1c([2H])c([2H])cc([2H])c1n2-c1cc(-c2cccc3c2oc2ccccc23)ccc1-c1nc(-c2c([2H])c([2H])c([2H])c([2H])c2[2H])nc(-c2c([2H])c([2H])c([2H])c([2H])c2[2H])n1 ; [2H]c1cc([2H])c2c(c1[2H])c1c([2H])c([2H])cc([2H])c1n2-c1c([2H])c(-c2cccc3c2oc2ccccc23)c([2H])c([2H])c1-c1nc(-c2ccccc2)nc(-c2ccccc2)n1]

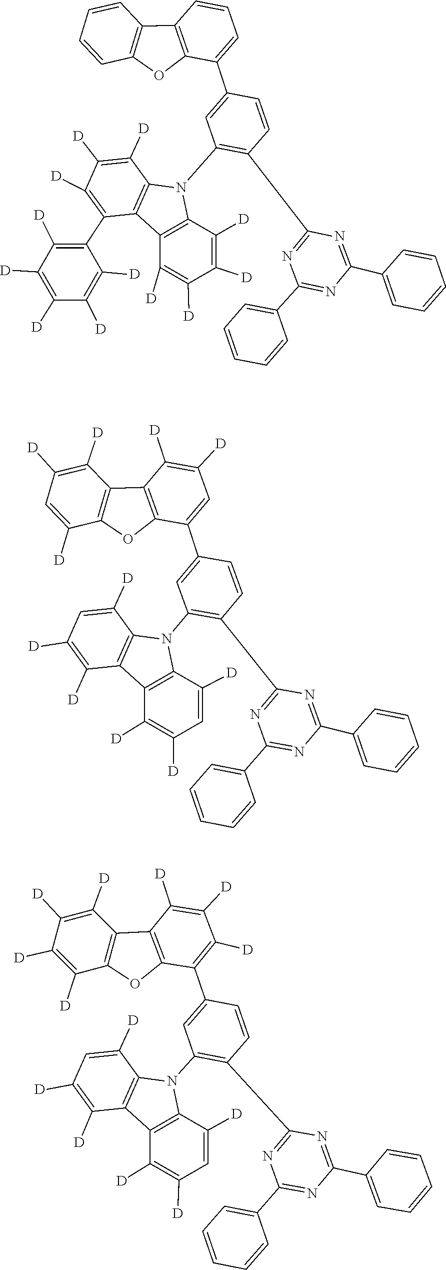 [SMILES: [2H]c1c([2H])c([2H])c(-c2c([2H])c([2H])c([2H])c3c2c2c([2H])c([2H])c([2H])c([2H])c2n3-c2cc(-c3cccc4c3oc3ccccc34)ccc2-c2nc(-c3ccccc3)nc(-c3ccccc3)n2)c([2H])c1[2H] ; [2H]c1cc([2H])c2oc3c(-c4ccc(-c5nc(-c6ccccc6)nc(-c6ccccc6)n5)c(-n5c6c([2H])cc([2H])c([2H])c6c6c([2H])c([2H])cc([2H])c65)c4)cc([2H])c([2H])c3c2c1[2H] ; [2H]c1cc([2H])c2c(c1[2H])c1c([2H])c([2H])cc([2H])c1n2-c1cc(-c2c([2H])c([2H])c([2H])c3c2oc2c([2H])c([2H])c([2H])c([2H])c23)ccc1-c1nc(-c2ccccc2)nc(-c2ccccc2)n1]

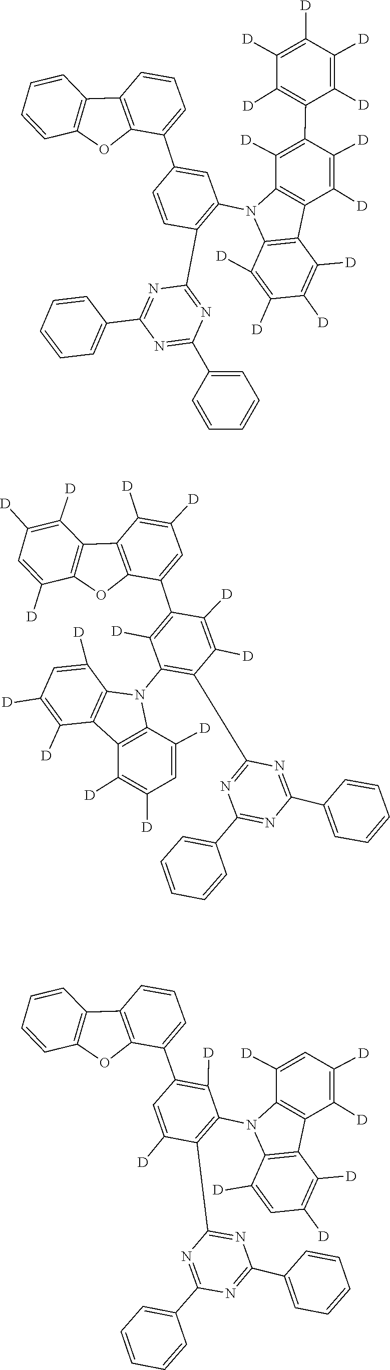 [SMILES: [2H]c1c([2H])c([2H])c(-c2c([2H])c([2H])c3c4c([2H])c([2H])c([2H])c([2H])c4n(-c4cc(-c5cccc6c5oc5ccccc56)ccc4-c4nc(-c5ccccc5)nc(-c5ccccc5)n4)c3c2[2H])c([2H])c1[2H] ; [2H]c1cc([2H])c2oc3c(-c4c([2H])c([2H])c(-c5nc(-c6ccccc6)nc(-c6ccccc6)n5)c(-n5c6c([2H])cc([2H])c([2H])c6c6c([2H])c([2H])cc([2H])c65)c4[2H])cc([2H])c([2H])c3c2c1[2H] ; [2H]c1cc(-c2cccc3c2oc2ccccc23)c([2H])c(-n2c3c([2H])cc([2H])c([2H])c3c3c([2H])c([2H])cc([2H])c32)c1-c1nc(-c2ccccc2)nc(-c2ccccc2)n1]

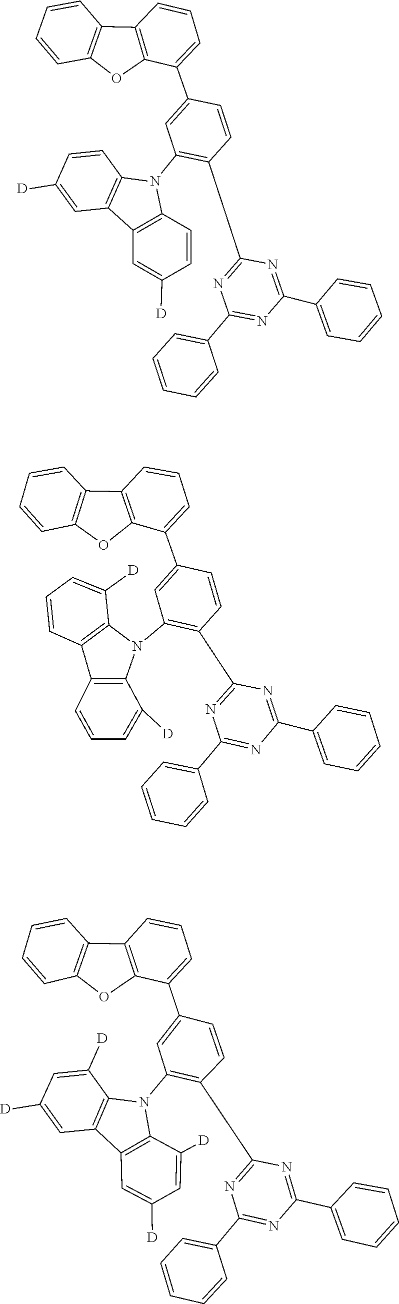 [SMILES: [2H]c1ccc2c(c1)c1cc([2H])ccc1n2-c1cc(-c2cccc3c2oc2ccccc23)ccc1-c1nc(-c2ccccc2)nc(-c2ccccc2)n1 ; [2H]c1cccc2c3cccc([2H])c3n(-c3cc(-c4cccc5c4oc4ccccc45)ccc3-c3nc(-c4ccccc4)nc(-c4ccccc4)n3)c12 ; [2H]c1cc([2H])c2c(c1)c1cc([2H])cc([2H])c1n2-c1cc(-c2cccc3c2oc2ccccc23)ccc1-c1nc(-c2ccccc2)nc(-c2ccccc2)n1]

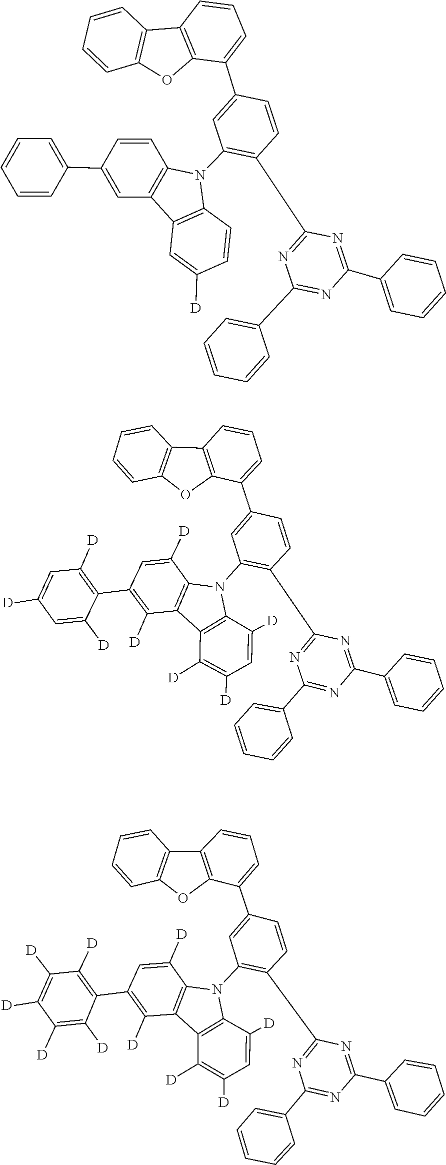 [SMILES: [2H]c1ccc2c(c1)c1cc(-c3ccccc3)ccc1n2-c1cc(-c2cccc3c2oc2ccccc23)ccc1-c1nc(-c2ccccc2)nc(-c2ccccc2)n1 ; [2H]c1cc([2H])c(-c2cc([2H])c3c(c2[2H])c2c([2H])c([2H])cc([2H])c2n3-c2cc(-c3cccc4c3oc3ccccc34)ccc2-c2nc(-c3ccccc3)nc(-c3ccccc3)n2)c([2H])c1 ; [2H]c1cc([2H])c2c(c1[2H])c1c([2H])c(-c3c([2H])c([2H])c([2H])c([2H])c3[2H])cc([2H])c1n2-c1cc(-c2cccc3c2oc2ccccc23)ccc1-c1nc(-c2ccccc2)nc(-c2ccccc2)n1]

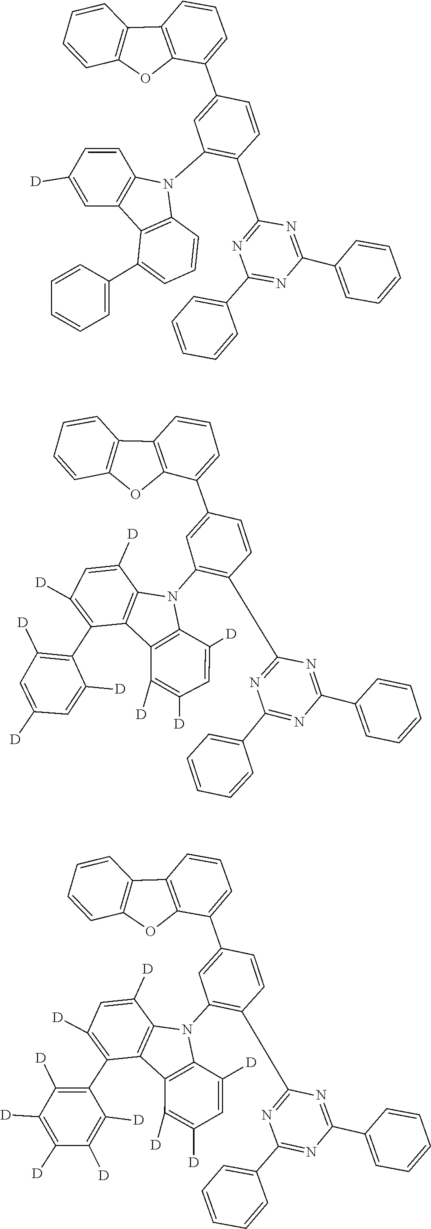 [SMILES: [2H]c1ccc2c(c1)c1c(-c3ccccc3)cccc1n2-c1cc(-c2cccc3c2oc2ccccc23)ccc1-c1nc(-c2ccccc2)nc(-c2ccccc2)n1 ; [2H]c1cc([2H])c(-c2c([2H])cc([2H])c3c2c2c([2H])c([2H])cc([2H])c2n3-c2cc(-c3cccc4c3oc3ccccc34)ccc2-c2nc(-c3ccccc3)nc(-c3ccccc3)n2)c([2H])c1 ; [2H]c1cc([2H])c2c(c1[2H])c1c(-c3c([2H])c([2H])c([2H])c([2H])c3[2H])c([2H])cc([2H])c1n2-c1cc(-c2cccc3c2oc2ccccc23)ccc1-c1nc(-c2ccccc2)nc(-c2ccccc2)n1]

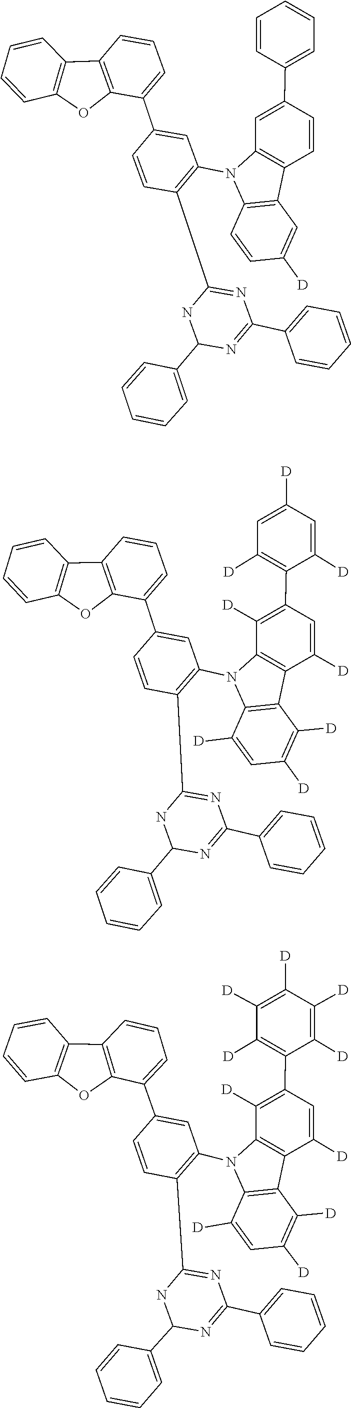

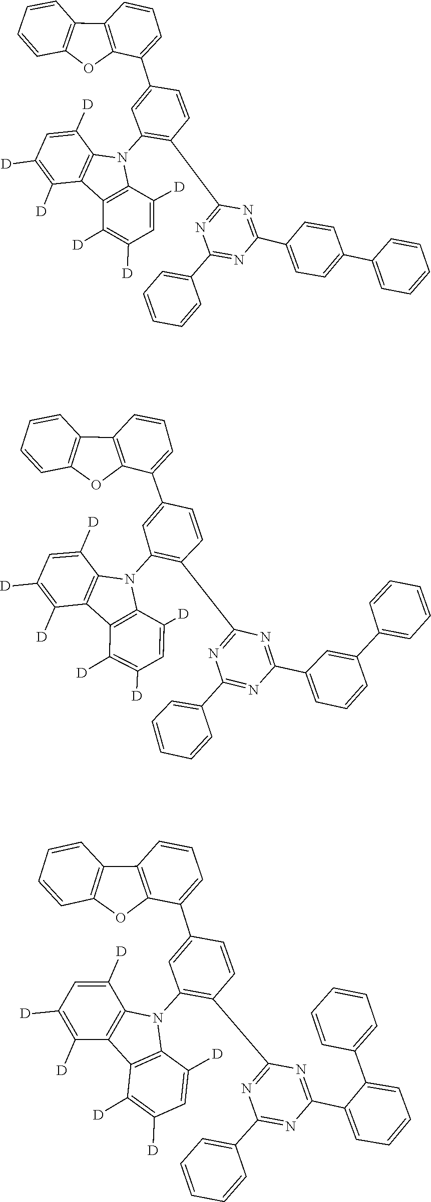 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc(-c3ccccc3)cc2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc(-c3ccccc3)c2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccccc2-c2ccccc2)n1]

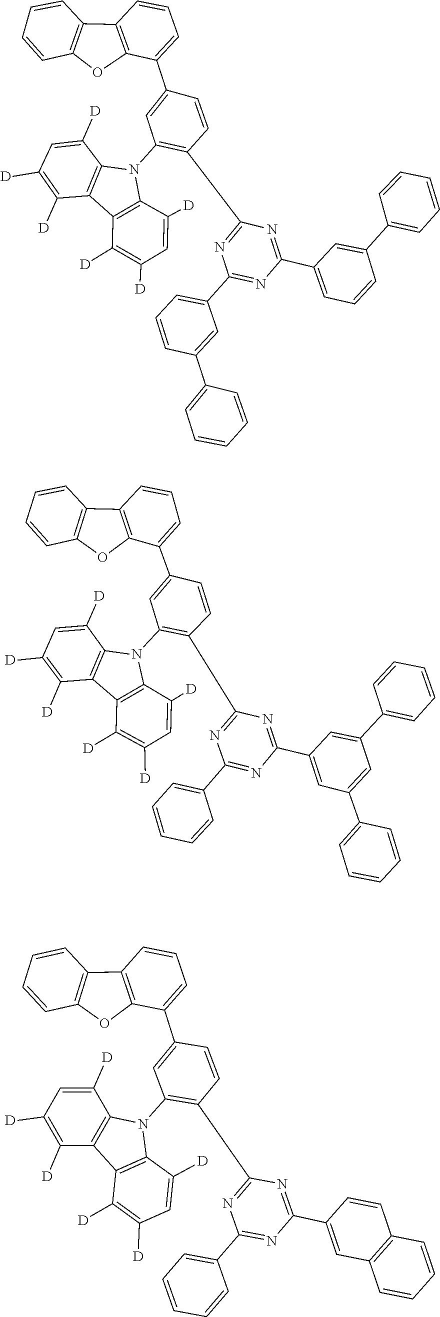 [SMILES: [2H]c1cc([2H])c2c(c1[2H])c1c([2H])c([2H])cc([2H])c1n2-c1cc(-c2cccc3c2oc2ccccc23)ccc1-c1nc(-c2cccc(-c3ccccc3)c2)nc(-c2cccc(-c3ccccc3)c2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cc(-c3ccccc3)cc(-c3ccccc3)c2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3ccccc3c2)n1]

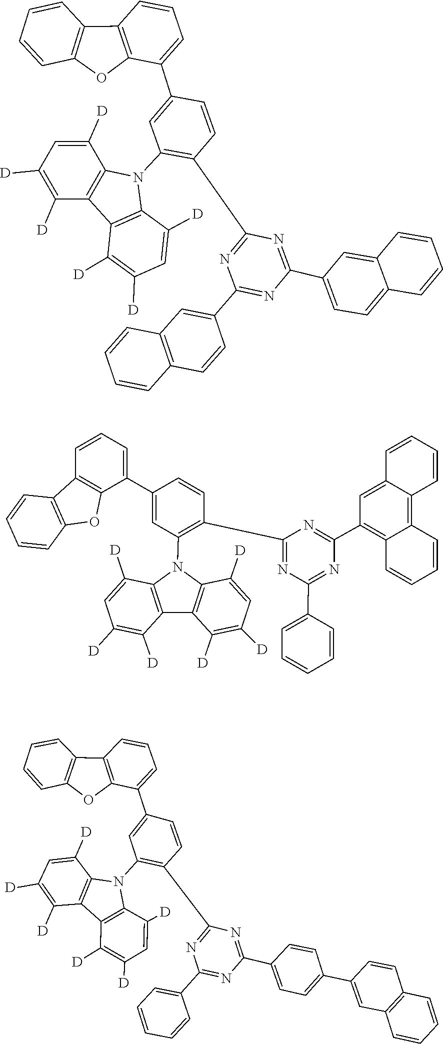 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccc3ccccc3c2)nc(-c2ccc3ccccc3c2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cc3ccccc3c3ccccc23)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc(-c3ccc4ccccc4c3)cc2)n1]

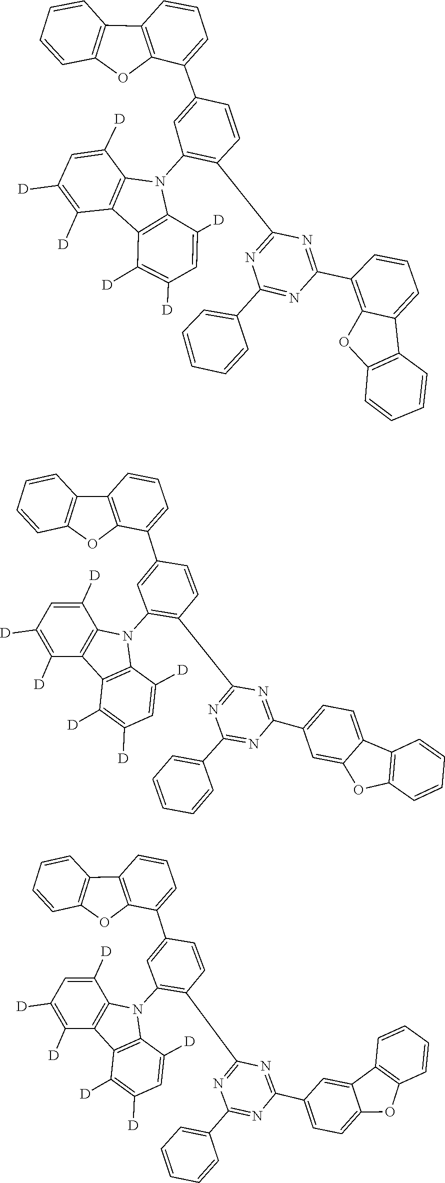 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc3c2oc2ccccc23)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3oc4ccccc4c3c2)n1]

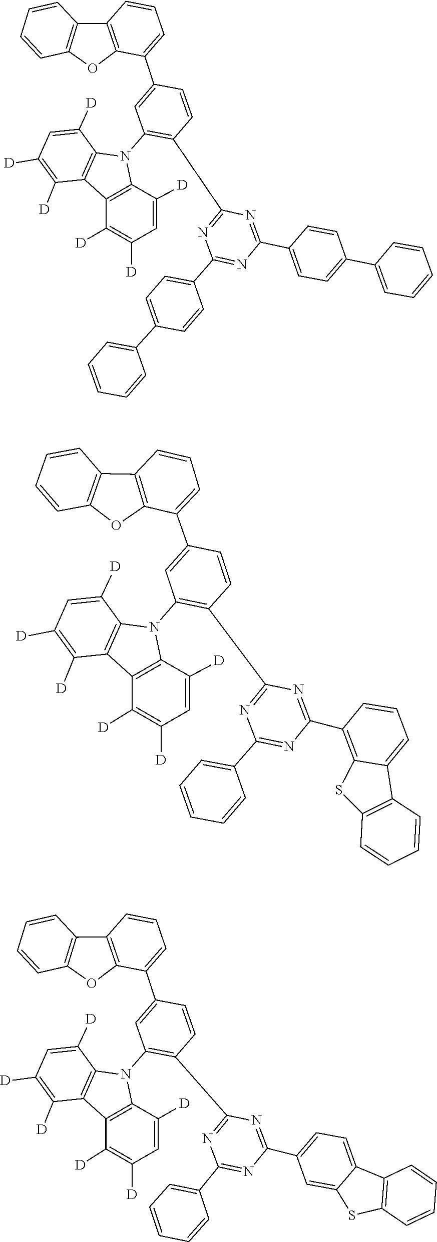 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccc(-c3ccccc3)cc2)nc(-c2ccc(-c3ccccc3)cc2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc3c2sc2ccccc23)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3c(c2)sc2ccccc23)n1]

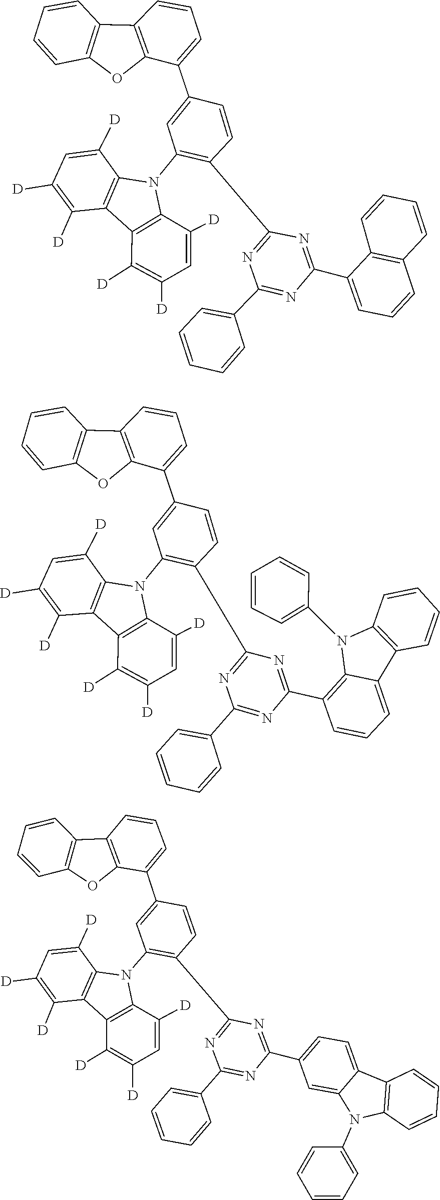

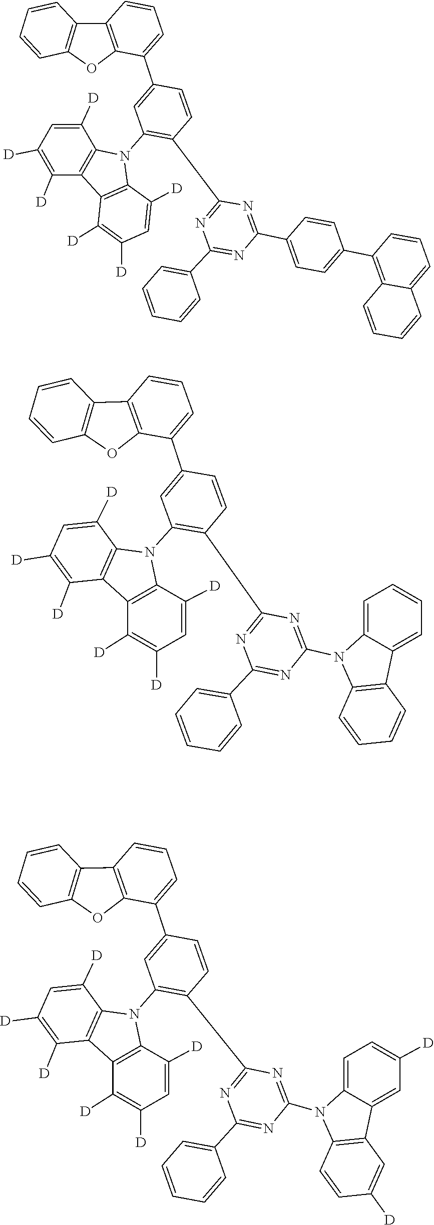 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc(-c3cccc4ccccc34)cc2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-n2c3ccccc3c3ccccc32)n1 ; [2H]c1ccc2c(c1)c1cc([2H])ccc1n2-c1nc(-c2ccccc2)nc(-c2ccc(-c3cccc4c3oc3ccccc34)cc2-n2c3c([2H])cc([2H])c([2H])c3c3c([2H])c([2H])cc([2H])c32)n1]

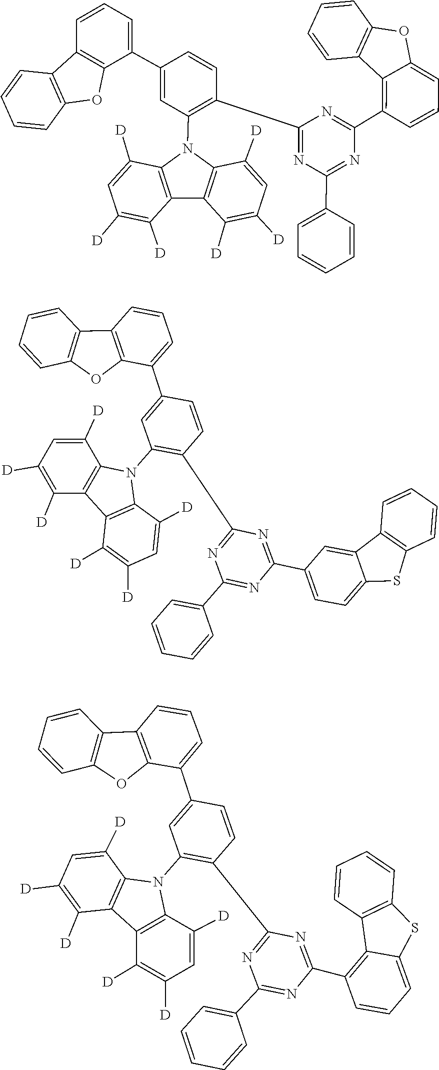 [SMILES: [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc3oc4ccccc4c23)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3sc4ccccc4c3c2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc3sc4ccccc4c23)n1]

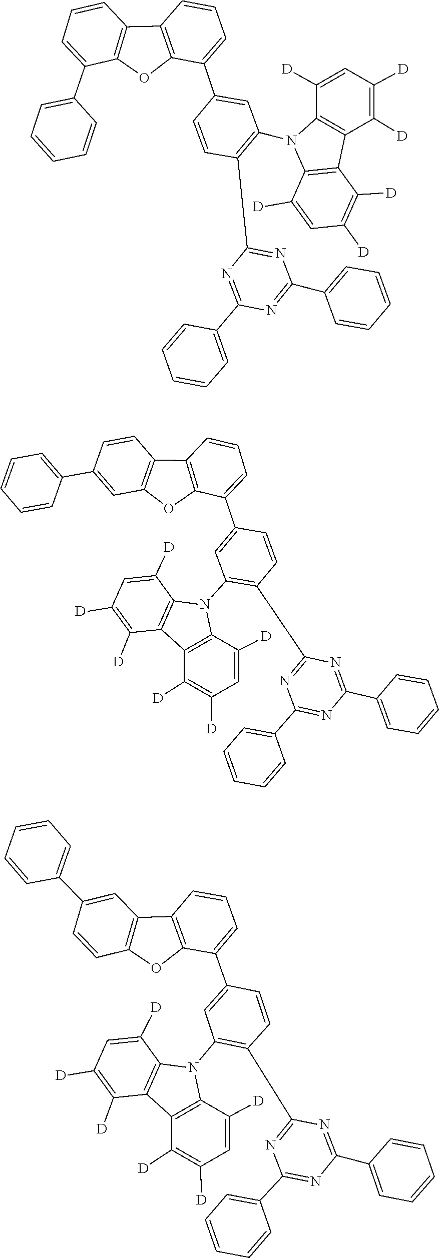 [SMILES: [2H]c1cc([2H])c2c(c1[2H])c1c([2H])c([2H])cc([2H])c1n2-c1cc(-c2cccc3c2oc2c(-c4ccccc4)cccc23)ccc1-c1nc(-c2ccccc2)nc(-c2ccccc2)n1 ; [2H]c1cc([2H])c2c(c1[2H])c1c([2H])c([2H])cc([2H])c1n2-c1cc(-c2cccc3c2oc2cc(-c4ccccc4)ccc23)ccc1-c1nc(-c2ccccc2)nc(-c2ccccc2)n1 ; [2H]c1cc([2H])c2c(c1[2H])c1c([2H])c([2H])cc([2H])c1n2-c1cc(-c2cccc3c2oc2ccc(-c4ccccc4)cc23)ccc1-c1nc(-c2ccccc2)nc(-c2ccccc2)n1]

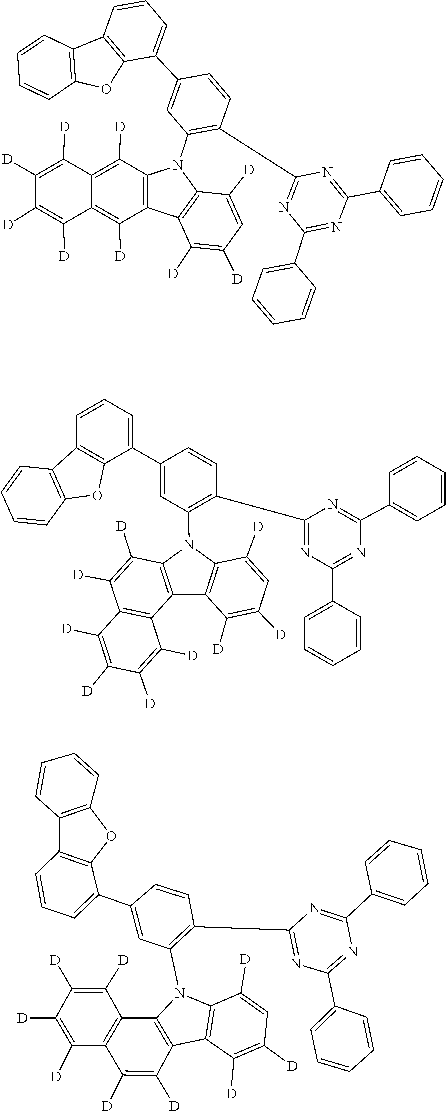 [SMILES: [2H]c1cc([2H])c2c(c1[2H])c1c([2H])c3c([2H])c([2H])c([2H])c([2H])c3c([2H])c1n2-c1cc(-c2cccc3c2oc2ccccc23)ccc1-c1nc(-c2ccccc2)nc(-c2ccccc2)n1 ; [2H]c1cc([2H])c2c(c1[2H])c1c3c([2H])c([2H])c([2H])c([2H])c3c([2H])c([2H])c1n2-c1cc(-c2cccc3c2oc2ccccc23)ccc1-c1nc(-c2ccccc2)nc(-c2ccccc2)n1 ; [2H]c1cc([2H])c2c(c1[2H])c1c([2H])c([2H])c3c([2H])c([2H])c([2H])c([2H])c3c1n2-c1cc(-c2cccc3c2oc2ccccc23)ccc1-c1nc(-c2ccccc2)nc(-c2ccccc2)n1]

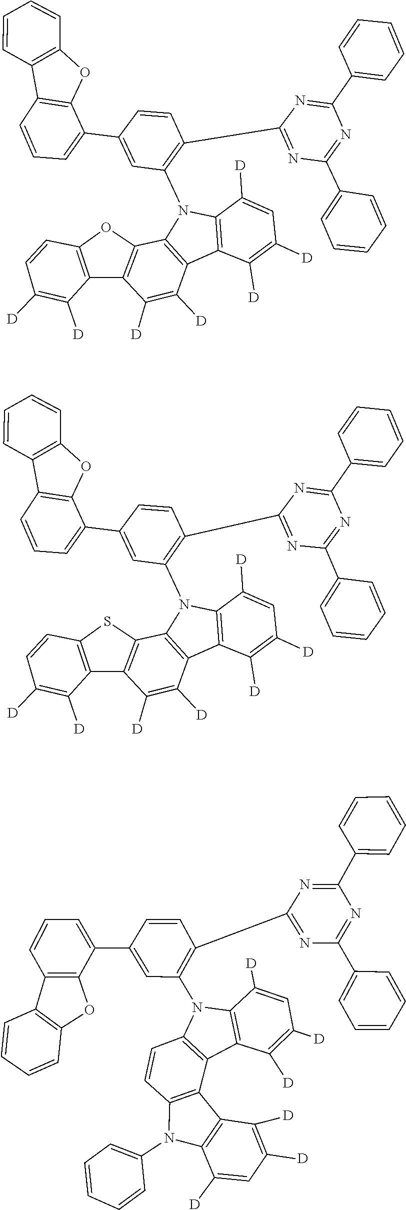 [SMILES: [2H]c1ccc2oc3c(c([2H])c([2H])c4c5c([2H])c([2H])cc([2H])c5n(-c5cc(-c6cccc7c6oc6ccccc67)ccc5-c5nc(-c6ccccc6)nc(-c6ccccc6)n5)c34)c2c1[2H] ; [2H]c1ccc2sc3c(c([2H])c([2H])c4c5c([2H])c([2H])cc([2H])c5n(-c5cc(-c6cccc7c6oc6ccccc67)ccc5-c5nc(-c6ccccc6)nc(-c6ccccc6)n5)c34)c2c1[2H] ; [2H]c1cc([2H])c2c(c1[2H])c1c3c4c([2H])c([2H])cc([2H])c4n(-c4cc(-c5cccc6c5oc5ccccc56)ccc4-c4nc(-c5ccccc5)nc(-c5ccccc5)n4)c3ccc1n2-c1ccccc1]

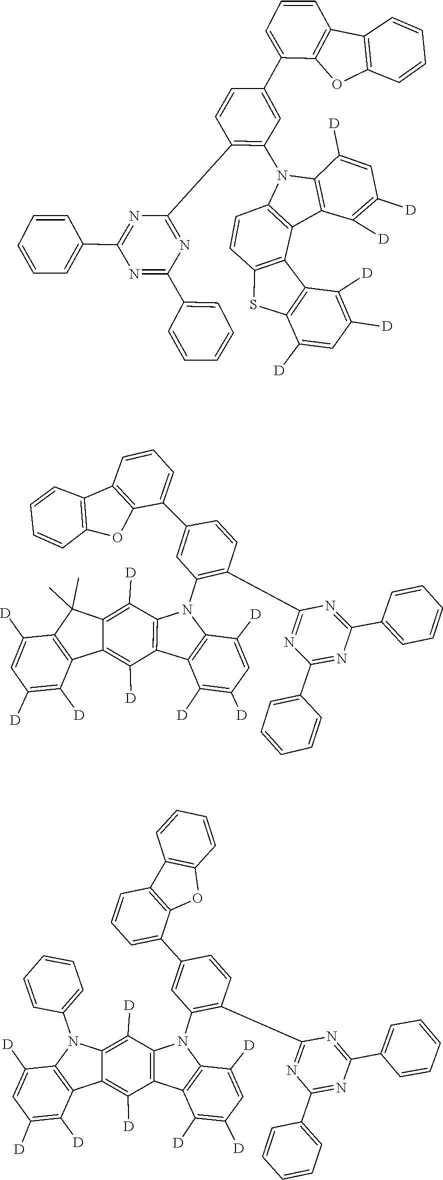 [SMILES: [2H]c1cc([2H])c2sc3ccc4c(c5c([2H])c([2H])cc([2H])c5n4-c4cc(-c5cccc6c5oc5ccccc56)ccc4-c4nc(-c5ccccc5)nc(-c5ccccc5)n4)c3c2c1[2H] ; [2H]c1cc([2H])c2c(c1[2H])-c1c(c([2H])c3c(c1[2H])c1c([2H])c([2H])cc([2H])c1n3-c1cc(-c3cccc4c3oc3ccccc34)ccc1-c1nc(-c3ccccc3)nc(-c3ccccc3)n1)C2(C)C ; [2H]c1cc([2H])c2c(c1[2H])c1c([2H])c3c4c([2H])c([2H])cc([2H])c4n(-c4cc(-c5cccc6c5oc5ccccc56)ccc4-c4nc(-c5ccccc5)nc(-c5ccccc5)n4)c3c([2H])c1n2-c1ccccc1]

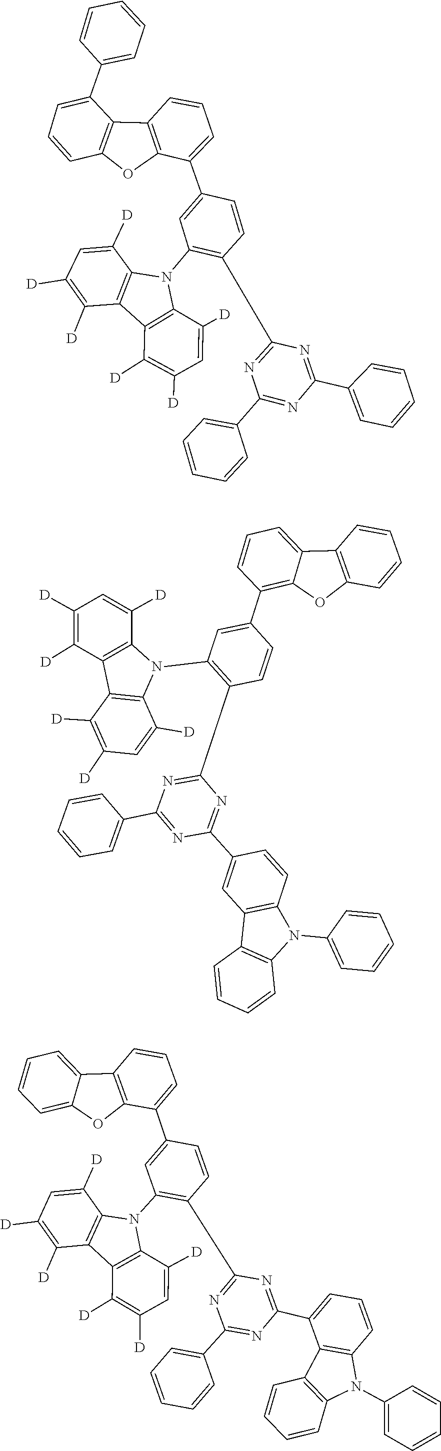 [SMILES: [2H]c1cc([2H])c2c(c1[2H])c1c([2H])c([2H])cc([2H])c1n2-c1cc(-c2cccc3c2oc2cccc(-c4ccccc4)c23)ccc1-c1nc(-c2ccccc2)nc(-c2ccccc2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2ccc3c(c2)c2ccccc2n3-c2ccccc2)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-c2cccc3c2c2ccccc2n3-c2ccccc2)n1]

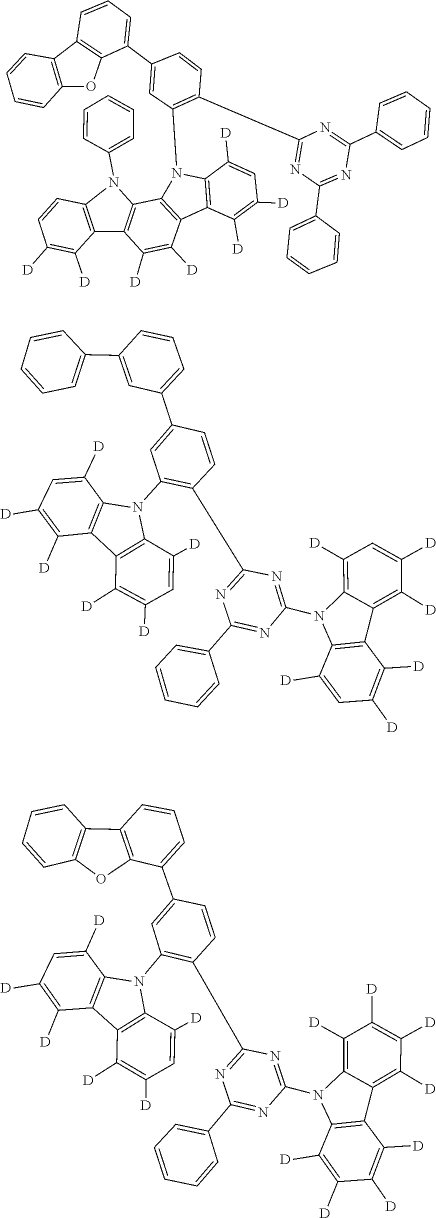 [SMILES: [2H]c1ccc2c(c1[2H])c1c([2H])c([2H])c3c4c([2H])c([2H])cc([2H])c4n(-c4cc(-c5cccc6c5oc5ccccc56)ccc4-c4nc(-c5ccccc5)nc(-c5ccccc5)n4)c3c1n2-c1ccccc1 ; [2H]c1cc([2H])c2c(c1[2H])c1c([2H])c([2H])cc([2H])c1n2-c1nc(-c2ccccc2)nc(-c2ccc(-c3cccc(-c4ccccc4)c3)cc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1cc(-c2cccc3c2oc2ccccc23)ccc1-c1nc(-c2ccccc2)nc(-n2c3c([2H])c([2H])c([2H])c([2H])c3c3c([2H])c([2H])c([2H])c([2H])c32)n1]

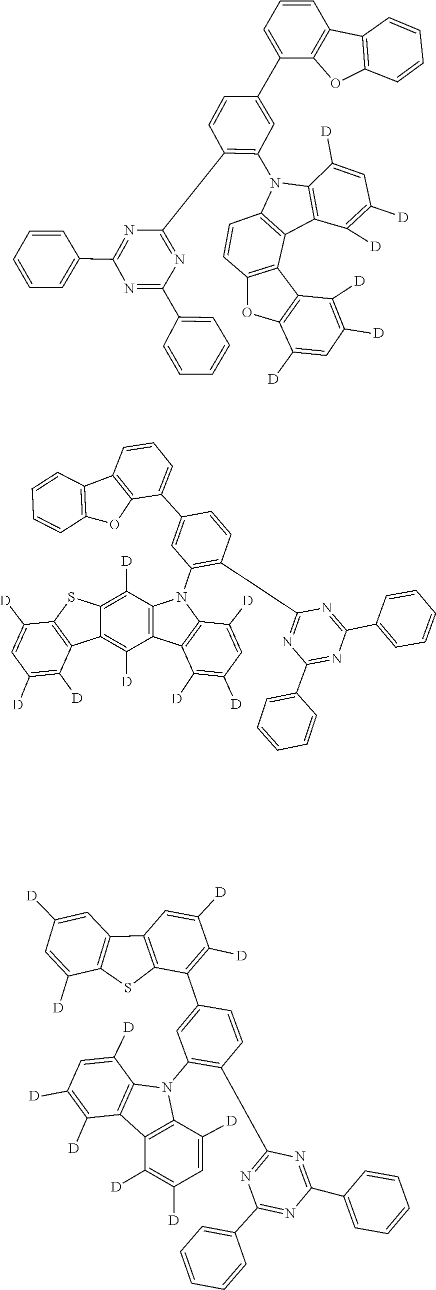 [SMILES: [2H]c1cc([2H])c2oc3ccc4c(c5c([2H])c([2H])cc([2H])c5n4-c4cc(-c5cccc6c5oc5ccccc56)ccc4-c4nc(-c5ccccc5)nc(-c5ccccc5)n4)c3c2c1[2H] ; [2H]c1cc([2H])c2sc3c([2H])c4c(c([2H])c3c2c1[2H])c1c([2H])c([2H])cc([2H])c1n4-c1cc(-c2cccc3c2oc2ccccc23)ccc1-c1nc(-c2ccccc2)nc(-c2ccccc2)n1 ; [2H]c1cc([2H])c2sc3c(-c4ccc(-c5nc(-c6ccccc6)nc(-c6ccccc6)n5)c(-n5c6c([2H])cc([2H])c([2H])c6c6c([2H])c([2H])cc([2H])c65)c4)c([2H])c([2H])cc3c2c1]

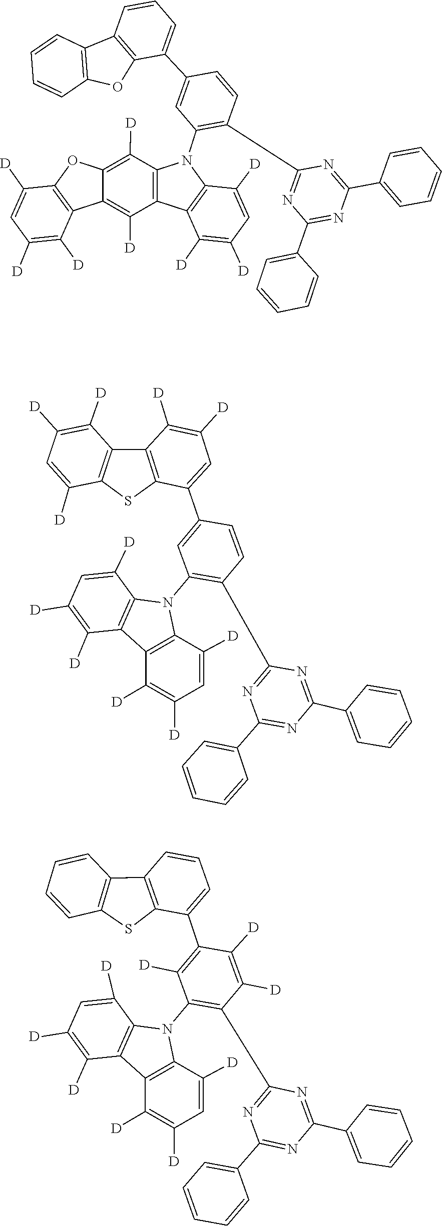 [SMILES: [2H]c1cc([2H])c2oc3c([2H])c4c(c([2H])c3c2c1[2H])c1c([2H])c([2H])cc([2H])c1n4-c1cc(-c2cccc3c2oc2ccccc23)ccc1-c1nc(-c2ccccc2)nc(-c2ccccc2)n1 ; [2H]c1cc([2H])c2sc3c(-c4ccc(-c5nc(-c6ccccc6)nc(-c6ccccc6)n5)c(-n5c6c([2H])cc([2H])c([2H])c6c6c([2H])c([2H])cc([2H])c65)c4)cc([2H])c([2H])c3c2c1[2H] ; [2H]c1cc([2H])c2c(c1[2H])c1c([2H])c([2H])cc([2H])c1n2-c1c([2H])c(-c2cccc3c2sc2ccccc23)c([2H])c([2H])c1-c1nc(-c2ccccc2)nc(-c2ccccc2)n1]

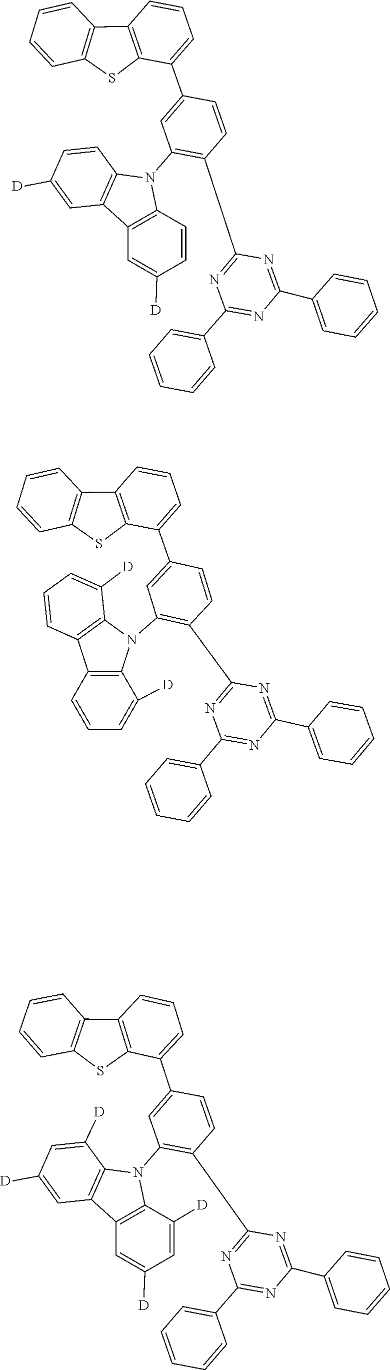 [SMILES: [2H]c1ccc2c(c1)c1cc([2H])ccc1n2-c1cc(-c2cccc3c2sc2ccccc23)ccc1-c1nc(-c2ccccc2)nc(-c2ccccc2)n1 ; [2H]c1cccc2c3cccc([2H])c3n(-c3cc(-c4cccc5c4sc4ccccc45)ccc3-c3nc(-c4ccccc4)nc(-c4ccccc4)n3)c12 ; [2H]c1cc([2H])c2c(c1)c1cc([2H])cc([2H])c1n2-c1cc(-c2cccc3c2sc2ccccc23)ccc1-c1nc(-c2ccccc2)nc(-c2ccccc2)n1]

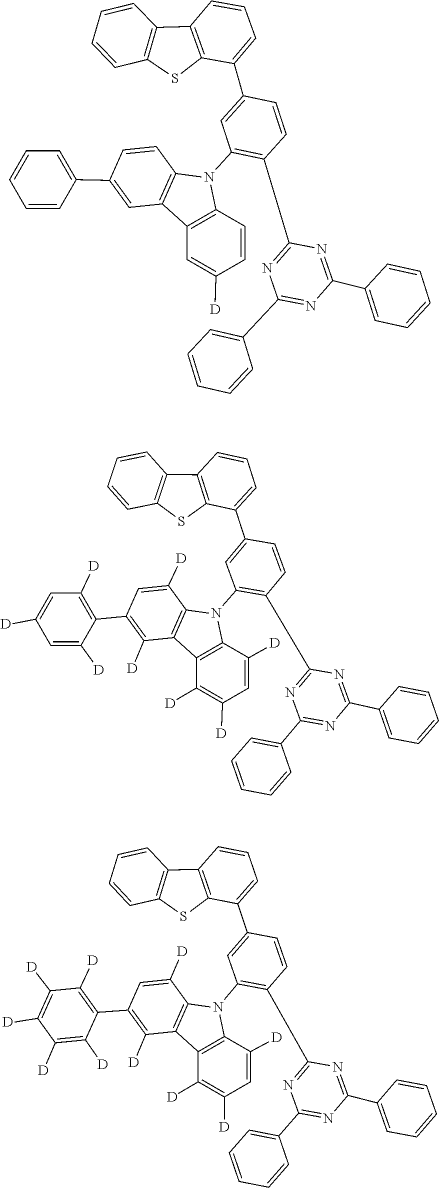 [SMILES: [2H]c1ccc2c(c1)c1cc(-c3ccccc3)ccc1n2-c1cc(-c2cccc3c2sc2ccccc23)ccc1-c1nc(-c2ccccc2)nc(-c2ccccc2)n1 ; [2H]c1cc([2H])c(-c2cc([2H])c3c(c2[2H])c2c([2H])c([2H])cc([2H])c2n3-c2cc(-c3cccc4c3sc3ccccc34)ccc2-c2nc(-c3ccccc3)nc(-c3ccccc3)n2)c([2H])c1 ; [2H]c1cc([2H])c2c(c1[2H])c1c([2H])c(-c3c([2H])c([2H])c([2H])c([2H])c3[2H])cc([2H])c1n2-c1cc(-c2cccc3c2sc2ccccc23)ccc1-c1nc(-c2ccccc2)nc(-c2ccccc2)n1]

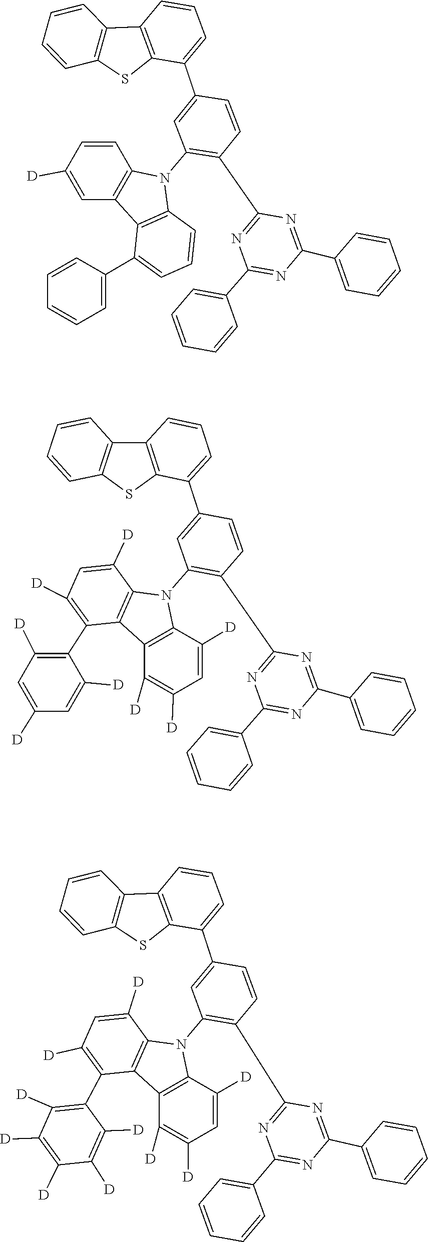 [SMILES: [2H]c1ccc2c(c1)c1c(-c3ccccc3)cccc1n2-c1cc(-c2cccc3c2sc2ccccc23)ccc1-c1nc(-c2ccccc2)nc(-c2ccccc2)n1 ; [2H]c1cc([2H])c(-c2c([2H])cc([2H])c3c2c2c([2H])c([2H])cc([2H])c2n3-c2cc(-c3cccc4c3sc3ccccc34)ccc2-c2nc(-c3ccccc3)nc(-c3ccccc3)n2)c([2H])c1 ; [2H]c1cc([2H])c2c(c1[2H])c1c(-c3c([2H])c([2H])c([2H])c([2H])c3[2H])c([2H])cc([2H])c1n2-c1cc(-c2cccc3c2sc2ccccc23)ccc1-c1nc(-c2ccccc2)nc(-c2ccccc2)n1]

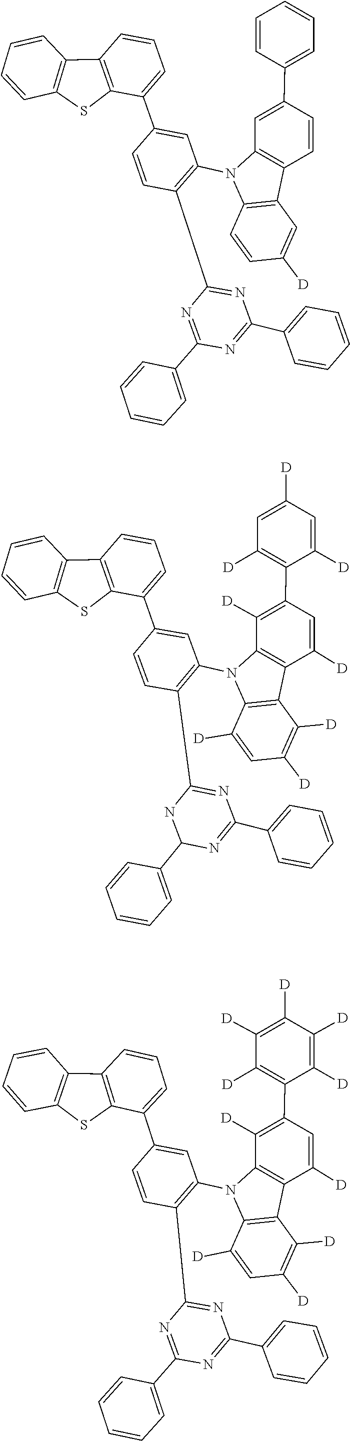

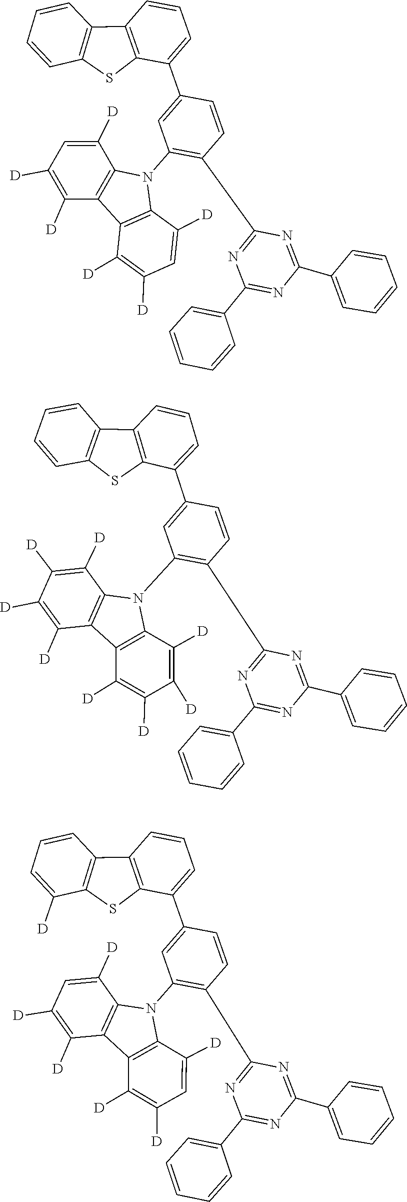 [SMILES: [2H]c1cc([2H])c2c(c1[2H])c1c([2H])c([2H])cc([2H])c1n2-c1cc(-c2cccc3c2sc2ccccc23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2ccccc2)n1 ; [2H]c1cc([2H])c2c(c1[2H])c1c([2H])c([2H])cc([2H])c1n2-c1cc(-c2cccc3c2sc2c([2H])cccc23)ccc1-c1nc(-c2ccccc2)nc(-c2ccccc2)n1]

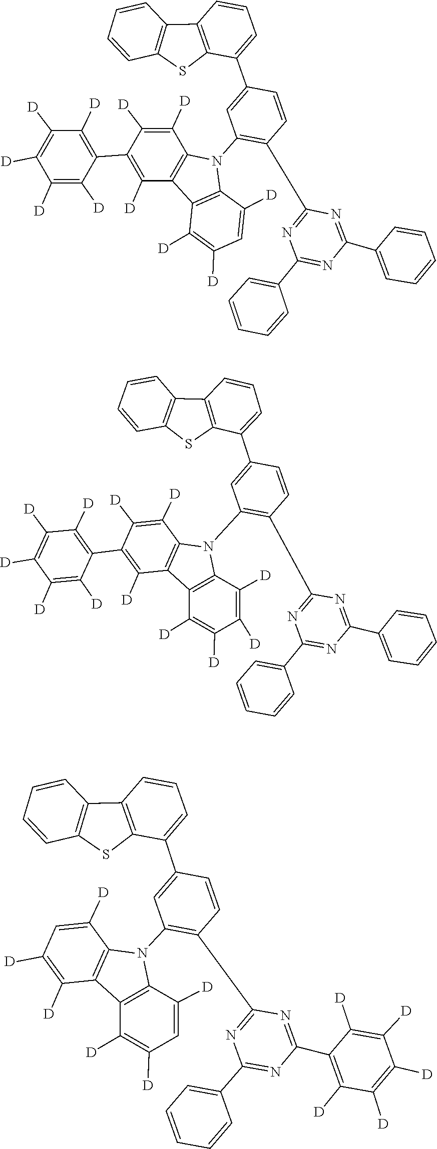 [SMILES: [2H]c1cc([2H])c2c(c1[2H])c1c([2H])c(-c3c([2H])c([2H])c([2H])c([2H])c3[2H])c([2H])c([2H])c1n2-c1cc(-c2cccc3c2sc2ccccc23)ccc1-c1nc(-c2ccccc2)nc(-c2ccccc2)n1 ; [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3ccccc3)n2)c([2H])c1[2H] ; [2H]c1cc([2H])c2c(c1[2H])c1c([2H])c([2H])cc([2H])c1n2-c1cc(-c2cccc3c2sc2ccccc23)ccc1-c1nc(-c2ccccc2)nc(-c2c([2H])c([2H])c([2H])c([2H])c2[2H])n1]

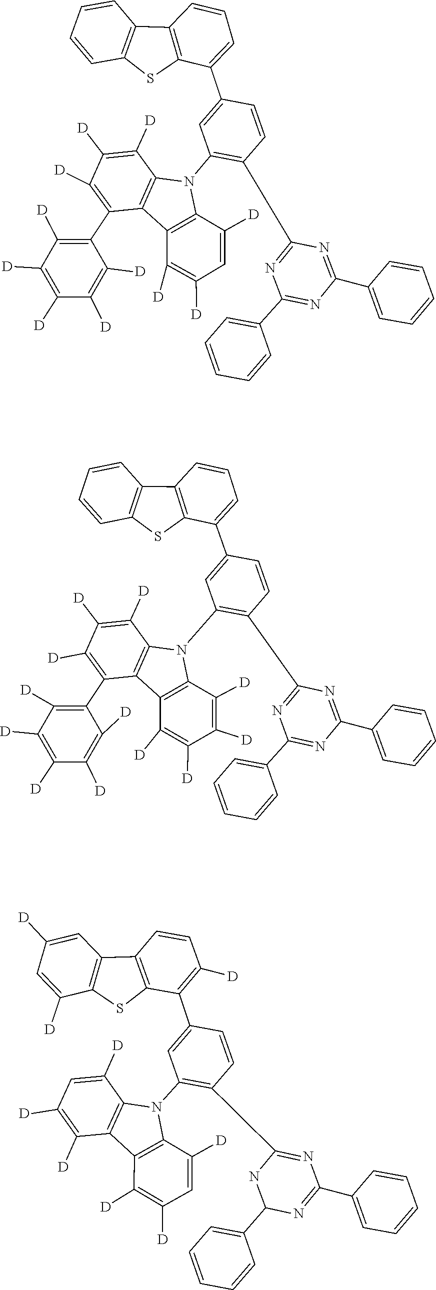

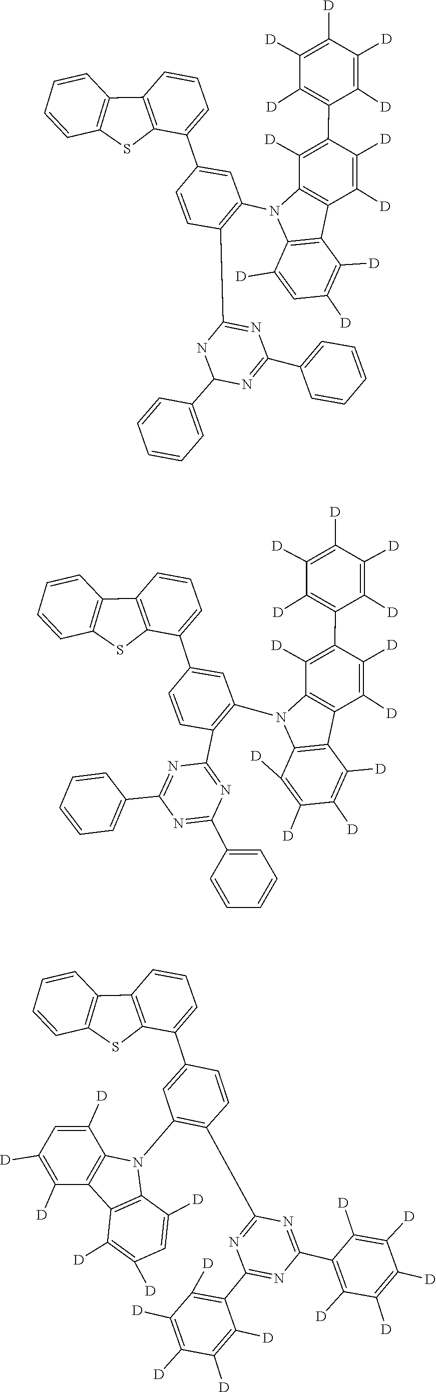

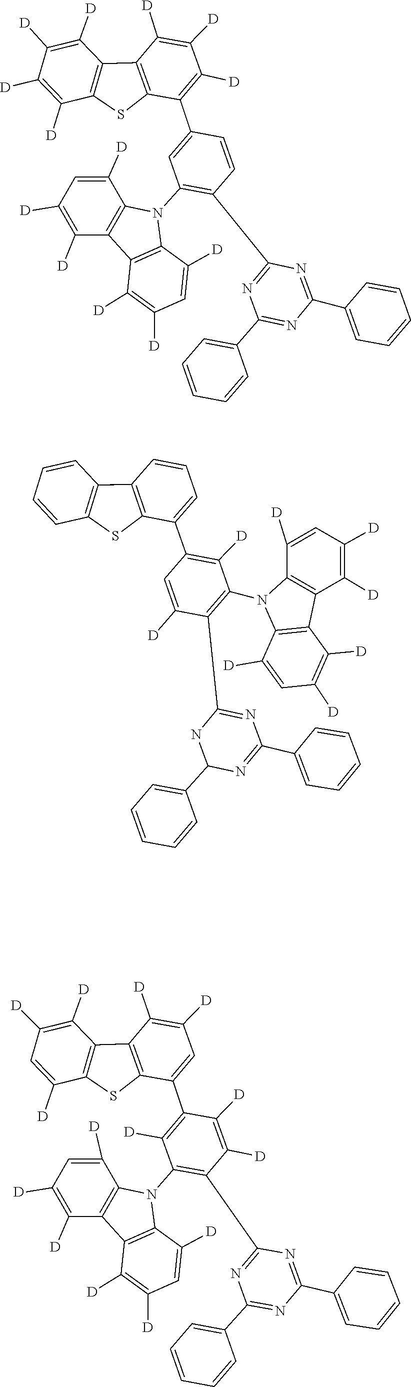

11. An organic light emitting device comprising:
a first electrode;
a second electrode that is opposite to the first electrode; and
an organic material layer that is between the first electrode and the second electrode, wherein the organic material layer comprises the compound of Chemical Formula 1 of claim 1.

12. The organic light emitting device according to claim 11,
wherein:
the organic material layer comprising the compound of Chemical Formula 1 is a light emitting layer.
